(19) United States
(12) Patent Application Publication
(10) Pub. No.: US 2026/0265712 A1
(43) Pub. Date: Sep. 10, 2026

(54) COMPOSITIONS AND METHODS FOR ENHANCING DRUG RESISTANCE IN CELLS

(71) Applicant: Medici Therapeutics, Inc., Boston, MA (US)

(72) Inventors: Nicholas P. RESTIFO, Chevy Chase, MD (US); David B. THOMPSON, Brookline, MA (US); Denitsa M. MILANOVA, Boston, MA (US); Matthew RABINOWITZ, Sunny Isles Beach, FL (US); Nadire COCHRAN, Wakefield, MA (US)

(21) Appl. No.: 19/685,382

(22) Filed: May 22, 2026

Related U.S. Application Data
(63) Continuation of application No. PCT/US24/57175, filed on Nov. 22, 2024.
(60) Provisional application No. 63/601,893, filed on Nov. 22, 2023.
(60) Provisional application No. 63/601,895, filed on Nov. 22, 2023.

(51) Int. Cl. C12N 9/12; A61P 35/00; C12N 9/04; C12N 9/10

(52) U.S. Cl. CPC C12N 9/1205; A61P 35/00; C12N 9/0006; C12N 9/1088; C12N 9/12; C12Y 101/01001; C12Y 205/01018; C12Y 207/01095; C12Y 207/11011

(57) ABSTRACT

Disclosed herein are compositions for engineering a human cell comprising a cell modulating agent or a vector encoding the cell modulating agent. Described herein are compositions comprising an engineered cell, wherein the engineered cell is an engineered human cell and comprises a modification such that the engineered cell is more resistant to an alkylating drug or a nucleoside analog relative to a non-engineered human cell. Various methods of treating a disease are provided, including a method of treating cancer in a subject by administering the cell modulating agents and/or engineered cells as a therapeutic.

### CROSS-REFERENCE TO RELATED APPLICATIONS

This application is a continuation of International Application No. PCT/US2024/057175, filed Nov. 22, 2024, which claims priority to, and the benefit of, U.S. Provisional Application No. 63/601,893, filed Nov. 22, 2023 and U.S. Provisional Application No. 63/601,895 filed Nov. 22, 2023, the contents of each of which is incorporated herein by reference in their entireties.

### SEQUENCE LISTING

The instant application contains a Sequence Listing which has been submitted electronically in XML format and is hereby incorporated by reference in its entirety. Said XML copy, created on Dec. 3, 2024, is named 227378-705601_SL.xml and is 110,957 bytes in size.

### BACKGROUND

Cell-based therapies and immunotherapies have undergone rapid clinical development for treatment of a variety of diseases and disorders. However, the therapeutic efficacy of cell-based therapies and immunotherapies can be affected by other treatment regimens which were previously administered or are concurrently administered to a patient. For example, patients treated with a cell-based immunotherapy may undergo a lymphodepletion preconditioning regimen, which can create a favorable in vivo environment for the cell-based therapeutic. Yet, the cell-based immunotherapy itself is also susceptible to elimination by lymphodepletion. Thus, there is a need to modify cells for enhanced drug resistance in both in vivo and ex vivo settings.

### A BRIEF SUMMARY

Provided herein are compositions for engineering a human cell, the composition comprising a cell modulating agent or a vector encoding the cell modulating agent, wherein the cell modulating agent modulates the activity or expression level of a target in the human cell by introducing a modification into the cell, thereby producing an engineered cell, wherein the engineered cell is more resistant to a drug relative to a corresponding non-engineered cell without the modification. In some embodiments, the engineered cell is more resistant to a cell depletion drug or a cell division inhibitor relative to a corresponding non-engineered cell without the modification. In some embodiments, the engineered cell is more resistant to an alkylating drug. In some embodiments, the engineered cell is more resistant to an alkylating drug relative to a corresponding non-engineered cell without the modification. In some embodiments, the alkylating drug is cyclophosphamide, ifosfamide, chlorambucil, altretamine, bendamustine, busulfan, carboquone, carmustine, chlormethine, chlorozotocin, dacarbazine, fotemustine, lomustine, melphalan, melphalan flufenamide, mitobronitol, nimustine, mitrosoureas, pipobroman, ranimustine, semustine, temozolomide, thiotepa, treosulfan, triaziquone, triethylenemelamine, trofosfamide, or uramustine. In some embodiments, the engineered cell is more resistant to cyclophosphamide relative to a corresponding non-engineered cell without the modification. In some embodiments, the engineered cell is more resistant to a nucleoside analogue or an antimetabolite relative to a corresponding non-engineered cell without the modification. In some embodiments, the nucleoside analogue or an antimetabolite is 5-aza-2′-deoxycytidine (decitabine), O₆-methylarabinofuranosyl guanine (nelarabine), 2′-fluoro-2′-deoxyarabinofuranosyl-2-chloroadenine (clofarabine), 5-aza-cytidine (vidaza), N4-pentyloxycarbonyl-5′-deoxy-5-fluorocytidine (capecitabine), 2,2-difluoro-2′-deoxycytidine (gemcitabine), 2-chloro-2′-deoxyadenosine (cladribine), arabinofuranosyl-2-fluoroadenine (fludarabine), 2′-deoxycoformycin (pentostatin), 5-fluoro-2′-deoxyuridine (floxuridine), arabinofura.nosylcytosine (cytarabine), 6-thioguanine, 5-fluorouracil, or 6-mercaptopurine. In some embodiments, the engineered cell is more resistant to fludarabine relative to a corresponding non-engineered cell without the modification.

Provided herein are compositions, wherein the compositions comprise: an engineered cell comprising one or more exogenous nucleic acids, wherein at least one exogenous nucleic acid that encodes for: a drug resistance protein selected from the group consisting of: an aminoglycoside phosphotransferase, a protein kinase cAMP-activated catalytic subunit alpha (PRKACA), an aldehyde dehydrogenase (ALDH), or a glutathione S-transferase, wherein the engineered cell is resistant to drug-mediated cell death relative to a comparable cell that does not have an exogenous nucleic acid encoding for the drug resistance protein.

Provided herein are compositions, wherein the compositions comprise: an engineered cell comprising one or more exogenous nucleic acids, wherein at least one exogenous nucleic acid comprises a sequence that is at least 80% identical, at least 85% identical, at least 90% identical, at least 95% identical, at least 96% identical, at least 97% identical, at least 98% identical, at least 99% identical, or is 100% identical to any one of SEQ ID NO: 1, SEQ ID NO: 2, SEQ ID NO: 8, SEQ ID NO: 9, SEQ ID NO: 15, SEQ ID NO: 16, SEQ ID NO: 19, SEQ ID NO: 20.

Provided herein are compositions, wherein the compositions comprise: an engineered cell comprising one or more exogenous nucleic acids, wherein at least one exogenous nucleic acid comprises a sequence that has complementarity to any one of SEQ ID NO: 27 or SEQ ID NO: 28.

Provided herein are compositions, wherein the compositions comprise: an engineered cell comprising one or more exogenous nucleic acids, wherein at least one exogenous nucleic acid encodes for an aminoglycoside-3′-phosphotransferase-IIa (APH(3′)-IIa) protein, wherein the engineered cell is resistant to aminoglycoside-mediated cell death relative to a comparable cell that does not have an exogenous nucleic acid encoding for an aminoglycoside-3′-phosphotransferase-IIa (APH(3′)-IIa) protein.

Provided herein are compositions, wherein the compositions comprise: an engineered cell comprising one or more exogenous nucleic acids, wherein at least one exogenous nucleic acid encodes for a protein kinase cAMP-activated catalytic subunit alpha (PRKACA) protein, wherein the engineered cell is resistant to aminoglycoside-mediated cell death relative to a comparable cell that does not have an exogenous nucleic acid encoding for protein kinase cAMP-activated catalytic subunit alpha (PRKACA) protein.

Provided herein are compositions, wherein the compositions comprise: an engineered cell comprising one or more exogenous nucleic acids, wherein at least one exogenous nucleic acid encodes for an aldehyde dehydrogenase (ALDH) protein, wherein the engineered cell is resistant to alkylating agent-mediated cell death relative to a comparable cell that does not have an exogenous nucleic acid encoding for an aldehyde dehydrogenase (ALDH) protein.

Provided herein are compositions, wherein the compositions comprise: an engineered cell comprising one or more exogenous nucleic acids, wherein at least one exogenous nucleic acid encodes for a glutathione S transferase A4 (GSTA4) protein, wherein the engineered cell is resistant to alkylating agent-mediated cell death relative to a comparable cell that does not have an exogenous nucleic acid encoding for glutathione S transferase A4 (GSTA4).

Provided herein are compositions, wherein the compositions comprise: a CD3 positive (CD3+) T cell comprising a genomic disruption in a target sequence of a deoxycytidine kinase (dCK) gene.

Provided herein are compositions, wherein the compositions comprise: a CD3 positive (CD3+) T cell comprising an siRNA-mediated disruption in a target sequence of a deoxycytidine kinase (dCK) RNA, wherein the CD3+ T cell has a reduced level of dCK RNA or dCK protein relative to a comparable CD3+ T cell that does not have an siRNA-mediated disruption.

Provided herein are compositions, wherein the compositions comprise: a carrier; and one or more nucleic acids, wherein at least one nucleic acid encodes for protein kinase cAMP-activated catalytic subunit alpha (PRKACA) protein. Provided herein are compositions, wherein the compositions comprise: a carrier; and one or more nucleic acids, wherein at least one nucleic acid comprises a sequence that is at least 80% identical to SEQ ID NO: 8. Provided herein are compositions, wherein the compositions comprise; and one or more nucleic acids, wherein at least one nucleic acid comprises a sequence that is at least 80% identical to SEQ ID NO: 9.

Provided herein are compositions, wherein the compositions comprise: a carrier; and one or more nucleic acids, wherein at least one nucleic acid encodes for an ALDH protein selected from the group consisting of: an aldehyde dehydrogenase 1 family member A1 protein, an aldehyde dehydrogenase 1 family member B1 protein, an aldehyde dehydrogenase 2 family member protein, and an aldehyde dehydrogenase 3 family member A1 protein. Provided herein are compositions, wherein the compositions comprise: a carrier; and one or more nucleic acids, wherein at least one nucleic acid comprises a sequence that is at least 80% identical to SEQ ID NO: 15. Provided herein are compositions, wherein the compositions comprise: a carrier; and one or more nucleic acids, wherein at least one nucleic acid comprises a sequence that is at least 80% identical to SEQ ID NO: 16.

Provided herein are compositions, wherein the compositions comprise: a carrier; and one or more nucleic acids, wherein at least one nucleic acid encodes for a GSTA4 protein. Provided herein are compositions, wherein the compositions comprise: a carrier; and one or more nucleic acids, wherein at least one nucleic acid comprises a sequence that is at least 80% identical to SEQ ID NO: 19. Provided herein are compositions, wherein the compositions comprise: a carrier; and one or more nucleic acids, wherein at least one nucleic acid comprises a sequence that is at least 80% identical to SEQ ID NO: 20.

Provided herein are compositions, wherein the compositions comprise: a carrier; and one or more nucleic acids, wherein at least one nucleic acid encodes for aminoglycoside-3′-phosphotransferase-IIa (APH(3′)-IIa) protein. Provided herein are compositions, wherein the compositions comprise: a carrier; and one or more nucleic acids, wherein at least one nucleic acid comprises a sequence that is at least 80% identical to SEQ ID NO: 1. Provided herein are compositions, wherein the compositions comprise: a carrier; and one or more nucleic acids, wherein at least one nucleic acid comprises a sequence that is at least 80% identical to SEQ ID NO: 2.

Provided herein are compositions, wherein the compositions comprise: a carrier; and a vector comprising: one or more nucleic acids, wherein at least one nucleic acid encodes for protein kinase cAMP-activated catalytic subunit alpha (PRKACA) protein, wherein the vector comprises a lentiviral vector, an adenoviral vector, an adeno-associated viral (AAV) vector, or a retroviral vector. Provided herein are compositions, wherein the compositions comprise: a carrier; and a vector comprising: a nucleic acid comprising a sequence is at least 80% identical to SEQ ID NO: 8, SEQ ID NO: 9, or SEQ ID NO: 24.

Provided herein are compositions, wherein the compositions comprise: a carrier; and a vector comprising: one or more nucleic acids, wherein at least one nucleic acid encodes for a GSTA4 protein, wherein the vector comprises a lentiviral vector, an adenoviral vector, an adeno-associated viral (AAV) vector, or a retroviral vector. Provided herein are compositions, wherein the compositions comprise: a carrier; and a vector comprising: a nucleic acid comprising a sequence is at least 80% identical to SEQ ID NO: 19, SEQ ID NO: 20, or SEQ ID NO: 26.

Provided herein are compositions, wherein the compositions comprise: a carrier; and a vector comprising: one or more nucleic acids, wherein at least one nucleic acid encodes for aminoglycoside-3′-phosphotransferase-IIa (APH(3′)-IIa) protein, wherein the vector comprises a lentiviral vector, an adenoviral vector, an adeno-associated viral (AAV) vector, or a retroviral vector. Provided herein are compositions, wherein the compositions comprise: a carrier; and a vector comprising: a nucleic acid comprising a sequence is at least 80% identical to SEQ ID NO: 1, SEQ ID NO: 2, or SEQ ID NO: 23.

Provided herein are compositions, wherein the compositions comprise: a carrier; and a vector comprising: one or more nucleic acids, wherein at least one nucleic acid encodes for an ALDH protein selected from the group consisting of: an aldehyde dehydrogenase 1 family member A1 protein, an aldehyde dehydrogenase 1 family member B1 protein, an aldehyde dehydrogenase 2 family member protein, and an aldehyde dehydrogenase 3 family member A1 protein, wherein the vector comprises a lentiviral vector, an adenoviral vector, an adeno-associated viral (AAV) vector, or a retroviral vector. The composition of any preceding embodiment, wherein the ALDH protein is an aldehyde dehydrogenase 3 family member A1 protein (ALDH3A1). Provided herein are compositions, wherein the compositions comprise: a carrier; and a vector comprising: a nucleic acid comprising a sequence is at least 80% identical to SEQ ID NO: 15, SEQ ID NO: 16, or SEQ ID NO: 25.

Provided herein are compositions, wherein the compositions comprise: a carrier; and a vector comprising: a guide nucleic acid that targets a sequence within a dCK gene or a dCK RNA. The composition of any preceding embodiment, wherein the guide nucleic acid has complementarity to the dCK gene or the dCK RNA.

Provided herein are vectors, wherein the vectors comprise: a nucleic acid that is at least 80% identical to any one of SEQ ID NO: 1, SEQ ID NO: 2, SEQ ID NO: 8, SEQ ID NO: 9, SEQ ID NO: 15, SEQ ID NO: 16, SEQ ID NO: 19, SEQ ID NO: 20, SEQ ID NO: 23, SEQ ID NO: 24, SEQ ID NO: 25, or SEQ ID NO: 26.

Provided herein are compositions, wherein the compositions comprise: an engineered cell, wherein the engineered cell is an engineered human cell and comprises a modification such that the engineered cell is more resistant to a nucleoside analogue or an antimetabolite relative to a corresponding non-engineered human cell without the modification, wherein the activity or expression level of a target in the engineered cell is modulated compared to the activity or expression level of the target in the non-engineered human cell.

Provided herein are compositions, wherein the compositions comprise: an engineered cell, wherein the engineered cell is an engineered immune cell and comprises a modification such that the engineered cell is more resistant to a cell depletion drug or a cell division inhibitor compared to a corresponding non-engineered immune cell without the modification.

Provided herein are compositions, wherein the compositions comprise: an engineered cell, wherein the engineered cell is an engineered human cell and comprises a modification such that the engineered cell is more resistant to an alkylating drug relative to a corresponding non-engineered human cell without the modification, wherein the activity or expression level of a target in the engineered cell is modulated compared to the activity or expression level of the target in the non-engineered human cell.

Provided herein are compositions, wherein the compositions comprise: an engineered cell, wherein the engineered cell is an engineered immune cell and comprises a modification such that the engineered cell is more resistant to a cell depletion drug or a cell division inhibitor compared to a corresponding non-engineered immune cell without the modification.

Provided herein are compositions for engineering a cell, the composition comprising a cell modulating agent or a vector encoding the cell modulating agent, wherein the cell modulating agent modulates the activity or expression level of a target in the cell by introducing a modification into the cell such that the engineered cell is more resistant to a drug relative to a corresponding non-engineered cell without the modification.

Provided herein are methods, wherein the methods comprise: engineering a cell provide herein, and wherein the methods comprise introducing a composition provided herein (e.g., a composition comprising a vector or a nucleic acid) into a cell.

Provided herein are pharmaceutical compositions, wherein the pharmaceutical compositions comprise: a composition provided herein, a vector provided herein, a nucleic acid provided herein, an engineered cell provided herein, an agent provided herein or any combination thereof; and a pharmaceutically acceptable carrier.

Provided herein are methods, wherein the methods comprise: treating a disease or condition in a subject in need thereof, wherein the methods comprise administering a therapeutically effective amount of a composition provided herein, a pharmaceutical composition provided herein, and/or an engineered cell provided herein to the subject, thereby treating the disease or condition.

Provided herein are methods, wherein the methods comprise: treating a cancer in a subject in need thereof, wherein the methods comprise: administering a therapeutically effective amount of a composition provided herein, a pharmaceutical composition provided herein, and/or an engineered cell provided herein to the subject, thereby treating the disease or condition.

Provided herein are methods of treating a cancer in a subject in need thereof, wherein the method comprises: administering to the subject in need thereof a therapeutically effective amount of a vector provided herein, thereby treating the disease or the condition.

Provided herein are methods of treating a cancer in a subject in need thereof, wherein the method comprises: ex vivo contacting leukocytes isolated from the subject with a vector or a composition provided herein to generate a population of genetically engineered leukocytes; and administering to the population of genetically engineered leukocytes to the subject, thereby treating the cancer.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIGS. 1A-1C show schematic diagrams of the lentiviral vectors used to modify CD3⁺ T Cells. FIG. 1A shows a vector map for APH(3′)-IIa (SEQ ID NO: 23) or PRKACA (SEQ ID NO: 24). FIG. 1B shows a vector map for ALDH3A1 (SEQ ID NO: 25). FIG. 1C shows a vector map for GSTA4 (SEQ ID NO: 26).

FIG. 2 is a plot a graph showing cell viability with increasing concentrations of fludarabine for wild-type (non-modified T cells), APH(3′)-IIa expressing T cells, and PRKACA-expressing T cells. X-axis: fludarabine concentration (micrograms/milliliter, μg/mL). Y-axis: relative luminescence (RLU) as determined by cell viability assay.

FIG. 3 is a bar graph showing the fold-change in cell viability of genetically engineered cells relative to wild-type cells following fludarabine challenge. X-axis: condition; Y-axis: fold change in viability.

FIG. 4 is a plot graph showing cell viability with increasing concentrations of cyclophosphamide. X-axis: cyclophosphamide concentration (micrograms/milliliter, μg/mL). Y-axis: % liv cells as determined by cell viability assay.

FIG. 5 is a bar graph showing the fold-change in cell viability of genetically engineered cells relative to wild-type cells following cyclophosphamide challenge. X-axis: condition; Y-axis: fold change in viability.

### DETAILED DESCRIPTION OF THE INVENTION

Immunotherapies, particularly cell-based therapies, have undergone rapid clinical development for treatment of a variety of diseases and disorders. However, the therapeutic efficacy of cell-based immunotherapies can be affected by other treatment regimens which were previously administered or are concurrently administered to a patient. For example, patients treated with a cell-based immunotherapy may undergo a lymphodepletion preconditioning regimen, which can create a favorable in vivo environment for the cell-based therapeutic. Lymphodepletion is a key component of cell-based immunotherapies, with the use of cyclophosphamide or fludarabine separately or in combination as clinically validated regimens. In the context of adoptive cell therapy, lymphodepletion refers to the intentional reduction or depletion of a patient's existing lymphocytes, which are a type of white blood cell, prior to the infusion of adoptively transferred cells. This process can create a more favorable environment for adoptively transferred cells to proliferate and function effectively within the patient's body. Lymphodepletion can provide several benefits to immunotherapeutic effector cells, including reducing resource competition, enhancing tumor targeting, increasing space within the lymphoid organs and tissues, activating antigen presenting cells, and improving persistence.

However, a challenge arises when the cell-based immunotherapy is susceptible to lymphodepletion, such as when the immunotherapy is a T-cell based immunotherapy. Yet, lymphodepletion can eliminate the cell-based immunotherapeutic itself Thus, there is a need to modify cells for enhanced drug resistance in both in vivo and ex vivo settings.

Provided herein are compositions for engineering a human cell, the composition comprising a cell modulating agent or a vector encoding the cell modulating agent, wherein the cell modulating agent modulates the activity or expression level of a target in the human cell by introducing a modification into the cell, thereby producing an engineered cell, wherein the engineered cell is more resistant to a drug relative to a corresponding non-engineered cell without the modification.

Provided herein are compositions comprising an engineered cell, wherein the engineered cell is an engineered human cell and comprises a modification such that the engineered cell is more resistant to an alkylating drug, a nucleoside analog, a derivative, or a metabolite thereof, relative to a corresponding non-engineered human cell without the modification, wherein the activity or expression level of a target in the engineered cell is modulated compared to the activity or expression level of the target in the non-engineered human cell.

Provided herein are compositions comprising an engineered cell, wherein the engineered cell is an engineered immune cell and comprises a modification such that the engineered cell is more resistant to a cell depletion drug or a cell division inhibitor compared to a corresponding non-engineered immune cell without the modification.

Briefly, provided herein are (1) compositions for genetically engineering cells and enhancing drug resistance; (2) genetically engineered cells; (3) alkylating agents and aminoglycosides; (4) combination compositions; (5) pharmaceutical compositions and formulations; (6) dosing and administration; (7) efficacy; (8) methods of treating a disease; and (9) kits, delivery systems, and dosing regimens.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### Definitions

The singular form “a”, “an”, and “the” include plural references unless the context clearly dictates otherwise. For example, the term “a cell” includes one or more cells, including mixtures thereof “A and/or B” is used herein to include all of the following alternatives: “A”, “B”, “A or B”, and “A and B.”

The terms “and/or” and “any combination thereof” and their grammatical equivalents as used herein, can be used interchangeably. These terms can convey that any combination is specifically contemplated. Solely for illustrative purposes, the following phrases “A, B, and/or C” or “A, B, C, or any combination thereof” can mean “A individually; B individually; C individually; A and B; Band C; A and C; and A, B, and C.”

The term “or” can be used conjunctively or disjunctively unless the context specifically refers to a disjunctive use.

The term “binding domain” or “binding moiety”, as used herein, refers to a protein, or polypeptide sequence, which specifically binds to a target.

As used herein, the term “CAR-T cells” means a T cell or population thereof, which has been modified through molecular biological methods to express a chimeric antigen receptor (CAR) on the T cell surface. The CAR is a polypeptide having a pre-defined binding specificity to a desired target which is operably connected to an intracellular portion of a T cell signaling domain. The specificity of a CAR engineered T cell can be redirected to a target molecule (e.g., a tumor-associated antigen) in a major histocompatibility complex (MHC)-independent manner. CARs can be engineered into both CD8⁺ and CD4⁺ T cells, among other T-cell subtypes. In some embodiments, CARs are a fusion of a ligand-binding domain (e.g., as a single-chain variable fragment (scFv) derived from a monoclonal antibody), a transmembrane domain, and an endodomain comprising a T-cell signaling domain (e.g., CD3-zeta (CD3s)). Such fusion molecules enable CAR engineered T cells to signal through a T-cell signaling pathway in response to a CAR-specific ligand binding event. In some instances, the T-cell signaling response is a zeta signal through CD3(CARs described herein encompass first-generation CARs, second-generation CARs, third-generation CARs, and any next-generation CAR (e.g., armored CARs).

As used herein, a “T cell receptor” or alternatively a TCR refers to a molecule found on the surface of T cells (or T lymphocytes) that is capable of recognizing antigens bound to major histocompatibility complex (MHC) molecules. A TCR can be found on the surface of a cell or in soluble fonn and may be comprised of a heterodimer having a and chains (also referred to as TCRa and TCR, respectively), or y and 6 chains (also referred to as TCRy and TCRcS, respectively). The extracellular portion of TCR chains (e.g., a-chain, -chain) contain two immunoglobulin domains, a variable domain (e.g., a-chain variable domain or Va, -chain variable domain or V13; amino acids 1 to 116 based on Kabat numbering (Kabat et al., “Sequences of Proteins of Immunological Interest, US Dept. Health and Human Services, Public Health Service National Institutes of Health, 1991, 5th ed.) at the N-terminus, and a constant domain (e.g., a-chain constant domain or Ca., amino acids 117 to 259 based on Kabat, -chain constant domain or C13, amino acids 117 to 295 based on Kabat) adjacent to the cell membrane. The variable domains contain complementary determining regions (CDRs) separated by framework regions (FRs) (see, e.g., Jores et al., Proc. Nat'l Acad Sci. USA. 87:9138, 1990; Chothia et al., EMBO J. 7:3745, 1988; see also Lefranc et al., Dev. Comp. Immunol. 27:55, 2003). The source of a TCR or TCR binding domain as used in the present disclosure may be from various animal species, such as a human, mouse, rat, rabbit or other mammal.

As used herein, a “synthetic receptor” refers to an engineered molecule or complex, where the engineered molecule or complex can bind to a target, that can be expressed at the cell surface. In some instances, a “synthetic receptor” may refer to a receptor that can trigger a cellular signaling response. In some instances, a “synthetic receptor” may be an endogenous receptor engineered such that the synthetic version of the receptor is signaling incompetent. In some instances, a “synthetic receptor” may comprise a set of engineered heterodimeric or multimeric receptor chains.

As used herein, “specifically binds” means that the binding domain preferentially binds the corresponding target over other proteins. In some instances, “specifically binds” means that the binding domains have a higher affinity for the target than for other proteins.

As used herein, a “therapeutically effective amount” of an agent is an amount sufficient to provide a therapeutic benefit in the treatment or management of a disease or disorder, or to delay or minimize one or more symptoms associated with the disease or disorder. A therapeutically effective amount of an agent means an amount of therapeutic agent, alone or in combination with other therapeutic agents, which provides a therapeutic benefit in the treatment or management of the disease. The term “therapeutically effective amount” can encompass an amount that improves overall therapy, reduces or avoids symptoms or causes of the disease or disorder, or enhances the therapeutic efficacy of another therapeutic agent. An example of an “effective amount” is an amount sufficient to contribute to the treatment, prevention, or reduction of a symptom or symptoms of a disease, which could also be referred to as a “therapeutically effective amount.” A “reduction” of a symptom means decreasing of the severity or frequency of the symptom(s), or elimination of the symptom(s). The exact amount of a composition including a “therapeutically effective amount” will depend on the purpose of the treatment, and will be ascertainable by one skilled in the art using known techniques.

As used herein, the term “treat,” “treating” or “treatment” of any disease or disorder refers, in one instance, to ameliorating the disease or disorder (i.e., slowing or arresting or reducing the development of the disease or at least one of the clinical symptoms thereof). In another instance, “treat”, “treating” or “treatment” refers to alleviating or ameliorating at least one physical parameter including those which may not be discernible by the patient. In yet another instance, “treat”, “treating” or “treatment” refers to modulating the disease or disorder, either physically, (e.g., stabilization of a discernible symptom), physiologically, (e.g., stabilization of a physical parameter), or both.

The term “complementary” or “complementarity” refers to the ability of a nucleic acid to form one or more bonds with a corresponding nucleic acid sequence by, for example, hydrogen bonding (e.g., traditional Watson-Crick), covalent bonding, or other similar methods. In Watson-Crick base pairing, a double hydrogen bond forms between nucleobases T and A, whereas a triple hydrogen bond forms between nucleobases C and G. For example, the sequence A-G-T can be complementary to the sequence T-C-A. A percent complementarity indicates the percentage of residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary, respectively). “Perfectly complementary” can mean that all the contiguous residues of a nucleic acid sequence will hydrogen bond with the same number of contiguous residues in a second nucleic acid sequence. “Substantially complementary” as used herein can refer to a degree of complementarity that can be at least 60%, 65%, 70%, 75%, 80%, 85%, 90%, 95%. 97%, 98%, 99%, or 100% over a region of 10, 15, 20, 25, 30, 35, 40, 45, 50, or more nucleotides, or can refer to two nucleic acids that hybridize under stringent conditions (i.e., stringent hybridization conditions). Nucleic acids can include nonspecific sequences. As used herein, the term “nonspecific sequence” or “not specific” can refer to a nucleic acid sequence that contains a series of residues that may not be designed to be complementary to or can be only partially complementary to any other nucleic acid sequence.

### (1) Compositions for Genetically Engineering Cells and Enhancing Drug Resistance

Provided herein are compositions for engineering a cell. In some embodiments, the compositions comprise a cell modulating agent or a vector encoding the cell modulating agent, wherein the cell modulating agent modulates the activity or expression level of a target in the human cell by introducing a modification into the cell, thereby producing an engineered cell, wherein the engineered cell is more resistant to a drug relative to a corresponding non-engineered cell without the modification. In some aspects, the present disclosure describes compositions for engineering a cell, the composition comprising a cell modulating agent or a vector encoding the cell modulating agent, wherein the cell modulating agent modulates the activity or expression level of a target in the cell by introducing a modification into the cell such that the engineered cell is more resistant to a drug relative to a corresponding non-engineered cell without the modification.

In some embodiments, the engineered cell is more resistant to a cell depletion drug or a cell division inhibitor relative to a corresponding non-engineered cell without the modification. In some embodiments, the engineered cell is more resistant to a nucleoside analogue or an antimetabolite relative to a corresponding non-engineered cell without the modification. In some embodiments, the nucleoside analogue or an antimetabolite is 5-aza-2′-deoxycytidine (decitabine), 06-methylarabinofuranosyl guanine (nelarabine), 2′-fluoro-2′-deoxyarabinofuranosyl-2-chloroadenine (clofarabine), 5-aza-cytidine (vidaza), N4-pentyloxycarbonyl-5′-deoxy-5-fluorocytidine(capeci tabine), 2,2-difluoro-2′-deoxy cytidine (gemcitabine), 2-chloro-2′-deoxyadenosine (cladribine), arabinofuranosyl-2-fluoroadenine (fludarabine), 2′-deoxycoformycin (pentostatin), 5-fluoro-2′-deoxyuridine (floxuridine), arabinofuranosylcytosine (cytarabine), 6-thioguanine, 5-fluorouracil, or 6-mercaptopurine. In some embodiments, the engineered cell is more resistant to fludarabine relative to a corresponding non-engineered cell without the modification.

In some embodiments, the engineered cell is more resistant to an alkylating drug relative to a corresponding non-engineered cell without the modification. In some embodiments, the alkylating drug is cyclophosphamide, ifosfamide, chlorambucil, altretamine, bendamustine, busulfan, carboquone, carmustine, chlormethine, chlorozotocin, dacarbazine, fotemustine, lomustine, melphalan, melphalan flufenamide, mitobronitol, nimustine, mitrosoureas, pipobroman, ranimustine, semustine, temozolomide, thiotepa, treosulfan, triaziquone, triethylenemelamine, trofosfamide, or uramustine. In some embodiments, the engineered cell is more resistant to cyclophosphamide relative to a corresponding non-engineered cell without the modification.

In some embodiments, the cell modulating agent reduces the activity or expression level of a target compared to the corresponding non-engineered cell without the modification. In some embodiments, the cell modulating agent increases the activity or expression level of the target compared to the corresponding non-engineered cell without the modification. In some embodiments, the cell modulating agent increases the activity or expression level of a first target compared to the corresponding non-engineered cell without the modification, and wherein the cell modulating agent reduces the activity or expression level of a second compared to the corresponding non-engineered cell without the modification.

In some embodiments, the target comprises an aminoglycoside resistance agent. In some embodiments, the aminoglycoside resistance agent is aminoglycoside-3′-phosphotransferase-Ila (APH(3′)-Ila), aminoglycoside resistance methyltransferase A (ArmA), 16S rRNA methylase A (RmtA), 16S rRNA methylase B (RmtB), 16S rRNA methylase C (RmtC), 16S rRNA methylase D (RmtD), 16S rRNA methylase D2 (RmtD2), 16S rRNA methylase E (Rmtf), 16S rRNA methylase F (RmtF), 16S rRNA methylase G (RmtG), 16S rRNA methylase H (RmtH), acetylating aminoglycoside-(6)-N-acetyltransferase (AAC[6′]-Ib-cr), aminoglycoside (3″) (9) adenylyltransferase (ANT(J″)-Ia), aminoglycoside N(3)-acetyltransferase (AAC(3)-Ila), aminoglycoside 3′-phosphotransferase (APH(3″)-Ib), or aminoglycoside nucleotidyltransferase (ANT(3″)-Ila). In some embodiments, the cell modulating agent increases the activity or expression level of the aminoglycoside resistance agent or APH(3′)-Ila.

In some embodiments, the target comprises protein kinase cAMP-activated catalytic subunit alpha (PRKACA). In some embodiments, the cell modulating agent increases the activity or expression level of PRKACA.

In some embodiments, the target comprises deoxycytidine kinase (dCK), thymidine kinase 1 (TK1), thymidine kinase 2 (TK2), UMP-CMP kinase (CMPK1), or adenosine kinase (ADK). In some embodiments, the target comprises dCK. In some embodiments, the cell modulating agent reduces the activity or expression level of dCK, TK1, TK2, CMPK1, or ADK. In some embodiments, the cell modulating agent reduces the activity or expression level of dCK.

### Cell Modulating Agents

In some embodiments, the cell modulating agent comprises an RNA molecule, a DNA molecule, a gene editing agent, a gene integration agent, a gene expression modulator, an epigenetic modulator, or a biomolecule activity modulator. In some embodiments, the RNA molecule comprises an mRNA, a siRNA, a miRNA, a gRNA, a crRNA, an snRNA, a lncRNA, or a shRNA.

In some embodiments, the gene editing agent comprises a CRISPR/Cas protein, a transcription activator-like effector nuclease (TALEN), a zinc finger (ZF) protein, a DNA polymerase protein, a reverse transcriptase, a retroviral protein, an RNA polymerase, a deaminase, or an RNA base editor protein. In some embodiments, the gene integration agent comprises a virus, an integrase, a transposase, a recombinase, or a homology-directed repair (HDR) system. In some embodiments, the gene expression modulator comprises a CRISPR/Cas protein, a CRISPRi enzyme, a transcription activator-like effector (TALE), a zinc finger (ZF), a transcription factor, an activator, a co-activator, a repressor, or an inhibitor. In some embodiments, the activator comprises Gal4, VP64, or p65. In some embodiments, the epigenetic modulator comprises an epigenetic reader, an epigenetic writer, or an epigenetic eraser. In some embodiments, the epigenetic writer comprises an acetyltransferase or a methyltransferase. In some embodiments, the epigenetic eraser comprises a deacetylase or a demethylase In some embodiments, the biomolecule activity modulator comprises a protease, a kinase, a phosphatase, an acetylase, a deacetylase, a methyltransferase, a demethylase, a ubiquitin-activating enzyme (E1), a ubiquitin-conjugating enzyme (E2), a ubiquitin ligase (E3), a deubiquitinating enzyme (DUB), a glycosyltransferase, a glycosidase, an inhibitor of the target, a co-factor of the target, or an activator of the target.

In some cases, the gene editing agent comprises a nucleic acid-binding moiety, a nucleic acid-modifying moiety, a fusion protein, or a nucleic acid. In some cases, the gene editing agent comprises a nucleic acid-binding domain, e.g., a DNA-binding protein domain or polypeptide or an RNA-binding domain or polypeptide e.g., an RNA-binding protein (RBP). In some embodiments, a nucleic acid-binding moiety is capable of binding a nucleic acid. A nucleic acid-binding domain can bind to a nucleic acid in a nonspecific or a site-specific manner.

In some cases, the nucleic acid-binding moiety binds to a nucleic acid in a site-specific manner. For example, a nucleic acid-binding moiety can comprise an aptamer binding domain that selectively binds to a specific target. In some cases, a nucleic acid-binding moiety recognizes a specific recognition sequence in the target nucleic acid. In some cases, a nucleic acid-binding moiety comprises an aptamer binding domain. In some cases, a nucleic acid binding moiety selectively binds to a sequence or a structural element in a nucleic acid molecule. In some cases, an RNA-binding domain selectively binds to a specific sequence motif in an RNA molecule. In some cases, a nucleic acid-binding moiety selectively binds to a structural element in a nucleic acid molecule. For example, a nucleic acid-binding domain can bind to a stem-loop in a nucleic acid molecule.

In some cases, a nucleic acid-binding moiety is or comprises a guidable polypeptide domain, a transcriptional regulatory domain, or a nucleic acid-modifying domain. A guidable polypeptide domain can be capable of binding to a polynucleotide (e.g., an RNA guide) that can direct the guidable polypeptide domain to a target site. In some cases, the polynucleotide that can direct the guidable polypeptide domain to a target site is a polynucleotide complex comprising a crRNA and a tracrRNA. In some cases, the polynucleotide that can direct the guidable polypeptide domain to a target site comprises crRNA and a tracrRNA. In some cases, In some cases, the guidable polypeptide domain forms a complex with the RNA guide and recognizes the target sequence through DNA-RNA base pairing. In some cases, a nucleic-acid binding moiety is or comprises a transcriptional regulatory domain. In other cases, a nucleic-binding moiety can help recruit a transcriptional repressor, inhibitor, or activator to a target site. In some cases, a nucleic acid-binding moiety is or comprises a nucleic acid-modifying moiety. In some cases, the present disclosure uses nucleic acid-binding moieties to recruit a nucleic acid-modifying moiety to a target site. In some cases, a nucleic-acid binding moiety comprises catalytic activity. In other cases, a nucleic acid-binding moiety is catalytically inactive. In some cases, a nucleic-acid binding moiety comprising catalytic activity is modified to have a reduced level of activity compared to its wild-type counterpart.

In some cases, the gene editing agent in the present disclosure comprises a nucleic acid modifying domain. A nucleic acid-modifying domain can comprise a polypeptide domain, a nucleic acid or a combination thereof (e.g., a ribonucleoprotein complex). A nucleic acid-modifying domain can be capable of modifying nucleic acid, such as cleaving double-stranded nucleic acid; nicking a single-stranded nucleic acid; introducing a mutation, deletion, or insertion in a nucleic acid; methylating or demethylating a nucleic acid, or altering the structure of DNA (e.g., changing chromatin structure through modifying histones). For example, a nucleic acid modifying domain can comprise a nuclease domain, a nickase domain, a deaminase domain, a polymerase, reverse transcriptase domain, a recombinase domain, a transposase domain, or an epigenetic modifying domain. A nuclease domain can be capable of cleaving phosphodiester bonds between nucleotides in nucleic acids. A nuclease domain can comprise an exonuclease (e.g., a nuclease capable of cleaving nucleic acids from the ends) or an endonuclease (e.g., a nuclease capable of cleaving nucleic acids in the middle). In some cases, a nucleic acid modifying effector or nucleic acid binding domain is a nickase, which can be capable of cleaving a single-strand in a double-stranded DNA. Nucleic acid modifying domains can be useful for gene editing, or for regulating, activating, or inhibiting gene expression.

In some cases, the gene editing agent in the present disclosure comprises a guidable polypeptide domain (e.g., a CRISPR-Cas protein domain). In some cases, a guidable polypeptide domain is capable of binding to a polynucleotide (e.g., an RNA guide) that directs it to a target site. In some cases, the guidable polypeptide domain forms a complex with the polynucleotide and recognizes the target sequence through DNA-RNA base pairing. In some cases, a guidable polypeptide domain is a CRISPR/CRISPR-associated (Cas) domain. A CRISPR domain can be a natural or an engineered domain. A Cas protein or domain can be derived from a CRISPR system or share structural and/or functional similarities to a protein involved in a CRISPR system. A CRISPR system is a system encoding DNA sequence arrays known as clustered regularly interspaced short palindromic repeats (CRISPRs), which can be found in microbial genomes or phage genomes. In some cases, CRISPR systems comprise genes encoding CRISPR-associated (Cas) proteins and/or small RNA guide molecules (e.g., crRNA or tracrRNA) that assemble with the CRISPR domain. In some cases, the CRISPR-Cas domain forms a complex with one or more RNA guide molecules to form an effector ribonucleoprotein complex. The effector ribonucleoprotein complex can recognize a target sequence through sequence specific DNA-RNA base pairing with a spacer sequence in the RNA guide. In some cases, target recognition activates one or more nuclease domains (e.g., a RuvC domain or HNH domain) in the CRISPR domain to make a double-stranded cut at the target DNA. A CRISPR-Cas domain complexed with an RNA guide can be capable of inactivating target gene through a gene knockout. In some cases, the CRISPR domain is used to enable gene insertion and/or deletion, which can inactivate, modify, or restore the gene's function.

A CRISPR system can comprise single subunit or multi-subunit effectors. In some cases, a CRISPR system is a Class 1 CRISPR system. A Class 1 CRISPR system can be a type I, type III, or a type IV system. A Class 1 type I CRISPR system can comprise a multi-subunit effector. In some cases, a Class 1 type I CRISPR system comprises a protein or domain in the Cascade-Cas3 protein complex. A Class 1 type I CRISPR system can comprise a Cas6, Cas7, Cass, Casl 1, Cas8, or Cas3 domain. A Class 1 type III CRISPR system can comprise a multi-subunit effector. In some cases, a Class 1 type III CRISPR system comprises a Csm complex or a Cmr complex. In some cases, a Class 1 type III CRISPR system comprises a Cas6, a Cas7 (Csm3 or Cmr4), a Cas7-related (CsmS, Cmr1, or Cmr6), a Cass (e.g., Csm4 or CmrS), a Casl 1 (e.g., Csm2 or Cmr3), or a CaslO (e.g., Csm1 or Cmr2) domain. A Class 1 type IV CRISPR system can comprise a Cas6, a Cas7, a Cass, a Casl 1, a Cas8 (e.g., Csfl), or a DinG or CysH domain.

In some cases, a CRISPR system is a Class 2 CRISPR system. A Class 2 CRISPR system can be a Class 2 type II CRISPR system, a Class 2 type V CRISPR system, or a Class 2 type VI CRISPR system. A Class 2 type II CRISPR system can comprise a Cas9 domain. A Cas9 domain can be a SpyCas9, a GeoCas9, a SauCas9, a KhuCas9, a AinCas9, an FmaCas9, a SgaCas9, a ScCas9, a SauriCas9 domain. A Cas9 domain can be a hyperactive Cas9 domain. A Class 2 type V CRISPR system can comprise a Cas12 domain. A Cas12 domain can be a Cas12a, a Cas12b, a Cas12c, a Cas12d, a Cas12e, a Cas12f, a Cas12g, a Cas12h, a Cas12i, a Cas12j, a Cas12k, a Cas121, or a Cas12m domain. A Class 2 type VI CRISPR system can comprise a Cas13 domain.

In some cases, a CRISPR-Cas domain comprises one or more subdomains. For example, a Cas9 domain can comprise a Rec1, a Rec2, a Rec3, a RuvC, an HNH, or a Wedge/PAM-interacting domain. A Cas12 domain can comprise a Reel, Rec2, a crRNA oligonucleotide binding domain (OBD), a Nuc domain, a PAM-interacting (PI) domain, or a RuvC domain. In some cases, the RuvC domain comprises nuclease activity. In some cases, the HNH domain comprises nuclease activity.

The PAM-interacting domain can bind to a protospacer adjacent motif (PAM) sequence that is next to a target sequence in a target nucleic acid molecule. PAM recognition can help activate a nuclease domain to make a cut at the target sequence. In some embodiments, the target sequence is at least 20 base pairs from a PAM sequence, at least 10 base pairs from a PAM sequence, or at least 5 base pairs from a PAM sequence.

In some cases, a CRISPR protein or domain is an engineered or mutated variant of a protein involved in a CRISPR system An engineered or mutated CRISPR domain can comprise a truncation, a deletion of a part of one or more domains or subdomains, or a mutation of an active site (e.g., a RuvC active site or HNH active site). In some cases, a CRISPR domain with a mutation of one or more active sites is catalytically inactive (e.g., dCas9). In some cases, a CRISPR domain with one or more mutated active sites comprises less than 90%, less than 80%, less than 70%, less than 60%, less than 50%, less than 40%, less than 30%, less than 20%, less than 10%, less than 5%, or less than 1% of the nuclease activity of its wildtype counterpart. For example, a dCas9 can result from the point mutations D10A in the RuvC domain and the point mutation H840A in the HNH domain. In other cases, a mutation can result in a CRISPR nickase. A nickase can generate nick or a single-stranded cut. A nickase can generate a nick in the strand complementary to the RNA guide (e.g., the targeting strand) or in the strand on the non-targeting strand. For example, a RuvC mutation D10A in a Cas9 domain can produce a Cas9 nickase domain that nicks the targeting strand. An HNH mutation H840A in a Cas9 domain can produce a Cas9 nickase domain that nicks the non-targeting strand. In some embodiments, the CRISPR system further comprises a DNA-dependent DNA polymerase or base editing enzyme.

In some embodiments, the methods provided herein comprise contacting a cell or a population of cells with a CRISPR interference (CRISPRi) system or a CRISPR activation (CRISPRa) system. In some embodiments, the CRISPRi system comprises a catalytically dead or deactivated endonuclease enzyme (e.g., a dead Cas9 or dCas9) fused to one or more repressor proteins (e.g., SALL1 or SDS3). In some embodiments, the CRISPRi system further comprises a guide RNA, wherein the guide RNAs binds to a target sequence of a polynucleotide that is upstream or downstream of a transcriptional start site (e.g., within 50 base pairs). The guide in a CRISPRi system can associate the enzyme-repressor protein fusion protein and direct the repressor domain of the protein to the target sequence in a polynucleotide, thereby inhibiting transcription of the polynucleotide. In some embodiments, the CRISPRa system comprises a catalytically dead or deactivated endonuclease enzyme (e.g., dCas9) fused to a transcriptional activator (e.g., Vp64). In some embodiments, the CRISPRa system comprises a guide RNA. In some embodiments, the guide RNA of the CRISPRa system binds to a target sequence in a polynucleotide, wherein the target sequence is upstream of a promoter or the transcriptional start site. The guide in a CRISPRa system can associate the enzyme-activator fusion protein domain of the fusion protein to the target activation sequence in a polynucleotide, thereby activating transcription of a target sequence.

In some cases, the gene editing agent in the present disclosure comprises a deaminase domain. The deaminase domain can be a natural or an engineered domain. A deaminase domain can be capable of carrying out deamination reactions in DNA. A deaminase domain can be capable of enabling the generation of base conversions or point mutations in a target nucleic acid. For example, a deaminase domain can be a cytidine deaminase domain or an adenosine deaminase domain. A cytidine deaminase domain can be capable of converting cytosine to uracil. A cytidine deaminase domain can be capable of enabling the conversion of a C-G base pair to a T-A base pair. For example, a cytidine deaminase can be or comprise a APOBEC 1 cytidine deaminase. An adenosine deaminase domain can be capable of converting an adenosine to hypoxanthine. An adenosine deaminase domain can be capable of converting an adenosine to an inosine. An adenosine deaminase can comprise TadA or a TadA mutant. An adenosine deaminase domain can be capable of enabling the conversion of an A-T base pair to a G-C base pair. A deaminase domain can be a mutated variant. In some cases, a deaminase domain enables the conversion of C to G, A to I, or C to U.

In some cases, the gene editing agent in the present disclosure comprises an RNA guide sequence and/or an adenosine deaminase that targets an RNA. In some embodiments, a guide RNA provided herein further comprises a small nuclear ribonucleic acid (snRNA) sequence. In some embodiments, a guide RNA facilitates RNA editing of one or more adenosines in a target RNA sequence (e.g., a dCK RNA sequence of SEQ ID NO: 28) by an adenosine Deaminase Acting on RNA or (ADAR).

In some cases, the cell modulating agent comprises a natural or an engineered transcriptional regulatory domain. A transcriptional regulatory domain can be capable of regulating, activating, or inhibiting gene expression and/or protein activity. For example, a transcriptional repressor can silence gene expression by binding to the promoter of a gene. A transcriptional activator can bind to enhancers or regulatory elements to activate expression of a gene. A transcriptional regulatory domain can comprise a transcription factor. A transcriptional regulatory domain can comprise a transcriptional activation domain or a transcriptional repression domain. For example, a transcriptional activation domain can be or comprise a CAP domain, a VP64 domain, a p65 domain, an Rta domain, a synergistic activation mediator (SAM) domain, a SunTag domain, a VPR domain, a DNA demethylase domain, a histone methyltransferase domain, a histone acetyltransferase domain, or a histone demethylase domain. A transcriptional repression domain can be or comprise a dCas9 domain, a KRAB domain, a Sin3 interacting domain (SID), or a MePC2 domain, a DNA methyltransferase domain, a histone deacetylase domain, a histone methyltransferase domain, or a histone demethylase domain. In some cases, a transcriptional regulatory domain comprises an epigenetic modifying effector domain. For example, an epigenetic modifying effector can be a DNA methyltransferase, a DNA demethylase, a histone methyltransferase, a histone demethylase, a histone acetyltransferase, or a histone deacetylase domain. A DNA methyltransferase domain can be capable of methylating a nucleic acid. A DNA demethylase domain can be capable of demethylating a nucleic acid. A histone methyltransferase domain can be capable of methylating a histone. A histone demethylase domain can be capable of demethylating a histone. A histone acetyltransferase domain can be capable of adding an acetyl group to a histone. A histone deacetylase domain can be capable of removing an acetyl group from a histone.

In some cases, the cell modulating agent comprises a zinc finger domain. A zinc finger domain can be a natural or an engineered domain. A zinc finger domain can bind to a specific DNA sequence in a target nucleic acid. A zinc finger domain can comprise from 1 to 10, from 2 to 10, from 3 to 10, from 4 to 10, from 5 to 10, from 6 to 10, from 7 to 10, from 8 to 10, from 9 to 10 zinc fingers, from 1 to 8, from 2 to 8, from 3 to 8, from 4 to 8, from 5 to 8, from 6 to 8, from 7 to 8, from 8 to 8, from 9 to 8 zinc fingers. In some cases, a zinc finger domain comprises a two-handed zinc finger domain. A two handed zinc finger domain can comprise two clusters of zinc finger domains that are separated by intervening amino acids. A two handed zinc finger domain can bind to two noncontiguous target DNA sequences. In some cases, the spacing between the two noncontiguous target sequences comprises from 1 to 15, from 1 to 12, from 1 to 10, from 1 to 8, or from 1 to 5 nucleotides. For example, a two handed type of zinc finger binding protein can be SIP1. A cluster of zinc finger domains in a two handed zinc finger domain can be capable of binding to a unique target nucleic acid sequence.

In some cases, the cell modulating agent comprises a TALE domain. A TALE domain can be a natural or an engineered domain. A TALE domain can bind to a specific DNA sequence. AT ALE domain can comprise one or more effector domains. AT ALE effector domain can comprise a central repeat domain comprising tandem repeats. A tandem repeat can comprise repeat variable residues (RVD). One or more RVDs can detect a specific DNA base. Different TALE effector domains may have a different number of repeats and a different order of their repeats. The C-terminal repeat is usually shorter in length (e.g., about 20 amino acids). Sequential repeats and their RVDs can recognize sequential DNA bases.

A TALE domain described herein can be derived from a TALE effector from a bacterial species. The TALE domain can be engineered to target a given nucleic acid sequence based on their DNA base specificities. The TALE domain can be engineered to remove or add a TALE effector domain. In some cases, the TALE domain corresponds to a perfect match to a nucleic acid target sequence. In some cases, the TALE domain of an epigenetic effector corresponds to one or more mismatches to a target base in the target nucleic acid.

In some embodiments, the cell modulating agent is encapsulated by a polymeric material. In some embodiments, the polymeric material is a nanocapsule. In some embodiments, the nanocapsule comprises synthetic polymers, wherein the synthetic polymer comprises poly(ethylene glycol) monomethyl ether (mPEG), poly-e-caprolactone (PCL), poly(lactide) (PLA), poly(lactide-co-glicolide) (PLGA), thiolated poly(methacrylic acid), and poly(N-vinyl Pyrrolidone). In some embodiments, the nanocapsule comprises a saccharide. In some embodiments, the nanocapsule comprises a lipid. In some embodiments, the nanocapsule is a liposome. In some embodiments, the nanocapsule is a lipid nanoparticle (LNP).

### Vectors

In some instances, the cell modulating agent is encoded by a vector. In some instances, the vector is an RNA molecule, a DNA molecule, or a viral vector. In some instances, the viral vector is a lentiviral vector, a retroviral vector, an adeno-associated viral (AAV) vector, or an adenoviral vector.

The term “viral vector” is widely used to refer either to a nucleic acid molecule that includes virus-derived nucleic acid elements that typically facilitate transfer of the nucleic acid molecule or integration into the genome of a cell, or to a viral particle that mediates nucleic acid transfer. Viral particles typically include viral components, and sometimes also host cell components, in addition to nucleic acid(s). Retroviral vectors used herein contain structural and functional genetic elements, or portions thereof, that are primarily derived from a retrovirus. Retroviral lentivirus vectors contain structural and functional genetic elements, or portions thereof including LTRs, that are primarily derived from a lentivirus (a sub-type of retrovirus).

Viral vectors that can be used in the disclosure include, for example, retrovirus vectors, lentivirus vectors, adenovirus vectors, and adeno-associated virus vectors, herpes virus, simian virus 40 (SV40), and bovine papilloma virus vectors (see, e.g., Gluzman (Ed.), Eukaryotic Viral Vectors, CSH Laboratory Press, Cold Spring Harbor, N.Y.). Exemplary viral vectors include, but are not limited to, lentiviral vectors, retroviral vectors, adeno-associated viral vectors (AAV), adenoviral vectors, herpes simplex viral vectors, alphaviral vectors, flaviviral vectors, rhabdoviral vectors, measles viral vectors, Newcastle disease viral vectors, poxviral vectors, picomaviral vectors, and oncolytic viral vectors.

In some instances, the cell modulating agent are delivered in the cell by viral or non-viral delivery vehicles known in the art. For example, the nucleic acid molecule can be stably integrated in the host genome, or can be episomally replicating, or present in the recombinant host cell as a mini-circle expression vector for stable or transient expression. Accordingly, in some instances, the nucleic acid molecule is maintained and replicated in the recombinant host cell as an episomal unit. In some instances, the nucleic acid molecule is stably integrated into the genome of the recombinant cell. Stable integration can also be accomplished using classical random genomic recombination techniques or with more precise genome editing techniques such as using guide RNA-directed CRISPR/Cas9, DNA-guided endonuclease genome editing NgAgo (Natronobacterium gregoryi Argonaute), or TALENs genome editing (transcription activator-like effector nucleases). In some instances, the nucleic acid molecule is present in the recombinant host cell as a mini circle expression vector for stable or transient expression.

In some embodiments, the viral vector is a lentiviral vector. In some embodiments, the lentiviral vector is selected from the group consisting of: a human immunodeficiency virus 1 (HIV-1); a human immunodeficiency virus 2 (HIV-2), a visna-maedi virus (VMV) virus; a caprine arthritis-encephalitis virus (CAEV); an equine infectious anemia virus (EIAV); a feline immunodeficiency virus (FIV); a bovine immune deficiency virus (BIV); and a simian immunodeficiency virus (SIV), fragments, derivatives, or variants thereof. Lentiviral systems are also useful for cell modulating agent delivery via viral transduction. Lentiviral vectors offer several attractive properties as gene-delivery vehicles, including: (i) sustained gene delivery through stable vector integration into the host cell genome; (ii) the ability to infect both dividing and non-dividing cells; (iii) broad tissue tropisms, including important gene- and cell-therapy-target cell types; (iv) no expression of viral proteins after vector transduction; (v) the ability to deliver complex genetic elements, such as polycistronic or intron-containing sequences; (vi) a potentially safer integration site profile (e.g., by targeting a site for integration that has little or no oncogenic potential); and (vii) a relatively easy system for vector manipulation and production.

In some embodiments, the viral vector comprises an AAV. AAVs can have one or more of the AAV wild-type genes deleted in whole or part, e.g., the rep and/or cap genes, but retain functional flanking ITR sequences. Functional ITR sequences are necessary for the rescue, replication, and packaging of the AAV virion. The ITRs need not be the wild-type nucleotide sequences, and may be altered, e.g., by the insertion, deletion or substitution of nucleotides, so long as the sequences provide for functional rescue, replication and packaging. A recombinant AAV vector (rAAV) comprises an infectious, replication-defective virus composed of an AAV protein shell encapsulating a heterologous nucleotide sequence of interest that is flanked on both sides by AAV ITRs. An rAAV vector is produced in a suitable host cell comprising an AAV vector, AAV helper functions, and accessory functions. In this manner, the host cell is rendered capable of encoding AAV polypeptides that are required for packaging the AAV vector (containing a recombinant nucleotide sequence of interest) into infectious recombinant virion particles for subsequent gene delivery. In some embodiments, the AAV or the rAAV provided herein comprises a serotype of AAV1, AAV2, AAV3, AAV4, AAV5, AAV6, AAV7, AAV8, AAV9, AAV10, AAV11, AAV12, AAV13, AAVrh10, or any combination thereof. In some embodiments, the delivery vehicle comprises a hybrid AAV-lipid nanoparticle delivery system.

Conventional viral and non-viral based gene transfer methods can be used to introduce polynucleotides encoding for a composition, system, guide polynucleotide, or an engineered protein provided herein to cells (e.g., mammalian cells) and target tissues. Exemplary non-viral vector delivery systems can include DNA plasmids, naked nucleic acid, and nucleic acids complexed with a delivery vehicle such as a liposome or poloxamer. Viral vector delivery systems can also include DNA and RNA viruses, which have either episomal or integrated genomes after delivery to the cell.

Methods of non-viral delivery of nucleic acids include electroporation, lipofection, nucleofection, gold nanoparticle delivery, microinjection, biolistics, virosomes, liposomes, immunoliposomes, polycation or lipid: nucleic acid conjugates, naked DNA, mRNA, artificial virions, and agent-enhanced uptake of DNA. Sonoporation using, e.g., the Sonitron 2000 system (Rich-Mar) can also be used for delivery of nucleic acids. Additional exemplary nucleic acid delivery systems include those provided by AMAXA® Biosystems (Cologne, Germany), Life Technologies (Frederick, Md.), MAXCYTE, Inc. (Rockville, Md.), BTX Molecular Delivery Systems (Holliston, Mass.) and Copernicus Therapeutics Inc. Lipofection reagents are sold commercially (e.g., TRANSFECTAM® and LIPOFECTIN®).

Delivery of the compositions and systems provided herein can be to cells (ex vivo administration) or target tissues (in vivo administration). Additional methods of delivery include the use of packaging the polynucleotides to be delivered into EnGeneIC delivery vehicles (EDVs). These EDVs are specifically delivered to target tissues using bispecific antibodies where one arm of the antibody has specificity for the target tissue and the other has specificity for the EDV. The antibody brings the EDVs to the target cell surface and then the EDV is brought into the cell by endocytosis.

### T Cell Marker Binding Moieties

In some embodiments, the compositions provided herein further comprise a cell marker binding moiety. In some embodiments, the cell marker is a T cell marker of prior tumor reactivity, a T cell lineage marker, a T cell differentiation marker, or an engineered cell marker.

In some embodiments, the T cell marker of prior tumor reactivity comprises PD-1, PD-LI, TIM1, LAG-3, TIGIT, CTLA4, CD39, CXCL13, CD25, CD69, 4-IBB, IL-2, IFN-γ, TNF, Fos, Jun, NFAT family proteins, NF-KB family proteins, Granzyme B, Granzyme A, Granzyme H, Granzyme K, Granzyme M, perforin, MHC class II components, HLA-DR, HLA-DP, HLA-DQ, OX40, OX40L, ICOS, RORC, T-bet, Blimp-I, GATAJ, FOXP3, BCL2, XIRPI, ILJ, CXCL8, MIR155HG, IFITI, XCL2, FLTI, IFI44L, SLC26A4, B3GNT5, PTGS2, ERRFII, MYOIB, CD200, INHBA, PROS I, IFIT3, PPP2R3A, MLF1, FEZI, NUDT4B, PLD1, TNFRSF9, MFSD2A, NR4A3, KCNH4, IL5, TLR4, DENNDSB, IFI44, RAPHI, IRF8, SMADI, PALM2-AKAP2, CD109, EPS8, SPPI, CRTAM, LAMP3, POLR3G, FAM1 14A1, XCL1, DOCK7, GNG4, ROBO1, SERPINE2, CLDN12, PFN2, ABTB2, ZNF704, CLIC4, NRCAM, SAMD4A, BCL2A1, EFNA1, TMTCI, B4GALT6, MOBJB, ARFGEF3, PLCBI, CHRNA5, ADAM23, NR4A2, SNX24, LMCDI, PLEKHGI, NIPAL4, ENCI, ARHGAP42, CTHRCI, CI5orf48, CEMIP, CDC42BPA, EXTL2, AASS, AFF3, ZC3H12C, GJCI, ST8SIAI, ENPPI, FABPS, SORBSI, SLC44A1, ZFYVE9, HERC5, NCSI, PON2, PTGFRN, EMPI, ARRDC4, PTPN13, KIAA0754, GNB4, TIAM2, TPMI, TEAD4, RAB38, or RGMB.

In some embodiments, the T cell lineage marker comprises CD3, CD4, or CD8. In some embodiments, the T cell differentiation marker comprises CCR7, CD45RA, CD45RO, CD62L, CD57, or CXCR3.

In some embodiments, the engineered cell marker comprises a tag, or an engineered receptor or a fragment thereof. In some embodiments, the engineered receptor comprises an scFv, an orthogonal polypeptide, an engineered T cell receptor, a chimeric antigen receptor, or fragments thereof.

In some embodiments, the cell marker binding moiety comprises a polypeptide. In some embodiments, the polypeptide comprises an antibody, an antibody-derived binding moiety, or an engineered binding moiety.

### Methods of Engineering a Cell

Provided herein are methods for engineering a cell comprising introducing a composition provided herein into a cell.

Provided herein are methods of modifying a target nucleic acid or generating an alteration in a target nucleic acid or a gene in a cell. In some embodiments, the methods comprise, administering to a cell, a tissue, or a subject a composition provided herein, or a cell provided herein wherein the administering generates an alteration in a target nucleic acid. In some embodiments, the number of alterations in the target nucleic acid is at least 1 alteration. In some embodiments, the percentage of target nucleic acid molecule alteration is at least 0.05%, at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 98%, or at least 99%. In some embodiments, the method of modifying the target nucleic acid comprises mutation of a target nucleic acid sequence. In some embodiments, the alteration or the modification of the target nucleic acid comprises: an insertion, a deletion, a substitution, a change in copy number, a point mutation, a frameshift mutation, a missense mutation, a nonsense mutation, a mutation in a stop codon, an epigenetic mark, or any combination thereof. In some embodiments, the alteration is made in the target nucleic acid to form an edited nucleic acid or to insert a transgene.

Vectors including viral and non-viral vectors containing nucleic acids encoding a nucleic editing systems, vectors, or nucleic acids provided herein can be administered directly to an organism for transduction of cells in vivo, delivered to cells isolated from a subject ex vivo, or administered to cells in vitro. Alternatively, naked DNA or RNA can be administered. Administration is by any of the routes normally used for introducing a molecule into ultimate contact with blood or tissue cells including, but not limited to, injection, infusion, topical application and electroporation. More than one route can be used to administer a particular composition.

In some embodiments, a composition, protein, guide polynucleotide, or a system provided herein can be shuttled to a cellular nucleus. For example, a vector can contain a nuclear localization sequence (NLS). A vector or any composition provided herein can also be shuttled by a protein or protein complex. In some embodiments, a composition or a system provided herein can be introduced to a cell or a target tissue by a minicircle vector.

In some embodiments, a vector or a polynucleotide provided herein can be pre-complexed with a system (e.g., a Cas9 enzyme) provided herein prior to electroporation into a cell. An engineered protein that can be used for shuttling can be a nickase or a catalytically dead Cas protein. A nuclease that can be used for shuttling can be a nuclease-competent protein. In some embodiments, an engineered protein herein can be pre-mixed with a guide polynucleotide provided herein an any additional elements (e.g., transgenes or other engineered proteins).

A system or a composition provided herein can also be introduced to a cell via electroporation techniques. The amount of polynucleotides that can be introduced into the cell by electroporation can be varied to optimize transfection efficiency and/or cell viability. In some embodiments, less than about 100 picograms of nucleic acid can be added to each cell sample (e.g., one or more cells being electroporated). In some embodiments, at least about 100 picograms, at least about 200 picograms, at least about 300 picograms, at least about 400 picograms, at least about 500 picograms, at least about 600 picograms, at least about 700 picograms, at least about 800 picograms, at least about 900 picograms, at least about 1 microgram, at least about 1.5 micrograms, at least about 2 micrograms, at least about 2.5 micrograms, at least about 3 micrograms, at least about 3.5 micrograms, at least about 4 micrograms, at least about 4.5 micrograms, at least about 5 micrograms, at least about 5.5 micrograms, at least about 6 micrograms, at least about 6.5 micrograms, at least about 7 micrograms, at least about 7.5 micrograms, at least about 8 micrograms, at least about 8.5 micrograms, at least about 9 micrograms, at least about 9.5 micrograms, at least about 10 micrograms, at least about 11 micrograms, at least about 12 micrograms, at least about 13 micrograms, at least about 14 micrograms, at least about 15 micrograms, at least about 20 micrograms, at least about 25 micrograms, at least about 30 micrograms, at least about 35 micrograms, at least about 40 micrograms, at least about 45 micrograms, or at least about 50 micrograms, of nucleic acid can be added to each cell sample (e.g., one or more cells being electroporated). For example, 1 microgram of dsDNA can be added to each cell sample for electroporation. In some embodiments, the amount of nucleic acid (e.g., dsDNA) required for optimal transfection efficiency and/or cell viability can be specific to the cell type. In some embodiments, the amount of nucleic acid (e.g., dsDNA) used for each sample can directly correspond to the transfection efficiency and/or cell viability. The transfection efficiency of cells with any of the nucleic acid delivery platforms described herein, for example, nucleofection or electroporation, can be or can be about 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, 99.5%, 99.9%, or more than 99.9%.

Viral particles, such as AAV, can be used to deliver a viral vector comprising a gene of interest or a transgene into a cell ex vivo or in vivo. In some embodiments, a mutated or chimeric adeno-associated viral vector as disclosed herein can be measured as pfu (plaque forming units). In some embodiments, the pfu of recombinant virus or mutated or chimeric adeno-associated viral vector of the compositions and methods of the disclosure can be about 10⁸ to about 5×10¹⁰ pfu. In some embodiments, recombinant viruses of this disclosure are at least about 1×10⁸, 2×10⁸, 3×10⁸, 4×10⁸f, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, and 5×10¹⁰ pfu. In some embodiments, recombinant viruses of this disclosure are at most about 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, and 5×10¹⁰ pfu. In some aspects, a mutated or chimeric adeno-associated viral vector of the disclosure can be measured as vector genomes. In some embodiments, recombinant viruses of this disclosure are 1×10¹⁰ to 3×10¹² vector genomes, or 1×10⁹ to 3×10¹³ vector genomes, or 1×10⁸ to 3×10¹⁴ vector genomes, or at least about 1×10, 1×10², 11×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, and 1×10¹⁸ vector genomes, or are 1×10⁸ to 3×10¹⁴ vector genomes, or are at most about 1×10, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, and 1×10¹⁸ vector genomes.

In some embodiments, a mutated or chimeric adeno-associated viral vector of the disclosure can be measured using multiplicity of infection (MOI). In some embodiments, MOI can refer to the ratio, or multiple of vector or viral genomes to the cells to which the nucleic can be delivered. In some embodiments, MOI can refer to the ratio, or multiple of vector or viral genomes to the cells to which the nucleic can be delivered. In some embodiments, the MOI can be 1×10⁶ GC/mL. In some embodiments, the MOI can be 1×10⁵ GC/mL to 1×10⁷ GC/mL. In some embodiments, the MOI can be 1×10⁴ GC/mL to 1×10⁸ GC/mL. In some embodiments, recombinant viruses of the disclosure are at least about 1×10¹ GC/mL, 1×10² GC/mL, 1×10³ GC/mL, 1×10⁴ GC/mL, 1×10⁵ GC/mL, 1×10⁶ GC/mL, 1×10⁷ GC/mL, 1×10⁸ GC/mL, 1×10⁹ GC/mL, 1×10¹⁰ GC/mL, 1×10¹¹ GC/mL, 1×10¹² GC/mL, 1×10¹³ GC/mL, 1×10¹⁴ GC/mL, 1×10¹⁵ GC/mL, 1×10¹⁶ GC/mL, 1×10¹⁷ GC/mL, and 1×10¹⁸ GC/mL MOI. In some embodiments, a mutated or chimeric adeno-associated viruses of this disclosure are from about 1×10⁸ GC/mL to about 3×10¹⁴ GC/mL MOI, or are at most about 1×10¹ GC/mL, 1×10² GC/mL, 1×10³ GC/mL, 1×10⁴ GC/mL, 1×10⁵ GC/mL, 1×10⁶ GC/mL, 1×10⁷ GC/mL, 1×10⁸ GC/mL, 1×10⁹ GC/mL, 1×10¹⁰ GC/mL, 1×10¹¹ GC/mL, 1×10¹² GC/mL, 1×10¹³ GC/mL, 1×10¹⁴ GC/mL, 1×10¹⁵ GC/mL, 1×10¹⁶ GC/mL, 1×10¹⁷ GC/mL, and 1×10¹⁸ GC/mL MOI.

In some aspects, a non-viral vector or nucleic acid can be delivered without the use of a mutated or chimeric adeno-associated viral vector and can be measured according to the quantity of nucleic acid. Generally, any suitable amount of nucleic acid can be used with the compositions and methods of this disclosure. In some embodiments, nucleic acid can be at least about 1 pg, 10 pg, 100 pg, 1 pg, 10 pg, 100 pg, 200 pg, 300 pg, 400 pg, 500 pg, 600 pg, 700 pg, 800 pg, 900 pg, 1 μg, 10 μg, 100 μg, 200 μg, 300 μg, 400 μg, 500 μg, 600 μg, 700 μg, 800 μg, 900 μg, 1 ng, 10 ng, 100 ng, 200 ng, 300 ng, 400 ng, 500 ng, 600 ng, 700 ng, 800 ng, 900 ng, 1 mg, 10 mg, 100 mg, 200 mg, 300 mg, 400 mg, 500 mg, 600 mg, 700 mg, 800 mg, 900 mg, 1 g, 2 g, 3 g, 4 g, or 5 g. In some embodiments, nucleic acid can be at most about 1 pg, 10 pg, 100 pg, 1 pg, 10 pg, 100 pg, 200 pg, 300 pg, 400 pg, 500 pg, 600 pg, 700 pg, 800 pg, 900 pg, 1 μg, 10 μg, 100 μg, 200 μg, 300 μg, 400 μg, 500 μg, 600 μg, 700 μg, 800 μg, 900 μg, 1 ng, 10 ng, 100 ng, 200 ng, 300 ng, 400 ng, 500 ng, 600 ng, 700 ng, 800 ng, 900 ng, 1 mg, 10 mg, 100 mg, 200 mg, 300 mg, 400 mg, 500 mg, 600 mg, 700 mg, 800 mg, 900 mg, 1 g, 2 g, 3 g, 4 g, or 5 g.

Nucleic acids, proteins, vectors, plasmids, compositions, systems, engineered proteins, and guide polynucleotides provided herein can be delivered by any suitable method, including transfection, electroporation, liposome delivery, membrane fusion techniques, high velocity DNA-coated pellets, viral infection and protoplast fusion.

In some embodiments, the methods comprise contacting a cell, a population of cells, or a mixed population of cells with a gene editing agent (also referred to herein as a gene editing system) or a nucleic acid encoding a gene editing agent provided herein. In some embodiments, the gene editing agent comprises: a Clustered Regularly Interspaced Short Palindromic Repeats (CRISPR)/Cas system, a catalytically inactive endonuclease, a Transcription Activator-Like Effector Nucleases (TALENS), a transposon systems (e.g., Sleeping Beauty), a zinc finger nuclease (ZFN), a meganuclease, a fusion protein, a derivative, a variant, or a mutant thereof. A database can identify regions of a target nucleic acid sequence that are permissive to genetic engineering depending on the method of editing. For example, a database can be ENCODE (encyclopedia of DNA Elements) (available on the world wide web at genome.gov/10005107), CHOP tool (available on the world wide web at chopchop.cbu.uib.no), GenomeCRISPR (available on the world wide web at genomecrispr.org), the Basic Local Alignment Search Tool (BLAST) (available on the world wide web at blast.ncbi.nlm.nih.gov/Blast.cgi), or the Sanger Institute Genome Editing (WGE) (available on the world wide web at wge.stemcell.sanger.ac.uk).

Targeted genetic perturbations (e.g, knock in, knock out, knock down) can begin from the generation of nuclease-induced double-stranded breaks (DSBs) or single stranded breaks, that lead to the stimulation of highly efficient recombination mechanisms of cellular DNA in mammalian cells. Double strand breaks are introduced into a cell using one or more endonucleases (e.g., S. pyogenes Cas9 deoxyribonucleic acid (DNA) endonuclease) and one or two nucleic acid guide sequences to affect a pair of double-strand breaks (DSBs). In some embodiments, the gene editing agent comprises one or more guide nucleic acid. In some embodiments, the one or more guide nucleic acid comprises an RNA, a DNA, or a combination thereof. In some embodiments, the one or more guide nucleic acid is a DNA-RNA hybrid. In some embodiments, the gene editing agent is a CRISPR system. CRISPR gene editing agents are discussed in further detail above.

In some embodiments, the guide nucleic acid for the gene editing agent is designed to have complementarity to a target DNA or RNA provided herein. In some embodiments, the guide nucleic acid has complementarity to, for example, any one of SEQ ID NO: 1, SEQ ID NO: 2, SEQ ID NO: 5, SEQ ID NO: 6, SEQ ID NO: 8, SEQ ID NO: 9, SEQ ID NO: 15, SEQ ID NO: 16, SEQ ID NO: 19, SEQ ID NO: 20, SEQ ID NO: 27 or SEQ ID NO: 28. In some embodiments, the guide nucleic acid has complementarity to a promoter sequence of a target provided herein (e.g., ALDH3A1, PRKACA, dCK, or GSTA4). The gene editing agent or system can be used to modulate target expression in a cell relative to a comparable cell that has not been contacted with a gene editing agent or a nucleic acid sequence provided herein.

In some embodiments, the method further comprises identifying the cell by the expression of a cell marker. In some embodiments, the cell marker is a T cell marker of prior tumor reactivity, a T cell lineage marker, a T cell differentiation marker, or an engineered cell marker.

In some embodiments, the T cell marker of prior tumor reactivity comprises PD-1, PD-L1, TIMJ, LAG-3, TIGIT, CTLA4, CD39, CXCL13, CD25, CD69, 4-1BB, IL-2, IFN-γ, TNF, Fos, Jun,

NFAT family proteins, NF-KB family proteins, Granzyme B, Granzyme A, Granzyme H, Granzyme K, Granzyme M, perform, WIC class II components, HLA-DR, HLA-DP, HLA-DQ, OX40, OX40L, ICOS, RORC, T-bet, Blimp-I, GATA3, FOXP3, BCL2, XIRP1, IL3, CXCL8, MIR155HG, IFIT1, XCL2, FLTI, IFI44L, SLC26A4, B3GNT5, PTGS2, ERRFll, MYOIB, CD200, INHBA, PROS I, IFITJ, PPP2R3A, MLF1, FEZI, NUDT4B, PLD1, TNFRSF9, MFSD2A, NR4A3, KCNH4, IL5, TLR4, DENNDSB, IF144, RAPHI, IRFS, SMADI, PALM2-AKAP2, CD109, EPSS, SPPI, CRTAM, LAMP3, POLRJG, FAM114A1, XCL1, DOCK7, GNG4, ROBO1, SERPINE2, CLDN12, PFN2, ABTB2, ZNF704, CLIC4, NRCAM, SAMD4A, BCL2A1, EFNA1, TMTC1, B4GALT6, MOBJB, ARFGEF3, PLCBI, CHRNA5, ADAM23, NR4A2, SNX24, LMCDI, PLEKHGI, NIPAL4, ENCI, ARHGAP42, CTHRC1, C15orf48, CEMIP, CDC42BPA, EXTL2, AASS, AFF3, ZC3H12C, GJC1, ST8SIA1, ENPPI, FABP5, SORBSI, SLC44A1, ZFYVE9, HERC5, NCSI, PON2, PTGFRN, EMPI, ARRDC4, PTPN13, KIAA0754, GNB4, TIAM2, TPM1, TEAD4, RAB38, or RGMB.

In some embodiments, the T cell lineage marker comprises CD3, CD4, or CD8. In some embodiments, the T cell differentiation marker comprises CCR7, CD45RA, CD45RO, CD62L, CD57, or CXCR3.

In some embodiments, the engineered cell marker comprises a tag. In some embodiments, the tag comprises an orthogonal polypeptide, or a synthetic receptor or fragment thereof In some embodiments, the cell is ex vivo. In some embodiments, the cell is in vivo. In some embodiments, the cell is in vitro. In some embodiments, the cell is a population of cells comprising at least about 1×10³ cells.

Simultaneously or sequentially, one or more modifications can be introduced into a cellular genome or transcriptome. The agents that introduce the modification (e.g., a guide RNA and CRISPR system; or an RNAi) can be controlled by a promoter. In the case of multiple modifications, the agents can be controlled under the same promoter or each agent can be controlled under different promoters. In some cases, the cell modification agents are on delivered to a population of cells via different nucleic acid strands, different plasmids, and/or different vectors.

### (2) Genetically Engineered Cells

Provided herein are compositions comprising an engineered cell, wherein the engineered cell is an engineered human cell and comprises a modification such that the engineered cell is more resistant to a nucleoside analogue or antimetabolite relative to a corresponding non-engineered human cell without the modification, wherein the activity or expression level of a target in the engineered cell is modulated compared to the activity or expression level of the target in the non-engineered human cell. In some aspects, the present disclosure describes compositions comprising an engineered cell, wherein the engineered cell comprises a modification such that the engineered cell is more resistant to a drug relative to a corresponding non-engineered cell without the modification, wherein the activity or expression level of a target in the engineered cell is modulated compared to the activity or expression level of the target in the non-engineered cell. In some embodiments, the drug is a cell depletion drug or a cell division inhibitor. In some aspects, the present disclosure describes compositions comprising an engineered cell, wherein the engineered cell comprises a modification such that the engineered cell is more resistant to a cell depletion drug or a cell division inhibitor compared to a corresponding non-engineered cell without the modification.

In some embodiments, the nucleoside analogue or the antimetabolite is 5-aza-2′-deoxycytidine (decitabine), 06-methylarabinofuranosyl guanine (nelarabine), 2′-fluoro-2′-deoxyarabinofuranosyl-2-chloroadenine (clofarabine), 5-aza-cytidine (vidaza), N4-pentyloxycarbonyl-5′-deoxy-5-fluorocytidine (capecitabine), 2,2-difluoro-2′-deoxycytidine (gemcitabine), 2-chloro-2′-deoxyadenosine (cladribine), arabinofuranosyl-2-fluoroadenine (fludarabine), 2′-deoxycoformycin (pentostatin), 5-fluoro-2′-deoxyuridine (floxuridine), arabinofuranosylcytosine (cytarabine), 6-thioguanine, 5-fluorouracil, or 6-mercaptopurine. In some embodiments, the nucleoside analogue or the antimetabolite is fludarabine.

In some embodiments, the activity or expression level of a target in the engineered cell is modulated compared to the activity or expression level of the target in the non-engineered cell. In some embodiments, the activity or expression level of a target in the engineered cell is increased compared to the activity or expression level of the target in the non-engineered cell. In some embodiments, the activity or expression level of a target in the engineered cell is reduced compared to the activity or expression level of the target in the non-engineered cell. In some embodiments, the activity or expression level of a first target in the engineered cell is increased compared to the activity or expression level of the first target in the non-engineered cell, and wherein the activity or expression level of a second target in the engineered cell is reduced compared to the activity or expression level of the second target in the non-engineered cell.

In some embodiments, the target comprises an aminoglycoside resistance agent. In some embodiments, the enzyme is an aldehyde dehydrogenase (ALDH). In some embodiments, the aminoglycoside resistance agent is aminoglycoside-3′-phosphotransferase-Ila (APH(3′)-Ila), aminoglycoside resistance methyltransferase A (ArmA), 16S rRNA methylase A (RmtA), 16S rRNA methylase B (RmtB), 16S rRNA methylase C (RmtC), 16S rRNA methylase D (RmtD), 16S rRNA methylase D2 (RmtD2), 16S rRNA methylase E (RmtE), 16S rRNA methylase F (RmtF), 16S rRNA methylase G (RmtG), 16S rRNA methylase H (RmtH), acetylating aminoglycoside-(6)-N-acetyltransferase (AAC[6′]-Ib-cr), aminoglycoside (3″) (9) adenylyltransferase (ANT(3″)-Ia), aminoglycoside N(3)-acetyltransferase (AAC(3)-Ila), aminoglycoside 3′-phosphotransferase (APH(3″)-Ib), or aminoglycoside nucleotidyltransferase (ANT(3″)-Ila). In some embodiments, the cell modulating agent increases the activity or expression level of the aminoglycoside resistance agent or APH(3′)-Ila.

In some embodiments, the target comprises protein kinase cAMP-activated catalytic subunit alpha (PRKACA). In some embodiments, the cell modulating agent increases the activity or expression level of PRKACA.

In some embodiments, the target comprises deoxycytidine kinase (dCK), thymidine kinase 1 (TK1), thymidine kinase 2 (TK2), UMP-CMP kinase (CMPK1), or adenosine kinase (ADK). In some embodiments, the target comprises dCK. In some embodiments, the cell modulating agent reduces the activity or expression level of dCK, TK1, TK2, CMPK1, or ADK. In some embodiments, the cell modulating agent reduces the activity or expression level of dCK.

In some embodiments, the cell depletion drug or the cell division inhibitor is a lymphodepletion agent. In some embodiments, the lymphodepletion agent is an anti-CD52 antibody or an anti-CD45 antibody. In some embodiments, the cell depletion drug or the cell division inhibitor is a cytotoxic drug, an alkylating drug, a nucleoside analog, a derivative, or a metabolite thereof.

In some embodiments, the activity or expression level of the target in the engineered cell is higher compared to the activity or expression level of the target in the non-engineered cell. In some embodiments, the activity or expression level of the target in the engineered cell is at least 1. I-fold, at least 1.5-fold, at least 2-fold, at least 3-fold, at least 4-fold, or at least 5-fold higher compared to the activity or expression level of the target in the non-engineered cell. In some embodiments, the activity or expression level of the target in the engineered cell is lower compared to the activity or expression level of the target in the non-engineered cell. In some embodiments, the activity or expression level of the target in the engineered cell is at least 1.1-fold, at least 1.5-fold, at least 2-fold, at least 3-fold, at least 4-fold, or at least 5-fold lower compared to the activity or expression level of the target in the non-engineered cell.

In some embodiments, the target is an endogenous target. In some embodiments, the target is a heterologous target. In some embodiments, the target is a gene, a nucleic acid molecule, an enzyme, a protein, a lipid, or a carbohydrate.

In some embodiments, the modification is a genetic modification, a post-transcriptional modification, a post-translational modification, an epigenetic modification, or a target activity modification.

In some embodiments, the genetic modification comprises a genetic insertion, a genetic deletion, a genetic substitution, a genetic translocation, a genetic inversion, homology-directed repair (HDR), non-homologous end joining (NHEJ), a genetic overexpression, or a genetic repression. In some embodiments, the engineered cell is more resistant to at least two cell depletion drugs or cell division inhibitors. In some embodiments, the modulated activity or expression level of the target phosphorylates the drug or a processed form of the drug, thereby inactivating the drug or the processed form of the drug. In some embodiments, the modulated activity or expression level of the target phosphorylates the drug or a processed form of the drug, thereby inhibiting binding of the drug or a processed form of the drug to a ribosome. In some embodiments, the modulated activity or expression level of the target inactivates the drug or a processed form of the drug. In some embodiments, the modulated activity or expression level of the target dephosphorylates a drug or a processed form of the drug, thereby inhibiting an enzymatic reaction involving the drug or a processed form of the drug. In some embodiments, the modulated activity or expression level of the target inhibits processing of the drug into an active form, thereby inhibiting drug incorporation into DNA replication, DNA synthesis, and DNA repair processes. In some embodiments, the modulated activity or expression level of the target inhibits cell cycle arrest, inhibition of cell division, and/or cell death.

Suitable cells that can contacted with a composition, a polynucleotide, an engineered protein, a guide polynucleotide, or a system provided herein include but are not limited to: epithelial cells, fibroblast cells, neural cells, keratinocytes, hematopoietic cells, melanocytes, chondrocytes, leukocytes, lymphocytes (B, NK, and T), macrophages, monocytes, mononuclear cells, cardiac muscle cells, other muscle cells, granulosa cells, cumulus cells, epidermal cells, endothelial cells, pancreatic islet cells, blood cells, blood precursor cells, bone cells, bone precursor cells, neuronal stem cells, primordial stem cells, hepatocytes, keratinocytes, umbilical vein endothelial cells, aortic endothelial cells, microvascular endothelial cells, fibroblasts, liver stellate cells, aortic smooth muscle cells, cardiac myocytes, neurons, Kupffer cells, smooth muscle cells, Schwann cells, and epithelial cells, erythrocytes, platelets, neutrophils, lymphocytes, monocytes, eosinophils, basophils, adipocytes, chondrocytes, pancreatic islet cells, thyroid cells, parathyroid cells, parotid cells, tumor cells, glial cells, astrocytes, red blood cells, white blood cells, macrophages, epithelial cells, somatic cells, pituitary cells, adrenal cells, hair cells, bladder cells, kidney cells, retinal cells, rod cells, cone cells, heart cells, pacemaker cells, spleen cells, antigen presenting cells, memory cells, T cells, B cells, plasma cells, muscle cells, ovarian cells, uterine cells, prostate cells, vaginal epithelial cells, sperm cells, testicular cells, germ cells, egg cells, Leydig cells, peritubular cells, Sertoli cells, lutein cells, cervical cells, endometrial cells, mammary cells, follicle cells, mucous cells, ciliated cells, nonkeratinized epithelial cells, keratinized epithelial cells, lung cells, goblet cells, columnar epithelial cells, dopaminergic cells, squamous epithelial cells, osteocytes, osteoblasts, osteoclasts, dopaminergic cells, embryonic stem cells, fibroblasts and fetal fibroblasts. Further, the one or more cells can be, for example, pancreatic islet cells and/or cell clusters or the like, including, but not limited to pancreatic a cells, pancreatic β cells, pancreatic δ cells, pancreatic F cells (e.g., PP cells), or pancreatic F cells.

Suitable cells also include stem cells such as, by way of example, embryonic stem cells, induced pluripotent stem cells, hematopoietic stem cells, neuronal stem cells and mesenchymal stem cells. Suitable cells can comprise any number of primary cells, such as human cells, non-human cells, and/or mouse cells. Suitable cells can be progenitor cells. Suitable cells can be derived from the subject to be treated (e.g., a subject with a disease, a subject in need of treatment, or a subject that is immunocompromised). Suitable cells can be derived from a human donor.

In some embodiments, the cell is genetically modified by the methods, systems, and compositions provided herein. Cells provided herein can be administered to a subject in need thereof, e.g., a subject with a disease or a condition in need of treatment.

In some embodiments, a cell provided herein is autologous. In some embodiments, the cell is allogeneic. In some embodiments, the cell is a stem cell. In some embodiments, the cell is an immune cell. In some embodiments, the immune cell is a tumor-infiltrating lymphocyte (TIL), a splenocyte, a T cell, a B cell, a natural killer (NK) cell, a monocyte, or a macrophage. In some embodiments, the T cell is a CAR-T cell, a TCR-T cell, a circulating T cell, a tissue resident T cell, a T cell from peripheral blood, or a T cell from the spleen.

In some embodiments, the immune cell comprises a synthetic receptor. In some embodiments, the synthetic receptor is a chimeric antigen receptor (CAR) or an engineered T-cell receptor (TCR). In some embodiments, the cell is not a cancer cell.

A method of attaining suitable cells, such as human primary cells, can comprise selecting cells. In some embodiments, a cell can comprise a marker that can be selected for the cell. For example, such marker can comprise GFP, a resistance gene (for example, a gene conferring antibiotic resistance), a cell surface marker, an endogenous tag. Cells can be selected using any endogenous marker. Suitable cells can be selected using any technology. Such technology can comprise flow cytometry and/or magnetic columns. The selected cells can also be expanded to large numbers.

In some instances, the cells can be genetically engineered (e.g., transduced, transformed, transfected, or nucleofected) with, for example, a viral vector. In some instances, the cell is an immune cell, a stem cell, a mammalian cell, a primate cell, or a human cell. Cells of the present disclosure may be autologous/autogeneic (“self’) or non-autologous (“non-self,” e.g., allogeneic, syngeneic or xenogeneic). “Autologous” as used herein, refers to cells derived from the same individual to which they are subsequently administered. “Allogeneic” as used herein refers to cells of the same species that differ genetically from the cell in comparison. “Syngeneic,” as used herein, refers to cells of a different individual that are genetically identical to the cell in comparison. In some instances, the recombinant cell is an immune system cell, e.g., a lymphocyte (for example without limitation, a T cell, natural killer cell or NK cell, natural killer T cell or NKT cell, a B cell, a plasma cell, tumor-infiltrating lymphocyte (TIL)), a monocyte or macrophage, or a dendritic cell. The immune cell can also be a precursor cell, i.e., a cell that is capable of differentiating into an immune cell. In some instances, the cell is a T cell. In some instances, the T cell is a CDS-positive T cell, a CD4-positive T cell, a regulatory T cell, a cytotoxic T cell, or a tumor infiltrating lymphocyte.

T cells can be obtained from a number of sources including, but not limited to, peripheral blood, peripheral blood mononuclear cells, bone marrow, lymph node tissue, cord blood, thymus tissue, tissue from a site of infection, ascites, pleural effusion, spleen tissue, and tumors. In some instances, T cells are obtained from a unit of blood collected from an individual using any number of known techniques such as sedimentation, e.g., FICOLL™ separation.

In some instances, an isolated or purified population of T cells is used. In some instances, cytotoxic lymphocytes (TCTL) and/or helper T cells (TH) are purified from peripheral blood mononuclear cells (PBMCs). In some instances, the T cells are sorted into naive (TN), memory (TMEM), stem cell memory (TSCM), central memory (TCM), effector memory (TEM), and effector (TEFF) T cell subpopulations either before or after activation, expansion, and/or genetic modification. In some instances, TCTL and TH lymphocytes are sorted into TN, TMEM, TSCM, TCM, TEM, and TEFF T cell subpopulations either before or after activation, expansion, and/or genetic modification. Suitable approaches for such sorting are known and include, e.g., magnetic-activated cell sorting (MACS), where m some instances TN are CD45RA+/CD62L+/CD95−; TSCM are CD45RA+/CD62L+/CD95+; TCM are CD45RO+/CD62L+/CD95+ and TEM are CD45RO+/CD62L−/CD95+. An exemplary approach for such sorting is described in Wang et al. (2016) Blood 127(24):2980-90.

In some instances, the cell is a population of cells. In some instances, the population of cells is a population of blood cells. The blood cells can be lymphocytes. The lymphocytes can be T cells. In some instances, the T cells are CDS-positive T cells, CD4-positive T cells, regulatory T cells (Tregs), cytotoxic T cells (CTLs), tumor infiltrating lymphocytes (TILs), or tumor reactive T lymphocytes. In some instances, the population of cells is a homogeneous mixture of cells of the same cell type. In some instances, the population of cells is a heterogeneous mixture of cells of different cell types. In some instances, the population of cells comprises at least about 1×10³ cells. In some instances, the population of cells comprises at least about 1×10⁴ cells. In some instances, the population of cells comprises at least about 1×10⁵ cells. In some instances, the population of cells comprises at least about 1×10⁶ cells. In some instances, the population of cells comprises at least about 1×10⁷ cells. In some instances, the population of cells comprises at least about 1×10⁸ cells. In some instances, the population of cells comprises at least about 1×10⁹ cells. In some embodiments, the population of cells comprises from about 1×10³ cells to about 1×10⁹ cells. In some embodiments, the population of cells comprises from about 1×10³ cells to about 1×10⁸ cells In some embodiments, the population of cells comprises from about 1×10³ cells to about 1×10⁷ cells. In some embodiments, the population of cells comprises from about 1×10³ cells to about 1×10⁶ cells. In some embodiments, the population of cells comprises from about 1×10³ cells to about 1×10⁵ cells. In some embodiments, the population of cells comprises from about 1×10³ cells to about 1×10⁴ cells. In some embodiments, the population of cells comprises from about 1×10⁴ cells to about 1×10⁹ cells. In some embodiments, the population of cells comprises from about 1×10⁴ cells to about 1×10⁸ cells. In some embodiments, the population of cells comprises from about 1×10⁴ cells to about 1×10⁷ cells. In some embodiments, the population of cells comprises from about 1×10⁴ cells to about 1×10⁶ cells. In some embodiments, the population of cells comprises from about 1×10⁴ cells to about 1×10⁵ cells. In some instances, the population of cells comprises from about 1×10⁵ cells to about 1×10⁹ cells. In some instances, the population of cells comprises from about 1×10⁵ cells to about 1×10⁸ cells. In some instances, the population of cells comprises from about 1×10⁵ cells to about 1×10⁷ cells. In some instances, the population of cells comprises from about 1×10⁵ cells to about 1×10⁶ cells.

### (3) Alkylating Agents and Aminoglycosides

In the embodiments described herein, alkylating agents are molecules that can keep the cell from replicating by damaging cellular DNA. Alkylating agents can work in all phases of the cell cycle and can be used to treat a variety of diseases and disorders, such as cancer. Cyclophosphamide is a chemotherapy drug that belongs to the class of alkylating agents. Other alkylating agents may include, but are not limited to, ifosfamide and chlorambucil, which can share similar mechanisms of action. Cyclophosphamide can exert its therapeutic effects through a complex mechanism involving both prodrug activation and direct cytotoxicity. A mechanism by which cyclophosphamide depletes lymphocytes and affects other rapidly dividing cells can be described as follows. Cyclophosphamide itself can be considered an inactive prodrug. Cyclophosphamide may require enzymatic conversion in the liver to its active form, 4-hydroxycyclophosphamide, by the hepatic cytochrome P450 system (e.g., the enzyme CYP2B6).

The 4-hydroxycyclophosphamide metabolite can spontaneously break down to generate two active metabolites: phosphoramide mustard and acrolein. Phosphoramide mustard can be an important cytotoxic agent that can enable DNA alkylation. Phosphoramide can enter rapidly dividing cells, including lymphocytes and cancer cells, and can form covalent bonds with DNA This alkylation of DNA can interfere with DNA replication and transcription processes. Phosphoramide mustard can cause cross-linking between DNA strands, resulting in DNA damage. This can lead to cell cycle arrest, inhibition of cell division, and ultimately cell death.

Phosphoramide mustard can undergo detoxification through complex cellular processes, such as through conjugation with glutathione (which comprises glutamate, cysteine, and glycine). In a process catalyzed by the enzyme glutathione S-transferase (GST), such as GSTA4, phosphoramide mustard can be covalently bound to glutathione. However, lymphocytes may not express GSTA4.

Acrolein can be a highly reactive and potentially toxic compound. Acrolein can be detoxified by aldehyde dehydrogenase (including ALDH3A1), which can convert acrolein into less toxic compounds that can be further metabolized and excreted. However, lymphocytes may not express ALDH. Taken together, the expression of ALDH and GST may make lymphocytes more resistant to the effects of alkylating agents, such as cyclophosphamide. In the embodiments described herein, antimetabolites and nucleoside analogues are molecules that can interfere with DNA and RNA by acting as a non-functional mimic of polynucleotide precursors. When an antimetabolite or nucleoside analogue is incorporated into DNA, DNA replication and transcription processes.

As described herein, fludarabine can be considered to be an antimetabolite or nucleoside analogue. Fludarabine is a chemotherapy medication that can be used in the treatment of various cancers, including hematological malignancies such as chronic lymphocytic leukemia (CLL), acute lymphoblastic leukemia (ALL), and non-Hodgkin lymphoma. Fludarabine can be used as part of the lymphodepletion regimen prior to adoptive cell transfer.

Fludarabine can interfere with DNA synthesis and repair within rapidly dividing cells. Fludarabine can be administered as a prodrug, where fludarabine can be metabolically activated within the body to its active form, 2-fluoro-ara-adenine triphosphate (F-ara-ATP). Once active, F-ara-ATP can be incorporated into DNA during replication, disrupting the DNA structure and inhibiting further DNA synthesis. This disruption can lead to cell death.

In some embodiments, fludarabine can be used in combination with other chemotherapy drugs or as part of a treatment regimen for specific cancer types. In some embodiments, when fludarabine is used as part of a lymphodepleting regimen, fludarabine can be used in combination with cyclophosphamide. Lymphodepleting regimens can cause transient immunosuppression, myelosuppression (reduction in blood cell counts), and increased susceptibility to infections. Thus, patients receiving this combination may need to be closely monitored. In some embodiments, fludarabine used as part of a lymphodepletion regimen can induce immunosuppression, which can create a more favorable environment for adoptively transferred cells, as there may be reduced competition and interference from the patient's own immune cells.

In some embodiments, fludarabine can reduce the number and activity of regulatory T cells (Tregs), which are a subset of T cells that suppress immune responses. By lowering the levels of Tregs, fludarabine can help mitigate immune tolerance and can enhance the anti-tumor activity of adoptively transferred cells. In some embodiments, fludarabine can be toxic for a subset of suppressive myeloid cells, called myeloid derived suppressor cells (MDSC). Tregs and MDSCs can be directly suppressive to adoptively transferred cells. In addition, the transient decline of these Tregs and MDSCs can result in the reduction of inhibitory factors that they produce in the tumor microenvironment (TME), which can allow the adoptively transferred cells to function more effectively in the presence of fewer inhibitory signals.

In some embodiments, patients treated with fludarabine can experience improved T cell expansion because of a potential increase the availability of cytokines and growth factors that support the expansion and survival of adoptively transferred cells. This can lead to a more robust and prolonged immune response against cancer cells. This can be in part due to reduced competition for homeostatic cytokines. In some embodiments, the immunosuppression can be accompanied by commensal microflora which can directly activate T cells, as well as antigen presenting cells, which can in turn activate T cells. In some embodiments, lymphodepletion can alter the chemokine environment systemically and in the tumor micro-environment (TME).

### Aminoglycoside and Alkylating Drug Resistance Agents and Engineered Cells

Provided herein are agents that enhance drug resistance in a cell. The cells can be in vivo engineered, ex vivo engineered, or in vitro engineered to have increased cell survival relative to a cell that has not been engineered according to the methods provided herein. Non-limiting examples of cell drug resistance can include the modulation in the levels (e.g., gene expression) or activity of APH(3′IIa, PRKACA, ALDH3A1, dCK, and GSTA4 in a cell. These cell resistance targets are discussed in further detail below.

### Aminoglycoside-3′-Phosphotransferase-Ila (APH(3′)-Ila)

Certain enzymes, including but not limited to aminoglycoside-3′-phosphotransferase-Ila (APH(3′)-Ila), may confer resistance to fludarabine. Fludarabine can be rapidly fully converted to the circulating metabolite F-ara-A, which can be distributed intracellularly. Intracellular phosphorylation can take place to the active metabolite fludarabine triphosphate (F-ara-ATP).

APH(3′)-IIa is an enzyme that can confer resistance to certain aminoglycoside antibiotics in bacteria. APH(3′)-IIa can phosphorylate the 3′ position of aminoglycoside antibiotics, which can render the antibiotics inactive and unable to bind to ribosomes. APH(3′)-IIa can also convert the active form of fludarabine- the tri-phosphorylated F-ara-ATP—into a monophosphate form which can no longer participate in enzymatic reactions which tri-phosphorylated F-ara-ATP can poison. Thus, expression of enzymes, including but not limited to APH(3′)-IIa, may confer cell resistance to fludarabine.

Provided herein are compositions comprising: an engineered cell comprising one or more exogenous nucleic acids, wherein at least one exogenous nucleic acid encodes for an aminoglycoside-3′-phosphotransferase-IIa (APH(3′)-IIa) protein. In some embodiments, the engineered cell is resistant to aminoglycoside-mediated cell death relative to a comparable cell that does not have an exogenous nucleic acid encoding for an aminoglycoside-3′-phosphotransferase-IIa (APH(3′)-IIa) protein. In some embodiments, the engineered cell is a human leukocyte, a human stem cell, or an in vitro-differentiated cell. In some embodiments, the engineered cell expresses at least a 10% increase, at least a 20% increase, at least a 30% increase, at least a 40% increase, at least a 50% increase, at least a 60% increase, at least a 70% increase, or at least an 80% increase in the level of aminoglycoside-3′-phosphotransferase-IIa (APH(3′)-IIa protein relative to a comparable cell that does not have an exogenous nucleic acid encoding for aminoglycoside-3′-phosphotransferase-IIa (APH(3′)-IIa) protein. In some embodiments, the engineered cell is an ex vivo-engineered cell, an in vivo-engineered cell, or an in vitro-engineered cell. In some embodiments, the human leukocyte is a human T cell, a human natural killer (NK) cell, a human natural killer T cell (NKT), a human macrophage, a human B cell, a human dendritic cell, or a human tumor infiltrating lymphocyte (TIL). In some embodiments, the human T cell is a CD3 positive (CD3⁺) T cell. In some embodiments, the human TIL is a CD3 positive (CD3⁺) TIL. In some embodiments, the human stem cell comprises an adult stem cell. In some embodiments, the human stem cell comprises an induced pluripotent stem cell (iPSC). In some embodiments, the human stem cell comprises an embryonic stem cell. In some embodiments, the in vitro-differentiated cell comprises an iPSC-derived natural killer T cell (iNKT), an iPSC-derived natural killer cell, an iPSC-derived T cell, an iPSC-derived macrophage, or an iPSC-derived B cell. In some embodiments, the one or more exogenous nucleic acids comprise a DNA, an RNA, or a combination thereof. In some embodiments, the RNA comprises a messenger RNA (mRNA), a guide RNA (gRNA), a microRNA (miRNA), a tRNA, a CRISPR RNA (crRNA), a short hairpin RNA (shRNA), a silencing RNA (siRNA), an antisense oligonucleotide, a non-coding RNA, an aptamer, or a combination thereof. In some embodiments, the DNA comprises a transgene encoding a protein, a regulatory element, a promoter, an enhancer, a repressor, an inhibitor, an antibiotic resistance gene, or a non-coding DNA. In some embodiments, the one or more nucleic acids further comprise a poly-A tail or a nucleoside modification. In some embodiments, the exogenous nucleic acid comprises a sequence that is at least 80% identical, at least 85% identical, at least 90% identical, at least 95% identical, at least 96% identical, at least 97% identical, at least 98% identical, at least 99% identical, or 100% identical to SEQ ID NO: 1. In some embodiments, the exogenous nucleic acid comprises a sequence that is at least 80% identical, at least 85% identical, at least 90% identical, at least 95% identical, at least 96% identical, at least 97% identical, at least 98% identical, at least 99% identical, or 100% identical to SEQ ID NO: 2. In some embodiments, the exogenous nucleic acid encodes for an amino acid sequence that is at least 80% identical, at least 85% identical, at least 90% identical, at least 95% identical, at least 96% identical, at least 97% identical, at least 98% identical, at least 99% identical, or 100% identical to SEQ ID NO: 3-SEQ ID NO: 7 (see SEQUENCES section).

Protein Kinase cAMP-Activated Catalytic Subunit Alpha

Protein kinase cAMP-activated catalytic subunit alpha (PRKACA) is a human protein that belongs to the family of protein kinases PRKACA is an enzyme that may be involved in cellular signaling pathways and may be responsible for phosphorylating target proteins in response to the presence of the signaling molecule cyclic AMP (cAMP). PRKACA can play a crucial role in many cellular processes, including gene expression, metabolism, and cell proliferation. PRKACA may confer resistance to fludarabine and fludarabine-derived metabolites in a similar manner as APH(3′)-Ila.

PRKACA may be expressed at low levels in T cells and may not appear to be dynamically regulated in T cells. Thus, enhanced expression and/or kinase activity of PRKACA may confer cell resistance to fludarabine.

Provided herein are compositions comprising: an engineered cell comprising one or more exogenous nucleic acids, wherein at least one exogenous nucleic acid encodes for a protein kinase cAMP-activated catalytic subunit alpha (PRKACA) protein. In some embodiments, the engineered cell is resistant to aminoglycoside-mediated cell death relative to a comparable cell that does not have an exogenous nucleic acid encoding for protein kinase cAMP-activated catalytic subunit alpha (PRKACA) protein. In some embodiments, the engineered cell is a human leukocyte, a human stem cell, or an in vitro-differentiated cell. In some embodiments, the engineered cell expresses at least a 10% increase in the level of PRKACA protein relative to a comparable cell that does not have an exogenous nucleic acid encoding for a PRKACA protein. In some embodiments, the engineered cell is an ex vivo-engineered cell, an in vivo-engineered cell, or an in vitro-engineered cell. In some embodiments, the human leukocyte is a human T cell, a human natural killer (NK) cell, a human natural killer T cell (NKT), a human macrophage, a human B cell, a human dendritic cell, or a human tumor infiltrating lymphocyte (TIL). In some embodiments, the human T cell is a CD3 positive (CD3+) T cell. In some embodiments, the human TIL is a CD3 positive (CD3+) TIL. In some embodiments, the human stem cell comprises an adult stem cell. In some embodiments, the human stem cell comprises an induced pluripotent stem cell (iPSC). In some embodiments, the human stem cell comprises an embryonic stem cell. In some embodiments, the in vitro-differentiated cell comprises an iPSC-derived natural killer T cell (iNKT), an iPSC-derived natural killer cell, an iPSC-derived T cell, an iPSC-derived macrophage, or an iPSC-derived B cell. In some embodiments, the one or more exogenous nucleic acids comprise a DNA, an RNA, or a combination thereof. In some embodiments, the RNA comprises a messenger RNA (mRNA), a guide RNA (gRNA), a microRNA (miRNA), a tRNA, a CRISPR RNA (crRNA), a short hairpin RNA (shRNA), a silencing RNA (siRNA), an antisense oligonucleotide, a non-coding RNA, an aptamer, or a combination thereof. In some embodiments, the DNA comprises a transgene encoding a protein, a regulatory element, a promoter, an enhancer, a repressor, an inhibitor, an antibiotic resistance gene, or a non-coding DNA. In some embodiments, the at least one exogenous nucleic acid comprises a sequence that is at least 80% identical, at least 85% identical, at least 90% identical, at least 95% identical, at least 96% identical, at least 97% identical, at least 98% identical, at least 99% identical, or 100% identical to SEQ ID NO: 8. In some embodiments, the at least one exogenous nucleic acid comprises a sequence that is at least 80% identical, at least 85% identical, at least 90% identical, at least 95% identical, at least 96% identical, at least 97% identical, at least 98% identical, at least 99% identical, or 100% identical to SEQ ID NO: 9. In some embodiments, the at least one exogenous nucleic acid encodes for an amino acid sequence that is at least 80% identical, at least 85% identical, at least 90% identical, at least 95% identical, at least 96% identical, at least 97% identical, at least 98% identical, at least 99% identical, or 100% identical to any one of SEQ ID NO: 10-SEQ ID NO: 14. (see SEQUENCES section).

### Aldehyde Dehydrogenase (ALDH)

Provided herein are compositions comprising: an engineered cell comprising one or more exogenous nucleic acids, wherein at least one exogenous nucleic acid encodes for an aldehyde dehydrogenase (ALDH) protein. In some embodiments, the engineered cell is resistant to alkylating agent-mediated cell death relative to a comparable cell that does not have an exogenous nucleic acid encoding for an aldehyde dehydrogenase (ALDH) protein. In some embodiments, the engineered cell is a mammalian cell. In some embodiments, the engineered cell expresses at least a 10% increase, at least a 20% increase, at least a 30% increase, at least a 40% increase, at least a 50% increase, at least a 60% increase, at least a 70% increase, or at least an 80% increase in the level of the ALDH protein relative to a comparable cell that does not have an exogenous nucleic acid encoding for a ALDH protein. In some embodiments, the engineered cell is an ex vivo-engineered cell, an in vivo-engineered cell, or an in vitro-engineered cell. In some embodiments, the engineered cell comprises a human leukocyte, a human stem cell, or an in vitro-differentiated cell. In some embodiments, the human leukocyte is a human T cell, a human natural killer (NK) cell, a human natural killer T cell (NKT), a human macrophage, a human B cell, a human dendritic cell, or a human tumor infiltrating lymphocyte (TIL). In some embodiments, the human T cell is a CD3 positive (CD3+) T cell. In some embodiments, the human TIL is a CD3 positive (CD3+) TIL. In some embodiments, the human stem cell comprises an adult stem cell. In some embodiments, the human stem cell comprises an induced pluripotent stem cell (iPSC). In some embodiments, the human stem cell comprises an embryonic stem cell. In some embodiments, the in vitro-differentiated cell comprises an iPSC-derived natural killer T cell (iNKT), an iPSC-derived natural killer cell, an iPSC-derived T cell, an iPSC-derived macrophage, or an iPSC-derived B cell. In some embodiments, the one or more exogenous nucleic acids comprise a DNA, an RNA, or a combination thereof. In some embodiments, the RNA comprises a messenger RNA (mRNA), a guide RNA (gRNA), a microRNA (miRNA), a tRNA, a CRISPR RNA (crRNA), a short hairpin RNA (shRNA), a silencing RNA (siRNA), an antisense oligonucleotide, a non-coding RNA, an aptamer, or a combination thereof. In some embodiments, the DNA comprises a transgene encoding a protein, a regulatory element, a promoter, an enhancer, a repressor, an inhibitor, an antibiotic resistance gene, or a non-coding DNA. In some embodiments, the ALDH protein is ALDH 1 Family Member A1 (ALDH1A1), ALDH 1 Family Member A2 (ALDH1A2), ALDH 1 Family Member A3 (ALDH1A3), ALDH 1 Family Member B1 (ALDH1B1), ALDH 1 Family Member L1 (ALDH1L1), ALDH 1 Family Member L2 (ALDH1L2), ALDH 2 Family Member (ALDH2), ALDH 3 Family Member A1 (ALDH3A1), ALDH 3 Family Member A2 (ALDH3A2), ALDH 3 Family Member B1 (ALDH3B1), ALDH 3 Family MemberB2 (ALDH3B2), ALDH 4 Family Member A1 (ALDH4A1), ALDH 5 Family Member A1 (ALDH5A1), ALDH 6 Family Member A1 (ALDH6A1), ALDH 7 Family Member A1 (ALDH7A1), ALDH 8 Family Member A1 (ALDHSAI), ALDH 9 Family Member A1 (ALDH9A1), ALDH 16 Family Member A1 (ALDH16A1), or ALDH 18 Family Member A1 (ALDH18A1). In some embodiments, the aldehyde dehydrogenase protein comprises an aldehyde dehydrogenase 1 family member A1 protein. In some embodiments, the aldehyde dehydrogenase protein comprises an aldehyde dehydrogenase 1 family member A2 protein. In some embodiments, the aldehyde dehydrogenase protein comprises an aldehyde dehydrogenase 1 family member B1 protein. In some embodiments, the aldehyde dehydrogenase protein comprises an aldehyde dehydrogenase 2 family member protein. In some embodiments, the aldehyde dehydrogenase protein comprises an aldehyde dehydrogenase 3 family member A1 protein. In some embodiments, the at least one exogenous nucleic acid comprises a sequence that is at least 80% identical, at least 85% identical, at least 90% identical, at least 95% identical, at least 96% identical, at least 97% identical, at least 98% identical, at least 99% identical, or 100% identical to SEQ ID NO: 15. In some embodiments, the at least one exogenous nucleic acid comprises a sequence that is at least 80% identical, at least 85% identical, at least 90% identical, at least 95% identical, at least 96% identical, at least 97% identical, at least 98% identical, at least 99% identical, or 100% identical to SEQ ID NO: 16. In some embodiments, the at least one exogenous nucleic acid encodes for an amino acid sequence that is at least 80% identical, at least 85% identical, at least 90% identical, at least 95% identical, at least 96% identical, at least 97% identical, at least 98% identical, at least 99% identical, or 100% identical to SEQ ID NO: 17 or SEQ ID NO: 18.

### Glutathione S Transferase

Provided herein are compositions comprising: an engineered cell comprising one or more exogenous nucleic acids, wherein at least one exogenous nucleic acid encodes for a glutathione S transferase protein. In some embodiments, the GST is GST Alpha 1 (GSTAI), GST Alpha 2 (GSTA2), GST Alpha 3 (GSTA3), GST Alpha 4 (GSTA4), GST Alpha 5 (GSTA5), GST Kappa 1 (GSTK1), GST Mu 1 (GSTM1), GST Mu I-like GSTMIL, GST Mu 2 (GSTM2), GST Mu 3 (GSTM3), GST Mu 4 (GSTM4), GST Mu 5 (GSTM5), GST Omega 1 (GSTO1), GST Omega 2 (GSTO2), GST Pi 1 (GSTP1), GST Theta 1 (GSTT1), GST Theta 2 (GSTT2), GST Theta 4 (GSTT4), GST Zeta 1 (GSTZ1), Microsomal GST 1 (MGST1), Microsomal GST 2 (MGST2), or Microsomal GST 3 (MGSTJ). In some embodiments, at least one exogenous nucleic acid encodes for a glutathione S transferase A4 (GSTA4) protein. In some embodiments, the engineered cell is resistant to alkylating agent-mediated cell death relative to a comparable cell that does not have an exogenous nucleic acid encoding for glutathione S transferase A4 (GSTA4). In some embodiments, the engineered cell is a human leukocyte, a human stem cell, or an in vitro-differentiated cell. In some embodiments, the engineered cell expresses at least a 10% increase, at least a 20% increase, at least a 30% increase, at least a 40% increase, at least a 50% increase, at least a 60% increase, at least a 70% increase, or at least an 80% increase in the level of GSTA4 protein relative to a comparable cell that does not have an exogenous nucleic acid encoding for a GSTA4 protein. In some embodiments, the engineered cell is an ex vivo-engineered cell, an in vivo-engineered cell, or an in vitro-engineered cell. In some embodiments, the human leukocyte is a human T cell, a human natural killer (NK) cell, a human natural killer T cell (NKT), a human macrophage, a human B cell, a human dendritic cell, or a human tumor infiltrating lymphocyte (TIL). In some embodiments, the human T cell is a CD3 positive (CD3+) T cell. In some embodiments, the human TIL is a CD3 positive (CD3+) TIL. In some embodiments, the human stem cell comprises an adult stem cell. In some embodiments, the human stem cell comprises an induced pluripotent stem cell (iPSC). In some embodiments, the human stem cell comprises an embryonic stem cell. In some embodiments, the in vitro-differentiated cell comprises an iPSC-derived natural killer T cell (iNKT), an iPSC-derived natural killer cell, an iPSC-derived T cell, an iPSC-derived macrophage, or an iPSC-derived B cell. In some embodiments, the one or more exogenous nucleic acids comprise a DNA, an RNA, or a combination thereof. In some embodiments, the RNA comprises a messenger RNA (mRNA), a guide RNA (gRNA), a microRNA (miRNA), a tRNA, a CRISPR RNA (crRNA), a short hairpin RNA (shRNA), a silencing RNA (siRNA), an antisense oligonucleotide, a non-coding RNA, an aptamer, or a combination thereof. In some embodiments, the DNA comprises a transgene encoding a protein, a regulatory element, a promoter, an enhancer, a repressor, an inhibitor, an antibiotic resistance gene, or a non-coding DNA. In some embodiments, the at least one exogenous nucleic acid comprises a sequence that is at least 80% identical, at least 85% identical, at least 90% identical, at least 95% identical, at least 96% identical, at least 97% identical, at least 98% identical, at least 99% identical, or 100% identical to SEQ ID NO: 19. In some embodiments, the at least one exogenous nucleic acid comprises a sequence that is at least 80% identical, at least 85% identical, at least 90% identical, at least 95% identical, at least 96% identical, at least 97% identical, at least 98% identical, at least 99% identical, or 100% identical to SEQ ID NO: 20. In some embodiments, the at least one exogenous nucleic acid encodes for an amino acid sequence that is at least 80% identical, at least 85% identical, at least 90% identical, at least 95% identical, at least 96% identical, at least 97% identical, at least 98% identical, at least 99% identical, or 100% identical to SEQ ID NO: 21 or SEQ ID NO: 22.

### Deoxycytidine Kinase

Provided herein are compositions comprising: a cell comprising a genomic disruption in a target sequence of a deoxycytidine kinase (dCK) gene. In some embodiments, the genomic disruption is a CRISPR-mediated genomic disruption. In some embodiments, the genomic disruption is an siRNA mediated disruption. In some embodiments, the cell is a CD3 positive (CD3+) T cell. In some embodiments, the one or more exogenous nucleic acids comprise a sequence encoding for an additional drug resistance gene. In some embodiments, the additional drug resistance gene comprises an aldehyde dehydrogenase gene, a glutathione S transferase gene, a protein kinase cAMP-activated catalytic subunit alpha (PRKACA), or a APH(3′)-IIa gene. In some embodiments, the composition further comprises a gene editing system provided herein (e.g., CRISPR-Cas). In some embodiments, the composition further comprises one or more guide RNAs.

Deoxycytidine kinase (dCK) is a homodimeric enzyme which is encoded by the human gene DCK, which is a member of the dCK/deoxyguanosine kinase (dGK) family of four kinases that may have similar activity (dCK, dGK, thymidine kinase I (TKI), and thymidine kinase 2 (TK2)). These enzymes can catalyze the 5′-phosphorylation of 2′-deoxyribonucleosides.

dCK can be responsible for the initial phosphorylation of fludarabine to form 2-fluoro-ara-adenine (F-ara-A). F-ara-A can be further phosphorylated to its diphosphate form (F-ara-ADP) and then to its triphosphate form (F-ara-ATP) by other cellular kinases. F-ara-ATP can be an active metabolite that can be incorporated into DNA during replication, disrupting DNA synthesis and repair processes, which can ultimately lead to cell death.

dCK may be dynamically regulated in lymphocytes. dCK may also be expressed at high levels in multiple T cell subsets including naive and activated T cells, NK T cells and regulatory T cells (Treg). dCK may be positively correlated to the sensitivity to nucleoside analogs such as nelarabine and fludarabine.

The dCK (and family members) can be active against nucleoside analog drugs. This enzyme which may be required for the phosphorylation of several deoxyribonucleosides and their nucleoside analogs. Deficiency of dCK may be associated with resistance to antiviral and anticancer chemotherapeutic agents. Conversely, increased deoxycytidine kinase activity may be associated with increased activation of these compounds to cytotoxic nucleoside triphosphate derivatives. dCK may be clinically important because of its relationship to drug resistance and sensitivity. Thus, genetic knockout, genetic knockdown, or inhibition of dCK activity can confer cell resistance to fludarabine.

Thymidine kinase 1 (TK1), thymidine kinase 2 (TK2), UMP-CMP kinase (CMPK1), and adenosine kinase (ADK) may be functionally similar to dCK. Accordingly, genetic knockout, genetic knockdown, or inhibition of genes that may be functionally similar to dCK, such as TKI, TK2, CMPK1, and ADK, may confer cell resistance to fludarabine.

### (4) Combination Compositions

Provided herein are compositions that confer drug resistance in a cell to one or more agents provided herein. In some embodiments, the compositions provided herein comprise one or more nucleic acids. In some embodiments, the one or more nucleic acids comprise the same transgene or different transgenes. In some embodiments, the compositions provided herein comprise: at least one nucleic acid that encodes for a first target (e.g. a DNA encoding for a first protein provided herein) and further comprises a nucleic acid encoding for the second target (e.g., a DNA encoding for a second protein provided herein) on the same nucleic acid strand (e.g., in cis). In some embodiments, the compositions provided herein comprise: a first nucleic acid encoding for a first target; and a second nucleic acid encoding for a second target. In some embodiments, the first target is on a different nucleic acid strand than the nucleic acid sequence encoding for the second target. (e.g., in trans).

In some embodiments, the compositions comprise: one or more nucleic acids, wherein at least one nucleic acid encodes for protein kinase cAMP-activated catalytic subunit alpha (PRKACA) protein. In some embodiments, the compositions comprise: one or more nucleic acids, wherein at least one nucleic acid encodes for an ALDH protein selected from the group consisting of: an aldehyde dehydrogenase 1 family member A1 protein, an aldehyde dehydrogenase 1 family member B1 protein, an aldehyde dehydrogenase 2 family member protein, and an aldehyde dehydrogenase 3 family member A1 protein. In some embodiments, the compositions comprise: one or more nucleic acids, wherein at least one nucleic acid encodes for a GSTA4 protein. In some embodiments, the compositions comprise: one or more nucleic acids, wherein at least one nucleic acid encodes for aminoglycoside-3′-phosphotransferase-IIa (APH(3′)-IIa) protein.

In some embodiments, the compositions comprise: a first nucleic acid encoding for a drug resistance protein selected from the group consisting of: GSTA4, APH(3′)-IIa, PRKACA, and ALDH3A1. In some embodiments, the compositions comprise: a second nucleic acid encoding for a drug resistance protein selected from the group consisting of: GSTA4, APH(3′)-IIa, PRKACA, and ALDH3A1. In some embodiments, the compositions comprise: a first nucleic acid encoding for a guide nucleic acid that targets dCK; and a second nucleic acid encoding for a gene editing protein (e.g., a Cas9, a TALEN, a ZFN, or a meganuclease). In some embodiments, the compositions comprise: a first nucleic acid encoding for a guide nucleic acid that targets dCK; a second nucleic acid encoding for a gene editing protein (e.g., a Cas9, a TALEN, a ZFN, or a meganuclease); and a third nucleic acid encoding for a drug resistance protein selected from the group consisting of: GSTA4, APH(3′)-IIa, PRKACA, or ALDH3A1.

In some embodiments, the compositions provided herein comprise: a nucleic acid that comprises a sequence that is at least 80% identical, at least 85% identical, at least 90% identical, at least 95% identical, at least 96% identical, at least 97% identical, at least 98% identical, at least 99% identical, or 100% identical to any one of SEQ ID NO: 1, SEQ ID NO: 2, SEQ ID NO: 8, SEQ ID NO: 9, SEQ ID NO: 15, SEQ ID NO: 16, SEQ ID NO: 19, SEQ ID NO: 20, or has complementarity to SEQ ID NO: 27 or SEQ ID NO: 28. In some embodiments, the compositions provided herein comprise: an engineered cell comprising one or more exogenous nucleic acids, wherein at least one exogenous nucleic acid comprises a sequence that is at least 80% identical, at least 85% identical, at least 90% identical, at least 95% identical, at least 96% identical, at least 97% identical, at least 98% identical, at least 99% identical, or 100% identical to any one of SEQ ID NO: 1, SEQ ID NO: 2, SEQ ID NO: 8, SEQ ID NO: 9, SEQ ID NO: 15, SEQ ID NO: 16, SEQ ID NO: 19, SEQ ID NO: 20, or has complementarity to SEQ ID NO: 27 or SEQ ID NO: 28. In some embodiments, the compositions provided herein comprise: a nucleic acid that comprises a sequence that is at least 90% identical to any one of SEQ ID NO: 1, SEQ ID NO: 2, SEQ ID NO: 8, SEQ ID NO: 9, SEQ ID NO: 15, SEQ ID NO: 16, SEQ ID NO: 19, SEQ ID NO: 20, or has complementarity to SEQ ID NO: 27 or SEQ ID NO: 28. In some embodiments, the compositions provided herein comprise: an engineered cell comprising one or more exogenous nucleic acids, wherein at least one exogenous nucleic acid comprises a sequence that is at least 90% identical to any one of SEQ ID NO: 1, SEQ ID NO: 2, SEQ ID NO: 8, SEQ ID NO: 9, SEQ ID NO: 15, SEQ ID NO: 16, SEQ ID NO: 19, SEQ ID NO: 20, or has complementarity to SEQ ID NO: 27 or SEQ ID NO: 28.

In some embodiments, the compositions provided herein comprise: a nucleic acid that comprises a sequence that is at least 95% identical to any one of SEQ ID NO: 1, SEQ ID NO: 2, SEQ ID NO: 8, SEQ ID NO: 9, SEQ ID NO: 15, SEQ ID NO: 16, SEQ ID NO: 19, SEQ ID NO: 20, or has complementarity to SEQ ID NO: 27 or SEQ ID NO: 28. In some embodiments, the compositions provided herein comprise: an engineered cell comprising one or more exogenous nucleic acids, wherein at least one exogenous nucleic acid comprises a sequence that is at least 95% identical to any one of SEQ ID NO: 1, SEQ ID NO: 2, SEQ ID NO: 8, SEQ ID NO: 9, SEQ ID NO: 15, SEQ ID NO: 16, SEQ ID NO: 19, SEQ ID NO: 20, or has complementarity to SEQ ID NO: 27 or SEQ ID NO: 28.

In some embodiments, the compositions provided herein comprise: a nucleic acid that comprises a sequence that is at least 98% identical to any one of SEQ ID NO: 1, SEQ ID NO: 2, SEQ ID NO: 8, SEQ ID NO: 9, SEQ ID NO: 15, SEQ ID NO: 16, SEQ ID NO: 19, SEQ ID NO: 20, or has complementarity to SEQ ID NO: 27 or SEQ ID NO: 28. In some embodiments, the compositions provided herein comprise: an engineered cell comprising one or more exogenous nucleic acids, wherein at least one exogenous nucleic acid comprises a sequence that is at least 98% identical to any one of SEQ ID NO: 1, SEQ ID NO: 2, SEQ ID NO: 8, SEQ ID NO: 9, SEQ ID NO: 15, SEQ ID NO: 16, SEQ ID NO: 19, SEQ ID NO: 20, or has complementarity to SEQ ID NO: 27 or SEQ ID NO: 28.

In some embodiments, the compositions provided herein comprise: a nucleic acid that comprises a sequence that is at least 99% identical to any one of SEQ ID NO: 1, SEQ ID NO: 2, SEQ ID NO: 8, SEQ ID NO: 9, SEQ ID NO: 15, SEQ ID NO: 16, SEQ ID NO: 19, SEQ ID NO: 20, or has complementarity to SEQ ID NO: 27 or SEQ ID NO: 28. In some embodiments, the compositions provided herein comprise: an engineered cell comprising one or more exogenous nucleic acids, wherein at least one exogenous nucleic acid comprises a sequence that is at least 99% identical to any one of SEQ ID NO: 1, SEQ ID NO: 2, SEQ ID NO: 8, SEQ ID NO: 9, SEQ ID NO: 15, SEQ ID NO: 16, SEQ ID NO: 19, SEQ ID NO: 20, or has complementarity to SEQ ID NO: 27 or SEQ ID NO: 28.

In some embodiments, the compositions provided herein comprise: a nucleic acid that comprises a sequence that is at least 100% identical to any one of SEQ ID NO: 1, SEQ ID NO: 2, SEQ ID NO: 8, SEQ ID NO: 9, SEQ ID NO: 15, SEQ ID NO: 16, SEQ ID NO: 19, SEQ ID NO: 20, or has complementarity to SEQ ID NO: 27 or SEQ ID NO: 28. In some embodiments, the compositions provided herein comprise: an engineered cell comprising one or more exogenous nucleic acids, wherein at least one exogenous nucleic acid comprises a sequence that is at least 100% identical to any one of SEQ ID NO: 1, SEQ ID NO: 2, SEQ ID NO: 8, SEQ ID NO: 9, SEQ ID NO: 15, SEQ ID NO: 16, SEQ ID NO: 19, SEQ ID NO: 20, or has complementarity to SEQ ID NO: 27 or SEQ ID NO: 28.

In some embodiments, the compositions comprise: a guide nucleic acid that targets a beta-2 microglobulin gene (B2M). In some embodiments, the compositions comprise: a guide nucleic acid that targets a class II major histocompatibility complex transactivator (CIITA) gene. In some embodiments, the compositions comprise: a guide nucleic acid that targets a programmed cell death 1 (PD1) gene. In some embodiments, the compositions comprise: a guide nucleic acid that targets T cell receptor constant region (TRAC). In some embodiments, the compositions comprise: a nucleic acid encoding for a gene editing protein or system provided herein (e.g., a Cas9, a TALEN, a ZFN, or a meganuclease). The nucleic acids provided herein can be delivered via a single carrier or multiple carriers.

Provided herein are engineered cells that comprise one or more modifications. In some embodiments, the engineered cells comprise one or more genomic disruptions in a target sequence of a gene. In some embodiments, the genomic disruption is in a target sequence of a dCK gene, a beta-2 microglobulin gene, a class II major histocompatibility complex transactivator (CIITA) gene, a programmed cell death 1 (PD1) gene, and T cell receptor constant region (TRAC).

In some embodiments, the engineered cells comprise an increased level or activity of one or more proteins provided herein. In some embodiments, the engineered cells provided herein comprise one or more exogenous nucleic acids encoding for a drug resistance protein selected from the group consisting of: GSTA4, APH(3′)-IIa, PRKACA, and ALDH3A1. In some embodiments, the engineered cells provided herein comprise a T cell receptor (TCR). In some embodiments, the TCR is engineered to bind to a neoantigen or an oncoprotein. In some embodiments, the engineered cells provided herein comprise a chimeric antigen receptor (CAR). In some embodiments, the engineered cells provided herein comprise an immune checkpoint inhibitor. In some embodiments, the engineered cells provided herein express an immune checkpoint inhibitor on the surface of the cells. In some embodiments, the engineered cells provided herein secretes an immune checkpoint inhibitor. Non-limiting examples of immune checkpoint inhibitors include CTLA-4 inhibitors (e.g, ipilimumab), PD-1 checkpoint inhibitors (e.g., pembrolizumab, nivolumab, avelumab, durvalumab, or atezolizumab), VISTA inhibitors, B7-H3 inhibitors, B7x inhibitors, JAK inhibitors LAG-3 inhibitors, TIGIT inhibitors, TIM-3 inhibitors, CD112R inhibitors, BTLA inhibitors, GITR inhibitors, and NKG2A inhibitors.

### (5) Pharmaceutical Compositions and Formulations

Provided herein are pharmaceutical compositions comprising: a composition, a genetically modified cell, a guide polynucleotide, an engineered protein, or a polynucleotide provided herein; and a pharmaceutically acceptable diluent, carrier, or excipient. Provided herein is a pharmaceutical composition comprising a vector provided herein; and a pharmaceutically acceptable diluent, carrier, or excipient.

In some embodiments, the present disclosure describes a pharmaceutical composition comprising the composition described herein, and a pharmaceutically acceptable excipient or carrier. The pharmaceutical composition of the present disclosure can be incorporated into a variety of formulations for therapeutic administration. More particularly, the pharmaceutical composition of the present disclosure can be formulated into pharmaceutical compositions by combination with appropriate, pharmaceutically acceptable excipients or diluents.

Formulations of the compositions suitable for administration to a patient (e.g., suitable for human administration) are generally sterile and may further be free of detectable pyrogens or other contaminants contraindicated for administration to a patient according to a selected route of administration.

The composition may be formulated for parenteral (e.g., intravenous, intra-arterial, intraosseous, intramuscular, intracerebral, intracerebroventricular, intrathecal, intratumoral, subcutaneous, etc.) administration, or any other suitable route of administration.

In some embodiments, pharmaceutical compositions that include the compositions of the present disclosure may be prepared by mixing the compositions having the desired degree of purity with optional physiologically acceptable carriers, excipients, stabilizers, surfactants, buffers and/or tonicity agents. Acceptable carriers, excipients and/or stabilizers are nontoxic to recipients at the dosages and concentrations employed, and include buffers such as phosphate, citrate, and other organic acids; antioxidants including ascorbic acid, glutathione, cysteine, methionine and citric acid; preservatives (such as ethanol, benzyl alcohol, phenol, m-cresol, p-chlor-m-cresol, methyl or propyl parabens, benzalkonium chloride, or combinations thereof); amino acids such as arginine, glycine, ornithine, lysine, histidine, glutamic acid, aspartic acid, isoleucine, leucine, alanine, phenylalanine, tyrosine, tryptophan, methionine, serine, proline and combinations thereof; monosaccharides, disaccharides and other carbohydrates; low molecular weight (less relative to about 10 residues) polypeptides; proteins, such as gelatin or serum albumin; chelating agents such as EDTA; sugars such as trehalose, sucrose, lactose, glucose, mannose, maltose, galactose, fructose, sorbose, raffinose, glucosamine, N-methylglucosamine, galactosamine, and neuraminic acid; and/or non-ionic surfactants such as Tween, Pluronics, Triton-X, or polyethylene glycol (PEG).

In some embodiments, an aqueous formulation of the recombinant polypeptides, proteases, nucleic acids, expression vectors, and/or cells may be prepared in a pH-buffered solution, e.g., at pH ranging from about 7.0 to 8.0, 4.0 to about 7.0, or from about 5.0 to about 6.0, or alternatively about 5.5. Examples of buffers that are suitable for a pH within this range include phosphate-, histidine-, citrate-, succinate-, acetate-buffers and other organic acid buffers. The buffer concentration can be from about 1 mM to about 100 mM, or from about 5 mM to about 50 mM, depending, e.g., on the buffer and the desired tonicity of the formulation.

In some embodiments, a tonicity agent may be included in the formulation to modulate the tonicity of the formulation. Example tonicity agents include sodium chloride, potassium chloride, glycerin and any component from the group of amino acids, sugars as well as combinations thereof. In some embodiments, the aqueous formulation is isotonic, although hypertonic or hypotonic solutions may be suitable. The term “isotonic” denotes a solution having the same tonicity as some other solution with which it is compared, such as physiological salt solution or serum. Tonicity agents may be used in an amount of about 5 mM to about 350 mM, e.g., in an amount of 100 mM to 350 mM.

In some embodiments, a surfactant may also be added to the formulation to reduce aggregation and/or minimize the formation of particulates in the formulation and/or reduce adsorption. Example surfactants include polyoxyethylensorbitan fatty acid esters (Tween), polyoxyethylene alkyl ethers (Brij), alkylphenylpolyoxyethylene ethers (Triton-X), polyoxyethylene-polyoxypropylene copolymer (Poloxamer, Pluronic), and sodium dodecyl sulfate (SDS). Examples of suitable polyoxyethylenesorbitan-fatty acid esters are polysorbate 20, (sold under the trademark Tween 20™) and polysorbate 80 (sold under the trademark Tween 80™). Examples of suitable polyethylene-polypropylene copolymers are those sold under the names Pluronic® F68 or Poloxamer 188™. Examples of suitable Polyoxyethylene alkyl ethers are those sold under the trademark Brij™. Example concentrations of surfactant may range from about 0.0010% to about 1% w/v.

In some instances, the pharmaceutical composition includes compositions of the present disclosure, and one or more of the above-identified agents (e.g., a surfactant, a buffer, a stabilizer, a tonicity agent) and is essentially free of one or more preservatives, such as ethanol, benzyl alcohol, phenol, m-cresol, p-chlor-m-cresol, methyl or propyl parabens, benzalkonium chloride, and combinations thereof. In other embodiments, a preservative is included in the formulation, e.g., at concentrations ranging from about 0.001 to about 2% (w/v).

In some embodiments, compositions provided herein (e.g., a nanoparticle comprising a vector or a nucleic acid provided herein) are combined with pharmaceutically acceptable salts, excipients, and/or carriers to form a pharmaceutical composition. Pharmaceutical salts, excipients, and carriers may be chosen based on the route of administration, the location of the target issue, and the time course of delivery of the drug. A pharmaceutically acceptable carrier or excipient may include solvents, dispersion media, coatings, antibacterial and antifungal agents, isotonic and absorption delaying agents, etc., compatible with pharmaceutical administration.

In some embodiments, the pharmaceutical composition is in the form of a solid, semi-solid, liquid or gas (aerosol). Injectable preparations, for example, sterile injectable aqueous or oleaginous suspensions may be formulated according to the known art using suitable dispersing or wetting agents and suspending agents. The sterile injectable preparation may also be a sterile injectable solution, suspension, or emulsion in a nontoxic parenterally acceptable diluent or solvent. Among the acceptable vehicles and solvents that may be employed are water, Ringer's solution, U.S.P., and isotonic sodium chloride solution. In addition, sterile, fixed oils are conventionally employed as a solvent or suspending medium. For this purpose, any bland fixed oil can be employed including synthetic mono- or diglycerides. In addition, fatty acids such as oleic acid are used in the preparation of injectables. The injectable formulations can be sterilized, for example, by filtration through a bacteria-retaining filter, or by incorporating sterilizing agents in the form of sterile solid compositions which can be dissolved or dispersed in sterile water or other sterile injectable medium prior to use.

### (6) Dosing and Administration

Provided herein are pharmaceutical compositions comprising: a composition, a guide polynucleotide, an engineered protein, or a polynucleotide provided herein; and a pharmaceutically acceptable diluent, carrier, or excipient. Provided herein is a pharmaceutical composition comprising a vector provided herein; and a pharmaceutically acceptable diluent, carrier, or excipient.

Provided herein are pharmaceutical compositions for administering a composition or a system to a subject in need thereof (e.g., as a treatment of a disease). In some embodiments, pharmaceutical compositions provided herein are in a form that allows for compositions provided herein to be administered to a subject. In some embodiments, the pharmaceutical composition is formulated for intratumoral delivery. In some embodiments, administration of a pharmaceutical composition provided herein is local administration or systemic administration. In some embodiments, a pharmaceutical composition provided herein is formulated for administration/for use in administration via an intratumoral, subcutaneous, intradermal, intramuscular, inhalation, intravenous, intraperitoneal, or intracranial route. In some embodiments, the administering is every 1, 2, 4, 6, 8, 12, 24, 36, or 48 hours. In some embodiments, the administering is at least about 5 hours, at least about 10 hours, at least about 12 hours, at least about 15 hours, at least about 20 hours, at least about 24 hours (1 day), at least about 48 hours (2 days), at least about 72 hours (3 days), at least about 96 hours (4 days), at least about 120 hours (5 days), at least about 144 hours (6 days), at least about 168 hours (7 days), at least about 336 hours (14 days), at least about 504 hours (21 days), at least about 672 hours (28 days), up to 744 hours (31 days). In some embodiments, the administering is every 744 hours (e.g., once per month) or once per year (365 days).

Vectors can be delivered in vivo by administration to an individual subject, typically by systemic administration (e.g., intravenous, intraperitoneal, intramuscular, subdermal, or intracranial infusion) or topical application, as described below. Alternatively, vectors can be delivered to cells ex vivo, such as cells explanted from an individual subject (e.g., lymphocytes, T cells, bone marrow aspirates, or tissue biopsy), followed by reimplantation of the cells into a subject, usually after selection for cells which have incorporated the vector. Prior to or after selection, the cells can be expanded.

Provided herein are cells expressing a system or a composition provided herein. In some embodiments, the cell is contacted in vitro or ex vivo with a nucleic acid encoding for a composition or a system provided herein. In some embodiments, the cell is contacted in vitro or ex vivo with a vector encoding for an engineered protein provided herein, a system provided herein, or a composition provided herein.

Compositions and systems provided herein may be formulated in dosage unit form for ease of administration and uniformity of dosage. A unit dosage form is a physically discrete unit of a composition.

### (7) Efficacy

Provided herein are methods of determining gene editing efficiency and selectivity of a gene editing system or gene editing agent; and/or a composition provided herein. In some embodiments, the target nucleic acid is cleaved by a nuclease or a nickase provided herein upon binding of the guide polynucleotide to the target nucleic acid sequence. In some embodiments, the target nucleic acid is a DNA. In some embodiments, the nickase region of the engineered protein provided herein cleaves the DNA resulting in a single-stranded break. In some embodiments, the nickase region of the engineered protein provided herein cleaves the target DNA via a staggered DNA double-stranded break. The ability of a nuclease or the nickase to recognize a PAM sequence can be determined by an in vitro selection assay.

The activity of a gene editing system provided herein to knock down or introduce a target gene provided herein into a cell may be assayed using a cell expressing a reporter protein or containing a reporter gene. For example, a reporter gene may be engineered to contain an obstruction, such as a stop codon, a frameshift mutation, a spacer, a linker, or a transcriptional terminator; the system may then be used to remove the obstruction and the resultant functional reporter protein may be detected. Similarly, a reporter gene can be introduced into the target nucleic acid for detection of the target. In some embodiments, the reporter gene may be designed such that a specific sequence modification is required to restore functionality of the reporter protein. In other embodiments, the reporter gene may be designed such that any insertion or deletion which results in a frame shift of one or two bases in the target nucleic acid may be sufficient to restore functionality of the reporter protein. Examples of reporter proteins encoded by a reporter gene include colorimetric enzymes, metabolic enzymes, fluorescent proteins, enzymes and transporters associated with antibiotic resistance, and luminescent enzymes. Examples of such reporter proteins include B-galactosidase, Chloramphenicol acetyltransferase, Green fluorescent protein, Red fluorescent protein, and Firefly and Renilla luciferase. Different detection methods may be used for different reporter proteins. For example, the reporter protein may affect cell viability, cell growth, fluorescence, luminescence, or expression of a detectable product. In some embodiments, the reporter protein may be detected using a colorimetric assay. In some embodiments, the reporter protein may be a fluorescent protein, and DNA editing may be assayed by measuring the degree of fluorescence in treated cells, or the number of treated cells with at least a threshold level of fluorescence. In some embodiments, transcript levels of a reporter gene may be assessed. In other embodiments, a reporter gene may be assessed by sequencing.

Integration of a new nucleic acid or transgene into a target nucleic acid can be measured using any technique, e.g., integration can be measured by denaturing urea polyacrylamide gel electrophoresis, PAGE gel electrophoresis, flow cytometry, a surveyor nuclease assay, tracking of indels by decomposition (TIDE), junction PCR, droplet digital PCR or any combination thereof. In other embodiments, transgene integration can be measured by PCR. A TIDE analysis can also be performed on engineered cells. Ex vivo cell transfection can also be used for diagnostics, research, or for gene therapy (e.g., via re-infusion of the transfected cells into the host organism). In some embodiments, cells are isolated from the subject organism, transfected with a nucleic acid (e.g., gene or cDNA), and re-infused back into the subject organism (e.g., subject).

Non-homologous end joining (NHEJ) and homology-directed repair (HDR) can be quantified using a variety of methods. For example, a percent of NHEJ, HDR, or a combination of both can be determined by co-delivering the gene editing molecules, for example a guide polynucleotide and an engineered protein provided herein, with a donor nucleic acid template that encodes a promoter-less tag or marker (e.g., GFP) into cells. After a duration of time (e.g., 72-96 hours), flow cytometry can be performed to quantify the total cell number (NTotal), tag-positive cell number and tag/GFP-negative cell number. Among the tag-negative cells, next-generation sequencing can be performed to identify cells without mutations and with mutations. HDR efficiency and NHEJ efficiency can be calculated from the assay.

Additional assays for determining gene editing efficiency of a system or composition provided herein can include but is not limited to: RT-PCR, nucleic acid sequencing, T7 endonuclease 1 (T7E1) mismatch detection assays, tracking of indels by decomposition (TIDE) assays, and indel detection by amplicon analysis (IDAA) assays. The indel pattern that is induced at the target site of a programmable nuclease or nickase may also be determined by PCR-amplifying the respective region and subsequent next generation sequencing. To obtain a quantitative single-cell view of gene editing efficiency, cell surface markers can be targeted and the loss of signal that occurs as a consequence of indel formation by flow cytometry can be quantified. Single-cell sequencing can also be employed to directly assess the gene-editing efficiency.

The amount of genetically modified cells that can be necessary to be therapeutically effective in a subject can vary depending on the viability of the cells, and the efficiency with which the cells have been genetically modified (e.g., the efficiency with which a transgene has been integrated into one or more cells). In some embodiments, the product (e.g., multiplication) of the viability of cells post genetic modification and the efficiency of integration of a transgene can correspond to the therapeutic aliquot of cells available for administration to a subject. In some embodiments, an increase in the viability of cells post-genetic modification can correspond to a decrease in the amount of cells that are necessary for administration to be therapeutically effective in a subject. In some embodiments, an increase in the efficiency with which a transgene has been integrated into one or more cells can correspond to a decrease in the amount of cells that are necessary for administration to be therapeutically effective in a subject. In some embodiments, determining an amount of cells that are necessary to be therapeutically effective can comprise determining a function corresponding to a change in the viability of cells over time. In some embodiments, determining an amount of cells that are necessary to be therapeutically effective can comprise determining a function corresponding to a change in the efficiency with which a transgene can be integrated into one or more cells with respect to time dependent variables (e.g., cell culture time, electroporation time, cell stimulation time).

The pharmaceutical compositions generally include a therapeutically effective amount of a composition described herein (e.g., a cell, a vector, a nanoparticle composition, or a nucleic acid provided herein). An effective amount can be administered in one or more administrations. A “therapeutically effective amount” disclosed herein may vary according to factors such as the disease state, age, sex, and weight of the individual, and the ability of the cells to elicit a desired response in the individual. A therapeutically effective amount is also one in which any toxic or detrimental effects of the cells are outweighed by the therapeutically beneficial effects. The term “therapeutically effective amount” includes an amount that is effective to “treat” an individual, e.g., a patient. When a therapeutic amount is indicated, the precise amount of the compositions contemplated in particular embodiments, to be administered, can be determined by a physician in view of the specification and with consideration of individual differences in age, weight, tumor size, extent of infection or metastasis, and condition of the patient (individual).

For any composition provided herein, the therapeutically effective dose can be estimated initially either in cell culture assays or in animal models, such as mice, rabbits, dogs, pigs, or non-human primates. The animal model is also used to achieve a desirable concentration range and route of administration. Such information can then be used to determine useful doses and routes for administration in humans. Therapeutic efficacy and toxicity of compositions provided herein can be determined by standard pharmaceutical procedures in cell cultures or experimental animals, e.g., ED₅₀ (the dose is therapeutically effective in 50% of the population) and LD₅₀ (the dose is lethal to 50% of the population). The dose ratio of toxic to therapeutic effects is the therapeutic index, and it can be expressed as the ratio, LD₅₀/ED₅₀. Pharmaceutical compositions which exhibit large therapeutic indices may be useful in some embodiments. The data obtained from cell culture assays and animal studies may be used in formulating a range of dosage for human use.

### (8) Methods of Treating a Disease

Provided herein are methods for treating a disease, disorder, or condition in a subject in need thereof, comprising administering a therapeutically effective amount of the pharmaceutical composition disclosed herein. In some embodiments, the subject has been treated with a lymphodepletion agent prior to administration of the pharmaceutical composition. In some embodiments, the subject is treated with a lymphodepletion agent after administration of the pharmaceutical composition. In some embodiments, the subject is treated with a lymphodepletion agent concomitantly with the pharmaceutical composition.

In some embodiments, lymphodepletion is performed on a subject, e.g., prior to administering one or more cells that express an engineered cell described herein. In some embodiments, the lymphodepletion regimen comprises administering cyclophosphamide. In some embodiments, the lymphodepletion regimen comprises administering fludarabine. In some embodiments, the lymphodepletion regimen comprises administering cyclophosphamide and fludarabine. In some embodiments, the lymphodepletion regimen comprises administering melphalan. In some embodiments, the lymphodepletion regimen comprises administering Cytoxan. In some embodiments, the lymphodepletion regimen comprises administering bendamustine.

In some embodiments, cyclophosphamide is administered for 1, 2, 3, 4 5, 6 or 7 days. In some embodiments cyclophosphamide is administered at a dosage of 100-1000 mg/m²/day, 200-1000 mg/m²/day, 300-1000 mg/m²/day, or 400-1000 mg/m²/day. In some embodiments cyclophosphamide is administered at a dosage of at least 100 mg/m²/day, 150 mg/m²/day, 200 mg/m²/day, 250 mg/m²/day, 300 mg/m²/day, 350 mg/m²/day, 400 mg/m²/day, 450 mg/m²/day, 500 mg/m²/day, 550 mg/m²/day, 600 mg/m²/day, 650 mg/m²/day, 700 mg/m²/day, 750 mg/m²/day, 800 mg/m²/day, 800 mg/m²/day or 1000 mg/m²/day. In some embodiments cyclophosphamide is administered at a dosage of at most 100 mg/m²/day, 150 mg/m²/day, 200 mg/m²/day, 250 mg/m²/day, 300 mg/m²/day, 350 mg/m²/day, 400 mg/m²/day, 450 mg/m²/day, 500 mg/m²/day, 550 mg/m²/day, 600 mg/m²/day, 650 mg/m²/day, 700 mg/m²/day, 750 mg/m²/day, 800 mg/m²/day, 800 mg/m²/day or 1000 mg/m²/day.

In some embodiments, cyclophosphamide is administered for 2 days. In some embodiments cyclophosphamide is administered for 3 days. In some embodiments, cyclophosphamide is administered for 4 days. In some embodiments, cyclophosphamide is administered for 5 days. In embodiments, the lymphodepletion regimen comprises administering fludarabine. In embodiments, fludarabine is administered for 1, 2, 3, 4 5, 6 or 7 days. In some embodiments fludarabine is administered at a dosage of 10-500 mg/m²/day, 20-500 mg/m²/day, 20-250 mg/m²/day, or 25-35 mg/m²/day. In some embodiments fludarabine is administered at a dosage of 10 mg/m²/day, 15 mg/m²/day, 20 mg/m²/day, 25 mg/m²/day, 30 mg/m²/day, 35 mg/m²/day, 40 mg/m²/day, 45 mg/m²/day, 50 mg/m²/day, 55 mg/m²/day, 60 mg/m²/day, 65 mg/m²/day, 70 mg/m²/day, or 75 mg/m²/day.

In some embodiments fludarabine is administered for 2 days. In some embodiments cyclophosphamide is fludarabine for 3 days. In some embodiments fludarabine is administered for 4 days. In some embodiments fludarabine is administered for 5 days. In some embodiments, the lymphodepletion regimen comprises administering cyclophosphamide prior to administering the engineered cells. In some embodiments, the lymphodepletion regimen comprises administering fludarabine prior to administering the engineered cells. In some embodiments, the lymphodepletion regimen comprises administering cyclophosphamide and fludarabine prior to administering the engineered cells.

In some embodiments, the lymphodepletion regimen comprises administering cyclophosphamide after administering the engineered cells. In some embodiments, the lymphodepletion regimen comprises administering fludarabine after administering the engineered cells. In some embodiments, the lymphodepletion regimen comprises administering cyclophosphamide and fludarabine after administering the engineered cells.

In some embodiments, the lymphodepletion regimen is initiated with the administration of the first dose of cyclophosphamide. In some embodiments, the lymphodepletion regimen is initiated with the administration of the first dose of fludarabine. In some embodiments, cyclophosphamide and fludarabine are administered on the same day In some embodiments, cyclophosphamide and fludarabine are not administered on the same day. In some embodiments, the daily dosages are administered on consecutive days.

In some embodiments, at least one does of cyclophosphamide is administered on the same day as a dose of fludarabine. In some embodiments, a first dose of cyclophosphamide is administered on the same day as a first dose of fludarabine. In some embodiments, second dose of cyclophosphamide is administered on the same day as a second dose of fludarabine. In some embodiments, a third dose of cyclophosphamide is administered on the same day as a third dose of fludarabine. In some embodiments, each dose of cyclophosphamide administered is administered on the same day that a dose of fludarabine is administered.

In some embodiments, the first does of cyclophosphamide is administered on day −20, −19, −18, −17, −16, −15, −14, −13, −12, −11, −10, −9, −8, −7, −6, −5, −4, −3, −2, or −1 and the engineered cells are administered on day 0. In some embodiments, the first does of fludarabine is administered on day −20, −19, −18, −17, −16, −15, −14, −13, −12, −11, −10, −9, −8, −7, −6, −5, −4, −3, −2, or −1 and the engineered cells are administered on day 0. In some embodiments, the first does of cyclophosphamide and the first dose of fludarabine are administered on day −20, −19, −18, −17, −16, −15, −14, −13, −12, −11, −10, −9, −8, −7, −6, −5, −4, −3, −2, or −1 and the engineered cells are administered on day 0.

In some embodiments, a dose of cyclophosphamide and a dose of fludarabine are administered on day −7 and day −6 and day −5, and the engineered cells are administered on day 0. In some embodiments, a dose of cyclophosphamide and a dose of fludarabine are administered on day −6 and day −5 and day −4, and the engineered cells are administered on day 0. In some embodiments, a dose of cyclophosphamide and a dose of fludarabine are administered on day −5 and day −4 and day −3, and the engineered cells are administered on day 0. In some embodiments, a dose of cyclophosphamide and a dose of fludarabine are administered on day −4 and day −3 and day −2, and the engineered cells are administered on day 0. In some embodiments, a dose of cyclophosphamide and a dose of fludarabine are administered on day −3 and day −2 and day −1, and the engineered cells are administered on day 0.

The pharmaceutical composition can be administered alone or in combination with other agents (e.g., an antibody or an antigen binding fragment thereof, or a molecule). In some embodiments, a vaccine, an oncolytic virus, a checkpoint inhibitor, a T cell agonist antibody, chemotherapy, and/or a bispecific antibody can be combined with the pharmaceutical composition disclosed herein. In some instances, the pharmaceutical composition is administered with other cells (e.g., CAR-T cells or other adoptively transferred T cells). Administration “in combination with” one or more additional cell depletion drug or cell division inhibitors includes simultaneous (concurrent) and consecutive administration in any order. In some embodiments, the one or more additional cell depletion drug or cell division inhibitors is selected from the group consisting of lymphodepletion agents, chemotherapy, radiotherapy, immunotherapy, and toxin therapy. “Chemotherapy” and “anti-cancer agent” are used interchangeably herein. Various classes of anti-cancer agents can be used. Non-limiting examples include: alkylating agents, antimetabolites, anthracyclines, plant alkaloids, topoisomerase inhibitors, podophyllotoxin, antibodies (e.g., monoclonal or polyclonal), checkpoint inhibitors, immunomodulators, cytokines, nanoparticles, radiation therapy, tyrosine kinase inhibitors (for example, imatinib mesylate), hormone treatments, soluble receptors and other antineoplastics.

In some embodiments, the disease or condition is an autoimmune disease, a cancer, or hematopoietic stem cell transplantation. In some embodiments, the cancer is a hematological malignancy or a solid tumor. In some embodiments, the disease or condition is a leukemia, a lymphoma, a melanoma, a neuroblastoma, a glioblastoma, a carcinoma, a breast cancer, a lung cancer, a colon cancer, a pancreatic cancer, a prostate cancer, a colorectal cancer, a stomach cancer, an ovarian cancer, a uterine cancer, or a bladder cancer. In some embodiments, the disease or condition is Myasthenia Gravis (MG), Neuromyelitis optica spectrum disorder (NMOSD), Sjorgen's syndrome (SS), scleroderma, immune nephritis, systemic lupus erythematosus (SLE), or mucosal-dominant PV (mPV), multiple sclerosis (MS), or Type 1 diabetes.

In some instances, the disease, disorder, or condition is a cancer, an inflammatory disease, a hematopoietic stem cell transplantation, a neuronal disorder, HIV/AIDS, diabetes, a cardiovascular disease, an infectious disease, an autoimmune disease, or aging. In some instances, the disease, disorder, or condition is a hyperproliferative disorder. Hyperproliferative disorders include cancers and hyperplasia characterized by the unregulated overgrowth of cells. Hyperproliferative disorders frequently display loss of genetic regulatory mechanisms, and may express native proteins inappropriately (including expression of proteins from other cell types or developmental stages, expression of mutated proteins, and expression of proteins at levels higher or lower than normal). [0169]B-cell hyperproliferative disorders include B-cell leukemias and lymphomas such as, but not limited to, acute lymphoblastic leukemia (ALL), chronic lymphocytic leukemia (CLL), B-cell prolymphocytic leukemia, precursor B lymphoblastic leukemia, hairy cell leukemia, diffuse large B-cell lymphoma (DLBCL), follicular lymphoma, marginal zone lymphoma, mantle cell lymphoma, Burkitt's lymphoma, MALT lymphoma, Waldenstrom's macroglobulinemia, and/or other disorders characterized by the overgrowth of B-lineage cells.

Hyperproliferative disorders include diseases such as, but not limited to, bladder cancer, including upper tract tumors and urothelial carcinoma of the prostate; bone cancer, including chondrosarcoma, Ewing's sarcoma, and osteosarcoma; breast cancer, including noninvasive, invasive, phyllodes tumor, Paget's disease, and breast cancer during pregnancy; central nervous system cancers, adult low-grade infiltrative supratentorial astrocytoma/oligodendroglioma, adult intracranial ependymoma, anaplastic astrocytoma/anaplastic oligodendroglioma/glioblastoma multiforme, carcinomatous lymphomatous meningitis, non-immunosuppressed primary CNS lymphoma, and metastatic spine tumors; cervical cancer; colon cancer, rectal cancer, anal carcinoma; esophageal cancer; gastric (stomach) cancer; head and neck cancers, including ethmoid sinus tumors, maxillary sinus tumors, salivary gland tumors, cancer of the lip, cancer of the oral cavity, cancer of the oropharynx, cancer of the hypopharynx, occult primary, cancer of the glottic larynx, cancer of the supraglottic larynx, cancer of the nasopharynx, and advanced head and neck cancer; hepatobiliary cancers, including hepatocellular carcinoma, gallbladder cancer, intrahepatic cholangiocarcinoma, and extrahepatic cholangiocarcinoma; Hodgkin disease/lymphoma; kidney cancer; melanoma; multiple myeloma, systemic light chain amyloidosis, Waldenstrom's macroglobulinemia; myelodysplastic syndromes; neuroendocrine tumors, including multiple endocrine neoplasia, type I, multiple endocrine neoplasia, type 2, carcinoid tumors, islet cell tumors, pheochromocytoma, poorly differentiated/small cell/atypical lung carcinoids; Non-Hodgkin's Lymphomas, including chronic lymphocytic leukemia/small lymphocytic lymphoma, follicular lymphoma, marginal zone lymphoma, mantle cell lymphoma, diffuse large B-Cell lymphoma, Burkitt's lymphoma, lymphoblastic lymphoma, AIDS-Related B-Cell lymphoma, peripheral T Cell lymphoma, and mycosis fungoides/Sezary Syndrome; non-melanoma skin cancers, including basal and squamous cell skin cancers, dermatofibrosarcoma protuberans, Merkel cell carcinoma; non-small cell lung cancer (NSCLC), including thymic malignancies; occult primary; ovarian cancer, including epithelial ovarian cancer, borderline epithelial ovarian cancer (Low Malignant Potential), and less common ovarian histologies; pancreatic adenocarcinoma; prostate cancer; small cell lung cancer and lung neuroendocrine tumors; soft tissue sarcoma, including soft-tissue extremity, retroperitoneal, intra-abdominal sarcoma, and desmoid; testicular cancer; thymic malignancies, including thyroid carcinoma, nodule evaluation, papillary carcinoma, follicular carcinoma, Hürthle cell neoplasm, medullary carcinoma, and anaplastic carcinoma; uterine neoplasms, including endometrial cancer and/or uterine sarcoma.

In some embodiments, the methods provided herein further comprise administering an additional chemotherapeutic agent or anti-cancer agent. A chemotherapeutic agent or compound is any agent or compound useful in the treatment of cancer. The chemotherapeutic cancer agents that can be used in combination with the compositions provided herein which include, but are not limited to, mitotic inhibitors (vinca alkaloids). These include vincristine, vinblastine, vindesine and Navelbine™ (vinorelbine, 5′-noranhydroblastine). In yet other cases, chemotherapeutic cancer agents include topoisomerase I inhibitors, such as camptothecin compounds. As used herein, “camptothecin compounds” include Camptosar™ (irinotecan HCL), Hycamtin™ (topotecan HCL) and other compounds derived from camptothecin and its analogues. Another category of chemotherapeutic cancer agents that can be used in the methods and compositions disclosed herein are podophyllotoxin derivatives, such as etoposide, teniposide and mitopodozide. The present disclosure further encompasses other chemotherapeutic cancer agents known as alkylating agents, which alkylate the genetic material in tumor cells. These include without limitation cisplatin, cyclophosphamide, nitrogen mustard, trimethylene thiophosphoramide, carmustine, busulfan, chlorambucil, belustine, uracil mustard, chlomaphazin, and dacarbazine. The disclosure encompasses antimetabolites as chemotherapeutic agents. Examples of these types of agents include cytosine arabinoside, fluorouracil, methotrexate, mercaptopurine, azathioprime, and procarbazine. An additional category of chemotherapeutic cancer agents that may be used in the methods and compositions disclosed herein include antibiotics. Examples include without limitation doxorubicin, bleomycin, dactinomycin, daunorubicin, mithramycin, mitomycin, mytomycin C, and daunomycin. There are numerous liposomal formulations commercially available for these compounds. The present disclosure further encompasses other chemotherapeutic cancer agents including without limitation anti-tumor antibodies, dacarbazine, azacytidine, amsacrine, melphalan, ifosfamide and mitoxantrone.

The compositions provided herein can be administered in combination with other anti-tumor agents, including cytotoxic/antineoplastic agents and anti-angiogenic agents. Cytotoxic/anti-neoplastic agents can be defined as agents who attack and kill cancer cells. Some cytotoxic/anti-neoplastic agents can be alkylating agents, which alkylate the genetic material in tumor cells, e.g., cis-platin, cyclophosphamide, nitrogen mustard, trimethylene thiophosphoramide, carmustine, busulfan, chlorambucil, belustine, uracil mustard, chlomaphazin, and dacabazine. Other cytotoxic/anti-neoplastic agents can be antimetabolites for tumor cells, e.g., cytosine arabinoside, fluorouracil, methotrexate, mercaptopuirine, azathioprime, and procarbazine. Other cytotoxic/anti-neoplastic agents can be antibiotics, e.g., doxorubicin, bleomycin, dactinomycin, daunorubicin, mithramycin, mitomycin, mytomycin C, and daunomycin. There are numerous liposomal formulations commercially available for these compounds. Still other cytotoxic/anti-neoplastic agents can be mitotic inhibitors (vinca alkaloids). These include vincristine, vinblastine and etoposide. Miscellaneous cytotoxic/anti-neoplastic agents include taxol and its derivatives, L-asparaginase, anti-tumor antibodies, dacarbazine, azacytidine, amsacrine, melphalan, VM-26, ifosfamide, mitoxantrone, and vindesine.

Anti-angiogenic agents can also be used. Suitable anti-angiogenic agents for use in the disclosed methods and compositions include anti-VEGF antibodies, including humanized and chimeric antibodies, anti-VEGF aptamers and antisense oligonucleotides. Other inhibitors of angiogenesis include angiostatin, endostatin, interferons, interleukin 1 (including a and 3) interleukin 12, retinoic acid, and tissue inhibitors of metalloproteinase-1 and -2. (TIMP-1 and -2). Small molecules, including topoisomerases such as razoxane, a topoisomerase II inhibitor with anti-angiogenic activity, can also be used.

Other anti-cancer agents that can be used in combination with the compositions provided herein can include, but are not limited to: acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; avastin; azacitidine; azetepa; azotomycin; batimastat; benzodepa; bevacizumab; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflomithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; folinic acid; fosquidone; fostriecin sodium; gemcitabine; gemcitabine hydrochloride; hydroxyurea; idarubicin hydrochloride; ifosfamide; ilmofosine; interleukin II (including recombinant interleukin II, or rIL2), interferon alfa-2a; interferon alfa-2b; interferon alfa-n1; interferon alfa-n3; interferon beta-I a; interferon gamma-I b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride. Other anti-cancer agents include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docetaxel; docosanol; dolasetron; doxifluridine; droloxifene; dronabinol; duocarmycin SA; ebselen; ecomustine; edelfosine; edrecolomab; eflomithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; 06-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perfiubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; erythrocyte gene therapy; velaresol; veramine; verdins; verteporfin; vinorelbine; vinxaltine; vitaxin; vorozole; zanoterone; zeniplatin; zilascorb; and zinostatin stimalamer. Any of the aforementioned chemotherapeutics can be administered at a clinically effective dose or amount. A chemotherapeutic can also be administered from about day: −14, −13, −12, −11, −10, −9, −8, −7, −6, −5, −4, −3, −2, −1, 0, 1, 2, 3, 4, 5, 6, 7, 8, 9, 10, 11, 12, 13, or up to about day 14 after administration of an agent provided herein. In some cases, a subject can have a refractory cancer that is unresponsive to a chemotherapeutic.

### (9) Kits, Delivery Systems, and Dosing Regimens

Provided herein are kits, delivery systems, and dosing regimen comprising the compositions provided herein for use in the manufacture and/or the treatment of a disease provided herein.

Provided herein are kits, wherein the kits comprise: a system, a composition, a polynucleotide, a vector, a guide polynucleotide, an engineered protein, or an engineered RNA encoding the engineered protein provided herein; and packaging and materials therefor. In some embodiments, the kit further comprises a scaffold provided herein. In some embodiments, the kit further comprises a cell-free system. In some embodiments, the kit further comprises a population of cells. In some embodiments, the cells are stored in a cryopreservation medium. In some embodiments, the cryopreservation medium comprises: dimethyl sulfoxide (DMSO). In some embodiments, the cryopreservation medium comprises a buffer, an isotonic agent or an apoptosis inhibitor. Non-limiting examples of buffer elements include: citrate, phosphate, succinate, tartrate, fumarate, gluconate, oxalate, lactate, acetate, histidine and tris. Non-limiting examples of isotonic agents include, for example, citrate, phosphate, succinate, tartrate, fumarate, gluconate, oxalate, lactate, acetate, histidine, and tris. Additional isotonic agents include sodium chloride, potassium chloride, boric acid, sodium borate, mannitol, glycerin, propylene glycol, polyethylene glycol, maltose, sucrose, erythritol, arabitol, xylitol, sorbitol trehalose, and glucose. The apoptosis inhibitor can include, for example, a Rho associated kinase (ROCK) inhibitor, catalase, and zVAD-fmk. In some embodiments, the kits comprise reagents. In some embodiments, the reagents comprise saccharides and saccharide derivatives (e.g., sodium carboxymethyl cellulose and cellulose acetate), detergents, glycols, polyols, esters, buffering agents, alginic acid, and organic solvents.

In some embodiments, a formulation of a composition described herein is prepared in a single container for administration to a cell, a cell-free system, or a subject. In some embodiments, a formulation of a composition provided herein is prepared two containers for administration, separating the guide polynucleotide or polynucleotide encoding the guide polynucleotide and/or the polynucleotide encoding the engineered protein provided herein. As used herein, “container” includes vessel, vial, ampule, tube, cup, box, bottle, flask, jar, dish, well of a single-well or multi-well apparatus, reservoir, tank, or the like, or other device in which the herein disclosed compositions may be placed, stored and/or transported, and accessed to remove the contents. Examples of such containers include glass and/or plastic sealed or re-sealable tubes and ampules, including those having a rubber septum or other sealing means that is compatible with withdrawal of the contents using a needle and syringe. In some embodiments, the containers are RNase free.

Provided herein are kits comprising: a first container comprising: a gene editing agent (e.g., CRISPR-Cas system); and a second container comprising: a vector provided herein. In some embodiments the kit further comprises a third container comprising: a population of cells provided herein.

Provided herein are kits comprising: a first container comprising: a population of engineered cells provided herein; and a second container comprising: an alkylating agent or an aminoglycoside agent.

Provided herein are delivery systems for use in the treatment of a disease, wherein the delivery systems comprise: an engineered cell provided herein; and an alkylating agent or an aminoglycoside agent, wherein the engineered cell is resistant to cell death mediated by the alkylating agent or the aminoglycoside agent. The compositions provided herein can be administered by controlled release formulations and/or delivery devices (see, e.g., in U.S. Pat. No. 5,733,566). In some embodiments, the delivery system comprises a microdevice configured to permit implantation into a tissue in a subject using a catheter, cannula, or biopsy needle comprising: (i) at least one microwell containing one or more active agents or a population of cells provided herein; (ii) a micro-dose of the one or more active agents in the at least one microwell; and (iii) a compound release mechanism comprising a polymeric matrix for controlling the release of the one or more active agents from the microwell into the tissue. In some embodiments, the drug delivery system comprises compositions that induce cell death in a tumor and do not induce cell death in an engineered cell provided herein.

Provided herein are dosing regimens, wherein the dosing regimens comprise: a first composition comprising an engineered cell provided herein; and a second composition comprising an alkylating agent provided herein. Provided herein are dosing regimens, wherein the dosing regimens comprise: a first composition comprising an engineered cell provided herein; and a second composition comprising an aminoglycoside agent provided herein or a metabolite thereof.

### (10) Exemplary Embodiments

A number of compositions, and methods are disclosed herein. Specific exemplary embodiments of these compositions and methods are disclosed below. The following embodiments recite non-limiting permutations of combinations of features disclosed herein. Other permutations of combinations of features are also contemplated. In particular, each of these numbered embodiments is contemplated as depending from or relating to every previous or subsequent numbered embodiment, independent of their order as listed.

Embodiment 1. A composition comprising: an engineered cell comprising one or more exogenous nucleic acids, wherein at least one exogenous nucleic acid that encodes for: a drug resistance protein selected from the group consisting of: an aminoglycoside phosphotransferase, a protein kinase cAMP-activated catalytic subunit alpha (PRKACA), an aldehyde dehydrogenase (ALDH), or a glutathione S-transferase, wherein the engineered cell is resistant to drug-mediated cell death relative to a comparable cell that does not have an exogenous nucleic acid encoding for a drug resistance protein. Embodiment 2. The composition of embodiment 1, wherein the engineered cell is a human leukocyte, a human stem cell, or an in vitro-differentiated cell. Embodiment 3. The composition of embodiment 1 or embodiment 2, wherein the engineered cell is an ex vivo-engineered cell, an in vivo-engineered cell, or an in vitro-engineered cell. Embodiment 4. The composition of any preceding embodiment, wherein the human leukocyte is a human T cell, a human natural killer (NK) cell, a human natural killer T cell (NKT), a human macrophage, a human B cell, a human dendritic cell, or a human tumor infiltrating lymphocyte (TIL). Embodiment 5. The composition of any preceding embodiment, wherein the human T cell is a CD3 positive (CD3+) T cell. Embodiment 6. The composition of any preceding embodiment, wherein the human TIL is a CD3 positive (CD3+) TIL. Embodiment 7. The composition of any preceding embodiment, wherein the human stem cell comprises an adult stem cell. Embodiment 8. The composition of any preceding embodiment, wherein the human stem cell comprises an induced pluripotent stem cell (iPSC). Embodiment 9. The composition of any preceding embodiment, wherein the human stem cell comprises an embryonic stem cell. Embodiment 10. The composition of any preceding embodiment, wherein the in vitro-differentiated cell comprises an iPSC-derived natural killer T cell (iNKT), an iPSC-derived natural killer cell, an iPSC-derived T cell, an iPSC-derived macrophage, or an iPSC-derived B cell. Embodiment 11. The composition of any preceding embodiment, wherein the one or more exogenous nucleic acids comprise a DNA, an RNA, or a combination thereof. Embodiment 12. The composition of any preceding embodiment, wherein the RNA comprises a messenger RNA (mRNA), a guide RNA (gRNA), a microRNA (miRNA), a tRNA, a CRISPR RNA (crRNA), a short hairpin RNA (shRNA), a silencing RNA (siRNA), an antisense oligonucleotide, a non-coding RNA, an aptamer, or a combination thereof. Embodiment 13. The composition of any preceding embodiment, wherein the DNA comprises a transgene encoding a protein, a regulatory element, a promoter, an enhancer, a repressor, an inhibitor, an antibiotic resistance gene, or a non-coding DNA. Embodiment 14. The composition of any preceding embodiment, wherein the one or more nucleic acids further comprise a poly-A tail or a nucleoside modification. Embodiment 15. The composition of any preceding embodiment, wherein the one or more exogenous nucleic acids comprise a sequence encoding for an additional drug resistance gene. Embodiment 16. The composition of any preceding embodiment, wherein the additional drug resistance gene comprises an aldehyde dehydrogenase gene, a glutathione S transferase gene, a protein kinase cAMP-activated catalytic subunit alpha (PRKACA), or a APH(3′)-IIa gene. Embodiment 17. The composition of any preceding embodiment, wherein the one or more exogenous nucleic acids comprise a sequence encoding for a T cell receptor (TCR). Embodiment 18. The composition of any preceding embodiment, wherein the one or more exogenous nucleic acids comprise a sequence encoding for a chimeric antigen receptor (CAR). Embodiment 19. The composition of any preceding embodiment, wherein the one or more exogenous nucleic acids comprise a sequence encoding for an immune checkpoint inhibitor. Embodiment 20. The composition of any preceding embodiment, further comprising a gene editing system or a gene editing agent provided herein. Embodiment 21. The composition of any preceding embodiment, further comprising one or more guide RNAs. Embodiment 22. The composition of any preceding embodiment, wherein the engineered cell comprises one or more genomic disruptions in a target sequence of a gene. Embodiment 23. The composition of any preceding embodiment, wherein the genomic disruption is in a target sequence of a dCK gene, a beta-2 microglobulin gene, a class II major histocompatibility complex transactivator (CIITA) gene, a programmed cell death 1 (PD1) gene, and T cell receptor constant region (TRAC). Embodiment 24. The composition of any preceding embodiment, wherein the composition further comprises a chemotherapeutic agent.

Embodiment 25. A composition comprising: an engineered cell comprising one or more exogenous nucleic acids, wherein at least one exogenous nucleic acid comprises a sequence that is at least 80% identical, at least 85% identical, at least 90% identical, at least 95% identical, at least 96% identical, at least 97% identical, at least 98% identical, at least 99% identical, or is 100% identical to any one of SEQ ID NO: 1, SEQ ID NO: 2, SEQ ID NO: 8, SEQ ID NO: 9, SEQ ID NO: 15, SEQ ID NO: 16, SEQ ID NO: 19, SEQ ID NO: 20. Embodiment 26. The composition of any preceding embodiment, wherein the one or more exogenous nucleic acids comprise a sequence encoding for a T cell receptor (TCR). Embodiment 27. The composition of any preceding embodiment, wherein the one or more exogenous nucleic acids comprise a sequence encoding for a chimeric antigen receptor (CAR). Embodiment 28. The composition of any preceding embodiment, wherein the one or more exogenous nucleic acids comprise a sequence encoding for an immune checkpoint inhibitor. Embodiment 29. The composition of any preceding embodiment, further comprising a gene editing system or a gene editing agent provided herein. Embodiment 30. The composition of any preceding embodiment, further comprising one or more guide RNAs. Embodiment 31. The composition of any preceding embodiment, wherein the engineered cell comprises one or more genomic disruptions in a target sequence of a gene. Embodiment 32. The composition of any preceding embodiment, wherein the genomic disruption is in a target sequence of a dCK gene, a beta-2 microglobulin gene, a class II major histocompatibility complex transactivator (CIITA) gene, a programmed cell death 1 (PD1) gene, and T cell receptor constant region (TRAC). Embodiment 33. The composition of any preceding embodiment, wherein the composition further comprises a chemotherapeutic agent.

Embodiment 34. A composition comprising: an engineered cell comprising one or more exogenous nucleic acids, wherein at least one exogenous nucleic acid comprises a sequence that has complementarity to any one of SEQ ID NO: 27 or SEQ ID NO: 28. Embodiment 35. The composition of embodiment 34, wherein the composition further comprises a gene editing system or a gene editing agent provided herein. Embodiment 36. The composition of embodiment 35, wherein the gene editing system or the gene editing agent comprises a CRISPR/Cas gene editing system, a Cas nuclease, a nickase, a base editor, a DNA-polymerase, a reverse transcriptase, an RNA polymerase, or any combination thereof. Embodiment 37. The composition of any preceding embodiment, wherein the one or more exogenous nucleic acids comprise a sequence encoding for a T cell receptor (TCR). Embodiment 38. The composition of any preceding embodiment, wherein the one or more exogenous nucleic acids comprise a sequence encoding for a chimeric antigen receptor (CAR). Embodiment 39. The composition of any preceding embodiment, wherein the one or more exogenous nucleic acids comprise a sequence encoding for an immune checkpoint inhibitor. Embodiment 40. The composition of any preceding embodiment, further comprising a gene editing system. Embodiment 41. The composition of any preceding embodiment, further comprising one or more guide RNAs. Embodiment 42. The composition of any preceding embodiment, wherein the engineered cell comprises one or more genomic disruptions in a target sequence of a gene. Embodiment 43. The composition of any preceding embodiment, wherein the genomic disruption is in a target sequence of a dCK gene, a beta-2 microglobulin gene, a class II major histocompatibility complex transactivator (CIITA) gene, a programmed cell death 1 (PD1) gene, and T cell receptor constant region (TRAC). Embodiment 44. The composition of any preceding embodiment, wherein the composition further comprises a chemotherapeutic agent.

Embodiment 45. A composition comprising: an engineered cell comprising one or more exogenous nucleic acids, wherein at least one exogenous nucleic acid encodes for an aminoglycoside-3′-phosphotransferase-IIa (APH(3′)-IIa) protein, wherein the engineered cell is resistant to aminoglycoside-mediated cell death relative to a comparable cell that does not have an exogenous nucleic acid encoding for an aminoglycoside-3′-phosphotransferase-IIa (APH(3′)-IIa) protein. Embodiment 46. The composition of any preceding embodiment, wherein the engineered cell is a human leukocyte, a human stem cell, or an in vitro-differentiated cell. Embodiment 47. The composition of any preceding embodiment, wherein the engineered cell expresses at least a 10% increase, at least a 20% increase, at least a 30% increase, at least a 40% increase, at least a 50% increase, at least a 60% increase, at least a 70% increase, or at least an 80% increase in the level of aminoglycoside-3′-phosphotransferase-IIa (APH(3′)-IIa protein relative to a comparable cell that does not have an exogenous nucleic acid encoding for aminoglycoside-3′-phosphotransferase-IIa (APH(3′)-IIa) protein. Embodiment 48. The composition of any preceding embodiment, wherein the engineered cell is an ex vivo-engineered cell, an in vivo-engineered cell, or an in vitro-engineered cell. Embodiment 49. The composition of any preceding embodiment, wherein the human leukocyte is a human T cell, a human natural killer (NK) cell, a human natural killer T cell (NKT), a human macrophage, a human B cell, a human dendritic cell, or a human tumor infiltrating lymphocyte (TIL). Embodiment 50. The composition of any preceding embodiment, wherein the human T cell is a CD3 positive (CD3+) T cell. Embodiment 51. The composition of any preceding embodiment, wherein the human TIL is a CD3 positive (CD3+) TIL. Embodiment 52. The composition of any preceding embodiment, wherein the human stem cell comprises an adult stem cell. Embodiment 53. The composition of any preceding embodiment, wherein the human stem cell comprises an induced pluripotent stem cell (iPSC). Embodiment 54. The composition of any preceding embodiment, wherein the human stem cell comprises an embryonic stem cell. Embodiment 55. The composition of any preceding embodiment, wherein the in vitro-differentiated cell comprises an iPSC-derived natural killer T cell (iNKT), an iPSC-derived natural killer cell, an iPSC-derived T cell, an iPSC-derived macrophage, or an iPSC-derived B cell. Embodiment 56. The composition of any preceding embodiment, wherein the one or more exogenous nucleic acids comprise a DNA, an RNA, or a combination thereof. Embodiment 57. The composition of any preceding embodiment, wherein the RNA comprises a messenger RNA (mRNA), a guide RNA (gRNA), a microRNA (miRNA), a tRNA, a CRISPR RNA (crRNA), a short hairpin RNA (shRNA), a silencing RNA (siRNA), an antisense oligonucleotide, a non-coding RNA, an aptamer, or a combination thereof. Embodiment 58. The composition of any preceding embodiment, wherein the DNA comprises a transgene encoding a protein, a regulatory element, a promoter, an enhancer, a repressor, an inhibitor, an antibiotic resistance gene, or a non-coding DNA. Embodiment 59. The composition of any preceding embodiment, wherein the one or more nucleic acids further comprise a poly-A tail or a nucleoside modification. Embodiment 60. The composition of any preceding embodiment, wherein the exogenous nucleic acid comprises a sequence that is at least 80% identical, at least 85% identical, at least 90% identical, at least 95% identical, at least 96% identical, at least 97% identical, at least 98% identical, at least 99% identical, or is 100% identical to SEQ ID NO: 1. Embodiment 61. The composition of any preceding embodiment, wherein the exogenous nucleic acid comprises a sequence that is at least 80% identical, at least 85% identical, at least 90% identical, at least 95% identical, at least 96% identical, at least 97% identical, at least 98% identical, at least 99% identical, or is 100% identical to SEQ ID NO: 2. Embodiment 62. The composition of any preceding embodiment, wherein the exogenous nucleic acid encodes for an amino acid sequence that is at least 80% identical, at least 85% identical, at least 90% identical, at least 95% identical, at least 96% identical, at least 97% identical, at least 98% identical, at least 99% identical, or is 100% identical to any one of SEQ ID NO: 3-SEQ ID NO: 7. Embodiment 63. The composition of any preceding embodiment, wherein the genomic disruption is in a target sequence of a dCK gene, a beta-2 microglobulin gene, a class II major histocompatibility complex transactivator (CIITA) gene, a programmed cell death 1 (PD1) gene, and T cell receptor constant region (TRAC). Embodiment 64. The composition of any preceding embodiment, wherein the composition further comprises a chemotherapeutic agent.

Embodiment 65. A composition comprising: an engineered cell comprising one or more exogenous nucleic acids, wherein at least one exogenous nucleic acid encodes for a protein kinase cAMP-activated catalytic subunit alpha (PRKACA) protein, wherein the engineered cell is resistant to aminoglycoside-mediated cell death relative to a comparable cell that does not have an exogenous nucleic acid encoding for protein kinase cAMP-activated catalytic subunit alpha (PRKACA) protein. Embodiment 66. The composition of any preceding embodiment, wherein the engineered cell is a human leukocyte, a human stem cell, or an in vitro-differentiated cell. Embodiment 67. The composition of any preceding embodiment, wherein the engineered cell expresses at least a 10% increase, at least a 20% increase, at least a 30% increase, at least a 40% increase, at least a 50% increase, at least a 60% increase, at least a 70% increase, or at least an 80% increase in the level of PRKACA protein relative to a comparable cell that does not have an exogenous nucleic acid encoding for a PRKACA protein. Embodiment 68. The composition of any preceding embodiment, wherein the engineered cell is an ex vivo-engineered cell, an in vivo-engineered cell, or an in vitro-engineered cell. Embodiment 69. The composition of any preceding embodiment, wherein the human leukocyte is a human T cell, a human natural killer (NK) cell, a human natural killer T cell (NKT), a human macrophage, a human B cell, a human dendritic cell, or a human tumor infiltrating lymphocyte (TIL). Embodiment 70. The composition of any preceding embodiment, wherein the human T cell is a CD3 positive (CD3+) T cell. Embodiment 71. The composition of any preceding embodiment, wherein the human TIL is a CD3 positive (CD3+) TIL. Embodiment 72. The composition of any preceding embodiment, wherein the human stem cell comprises an adult stem cell. Embodiment 73. The composition of any preceding embodiment, wherein the human stem cell comprises an induced pluripotent stem cell (iPSC). Embodiment 74. The composition of any preceding embodiment, wherein the human stem cell comprises an embryonic stem cell. Embodiment 75. The composition of any preceding embodiment, wherein the in vitro-differentiated cell comprises an iPSC-derived natural killer T cell (iNKT), an iPSC-derived natural killer cell, an iPSC-derived T cell, an iPSC-derived macrophage, or an iPSC-derived B cell. Embodiment 76. The composition of any preceding embodiment, wherein the one or more exogenous nucleic acids comprise a DNA, an RNA, or a combination thereof. Embodiment 77. The composition of any preceding embodiment, wherein the RNA comprises a messenger RNA (mRNA), a guide RNA (gRNA), a microRNA (miRNA), a tRNA, a CRISPR RNA (crRNA), a short hairpin RNA (shRNA), a silencing RNA (siRNA), an antisense oligonucleotide, a non-coding RNA, an aptamer, or a combination thereof. Embodiment 78. The composition of any preceding embodiment, wherein the DNA comprises a transgene encoding a protein, a regulatory element, a promoter, an enhancer, a repressor, an inhibitor, an antibiotic resistance gene, or a non-coding DNA. Embodiment 79. The composition of any preceding embodiment, wherein the at least one exogenous nucleic acid comprises a sequence that is at least 80% identical, at least 85% identical, at least 90% identical, at least 95% identical, at least 96% identical, at least 97% identical, at least 98% identical, at least 99% identical, or 100% identical to SEQ ID NO: 8. Embodiment 80. The composition of any preceding embodiment, wherein the at least one exogenous nucleic acid comprises a sequence that is at least 80% identical, at least 85% identical, at least 90% identical, at least 95% identical, at least 96% identical, at least 97% identical, at least 98% identical, at least 99% identical, or 100% identical to SEQ ID NO: 9. Embodiment 81. The composition of any preceding embodiment, wherein the at least one exogenous nucleic acid encodes for an amino acid sequence that is at least 80% identical, at least 85% identical, at least 90% identical, at least 95% identical, at least 96% identical, at least 97% identical, at least 98% identical, at least 99% identical, or 100% identical to SEQ ID NO: 10-SEQ ID NO: 14. Embodiment 82. The composition of any preceding embodiment, wherein the one or more exogenous nucleic acids comprise a sequence encoding for a T cell receptor (TCR). Embodiment 83. The composition of any preceding embodiment, wherein the one or more exogenous nucleic acids comprise a sequence encoding for a chimeric antigen receptor (CAR). Embodiment 84. The composition of any preceding embodiment, wherein the one or more exogenous nucleic acids comprise a sequence encoding for an immune checkpoint inhibitor. Embodiment 85. The composition of any preceding embodiment, further comprising a gene editing system or a gene editing agent provided herein. Embodiment 86. The composition of any preceding embodiment, further comprising one or more guide RNAs. Embodiment 87. The composition of any preceding embodiment, wherein the engineered cell comprises one or more genomic disruptions in a target sequence of a gene. Embodiment 88. The composition of any preceding embodiment, wherein the genomic disruption is in a target sequence of a dCK gene, a beta-2 microglobulin gene, a class II major histocompatibility complex transactivator (CIITA) gene, a programmed cell death 1 (PD1) gene, and T cell receptor constant region (TRAC). Embodiment 89. The composition of any preceding embodiment, wherein the composition further comprises a chemotherapeutic agent.

Embodiment 90. A composition comprising: an engineered cell comprising one or more exogenous nucleic acids, wherein at least one exogenous nucleic acid encodes for an aldehyde dehydrogenase (ALDH) protein, wherein the engineered cell is resistant to alkylating agent-mediated cell death relative to a comparable cell that does not have an exogenous nucleic acid encoding for an aldehyde dehydrogenase (ALDH) protein. Embodiment 91. The composition of any preceding embodiment, wherein the engineered cell is a mammalian cell. Embodiment 92. The composition of any preceding embodiment, wherein the engineered cell expresses at least a 10% increase, at least a 20% increase, at least a 30% increase, at least a 40% increase, at least a 50% increase, at least a 60% increase, at least a 70% increase, or at least an 80% increase in the level of the ALDH protein relative to a comparable cell that does not have an exogenous nucleic acid encoding for a ALDH protein. Embodiment 92. The composition of any preceding embodiment, wherein the engineered cell is an ex vivo-engineered cell, an in vivo-engineered cell, or an in vitro-engineered cell. Embodiment 93. The composition of any preceding embodiment, wherein the engineered cell comprises a human leukocyte, a human stem cell, or an in vitro-differentiated cell. Embodiment 94. The composition of any preceding embodiment, wherein the human leukocyte is a human T cell, a human natural killer (NK) cell, a human natural killer T cell (NKT), a human macrophage, a human B cell, a human dendritic cell, or a human tumor infiltrating lymphocyte (TIL). The composition of any preceding embodiment, wherein the human T cell is a CD3 positive (CD3+) T cell. Embodiment 95. The composition of any preceding embodiment, wherein the human TIL is a CD3 positive (CD3+) TIL. Embodiment 96. The composition of any preceding embodiment, wherein the human stem cell comprises an adult stem cell. Embodiment 97. The composition of any preceding embodiment, wherein the human stem cell comprises an induced pluripotent stem cell (iPSC). Embodiment 98. The composition of any preceding embodiment, wherein the human stem cell comprises an embryonic stem cell. Embodiment 99. The composition of any preceding embodiment, wherein the in vitro-differentiated cell comprises an iPSC-derived natural killer T cell (iNKT), an iPSC-derived natural killer cell, an iPSC-derived T cell, an iPSC-derived macrophage, or an iPSC-derived B cell. Embodiment 100. The composition of any preceding embodiment, wherein the one or more exogenous nucleic acids comprise a DNA, an RNA, or a combination thereof. Embodiment 101. The composition of any preceding embodiment, wherein the RNA comprises a messenger RNA (mRNA), a guide RNA (gRNA), a microRNA (miRNA), a tRNA, a CRISPR RNA (crRNA), a short hairpin RNA (shRNA), a silencing RNA (siRNA), an antisense oligonucleotide, a non-coding RNA, an aptamer, or a combination thereof. Embodiment 102. The composition of any preceding embodiment, wherein the DNA comprises a transgene encoding a protein, a regulatory element, a promoter, an enhancer, a repressor, an inhibitor, an antibiotic resistance gene, or a non-coding DNA. Embodiment 103. The composition of any preceding embodiment, wherein the aldehyde dehydrogenase protein comprises an aldehyde dehydrogenase 1 family member A1 protein. Embodiment 104. The composition of any preceding embodiment, wherein the aldehyde dehydrogenase protein comprises an aldehyde dehydrogenase 1 family member A2 protein. Embodiment 105. The composition of any preceding embodiment, wherein the aldehyde dehydrogenase protein comprises an aldehyde dehydrogenase 1 family member B1 protein. Embodiment 106. The composition of any preceding embodiment, wherein the aldehyde dehydrogenase protein comprises an aldehyde dehydrogenase 2 family member protein. Embodiment 107. The composition of any preceding embodiment, wherein the aldehyde dehydrogenase protein comprises an aldehyde dehydrogenase 3 family member A1 protein. Embodiment 108. The composition of any preceding embodiment, wherein the at least one exogenous nucleic acid comprises a sequence that is at least 80% identical, at least 85% identical, at least 90% identical, at least 95% identical, at least 96% identical, at least 97% identical, at least 98% identical, at least 99% identical, or 100% identical to SEQ ID NO: 15. Embodiment 109. The composition of any preceding embodiment, wherein the at least one exogenous nucleic acid comprises a sequence that is at least 80% identical, at least 85% identical, at least 90% identical, at least 95% identical, at least 96% identical, at least 97% identical, at least 98% identical, at least 99% identical, or 100% identical to SEQ ID NO: 16. Embodiment 110. The composition of any preceding embodiment, wherein the at least one exogenous nucleic acid encodes for an amino acid sequence that is at least 80% identical, at least 85% identical, at least 90% identical, at least 95% identical, at least 96% identical, at least 97% identical, at least 98% identical, at least 99% identical, or 100% identical to SEQ ID NO: 17 or SEQ ID NO: 18. Embodiment 111. The composition of any preceding embodiment, wherein the one or more exogenous nucleic acids comprise a sequence encoding for a T cell receptor (TCR). Embodiment 112. The composition of any preceding embodiment, wherein the one or more exogenous nucleic acids comprise a sequence encoding for a chimeric antigen receptor (CAR). Embodiment 113. The composition of any preceding embodiment, wherein the one or more exogenous nucleic acids comprise a sequence encoding for an immune checkpoint inhibitor. Embodiment 114. The composition of any preceding embodiment, further comprising a gene editing system or a gene editing agent provided herein. Embodiment 115. The composition of any preceding embodiment, further comprising one or more guide RNAs. Embodiment 116. The composition of any preceding embodiment, wherein the engineered cell comprises one or more genomic disruptions in a target sequence of a gene. Embodiment 117. The composition of any preceding embodiment, wherein the genomic disruption is in a target sequence of a dCK gene, a beta-2 microglobulin gene, a class II major histocompatibility complex transactivator (CIITA) gene, a programmed cell death 1 (PD1) gene, and T cell receptor constant region (TRAC). Embodiment 118. The composition of any preceding embodiment, wherein the composition further comprises a chemotherapeutic agent.

Embodiment 119. A composition comprising: an engineered cell comprising one or more exogenous nucleic acids, wherein at least one exogenous nucleic acid encodes for a glutathione S transferase A4 (GSTA4) protein, wherein the engineered cell is resistant to alkylating agent-mediated cell death relative to a comparable cell that does not have an exogenous nucleic acid encoding for glutathione S transferase A4 (GSTA4). Embodiment 120. The composition of any preceding embodiment, wherein the engineered cell is a human leukocyte, a human stem cell, or an in vitro-differentiated cell. Embodiment 121. The composition of any preceding embodiment, wherein the engineered cell expresses at least a 10% increase, at least a 20% increase, at least a 30% increase, at least a 40% increase, at least a 50% increase, at least a 60% increase, at least a 70% increase, or at least an 80% increase in the level of GSTA4 protein relative to a comparable cell that does not have an exogenous nucleic acid encoding for a GSTA4 protein. Embodiment 122. The composition of any preceding embodiment, wherein the engineered cell is an ex vivo-engineered cell, an in vivo-engineered cell, or an in vitro-engineered cell. Embodiment 123. The composition of any preceding embodiment, wherein the human leukocyte is a human T cell, a human natural killer (NK) cell, a human natural killer T cell (NKT), a human macrophage, a human B cell, a human dendritic cell, or a human tumor infiltrating lymphocyte (TIL). Embodiment 124. The composition of any preceding embodiment, wherein the human T cell is a CD3 positive (CD3+) T cell. Embodiment 125. The composition of any preceding embodiment, wherein the human TIL is a CD3 positive (CD3+) TIL. Embodiment 125. The composition of any preceding embodiment, wherein the human stem cell comprises an adult stem cell. Embodiment 126. The composition of any preceding embodiment, wherein the human stem cell comprises an induced pluripotent stem cell (iPSC). Embodiment 127. The composition of any preceding embodiment, wherein the human stem cell comprises an embryonic stem cell. Embodiment 128. The composition of any preceding embodiment, wherein the in vitro-differentiated cell comprises an iPSC-derived natural killer T cell (iNKT), an iPSC-derived natural killer cell, an iPSC-derived T cell, an iPSC-derived macrophage, or an iPSC-derived B cell. Embodiment 129. The composition of any preceding embodiment, wherein the one or more exogenous nucleic acids comprise a DNA, an RNA, or a combination thereof. Embodiment 130. The composition of any preceding embodiment, wherein the RNA comprises a messenger RNA (mRNA), a guide RNA (gRNA), a microRNA (miRNA), a tRNA, a CRISPR RNA (crRNA), a short hairpin RNA (shRNA), a silencing RNA (siRNA), an antisense oligonucleotide, a non-coding RNA, an aptamer, or a combination thereof. Embodiment 131. The composition of any preceding embodiment, wherein the DNA comprises a transgene encoding a protein, a regulatory element, a promoter, an enhancer, a repressor, an inhibitor, an antibiotic resistance gene, or a non-coding DNA. Embodiment 132. The composition of any preceding embodiment, wherein the at least one exogenous nucleic acid comprises a sequence that is at least 80% identical, at least 85% identical, at least 90% identical, at least 95% identical, at least 96% identical, at least 97% identical, at least 98% identical, at least 99% identical, or 100% identical to SEQ ID NO: 19. Embodiment 133. The composition of any preceding embodiment, wherein the at least one exogenous nucleic acid comprises a sequence that is at least 80% identical, at least 85% identical, at least 90% identical, at least 95% identical, at least 96% identical, at least 97% identical, at least 98% identical, at least 99% identical, or 100% identical to SEQ ID NO: 20. Embodiment 134. The composition of any preceding embodiment, wherein the at least one exogenous nucleic acid encodes for an amino acid sequence that is at least 80% identical, at least 85% identical, at least 90% identical, at least 95% identical, at least 96% identical, at least 97% identical, at least 98% identical, at least 99% identical, or 100% identical to SEQ ID NO: 21 or SEQ ID NO: 22. Embodiment 135. The composition of any preceding embodiment, wherein the one or more exogenous nucleic acids comprise a sequence encoding for a T cell receptor (TCR). Embodiment 136. The composition of any preceding embodiment, wherein the one or more exogenous nucleic acids comprise a sequence encoding for a chimeric antigen receptor (CAR). Embodiment 137. The composition of any preceding embodiment, wherein the one or more exogenous nucleic acids comprise a sequence encoding for an immune checkpoint inhibitor. Embodiment 138. The composition of any preceding embodiment, further comprising a gene editing system or a gene editing agent provided herein. Embodiment 139. The composition of any preceding embodiment, further comprising one or more guide RNAs. Embodiment 140. The composition of any preceding embodiment, wherein the engineered cell comprises one or more genomic disruptions in a target sequence of a gene. Embodiment 141. The composition of any preceding embodiment, wherein the genomic disruption is in a target sequence of a dCK gene, a beta-2 microglobulin gene, a class II major histocompatibility complex transactivator (CIITA) gene, a programmed cell death 1 (PD1) gene, and T cell receptor constant region (TRAC).

Embodiment 142. A composition comprising: a CD3 positive (CD3+) T cell comprising a genomic disruption in a target sequence of a deoxycytidine kinase (dCK) gene. The composition of any preceding embodiment, wherein the genomic disruption is a CRISPR-mediated genomic disruption. The composition of any preceding embodiment, wherein the target sequence comprises at least 10 contiguous nucleotides of SEQ ID NO: 27. The composition of any preceding embodiment, wherein the target sequence comprises at least 15 contiguous nucleotides of SEQ ID NO: 27. The composition of any preceding embodiment, wherein the target sequence comprises at least 18 contiguous nucleotides of SEQ ID NO: 27. The composition of any preceding embodiment, wherein the target sequence comprises at least 19 contiguous nucleotides of SEQ ID NO: 27. The composition of any preceding embodiment, wherein the target sequence comprises at least 20 contiguous nucleotides of SEQ ID NO: 27. The composition of any preceding embodiment, wherein the target sequence is at least 20 base pairs from a protospacer adjacent motif (PAM). The composition of any preceding embodiment, wherein the target sequence is at least 10 base pairs from a PAM. The composition of any preceding embodiment, wherein the composition further comprises a chemotherapeutic agent.

Embodiment 143. A composition comprising: a CD3 positive (CD3+) T cell comprising an siRNA-mediated disruption in a target sequence of a deoxycytidine kinase (dCK) RNA, wherein the CD3+ T cell has a reduced level of dCK RNA or dCK protein relative to a comparable CD3+ T cell that does not have an siRNA-mediated disruption. The composition of any preceding embodiment, the target sequence of dCK comprises at least 15 contiguous nucleotides of SEQ ID NO: 28. The composition of any preceding embodiment, wherein the one or more exogenous nucleic acids comprise a sequence encoding for a T cell receptor (TCR). The composition of any preceding embodiment, wherein the one or more exogenous nucleic acids comprise a sequence encoding for a chimeric antigen receptor (CAR). The composition of any preceding embodiment, wherein the one or more exogenous nucleic acids comprise a sequence encoding for an immune checkpoint inhibitor. The composition of any preceding embodiment, further comprising a gene editing system or a gene editing agent provided herein. The composition of any preceding embodiment, further comprising one or more guide RNAs. The composition of any preceding embodiment, wherein the engineered cell comprises one or more genomic disruptions in a target sequence of a gene. The composition of any preceding embodiment, wherein the genomic disruption is in a target sequence of a dCK gene, a beta-2 microglobulin gene, a class II major histocompatibility complex transactivator (CIITA) gene, a programmed cell death 1 (PD1) gene, and T cell receptor constant region (TRAC). The composition of any preceding embodiment, wherein the composition further comprises a chemotherapeutic agent.

Embodiment 144. A composition comprising: a carrier; and one or more nucleic acids, wherein at least one nucleic acid encodes for protein kinase cAMP-activated catalytic subunit alpha (PRKACA) protein. A composition comprising: a carrier; and one or more nucleic acids, wherein at least one nucleic acid comprises a sequence that is at least 80% identical, at least 85% identical, at least 90% identical, at least 95% identical, at least 96% identical, at least 97% identical, at least 98% identical, at least 99% identical, or 100% identical to SEQ ID NO: 8. A composition comprising: a carrier; and one or more nucleic acids, wherein at least one nucleic acid comprises a sequence that is at least 80% identical, at least 85% identical, at least 90% identical, at least 95% identical, at least 96% identical, at least 97% identical, at least 98% identical, at least 99% identical, or 100% identical to SEQ ID NO: 9. The composition of any preceding embodiment, wherein the at least one nucleic acid is comprised in a vector. The composition of any preceding embodiment, wherein the carrier comprises a lipid nanoparticle, a cationic nanoparticle, an inorganic nanoparticle, a virus-like particle, an extracellular vesicle, a liposome, a polymer, a viral vector, a microcapsule, a lipid emulsion, a virosome, a micelle, an exosome, an endosome, a dendrimer, a cell-penetrating peptide, or a metal. The composition of any preceding embodiment, wherein the one or more nucleic acids are comprised within a minicircle vector or a non-integrating plasmid. The composition of any preceding embodiment, wherein the one or more nucleic acids comprise DNA, RNA, or a combination thereof. The composition of any preceding embodiment, wherein the composition further comprises a chemotherapeutic agent.

Embodiment 145. A composition comprising: a carrier; and one or more nucleic acids, wherein at least one nucleic acid encodes for an ALDH protein selected from the group consisting of: an aldehyde dehydrogenase 1 family member A1 protein, an aldehyde dehydrogenase 1 family member B1 protein, an aldehyde dehydrogenase 2 family member protein, and an aldehyde dehydrogenase 3 family member A1 protein. A composition comprising: a carrier; and one or more nucleic acids, wherein at least one nucleic acid comprises a sequence that is at least 80% identical, at least 85% identical, at least 90% identical, at least 95% identical, at least 96% identical, at least 97% identical, at least 98% identical, at least 99% identical, or 100% identical to SEQ ID NO: 15. A composition comprising: a carrier; and one or more nucleic acids, wherein at least one nucleic acid comprises a sequence that is at least 80% identical, at least 85% identical, at least 90% identical, at least 95% identical, at least 96% identical, at least 97% identical, at least 98% identical, at least 99% identical, or 100% identical to SEQ ID NO: 16. The composition of any preceding embodiment, wherein the at least one nucleic acid is comprised in a vector. The composition of any preceding embodiment, wherein the carrier comprises a lipid nanoparticle, a cationic nanoparticle, an inorganic nanoparticle, a virus-like particle, an extracellular vesicle, a liposome, a polymer, a viral vector, a microcapsule, a lipid emulsion, a virosome, a micelle, an exosome, an endosome, a dendrimer, a cell-penetrating peptide, or a metal. The composition of any preceding embodiment, wherein the one or more nucleic acids are comprised within a minicircle vector or a non-integrating plasmid. The composition of any preceding embodiment, wherein the one or more nucleic acids comprise DNA, RNA, or a combination thereof. The composition of any preceding embodiment, wherein the composition further comprises a chemotherapeutic agent.

Embodiment 146. A composition comprising: a carrier; and one or more nucleic acids, wherein at least one nucleic acid encodes for a GSTA4 protein. A composition comprising: a carrier; and one or more nucleic acids, wherein at least one nucleic acid comprises a sequence that is at least 80% identical, at least 85% identical, at least 90% identical, at least 95% identical, at least 96% identical, at least 97% identical, at least 98% identical, at least 99% identical, or 100% identical to SEQ ID NO: 19. A composition comprising: a carrier; and one or more nucleic acids, wherein at least one nucleic acid comprises a sequence that is at least 80% identical, at least 85% identical, at least 90% identical, at least 95% identical, at least 96% identical, at least 97% identical, at least 98% identical, at least 99% identical, or 100% identical to SEQ ID NO: 20. The composition of any preceding embodiment, wherein the at least one nucleic acid is comprised in a vector. The composition of any preceding embodiment, wherein the carrier comprises a lipid nanoparticle, a cationic nanoparticle, an inorganic nanoparticle, a virus-like particle, an extracellular vesicle, a liposome, a polymer, a viral vector, a microcapsule, a lipid emulsion, a virosome, a micelle, an exosome, an endosome, a dendrimer, a cell-penetrating peptide, or a metal. The composition of any preceding embodiment, wherein the one or more nucleic acids are comprised within a minicircle vector or a non-integrating plasmid. The composition of any preceding embodiment, wherein the one or more nucleic acids comprise DNA, RNA, or a combination thereof. The composition of any preceding embodiment, wherein the composition further comprises a chemotherapeutic agent.

Embodiment 147. A composition comprising: a carrier; and one or more nucleic acids, wherein at least one nucleic acid encodes for aminoglycoside-3′-phosphotransferase-IIa (APH(3′)-IIa) protein. A composition comprising: a carrier; and one or more nucleic acids, wherein at least one nucleic acid comprises a sequence that is at least 80% identical, at least 85% identical, at least 90% identical, at least 95% identical, at least 96% identical, at least 97% identical, at least 98% identical, at least 99% identical, or 100% identical to SEQ ID NO: 1. A composition comprising: a carrier; and one or more nucleic acids, wherein at least one nucleic acid comprises a sequence that is at least 80% identical, at least 85% identical, at least 90% identical, at least 95% identical, at least 96% identical, at least 97% identical, at least 98% identical, at least 99% identical, or 100% identical to SEQ ID NO: 2. The composition of any preceding embodiment, wherein the at least one nucleic acid is comprised in a vector. The composition of any preceding embodiment, wherein the carrier comprises a lipid nanoparticle, a cationic nanoparticle, an inorganic nanoparticle, a virus-like particle, an extracellular vesicle, a liposome, a polymer, a viral vector, a microcapsule, a lipid emulsion, a virosome, a micelle, an exosome, an endosome, a dendrimer, a cell-penetrating peptide, or a metal. The composition of any preceding embodiment, wherein the one or more nucleic acids are comprised within a minicircle vector or a non-integrating plasmid. The composition of any preceding embodiment, wherein the one or more nucleic acids comprise DNA, RNA, or a combination thereof. The composition of any preceding embodiment, wherein the composition further comprises a chemotherapeutic agent.

Embodiment 148. A composition comprising: a carrier; and a vector comprising: one or more nucleic acids, wherein at least one nucleic acid encodes for protein kinase cAMP-activated catalytic subunit alpha (PRKACA) protein, wherein the vector comprises a lentiviral vector, an adenoviral vector, an adeno-associated viral (AAV) vector, or a retroviral vector. A composition comprising: a carrier; and a vector comprising: a nucleic acid comprising a sequence is at least 80% identical, at least 85% identical, at least 90% identical, at least 95% identical, at least 96% identical, at least 97% identical, at least 98% identical, at least 99% identical, or 100% identical to SEQ ID NO: 8, SEQ ID NO: 9, or SEQ ID NO: 24. The composition of any preceding embodiment, wherein the carrier comprises a lipid nanoparticle, a cationic nanoparticle, an inorganic nanoparticle, a virus-like particle, an extracellular vesicle, a liposome, a polymer, a viral vector, a microcapsule, a lipid emulsion, a virosome, a micelle, an exosome, an endosome, a dendrimer, a cell-penetrating peptide, or a metal. The composition of any preceding embodiment, wherein the one or more nucleic acids are comprised within a minicircle vector or a non-integrating plasmid. The composition of any preceding embodiment, wherein the one or more nucleic acids comprise DNA, RNA, or a combination thereof. The composition of any preceding embodiment, wherein the composition further comprises a chemotherapeutic agent.

Embodiment 149. A composition comprising: a carrier; and a vector comprising: one or more nucleic acids, wherein at least one nucleic acid encodes for a GSTA4 protein, wherein the vector comprises a lentiviral vector, an adenoviral vector, an adeno-associated viral (AAV) vector, or a retroviral vector. A composition comprising: a carrier; and a vector comprising: a nucleic acid comprising a sequence is at least 80% identical, at least 85% identical, at least 90% identical, at least 95% identical, at least 96% identical, at least 97% identical, at least 98% identical, at least 99% identical, or 100% identical to SEQ ID NO: 19, SEQ ID NO: 20, or SEQ ID NO: 26. The composition of any preceding embodiment, wherein the carrier comprises a lipid nanoparticle, a cationic nanoparticle, an inorganic nanoparticle, a virus-like particle, an extracellular vesicle, a liposome, a polymer, a viral vector, a microcapsule, a lipid emulsion, a virosome, a micelle, an exosome, an endosome, a dendrimer, a cell-penetrating peptide, or a metal. The composition of any preceding embodiment, wherein the one or more nucleic acids are comprised within a minicircle vector or a non-integrating plasmid. The composition of any preceding embodiment, wherein the one or more nucleic acids comprise DNA, RNA, or a combination thereof. The composition of any preceding embodiment, wherein the composition further comprises a chemotherapeutic agent.

Embodiment 150. A composition comprising: a carrier; and a vector comprising: one or more nucleic acids, wherein at least one nucleic acid encodes for aminoglycoside-3′-phosphotransferase-IIa (APH(3′)-IIa) protein, wherein the vector comprises a lentiviral vector, an adenoviral vector, an adeno-associated viral (AAV) vector, or a retroviral vector. A composition comprising: a carrier; and a vector comprising: a nucleic acid comprising a sequence is at least 80% identical, at least 85% identical, at least 90% identical, at least 95% identical, at least 96% identical, at least 97% identical, at least 98% identical, at least 99% identical, or 100% identical to SEQ ID NO: 1, SEQ ID NO: 2, or SEQ ID NO: 23. The composition of any preceding embodiment, wherein the carrier comprises a lipid nanoparticle, a cationic nanoparticle, an inorganic nanoparticle, a virus-like particle, an extracellular vesicle, a liposome, a polymer, a viral vector, a microcapsule, a lipid emulsion, a virosome, a micelle, an exosome, an endosome, a dendrimer, a cell-penetrating peptide, or a metal. The composition of any preceding embodiment, wherein the one or more nucleic acids are comprised within a minicircle vector or a non-integrating plasmid. The composition of any preceding embodiment, wherein the one or more nucleic acids comprise DNA, RNA, or a combination thereof. The composition of any preceding embodiment, wherein the composition further comprises a chemotherapeutic agent.

Embodiment 151. A composition comprising: a carrier; and a vector comprising: one or more nucleic acids, wherein at least one nucleic acid encodes for an ALDH protein selected from the group consisting of: an aldehyde dehydrogenase 1 family member A1 protein, an aldehyde dehydrogenase 1 family member B1 protein, an aldehyde dehydrogenase 2 family member protein, and an aldehyde dehydrogenase 3 family member A1 protein, wherein the vector comprises a lentiviral vector, an adenoviral vector, an adeno-associated viral (AAV) vector, or a retroviral vector. The composition of any preceding embodiment, wherein the ALDH protein is an aldehyde dehydrogenase 3 family member A1 protein (ALDH3A1). A composition comprising: a carrier; and a vector comprising: a nucleic acid comprising a sequence is at least 80% identical, at least 85% identical, at least 90% identical, at least 95% identical, at least 96% identical, at least 97% identical, at least 98% identical, at least 99% identical, or 100% identical to SEQ ID NO: 15, SEQ ID NO: 16, or SEQ ID NO: 25. The composition of any preceding embodiment, wherein the carrier comprises a lipid nanoparticle, a cationic nanoparticle, an inorganic nanoparticle, a virus-like particle, an extracellular vesicle, a liposome, a polymer, a viral vector, a microcapsule, a lipid emulsion, a virosome, a micelle, an exosome, an endosome, a dendrimer, a cell-penetrating peptide, or a metal. The composition of any preceding embodiment, wherein the one or more nucleic acids are comprised within a minicircle vector or a non-integrating plasmid. The composition of any preceding embodiment, wherein the one or more nucleic acids comprise DNA, RNA, or a combination thereof. The composition of any preceding embodiment, wherein the composition further comprises a chemotherapeutic agent.

Embodiment 152. A composition comprising: a carrier; and a vector comprising: a guide nucleic acid that targets a sequence within a dCK gene or a dCK RNA. The composition of any preceding embodiment, wherein the guide nucleic acid has complementarity to the dCK gene or the dCK RNA. The composition of any preceding embodiment, further comprising a nucleic acid encoding for a gene editing system or a gene editing agent provided herein. The composition of any preceding embodiment, wherein the gene editing system comprises a CRISPR-Cas gene editing agent, a TALEN gene editing agent, a base editing system, a zinc finger gene editing agent, a meganuclease gene editing agent, a DNA-polymerase gene editing agent, an RNA polymerase gene editing agent, or any combination thereof. The composition of any preceding embodiment, wherein the carrier comprises a lipid nanoparticle, a cationic nanoparticle, an inorganic nanoparticle, a virus-like particle, an extracellular vesicle, a liposome, a polymer, a viral vector, a microcapsule, a lipid emulsion, a virosome, a micelle, an exosome, an endosome, a dendrimer, a cell-penetrating peptide, or a metal. The composition of any preceding embodiment, wherein the one or more nucleic acids are comprised within a minicircle vector or a non-integrating plasmid. The composition of any preceding embodiment, wherein the one or more nucleic acids comprise DNA, RNA, or a combination thereof. The composition of any preceding embodiment, wherein the composition further comprises a chemotherapeutic agent.

Embodiment 153. A vector comprising: a nucleic acid that is at least 80% identical, at least 85% identical, at least 90% identical, at least 95% identical, at least 96% identical, at least 97% identical, at least 98% identical, at least 99% identical, or 100% identical to any one of SEQ ID NO: 1, SEQ ID NO: 2, SEQ ID NO: 8, SEQ ID NO: 9, SEQ ID NO: 15, SEQ ID NO: 16, SEQ ID NO: 19, SEQ ID NO: 20, SEQ ID NO: 23, SEQ ID NO: 24, SEQ ID NO: 25, or SEQ ID NO: 26.

Embodiment 154. A composition comprising a vector provided herein; and a carrier. The composition of any preceding embodiment, wherein the carrier comprises a lipid nanoparticle, a cationic nanoparticle, an inorganic nanoparticle, a virus-like particle, an extracellular vesicle, a liposome, a polymer, a viral vector, a microcapsule, a lipid emulsion, a virosome, a micelle, an exosome, an endosome, a dendrimer, a cell-penetrating peptide, or a metal. The composition of any preceding embodiment, wherein the one or more nucleic acids are comprised within a minicircle vector or a non-integrating plasmid. The composition of any preceding embodiment, wherein the one or more nucleic acids comprise DNA, RNA, or a combination thereof. The composition of any preceding embodiment, wherein the composition further comprises a chemotherapeutic agent.

Embodiment 155. A composition for engineering a human cell, the composition comprising a cell modulating agent or a vector encoding the cell modulating agent, wherein the cell modulating agent modulates the activity or expression level of a target in the human cell by introducing a modification into the cell, thereby producing an engineered cell, wherein the engineered cell is more resistant to a drug relative to a corresponding non-engineered cell without the modification. The composition of any preceding embodiment, wherein the engineered cell is more resistant to a cell depletion drug or a cell division inhibitor relative to a corresponding non-engineered cell without the modification. The composition of any preceding embodiment, wherein the engineered cell is more resistant to a nucleoside analogue or an antimetabolite relative to a corresponding non-engineered cell without the modification. The composition of any preceding embodiment, wherein the nucleoside analogue or the antimetabolite is 5-aza-2′-deoxycytidine (decitabine), O6-methylarabinofuranosyl guanine (nelarabine), 2′-fluoro-2′-deoxyarabinofuranosyl-2-chloroadenine (clofarabine), 5-aza-cytidine (vidaza), N4-pentyloxycarbonyl-5′-deoxy-5-fluorocytidine (capecitabine), 2,2-difluoro-2′-deoxycytidine (gemcitabine), 2-chloro-2′-deoxyadenosine (cladribine), arabinofuranosyl-2-fluoroadenine (fludarabine), 2′-deoxycoformycin (pentostatin), 5-fluoro-2′-deoxyuridine (floxuridine), arabinofuranosylcytosine (cytarabine), 6-thioguanine, 5-fluorouracil, or 6-mercaptopurine. The composition of any preceding embodiment, wherein the engineered cell is more resistant to fludarabine relative to a corresponding non-engineered cell without the modification. The composition of any preceding embodiment, wherein the cell modulating agent reduces or eliminates the activity or expression level of the target compared to the corresponding non-engineered cell without the modification. The composition of any preceding embodiment, wherein the cell modulating agent increases the activity or expression level of the target compared to the corresponding non-engineered cell without the modification. The composition of any preceding embodiment, wherein the cell modulating agent increases the activity or expression level of a first target compared to the corresponding non-engineered cell without the modification, and wherein the cell modulating agent reduces or eliminates the activity or expression level of a second compared to the corresponding non-engineered cell without the modification. The composition of any preceding embodiment, wherein the target comprises an aminoglycoside resistance agent. The composition of any preceding embodiment, wherein the aminoglycoside resistance agent is aminoglycoside-3′-phosphotransferase-Ila (APH(3′)-Ila), aminoglycoside resistance methyltransferase A (ArmA), 16S rRNA methylase A (RmtA), 16S rRNA methylase B (RmtB), 16S rRNA methylase C (RmtC), 16S rRNA methylase D (RmtD), 16S rRNA methylase D2 (RmtD2), 16S rRNA methylase E (RmtE), 16S rRNA methylase F (RmtF), 16S rRNA methylase G (RmtG), 16S rRNA methylase H (RmtH), acetylating aminoglycoside-(6)-N-acetyltransferase (AAC[6′]-Ib-cr), aminoglycoside (3″) (9) adenylyltransferase (ANT(3″)-Ia), aminoglycoside N(3)-acetyltransferase (AAC(3)-Ila), aminoglycoside 3′-phosphotransferase (APH(3″)-lb), or aminoglycoside nucleotidyltransferase (ANT(3″)-Ila). The composition of any preceding embodiment, wherein the cell modulating agent increases the activity or expression level of the aminoglycoside resistance agent or APH(3′)-Ila. The composition of any preceding embodiment, wherein the target comprises protein kinase cAMP-activated catalytic subunit alpha (PRKACA). The composition of any preceding embodiment, wherein the cell modulating agent increases the activity or expression level of PRKACA. The composition of any preceding embodiment, wherein the target comprises deoxycytidine kinase (dCK), thymidine kinase 1 (TK1), thymidine kinase 2 (TK2), UMP-CMP kinase (CMPK), or adenosine kinase (ADK). The composition of any preceding embodiment, wherein the cell modulating agent reduces or eliminates the activity or expression level of dCK, TKI, TK2, CMPK, or ADK. The composition of any preceding embodiment, the cell modulating agent comprises an RNA molecule, a DNA molecule, a gene editing agent, a gene integration agent, a gene expression modulator, an epigenetic modulator, or a biomolecule activity modulator. The composition of any preceding embodiment, wherein the RNA molecule comprises a siRNA, a miRNA, or a shRNA. The composition of any preceding embodiment, wherein the gene editing agent comprises a CRISPR/Cas protein, a transcription activator-like effector nuclease (TALEN), or a zinc finger (ZF) protein. The composition of any preceding embodiment, wherein the gene integration agent comprises a virus, an integrase, a transposase, a recombinase, or a homology-directed repair (HDR) system. The composition of any preceding embodiment, wherein the gene expression modulator comprises a CRISPR/Cas protein, a transcription activator-like effector (TALE), a zinc finger (ZF), a transcription factor, an activator, a co-activator, a repressor, or an inhibitor. The composition of any preceding embodiment, wherein the activator comprises Gal4, VP64, or p65. The composition of any preceding embodiment, wherein the epigenetic modulator comprises an epigenetic reader, an epigenetic writer, or an epigenetic eraser. The composition of any preceding embodiment, wherein the epigenetic writer comprises an acetyltransferase or a methyltransferase. The composition of any preceding embodiment, wherein the epigenetic eraser comprises a deacetylase or a demethylase. The composition of any preceding embodiment, wherein the biomolecule activity modulator comprises a protease, a kinase, a phosphatase, an acetylase, a deacetylase, a methyltransferase, a demethylase, a ubiquitin-activating enzyme (E1), a ubiquitin-conjugating enzyme (E2), a ubiquitin ligase (E3), a deubiquitinating enzyme (DUB), a glycosyltransferase, a glycosidase, an inhibitor of the target, a co-factor of the target, or an activator of the target. The composition of any preceding embodiment, wherein the cell modulating agent is encapsulated by a polymeric material. The composition of any preceding embodiment, wherein the polymeric material is a nanocapsule. The composition of any preceding embodiment, wherein the nanocapsule comprises synthetic polymers, wherein the synthetic polymer comprises poly(ethylene glycol) monomethyl ether (mPEG), poly-e-caprolactone (PCL), poly(lactide) (PLA), poly(lactide-co-glicolide) (PLGA), thiolated poly(methacrylic acid), and poly(N-vinyl Pyrrolidone). The composition of any preceding embodiment, wherein the nanocapsule comprises a saccharide. The composition of any preceding embodiment, wherein the nanocapsule comprises a lipid. The composition of any preceding embodiment, the nanocapsule is a liposome. The composition of any preceding embodiment, wherein the nanocapsule is a lipid nanoparticle (LNP). The composition of any preceding embodiment, wherein the cell modulating agent is encoded by a vector. The composition of any preceding embodiment, wherein the vector is an RNA molecule, a DNA molecule, or a viral vector. The composition of any preceding embodiment, wherein the viral vector is a lentiviral vector, a retroviral vector, an adeno-associated viral (AAV) vector, or an adenoviral vector. The composition of any preceding embodiment, wherein the polymeric material further comprises a cell marker binding moiety. The composition of any preceding embodiment, wherein the cell marker is a T cell marker of prior tumor reactivity, a T cell lineage marker, a T cell differentiation marker, or an engineered cell marker. The composition of any preceding embodiment, wherein the T cell marker of prior tumor reactivity comprises PD-1, PD-LI, TIM3, LAG-3, TIGIT, CTLA4, CD39, CXCL13, CD25, CD69, 4-IBB, IL-2, IFN-γ, TNF, Fos, Jun, NFAT family proteins, NF-KB family proteins, Granzyme B, Granzyme A, Granzyme H, Granzyme K, Granzyme M, perforin, :tvIHC class II components, HLA-DR, HLA-DP, HLA-DQ, OX40, OX40L, ICOS, RORC, T-bet, Blimp-I, GATA3, FOXP3, BCL2, XIRPI, IL3, CXCLS, MIR155HG, IFITI, XCL2, FLTI, IFI44L, SLC26A4, B3GNT5, PTGS2, ERRFII, MYOIB, CD200, INHBA, PROS I, IFIT3, PPP2R3A, MLFI, FEZI, NUDT4B, PLDI, TNFRSF9, MFSD2A, NR4A3, KCNH4, IL5, TLR4, DENND5B, IF144, RAPHI, IRFS, SMADI, PALM2-AKAP2, CD109, EPSS, SPPI, CRTAM, LAMP3, POLR3G, FAMI 14A1, XCLI, DOCK7, GNG4, ROBOI, SERPINE2, CLDN12, PFN2, ABTB2, ZNF704, CLIC4, NRCAM, SAMD4A, BCL2A1, EFNAI, TMTCI, B4GALT6, MOB3B, ARFGEF3, PLCBI, CHRNA5, ADAM23, NR4A2, SNX24, LMCDI, PLEKHGI, NIPAL4, ENCI, ARHGAP42, CTHRCI, Cl5orf48, CEMIP, CDC42BPA, EXTL2, AASS, AFF3, ZC3H12C, GJCI, ST8SIAI, ENPPI, FABP5, SORBSI, SLC44A1, ZFYVE9, HERC5, NCSI, PON2, PTGFRN, EMPI, ARRDC4, PTPN13, KIAA0754, GNB4, TIAM2, TPMI, TEAD4, RAB38, or RGMB. The composition of any preceding embodiment, wherein the T cell lineage marker comprises CD3, CD4, or CD8. The composition of any preceding embodiment, wherein the T cell differentiation marker comprises CCR7, CD45RA, CD45RO, CD62L, CD57, or CXCR3. The composition of any preceding embodiment, wherein the engineered cell marker comprises a tag, or an engineered receptor or a fragment thereof. The composition of any preceding embodiment, wherein the engineered receptor comprises an scFv, an orthogonal polypeptide, an engineered T cell receptor, a chimeric antigen receptor, or fragments thereof. The composition of any preceding embodiment, wherein the cell marker binding moiety comprises a polypeptide. The composition of any preceding embodiment, wherein the polypeptide comprises an antibody, an antibody-derived binding moiety, or an engineered binding moiety.

Embodiment 156. A composition comprising an engineered cell, wherein the engineered cell is an engineered human cell and comprises a modification such that the engineered cell is more resistant to a nucleoside analogue or an antimetabolite relative to a corresponding non-engineered human cell without the modification, wherein the activity or expression level of a target in the engineered cell is modulated compared to the activity or expression level of the target in the non-engineered human cell. The composition of any preceding embodiment, wherein the nucleoside analogue or the antimetabolite is 5-aza-2′-deoxycytidine (decitabine), 06-methylarabinofuranosyl guanine (nelarabine), 2′-fluoro-2′-deoxyarabinofuranosyl-2-chloroadenine (clofarabine), 5-aza-cytidine (vidaza), N4-pentyloxycarbonyl-5′-deoxy-5-fluorocytidine (capecitabine), 2,2-difluoro-2′-deoxycytidine (gemcitabine), 2-chloro-2′-deoxyadenosine (cladribine), arabinofuranosyl-2-fluoroadenine (fludarabine), 2′-deoxycoformycin (pentostatin), 5-fluoro-2′-deoxyuridine (floxuridine), arabinofuranosylcytosine (cytarabine), 6-thioguanine, 5-fluorouracil, or 6-mercaptopurine. The composition of any preceding embodiment, wherein the nucleoside analogue or the antimetabolite is fludarabine. The composition of any preceding embodiment, wherein the engineered human cell is an immune cell.

Embodiment 157. A composition comprising an engineered cell, wherein the engineered cell is an engineered immune cell and comprises a modification such that the engineered cell is more resistant to a cell depletion drug or a cell division inhibitor compared to a corresponding non-engineered immune cell without the modification. The composition of any preceding embodiment, wherein the cell depletion drug or the cell division inhibitor is a nucleoside analogue or an antimetabolite. The composition of any preceding embodiment, wherein the cell depletion drug or the cell division inhibitor is 5-aza-2′-deoxycytidine (decitabine), 06-methylarabinofuranosyl guanine (nelarabine), 2′-fluoro-2′-deoxyarabinofuranosyl-2-chloroadenine (clofarabine), 5-aza-cytidine (vidaza), N4-pentyloxycarbonyl-5′-deoxy-5-fluorocytidine (capecitabine), 2,2-difluoro-2′-deoxycytidine (gemcitabine), 2-chloro-2′-deoxyadenosine (cladribine), arabinofuranosyl-2-fluoroadenine (fludarabine), 2′-deoxycoformycin (pentostatin), 5-fluoro-2′-deoxyuridine (floxuridine), arabinofuranosylcytosine (cytarabine), 6-thioguanine, 5-fluorouracil, or 6-mercaptopurine. The composition of any preceding embodiment, wherein the cell depletion drug or the cell division inhibitor is fludarabine. The composition of any preceding embodiment, wherein the activity or expression level of a target in the engineered cell is modulated compared to the activity or expression level of the target in the non-engineered cell. The composition of any preceding embodiment, wherein the activity or expression level of a target in the engineered cell is increased compared to the activity or expression level of the target in the non-engineered cell. The composition of any preceding embodiment, wherein the activity or expression level of a target in the engineered cell is reduced compared to the activity or expression level of the target in the non-engineered cell. The composition of any preceding embodiment, wherein the activity or expression level of a first target in the engineered cell is increased compared to the activity or expression level of the first target in the non-engineered cell, and wherein the activity or expression level of a second target in the engineered cell is reduced compared to the activity or expression level of the second target in the non-engineered cell. The composition of any preceding embodiment, wherein the target comprises an aminoglycoside resistance agent. The composition of any preceding embodiment, wherein the aminoglycoside resistance agent is aminoglycoside-3′-phosphotransferase-Ila (APH(3′)-Ila), aminoglycoside resistance methyltransferase A (ArmA), 16S rRNA methylase A (RmtA), 16S rRNA methylase B (RmtB), 16S rRNA methylase C (RmtC), 16S rRNA methylase D (RmtD), 16S rRNA methylase D2 (RmtD2), 16S rRNA methylase E (RmtE), 16S rRNA methylase F (RmtF), 16S rRNA methylase G (RmtG), 16S rRNA methylase H (RmtH), acetylating aminoglycoside-(6)-N-acetyltransferase (AAC[6′]-Ib-cr), aminoglycoside (3″) (9) adenylyltransferase (ANT(3″)-Ia), aminoglycoside N(3)-acetyltransferase (AAC(3)-Ila), aminoglycoside 3′-phosphotransferase (APH(3″)-Ib), or aminoglycoside nucleotidyltransferase (ANT(3″)-Ila). The composition of any preceding embodiment, wherein the cell modulating agent increases the activity or expression level of the aminoglycoside resistance agent or APH(3′)-Ila. The composition of any preceding embodiment, wherein the target comprises protein kinase cAMP-activated catalytic subunit alpha (PRKACA). The composition of any preceding embodiment, wherein the cell modulating agent increases the activity or expression level of PRKACA. The composition of any preceding embodiment, wherein the target comprises deoxycytidine kinase (dCK), thymidine kinase I (TKI), thymidine kinase 2 (TK2), UMP-CMP kinase (CMPK1), or adenosine kinase (ADK). The composition of any preceding embodiment, wherein the cell modulating agent reduces the activity or expression level of dCK, TK1, TK2, CMPK1, or ADK. The composition of any preceding embodiment, wherein the cell is autologous. The composition of any preceding embodiment, wherein the cell is allogeneic. The composition of any preceding embodiment, wherein the cell is a stem cell. The composition of any preceding embodiment, wherein the cell is an immune cell. The composition of any preceding embodiment, wherein the immune cell is a tumor-infiltrating lymphocyte (TIL), a splenocyte, a T cell, a B cell, a natural killer (NK) cell, a monocyte, or a macrophage. The composition of any preceding embodiment, wherein the T cell is a CAR-T cell, a TCR-T cell, a circulating T cell, a tissue resident T cell, a T cell from peripheral blood, or a T cell from the spleen. The composition of any preceding embodiment, wherein the immune cell comprises a synthetic receptor. The composition of any preceding embodiment, wherein the synthetic receptor is a chimeric antigen receptor (CAR) or an engineered T-cell receptor (TCR). The composition of any preceding embodiment, wherein the cell is not a cancer cell. The composition of any preceding embodiment, wherein the cell depletion drug or the cell division inhibitor is a lymphodepletion agent. The composition of any preceding embodiment, wherein the lymphodepletion agent is an anti-CD52 antibody or an anti-CD45 antibody. The composition of any preceding embodiment, wherein the cell depletion drug or the cell division inhibitor is a cytotoxic drug or an alkylating agent. The composition of any preceding embodiment, wherein the activity or expression level of the target in the engineered cell is higher compared to the activity or expression level of the target in the non-engineered cell. The composition of any preceding embodiment, wherein the activity or expression level of the target in the engineered cell is at least 1.1-fold, at least 1.5-fold, at least 2-fold, at least 3-fold, at least 4-fold, or at least 5-fold higher compared to the activity or expression level of the target in the non-engineered cell. The composition of any preceding embodiment, wherein the activity or expression level of the target in the engineered cell is lower compared to the activity or expression level of the target in the non-engineered cell. The composition of any preceding embodiment, wherein the activity or expression level of the target in the engineered cell is at least 1.1-fold, at least 1.5-fold, at least 2-fold, at least 3-fold, at least 4-fold, or at least 5-fold lower compared to the activity or expression level of the target in the non-engineered cell. The composition of any preceding embodiment, wherein the target is an endogenous target. The composition of any preceding embodiment, wherein the target is a heterologous target. The composition of any preceding embodiment, wherein the target is a gene, a nucleic acid molecule, an enzyme, a protein, a lipid, or a carbohydrate. The composition of any preceding embodiment, wherein the modification is a genetic modification, a post-transcriptional modification, a post-translational modification, an epigenetic modification, or a target activity modification. The composition of any preceding embodiment, wherein the genetic modification comprises a genetic insertion, a genetic deletion, a genetic substitution, a genetic translocation, a genetic inversion, homology-directed repair (HDR), non-homologous end joining (NHEJ), a genetic overexpression, or a genetic repression. The composition of any preceding embodiment, wherein the engineered cell is more resistant to at least two cell depletion drugs or cell division inhibitors. The composition of any preceding embodiment, wherein the modulated activity or expression level of the target phosphorylates the drug or a processed fonn of the drug, thereby inactivating the drug or the processed form of the drug. The composition of any preceding embodiment, wherein the modulated activity or expression level of the target phosphorylates the drug or a processed form of the drug, thereby inhibiting binding of the drug or a processed form of the drug to a ribosome. The composition of any preceding embodiment, wherein the modulated activity or expression level of the target inactivates the drug or a processed form of the drug. The composition of any preceding embodiment, wherein the modulated activity or expression level of the target dephosphorylates a drug or a processed form of the drug, thereby inhibiting an enzymatic reaction involving the drug or a processed form of the drug. The composition of any preceding embodiment, wherein the modulated activity or expression level of the target inhibits processing of the drug into an active form, thereby inhibiting drug incorporation into DNA replication, DNA synthesis, and DNA repair processes. The composition of any preceding embodiment, wherein the modulated activity or expression level of the target inhibits cell cycle arrest, inhibition of cell division, and/or cell death. The composition of any preceding embodiment, wherein the cell is a population of cells comprising at least 1×10³ cells.

Embodiment 158. A method of engineering a cell comprising introducing a composition provided herein into a cell. The method of any preceding embodiment, wherein the method further comprises identifying the cell by the expression of a cell marker. The method of any preceding embodiment, wherein the cell marker is a T cell marker of prior tumor reactivity, a T cell lineage marker, a T cell differentiation marker, or an engineered cell marker. The method of any preceding embodiment, wherein the T cell marker of prior tumor reactivity comprises PD-1, PD-L1, TIM3, LAG-3, TIGIT, CTLA4, CD39, CXCL13, CD25, CD69, 4-1BB, IL-2, IFN-γ, TNF, Fos, Jun, NFAT family proteins, NF-KB family proteins, Granzyme B, Granzyme A, Granzyme H, Granzyme K, Granzyme M, perform, :MHC class II components, HLA-DR, HLA-DP, HLA-DQ, OX40, OX40L, ICOS, RORC, T-bet, Blimp-I, GATA3, FOXP3, BCL2, XIRP1, ILJ, CXCL8, MIR155HG, IFIT1, XCL2, FLTI, IFI44L, SLC26A4, B3GNT5, PTGS2, ERRFi1, MYO1B, CD200, INHBA, PROS1, IFITJ, PPP2R3A, MLF1, FEZI, NUDT4B, PLD1, TNFRSF9, 1\11FSD2A, NR4A3, KCNH4, IL5, TLR4, DENND5B, IF144, RAPHI, IRF8, SMADI, PALM2-AKAP2, CD109, EPS8, SPPI, CRTAM, LAMP3, POLR3G, FAMI 14A1, XCL1, DOCK7, GNG4, ROBO1, SERPINE2, CLDN12, PFN2, ABTB2, ZNF704, CLIC4, NRCAM, SAMD4A, BCL2A1, EFNA1, TMTCI, B4GALT6, MOB3B, ARFGEF3, PLCB1, CHRNA5, ADAM23, NR4A2, SNX24, LMCD1, PLEKHGI, NIPAL4, ENCI, ARHGAP42, CTHRCI, C15orf48, CEMIP, CDC42BPA, EXTL2, AASS, AFFJ, ZC3H12C, GJC1, ST8SIA1, ENPPI, FABP5, SORBSI, SLC44A1, ZFYVE9, HERC5, NCSI, PON2, PTGFRN, EMPI, ARRDC4, PTPN13, KIAA0754, GNB4, TIAM2, TPMI, TEAD4, RAB38, or RGMB. The method of any preceding embodiment, wherein the T cell lineage marker comprises CD3, CD4, or CD8. The method of any preceding embodiment, wherein the T cell differentiation marker comprises CCR7, CD45RA, CD45RO, CD62L, CD57, or CXCR3. The method of any preceding embodiment, wherein the engineered cell marker comprises a tag. The method of any preceding embodiment, wherein the tag comprises an orthogonal polypeptide, or a synthetic receptor or fragment thereof. The method of any preceding embodiment, wherein the cell is ex vivo. The method of any preceding embodiment, wherein the cell is in vivo. The method of any preceding embodiment, wherein the cell is a population of cells comprising at least 1×10³ cells.

Embodiment 159. A composition for engineering a human cell, the composition comprising a cell modulating agent or a vector encoding the cell modulating agent, wherein the cell modulating agent modulates the activity or expression level of a target in the human cell by introducing a modification into the cell, thereby producing an engineered cell, wherein the engineered cell is more resistant to a drug relative to a corresponding non-engineered cell without the modification. The composition of any preceding embodiment, wherein the engineered cell is more resistant to a cell depletion drug or a cell division inhibitor relative to a corresponding non-engineered cell without the modification. The composition of any preceding embodiment, wherein the engineered cell is more resistant to an alkylating drug relative to a corresponding non-engineered cell without the modification. The composition of any preceding embodiment, wherein the alkylating drug is cyclophosphamide, ifosfamide, chlorambucil, altretamine, bendamustine, busulfan, carboquone, carmustine, chlormethine, chlorozotocin, dacarbazine, fotemustine, lomustine, melphalan, melphalan flufenamide, mitobronitol, nimustine, mitrosoureas, pipobroman, ranimustine, semustine, temozolomide, thiotepa, treosulfan, triaziquone, triethylenemelamine, trofosfamide, or uramustine. The composition of any preceding embodiment, wherein the engineered cell is more resistant to cyclophosphamide relative to a corresponding non-engineered cell without the modification. The composition of any preceding embodiment, wherein the cell modulating agent reduces the activity or expression level of the target compared to the corresponding non-engineered cell without the modification. The composition of any preceding embodiment, wherein the cell modulating agent increases the activity or expression level of the target compared to the corresponding non-engineered cell without the modification. The composition of any preceding embodiment, wherein the cell modulating agent increases the activity or expression level of a first target compared to the corresponding non-engineered cell without the modification, and wherein the cell modulating agent reduces the activity or expression level of a second compared to the corresponding non-engineered cell without the modification. The composition of any preceding embodiment, wherein the target comprises an enzyme. The composition of any preceding embodiment, wherein the enzyme is an aldehyde dehydrogenase (ALDH). The composition of any preceding embodiment, wherein the cell modulating agent increases the activity or expression level of the enzyme or the ALDH. The composition of any preceding embodiment, wherein the ALDH is ALDH 1 Family Member A1 (ALDHIAI), ALDH I Family Member A2 (ALDHIA2), ALDH I Family Member A3 (ALDHIA3), ALDH I Family Member B1 (ALDHIBI), ALDH I Family Member LI (ALDHILI), ALDH I Family Member L2 (ALDHIL2), ALDH 2 Family Member (ALDH2), ALDH 3 Family Member A1 (ALDH3A1), ALDH 3 Family Member A2 (ALDH3A2), ALDH 3 Family Member B1 (ALDH3B 1), ALDH 3 Family Member B2 (ALDH3B2), ALDH 4 Family Member A1 (ALDH4A 1), ALDH 5 Family Member A1 (ALDHSA1), ALDH 6 Family Member A1 (ALDH6A1), ALDH 7 Family Member A1 (ALDH7A1), ALDH 8 Family Member A1 (ALDH8A1), ALDH 9 Family Member A1 (ALDH9A1), ALDH 16 Family Member A1 (ALDH16A1), or ALDH 18 Family Member A1 (ALDH18A1). The composition of any preceding embodiment, wherein the target comprises a glutathione S-transferase (GST). The composition of any preceding embodiment, wherein the cell modulating agent increases the activity or expression level of the GST. The composition of any preceding embodiment, wherein the GST is GST Alpha 1 (GSTA1), GST Alpha 2 (GSTA2), GST Alpha 3 (GSTA3), GST Alpha 4 (GSTA4), GST Alpha 5 (GSTAS), GST Kappa 1 (GSTK1), GST Mu 1 (GSTM1), GST Mu 1-like GSTM1L, GST Mu 2 (GSTM2), GST Mu 3 (GSTM3), GST Mu 4 (GSTM4), GST Mu 5 (GSTM5), GST Omega 1 (GSTOI), GST Omega 2 (GSTO2), GST Pi I (GSTPI), GST Theta I (GSTTI), GST Theta 2 (GSTT2), GST Theta 4 (GSTT4), GST Zeta I (GSTZI), Microsomal GST I (MGSTI), Microsomal GST 2 (MGST2), or Microsomal GST 3 (MGST3). The composition of any preceding embodiment, wherein the cell modulating agent comprises an RNA molecule, a DNA molecule, a gene editing agent, a gene integration agent, a gene expression modulator, an epigenetic modulator, or a biomolecule activity modulator. The composition of any preceding embodiment, wherein the RNA molecule comprises a siRNA, a miRNA, or a shRNA. The composition of any preceding embodiment, wherein the gene editing agent comprises a CRISPR/Cas protein, a transcription activator-like effector nuclease (TALEN), or a zinc finger (ZF) protein. The composition of any preceding embodiment, wherein the gene integration agent comprises a virus, an integrase, a transposase, a recombinase, or a homology-directed repair (HDR) system. The composition of any preceding embodiment, wherein the gene expression modulator comprises a CRISPR/Cas protein, a transcription activator-like effector (TALE), a zinc finger (ZF), a transcription factor, an activator, a co-activator, a repressor, or an inhibitor. The composition of any preceding embodiment, wherein the activator comprises Gal4, VP64, or p65. The composition of any preceding embodiment, wherein the epigenetic modulator comprises an epigenetic reader, an epigenetic writer, or an epigenetic eraser. The composition of any preceding embodiment, wherein the epigenetic writer comprises an acetyltransferase or a methyltransferase. The composition of any preceding embodiment, wherein the epigenetic eraser comprises a deacetylase or a demethylase. The composition of any preceding embodiment, wherein the biomolecule activity modulator comprises a protease, a kinase, a phosphatase, an acetylase, a deacetylase, a methyltransferase, a demethylase, a ubiquitin-activating enzyme (E1), a ubiquitin-conjugating enzyme (E2), a ubiquitin ligase (E3), a deubiquitinating enzyme (DUB), a glycosyltransferase, a glycosidase, an inhibitor of the target, a co-factor of the target, or an activator of the target. The composition of any preceding embodiment, wherein the cell modulating agent is encapsulated by a polymeric material. The composition of any preceding embodiment, wherein the polymeric material is a nanocapsule. The composition of any preceding embodiment, wherein the nanocapsule comprises synthetic polymers, wherein the synthetic polymer comprises poly(ethylene glycol) monomethyl ether (mPEG), poly-e-caprolactone (PCL), poly(lactide) (PLA), poly(lactide-co-glicolide) (PLGA), thiolated poly(methacrylic acid), and poly(N-vinyl Pyrrolidone). The composition of any preceding embodiment, wherein the nanocapsule comprises a saccharide. The composition of any preceding embodiment, wherein the nanocapsule comprises a lipid. The composition of any preceding embodiment, wherein the nanocapsule is a liposome. The composition of any preceding embodiment, wherein the nanocapsule is a lipid nanoparticle (LNP). The composition of any preceding embodiment, wherein the cell modulating agent is encoded by a vector. The composition of any preceding embodiment, wherein the vector is an RNA molecule, a DNA molecule, or a viral vector. The composition of any preceding embodiment, wherein the viral vector is a lentiviral vector, a retroviral vector, an adeno-associated viral (AAV) vector, or an adenoviral vector. The composition of any preceding embodiment, wherein the polymeric material further comprises a cell marker binding moiety. The composition of any preceding embodiment, wherein the cell marker is a T cell marker of prior tumor reactivity, a T cell lineage marker, a T cell differentiation marker, or an engineered cell marker. The composition of any preceding embodiment, wherein the T cell marker of prior tumor reactivity comprises PD-1, PD-LI, TIM3, LAG-3, TIGIT, CTLA4, CD39, CXCL13, CD25, CD69, 4-IBB, IL-2, IFN-γ, TNF, Fos, Jun, NFAT family proteins, NF-KB family proteins, Granzyme B, Granzyme A, Granzyme H, Granzyme K, Granzyme M, perform, :tvIHC class II components, HLA-DR, HLA-DP, HLA-DQ, OX40, OX40L, ICOS, RORC, T-bet, Blimp-I, GATA3, FOXP3, BCL2, XIRPI, IL3, CXCLS, MIR155HG, IFITI, XCL2, FLTI, IFI44L, SLC26A4, B3GNT5, PTGS2, ERRFII, MYOIB, CD200, INHBA, PROS I, IFIT3, PPP2R3A, MLFI, FEZI, NUDT4B, PLDI, TNFRSF9, MFSD2A, NR4A3, KCNH4, IL5, TLR4, DENND5B, IFI44, RAPHI, IRFS, SMADI, PALM2-AKAP2, CD109, EPSS, SPPI, CRTAM, LAMP3, POLR3G, FAMI 14A1, XCLI, DOCK7, GNG4, ROBOI, SERPINE2, CLDN12, PFN2, ABTB2, ZNF704, CLIC4, NRCAM, SAMD4A, BCL2A1, EFNAI, TMTCI, B4GALT6, MOB3B, ARFGEF3, PLCBI, CHRNA5, ADAM23, NR4A2, SNX24, LMCDI, PLEKHGI, NIPAL4, ENCI, ARHGAP42, CTHRCI, Cl5orf48, CEMIP, CDC42BPA, EXTL2, AASS, AFF3, ZC3H12C, GJCI, ST8SIAI, ENPPI, FABP5, SORBSI, SLC44A1, ZFYVE9, HERC5, NCSI, PON2, PTGFRN, EMPI, ARRDC4, PTPN13, KIAA0754, GNB4, TIAM2, TPMI, TEAD4, RAB38, or RGMB. The composition of any preceding embodiment, wherein the T cell lineage marker comprises CD3, CD4, or CD8. The composition of any preceding embodiment, wherein the T cell differentiation marker comprises CCR7, CD45RA, CD45RO, CD62L, CD57, or CXCR3. The composition of any preceding embodiment, wherein the engineered cell marker comprises a tag, or an engineered receptor or a fragment thereof. The composition of any preceding embodiment, wherein the engineered receptor comprises an scFv, an orthogonal polypeptide, an engineered T cell receptor, a chimeric antigen receptor, or fragments thereof. The composition of any preceding embodiment, wherein the cell marker binding moiety comprises a polypeptide. The composition of any preceding embodiment, wherein the polypeptide comprises an antibody, an antibody-derived binding moiety, or an engineered binding moiety.

Embodiment 160. A composition comprising an engineered cell, wherein the engineered cell is an engineered human cell and comprises a modification such that the engineered cell is more resistant to an alkylating drug relative to a corresponding non-engineered human cell without the modification, wherein the activity or expression level of a target in the engineered cell is modulated compared to the activity or expression level of the target in the non-engineered human cell. The composition of any preceding embodiment, wherein the alkylating drug is cyclophosphamide, ifosfamide, chlorambucil, altretamine, bendamustine, busulfan, carboquone, carmustine, chlormethine, chlorozotocin, dacarbazine, fotemustine, lomustine, melphalan, melphalan flufenamide, mitobronitol, nimustine, mitrosoureas, pipobroman, ranimustine, semustine, temozolomide, thiotepa, treosulfan, triaziquone, triethylenemelamine, trofosfamide, or uramustine. The composition of any preceding embodiment, wherein the alkylating drug is cyclophosphamide. The composition of any preceding embodiment, wherein the engineered human cell is an immune cell.

Embodiment 161. A composition comprising an engineered cell, wherein the engineered cell is an engineered immune cell and comprises a modification such that the engineered cell is more resistant to a cell depletion drug or a cell division inhibitor compared to a corresponding non-engineered immune cell without the modification. The composition of any preceding embodiment, wherein the cell depletion drug or the cell division inhibitor is an alkylating drug. The composition of any preceding embodiment, wherein the cell depletion drug or the cell division inhibitor is cyclophosphamide, ifosfamide, chlorambucil, altretamine, bendamustine, busulfan, carboquone, carmustine, chlormethine, chlorozotocin, dacarbazine, fotemustine, lomustine, melphalan, melphalan flufenamide, mitobronitol, nimustine, mitrosoureas, pipobroman, ranimustine, semustine, temozolomide, thiotepa, treosulfan, triaziquone, triethylenemelamine, trofosfamide, or uramustine. The composition of any preceding embodiment, wherein the cell depletion drug or the cell division inhibitor is cyclophosphamide. The composition of any preceding embodiment, wherein the activity or expression level of a target in the engineered cell is modulated compared to the activity or expression level of the target in the non-engineered cell. The composition of any preceding embodiment, wherein the activity or expression level of a target in the engineered cell is increased compared to the activity or expression level of the target in the non-engineered cell. The composition of any preceding embodiment, wherein the activity or expression level of a target in the engineered cell is reduced compared to the activity or expression level of the target in the non-engineered cell. The composition of any preceding embodiment, wherein the activity or expression level of a first target in the engineered cell is increased compared to the activity or expression level of the first target in the non-engineered cell, and wherein the activity or expression level of a second target in the engineered cell is reduced compared to the activity or expression level of the second target in the non-engineered cell. The composition of any preceding embodiment, wherein the target comprises an enzyme. The composition of any preceding embodiment, wherein the enzyme is an aldehyde dehydrogenase (ALDH). The composition of any preceding embodiment, wherein the cell modulating agent increases the activity or expression level of the enzyme or the ALDH. The composition of any preceding embodiment, wherein the ALDH is ALDH 1 Family Member A1 (ALDH1A1), ALDH 1 Family Member A2 (ALDH1A2), ALDH 1 Family Member A3 (ALDH1A3), ALDH 1 Family Member B1 (ALDH1B1), ALDH 1 Family Member LI (ALDH1L1), ALDH 1 Family Member L2 (ALDH1L2), ALDH 2 Family Member (ALDH2), ALDH 3 Family Member A1 (ALDH3A1), ALDH 3 Family Member A2 (ALDH3A2), ALDH 3 Family Member B1 (ALDH3B1), ALDH 3 Family Member B2 (ALDH3B2), ALDH 4 Family Member A1 (ALDH4A 1), ALDH 5 Family Member A1 (ALDH5A1), ALDH 6 Family Member A1 (ALDH6A1), ALDH 7 Family Member A1 (ALDH7A1), ALDH 8 Family Member A1 (ALDH8A1), ALDH 9 Family Member A1 (ALDH9A1), ALDH 16 Family Member A1 (ALDH16A1), or ALDH 18 Family Member A1 (ALDH18A1). The composition of any preceding embodiment, wherein the target comprises a glutathione S-transferase (GST). The composition of any preceding embodiment, wherein the cell modulating agent increases the activity or expression level of the GST. The composition of any preceding embodiment, wherein the GST is GST Alpha 1 (GSTA1), GST Alpha 2 (GSTA2), GST Alpha 3 (GSTA3), GST Alpha 4 (GSTA4), GST Alpha 5 (GSTA5), GST Kappa 1 (GSTK1), GST Mu 1 (GSTM1), GST Mu I-like GSTMIL, GST Mu 2 (GSTM2), GST Mu 3 (GSTM3), GST Mu 4 (GSTM4), GST Mu 5 (GSTM5), GST Omega 1 (GSTOI), GST Omega 2 (GSTO2), GST Pi 1 (GSTP1), GST Theta 1 (GSTTI), GST Theta 2 (GSTT2), GST Theta 4 (GSTT4), GST Zeta 1 (GSTZ1), Microsomal GST 1 (MGST1), Microsomal GST 2 (MGST2), or Microsomal GST 3 (MGST3). The composition of any preceding embodiment, wherein the cell is autologous. The composition of any preceding embodiment, wherein the cell is allogeneic. The composition of any preceding embodiment, wherein the cell is a stem cell. The composition of any preceding embodiment, wherein the cell is an immune cell. The composition of any preceding embodiment, wherein the immune cell is a tumor-infiltrating lymphocyte (TIL), a splenocyte, a T cell, a B cell, a natural killer (NK) cell, a monocyte, or a macrophage. The composition of any preceding embodiment, wherein the T cell is a CAR-T cell, a TCR-T cell, a circulating T cell, a tissue resident T cell, a T cell from peripheral blood, or a T cell from the spleen. The composition of any preceding embodiment, wherein the immune cell comprises a synthetic receptor. The composition of any preceding embodiment, wherein the synthetic receptor is a chimeric antigen receptor (CAR) or an engineered T-cell receptor (TCR). The composition of any preceding embodiment, wherein the cell is not a cancer cell. The composition of any preceding embodiment, wherein the cell depletion drug or the cell division inhibitor is a lymphodepletion agent. The composition of any preceding embodiment, wherein the lymphodepletion agent is an anti-CD52 antibody or an anti-CD45 antibody. The composition of any preceding embodiment, wherein the cell depletion drug or the cell division inhibitor is a cytotoxic drug, a nucleoside analogue or an antimetabolite. The composition of any preceding embodiment, wherein the activity or expression level of the target in the engineered cell is higher compared to the activity or expression level of the target in the non-engineered cell. The composition of any preceding embodiment, wherein the activity or expression level of the target in the engineered cell is at least 1.1-fold, at least 1.5-fold, at least 2-fold, at least 3-fold, at least 4-fold, or at least 5-fold higher compared to the activity or expression level of the target in the non-engineered cell. The composition of any preceding embodiment, wherein the activity or expression level of the target in the engineered cell is lower compared to the activity or expression level of the target in the non-engineered cell. The composition of any preceding embodiment, wherein the activity or expression level of the target in the engineered cell is at least 1.1-fold, at least 1.5-fold, at least 2-fold, at least 3-fold, at least 4-fold, or at least 5-fold lower compared to the activity or expression level of the target in the non-engineered cell. The composition of any preceding embodiment, wherein the target is an endogenous target. The composition of any preceding embodiment, wherein the target is a heterologous target. The composition of any preceding embodiment, wherein the target is a gene, a nucleic acid molecule, an enzyme, a protein, a lipid, or a carbohydrate. The composition of any preceding embodiment, wherein the modification is a genetic modification, a post-transcriptional modification, a post-translational modification, an epigenetic modification, or a target activity modification. The composition of any preceding embodiment, wherein the genetic modification comprises a genetic insertion, a genetic deletion, a genetic substitution, a genetic translocation, a genetic inversion, homology-directed repair (HDR), non-homologous end joining (NHEJ), a genetic overexpression, or a genetic repression. The composition of any preceding embodiment, wherein the engineered cell is more resistant to at least two cell depletion drugs or cell division inhibitors. The composition of any preceding embodiment, wherein the modulated activity or expression level of the target phosphorylates the drug or a processed form of the drug, thereby inactivating the drug or the processed form of the drug. The composition of any preceding embodiment, wherein the modulated activity or expression level of the target phosphorylates the drug or a processed form of the drug, thereby inhibiting binding of the drug or a processed form of the drug to a ribosome. The composition of any preceding embodiment, wherein the modulated activity or expression level of the target inactivates the drug or a processed form of the drug. The composition of any preceding embodiment, wherein the modulated activity or expression level of the target dephosphorylates a drug or a processed fonn of the drug, thereby inhibiting an enzymatic reaction involving the drug or a processed form of the drug. The composition of any preceding embodiment, wherein the modulated activity or expression level of the target inhibits processing of the drug into an active form, thereby inhibiting drug incorporation into DNA replication, DNA synthesis, and DNA repair processes. The composition of any preceding embodiment, wherein the modulated activity or expression level of the target inhibits cell cycle arrest, inhibition of cell division, and/or cell death. The composition of any preceding embodiment, wherein the cell is comprised within a population of cells comprising at least 1×10³ cells.

Embodiment 162. A composition for engineering a cell, the composition comprising a cell modulating agent or a vector encoding the cell modulating agent, wherein the cell modulating agent modulates the activity or expression level of a target in the cell by introducing a modification into the cell such that the engineered cell is more resistant to a drug relative to a corresponding non-engineered cell without the modification.

Embodiment 163. A composition comprising an engineered cell, wherein the engineered cell comprises a modification such that the engineered cell is more resistant to a drug relative to a corresponding non-engineered cell without the modification, wherein the activity or expression level of a target in the engineered cell is modulated compared to the activity or expression level of the target in the non-engineered cell. The composition of any preceding embodiment, wherein the drug is a cell depletion drug or a cell division inhibitor.

Embodiment 164. A composition comprising an engineered cell, wherein the engineered cell comprises a modification such that the engineered cell is more resistant to a cell depletion drug or a cell division inhibitor compared to a corresponding non-engineered cell without the modification.

Embodiment 165. A pharmaceutical composition comprising: a composition provided herein and a pharmaceutically acceptable carrier.

Embodiment 166. A method of engineering a cell comprising introducing a composition provided herein into a cell.

Embodiment 167. A method of treating a disease or condition in a subject in need thereof comprising administering a therapeutically effective amount of a composition provided herein, a pharmaceutical composition provided herein, and/or an engineered cell provided herein to the subject, thereby treating the disease or condition. A method of treating a cancer in a subject in need thereof comprising administering a therapeutically effective amount of a composition provided herein, a pharmaceutical composition provided herein, and/or an engineered cell provided herein to the subject, thereby treating the disease or condition. The method of any preceding embodiment, wherein the subject has been treated with a lymphodepletion agent prior to administration of the pharmaceutical composition. The method of any preceding embodiment, wherein the subject is treated with a lymphodepletion agent after administration of the pharmaceutical composition. The method of any preceding embodiment, wherein the subject is treated with one or more lymphodepletion agents concomitantly with the pharmaceutical composition. The method of any preceding embodiment, the one or more lymphodepletion agent comprises fludarabine or a derivative thereof. The method of any preceding embodiment, the one or more lymphodepletion agent comprises cyclophosphamide or a derivative thereof. The method of any preceding embodiment, the one or more lymphodepletion agent comprises fludarabine and cyclophosphamide. The method of any preceding embodiment, wherein the disease or condition is an autoimmune disease, a cancer, or hematopoietic stem cell transplantation. The method of any preceding embodiment, wherein the cancer is a hematological malignancy or a solid tumor. The method of any preceding embodiment, wherein the disease or condition is a leukemia, a lymphoma, a melanoma, a neuroblastoma, a glioblastoma, a carcinoma, a breast cancer, a lung cancer, a colon cancer, a pancreatic cancer, a prostate cancer, a colorectal cancer, a stomach cancer, an ovarian cancer, a uterine cancer, or a bladder cancer. The method of any preceding embodiment, wherein the disease or condition is Myasthenia Gravis (MG), Neuromyelitis optica spectrum disorder (NMOSD), Sjorgen's syndrome (SS), scleroderma, immune nephritis, systemic lupus erythematosus (SLE), or mucosal-dominant PV (mPV), multiple sclerosis (MS), or Type 1 diabetes. The method of any preceding embodiment, wherein the administering is intravenous administration, subcutaneous administration, intramuscular administration, intratumoral administration, vaginal administration, intracerebral administration, intrathecal administration, rectal administration, oral administration, ocular administration, otic administration, intranasal administration, intra-arterial administration, intraosseous administration, intradermal administration, intraperitoneal administration, or inhalation. The method of any preceding embodiment, method further comprises administering to the subject in need thereof fludarabine and/or cyclophosphamide.

Embodiment 168. A method of treating a cancer in a subject in need thereof, the method comprising: administering to the subject in need thereof a therapeutically effective amount of the composition of any preceding embodiment, thereby treating the disease or the condition.

Embodiment 169. A method of treating a cancer in a subject in need thereof, the method comprising: administering to the subject in need thereof a therapeutically effective amount of a vector of any preceding embodiment, thereby treating the disease or the condition.

Embodiment 170. A method of treating a cancer in a subject in need thereof, the method comprising: ex vivo contacting leukocytes isolated from the subject with a vector provided herein or a composition provided herein to generate a population of genetically engineered leukocytes; and administering to the population of genetically engineered leukocytes to the subject, thereby treating the cancer. The method of any preceding embodiment, wherein the leukocytes isolated from the subject comprise a population of T cells. The method of any preceding embodiment, wherein the leukocytes isolated from the subject comprise a population of tumor infiltrating lymphocytes (TILs). The method of any preceding embodiment, wherein the population of genetically engineered leukocytes are resistant to fludarabine-mediated cell death relative to a population of leukocytes that have not be contacted with the vector. The method of any preceding embodiment, wherein the population of genetically engineered leukocytes are resistant to cyclophosphamide-mediated cell death relative to a population of leukocytes that have not be contacted with the vector. The method of any preceding embodiment, wherein the administering is intravenous administration. The method of any preceding embodiment, wherein the administering is intratumoral administration. The method of any preceding embodiment, wherein the cancer is a blood cancer. The method of any preceding embodiment, wherein the cancer is a solid tumor cancer. The method of any preceding embodiment, wherein the subject is a human subject.

### EXAMPLES

The following illustrative examples are representative of embodiments of compositions and methods described herein and are not meant to be limiting in any way.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

The breadth of combinations of cell modifications, cell modulating agents, and methods of cell modification can be applicable to all extant forms of ex vivo and in vivo T cell genetic engineering and adoptive therapeutic transfer, in addition to novel cellular immunotherapeutic approaches not currently in development.

### Example 1: CD3⁺ T Cells Engineered to Express Fludarabine Resistance Genes have Increased Cell Survival

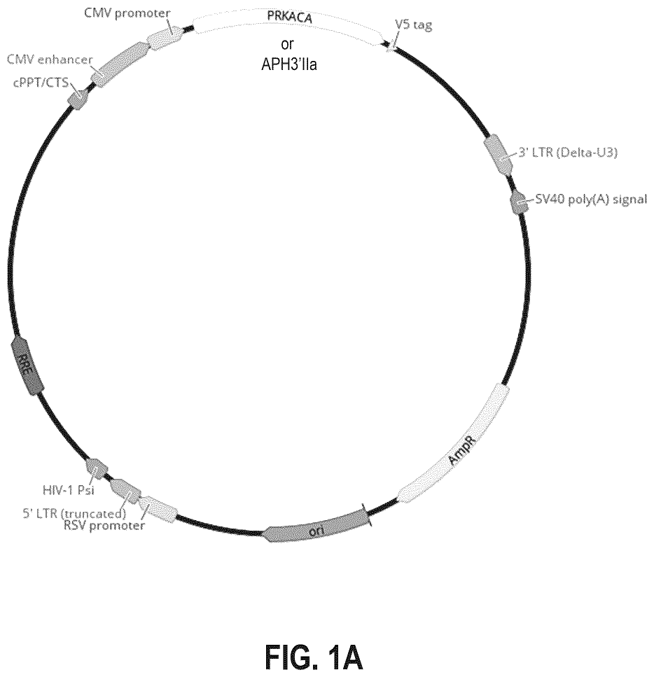

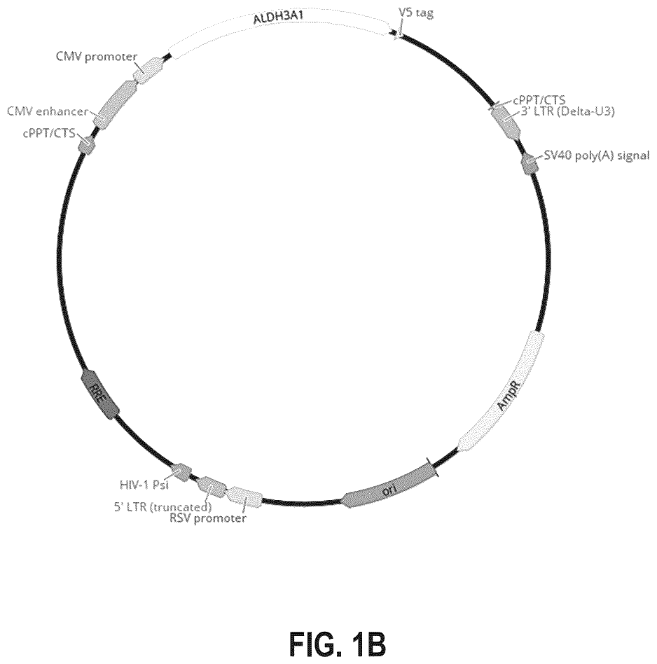

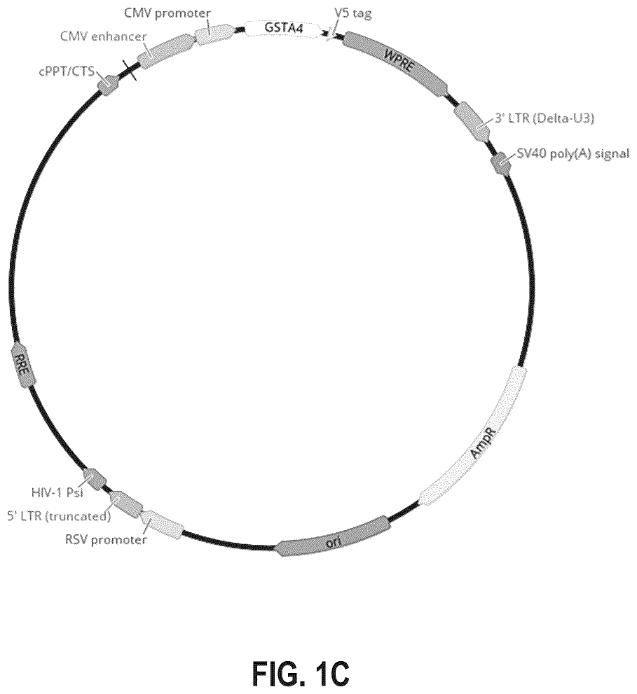

Gene sequences for APH(3′)-IIa or PRKACA were cloned into a lentiviral transfer vector plasmid downstream of a CMV promoter (FIG. 1A). Lentiviral vectors were produced by co-transfection of 293T cells (ATCC) using PEImax (Polysciences) of each respective gene transfer vector with a 2nd Generation commercial lentiviral packaging plasmid system (Cellecta) to provide lentiviral packaging functions and VSV-G pseudotype expression. Three days after transfection, Lentiviral culture supernatants were collected, filtered to remove particulates and cell debris and aliquots were frozen at −80° C. until needed for cell assays.

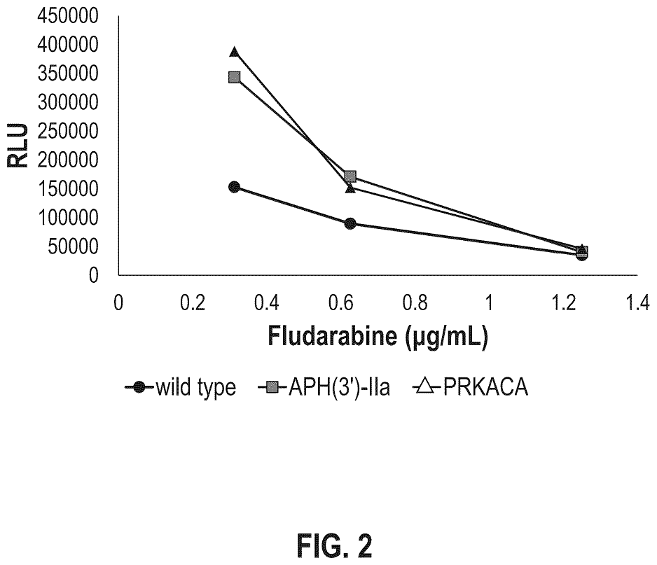

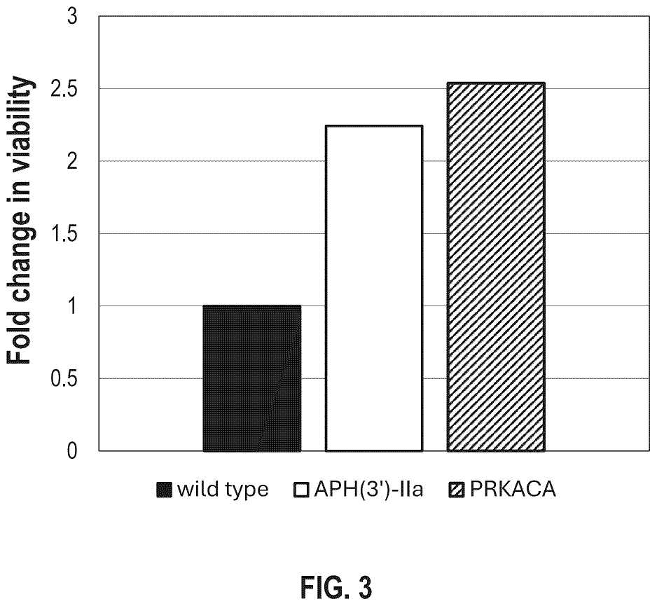

Activated Human CD3⁺ T cells were transduced with lentiviral constructs to drive expression of the APH(3′)-IIa or PRKACA candidate fludarabine resistance genes. On day 6 post-activation, fludarabine was added to cell culture media and cells were grown in the presence of fludarabine for a further 4 days. Cell survival was measured using CellTiter-Glo® Luminescent Cell Viability Assay (Promega). Dose-responsive changes in live cell abundance (FIG. 2) are shown as a function of Gene identity (wildtype, APH(3′)-IIa, or PRKACA). And the fold increase in cell survival (FIG. 3) is shown normalized to wildtype. Compared to wild type cells in the presence of fludarabine expression of both APH(3′)-IIa or PRKACA enables a >2-fold or >2.5-fold increase in survival of T cells, respectively.

### Example 2: CD3⁺ T Cells Engineered to Express Cyclophosphamide Resistance Genes have Increased Cell Survival

Gene sequences for ALDH3A1 or GSTA4 were cloned into a lentiviral transfer vector plasmid downstream of a CMV promoter (FIG. 1B and FIG. 1C). Lentiviral vectors were produced by co-transfection of 293T cells (ATCC) using PEImax (Polysciences) of each respective gene transfer vector with a 2nd Generation commercial lentiviral packaging plasmid system (Cellecta) to provide lentiviral packaging functions and VSV-G pseudotype expression. Three days after transfection, Lentiviral culture supernatants were collected, filtered to remove particulates and cell debris and aliquots were frozen at −80° C. until needed for cell assays.

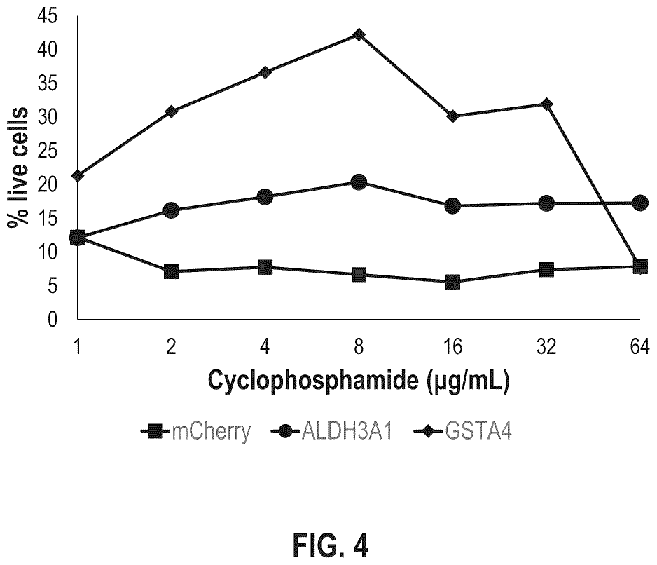

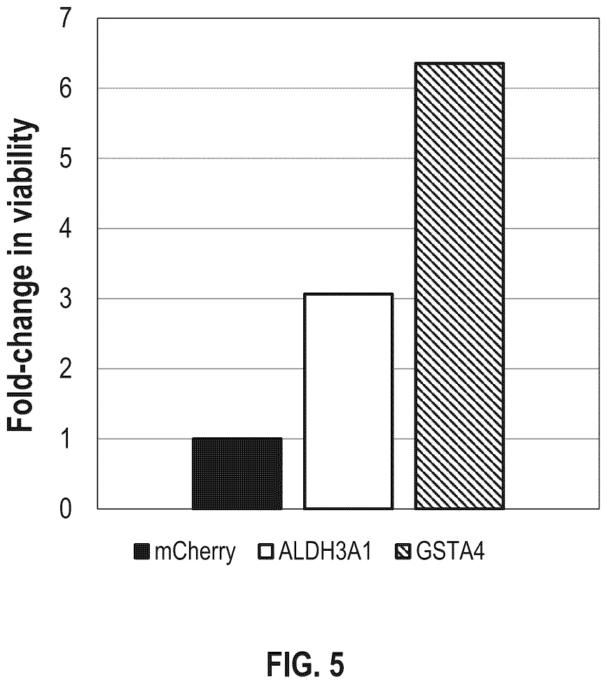

Activated Human CD3⁺ T cells were transduced with lentiviral constructs to drive expression of the ALDH3A1 or GSTA4 candidate cyclophosphamide resistance genes. On day 5 post-activation, cyclophosphamide was added to cell culture media and cells were grown in the presence of cyclophosphamide for a further 3 days. Cell survival was measured using flow cytometry. Dose-responsive changes in live cell % (FIG. 3) are shown as a function of gene identity (mCherry as an inert control gene, ALDH3A1, or GSTA4). And the fold increase in cell survival of gene-modified cells at a dose of 8 μg/mL cyclophosphamide (FIG. 4) is shown normalized to mCherry-expressing cells. Compared to mCherry cells in the presence of cyclophosphamide expression of either ALDH3A1 or GSTA4 enables a 3-fold or >6-fold increase in survival of T cells, respectively.

### Example 3: Modulating dCK Expression in Autologous Car-T Cells For Patients with Preconditioning Regimen

A leukapheresis product comprising immune cells is collected from a patient afflicted with cancer (e.g., a lymphoma). T cells are enriched from the leukapheresis product and cultivated ex vivo. The T cells are activated through the endogenous T cell receptor (TCR) using TCR stimulatory reagents. The activated T cells are transduced with virus (e.g., lentivirus) encoding a chimeric antigen receptor (CAR), protein kinase cAlvIP-activated catalytic subunit alpha (PRKACA), and a cell modulating agent that facilitates deoxycytidine kinase (DCK) knockout or knockdown (e.g., a DCK-targeting short interfering RNA (siRNA)). The CAR-T cells are cultivated ex vivo with cytokine supplementation, expanded, and collected.

The cancer patient undergoes a preconditioning lymphodepletion regimen of fludarabine alone or in combination with cyclophosphamide prior to infusion of the manufactured CAR-T cells. The CAR-T cells are administered to the cancer patient. The tumor burden of the cancer patient can be monitored using positron emission tomography (PET)-computed tomography (CT), tumor biopsies, and peripheral blood draws. The status of the CAR-T cells can be monitored using tumor biopsies and peripheral blood draws. Subsequent rounds of lymphodepletion via fludarabine alone or in combination with cyclophosphamide can be administered to the cancer patient to expand and prolong the persistence of CAR-T cells inside the cancer patient.

### Example 4: Modulating dCK and PRKACA Expression in Autologous Car-T Cells for Patients Concurrently Undergoing Lymphodepletion

A leukapheresis product comprising immune cells is collected from a patient afflicted with cancer (e.g., a melanoma) having undergone multiple lines of prior therapy. T cells are enriched from the leukapheresis product and cultivated ex vivo. The T cells are activated through the endogenous T cell receptor (TCR) using TCR stimulatory reagents. The activated T cells are transduced with virus (e.g., retrovirus) encoding a chimeric antigen receptor (CAR), a cell modulating agent that facilitates deoxycytidine kinase (DCK) knockout or knockdown (e.g., aDCK-targeting microRNA (miRNA)), and protein kinase cAlvIP-activated catalytic subunit alpha (PRKACA). The CAR-T cells are cultivated ex vivo with cytokine supplementation, expanded, and collected.

The cancer patient undergoes a preconditioning lymphodepletion regimen of fludarabine alone or in combination with cyclophosphamide during ex vivo manufacture of the CAR-T cells. After ex vivo manufacturing, the CAR-T cells are administered to the patient concurrent with lymphodepletion using fludarabine alone or in combination with cyclophosphamide. The tumor burden of the cancer patient can be monitored using positron emission tomography (PET)-computed tomography (CT), tumor biopsies, and peripheral blood draws. The status of the CAR-T cells can be monitored using tumor biopsies and peripheral blood draws. Subsequent rounds of lymphodepletion via fludarabine alone or in combination with cyclophosphamide can be administered to the cancer patient to expand and prolong the persistence of CAR-T cells inside the cancer patient.

### Example 5: Modulating APH(J′)-ILA Expression in Allogeneic TCR-T Cells for Cancer Patients with Preconditioning Regimen

A leukapheresis product comprising immune cells is collected from a healthy donor. T cells are enriched from the leukapheresis product and cultivated ex vivo. The T cells are activated through the endogenous T cell receptor (TCR) using TCR stimulatory reagents. The activated T cells are genetically edited (e.g., via nucleofection of ribonucleoprotein (RNP) complexes comprising CRISPR/Cas9 and guide RNAs targeting beta-2 microglobulin (B2M) and T-cell receptor a constant (TRAC)) to generate an allogeneic T cell population. The allogeneic T cells are transduced with virus (e.g., lentivirus) encoding an engineered T-cell receptor (TCR) and aminoglycoside-3′-phosphotransferase-Ila (APH(J′)-Ila). The TCR-T cells are cultivated ex vivo with cytokine supplementation, expanded, and collected.

The cancer patient undergoes a preconditioning lymphodepletion regimen of fludarabine alone or in combination with cyclophosphamide prior to infusion of the allogenic TCR-T cells. The TCR-T cells are administered to the cancer patient. The tumor burden of the cancer patient can be monitored using positron emission tomography (PET)-computed tomography (CD, tumor biopsies, and peripheral blood draws. The status of the TCR-T cells can be monitored using tumor biopsies and peripheral blood draws. Subsequent rounds of lymphodepletion via fludarabine alone or in combination with cyclophosphamide can be administered to the cancer patient to expand and prolong the persistence of TCR-T cells inside the cancer patient.

### Example 6: In Vivo CAR-T Cell Modification Using Lipid Nanoparticles

A cancer patient having been treated with CAR-T cells is selected for in vivo CAR-T cell modification. Lipid nanoparticles (LNPs) comprising an anti-CAR antibody on the lipid membrane and a non-viral DNA cargo encoding for protein kinase cAMP-activated catalytic subunit alpha (PRKACA) and aminoglycoside-3′-phosphotransferase-Ila (APH(3′)-Ila) are administered to the cancer patient. The LNPs preferentially bind to the CARs on the CAR-T cell surface via the anti-CAR antibody and deliver the non-viral DNA cargo encoding PRKACA and APH(3′)-Ila into the CAR-T cells. In vivo payload delivery by CAR-targeting LNPs can enable expression of PRKACA and APH(3′)-Ila. After administration of the LNPs, the cancer patient can undergo new courses of lymphodepletion using fludarabine alone or in combination with cyclophosphamide. CAR-T cells receiving the non-viral DNA cargo can be re-expanded following the subsequent courses of fludarabine-based lymphodepletion, prolonging the durability and performance of CAR-T cells in vivo.

### Example 7: In Vivo TIL-Modification Using Lipid Nanoparticles

A cancer patient is selected for transient in vivo TIL-modification. Lipid nanoparticles (LNPs) comprising an anti-PD-LI antibody on the lipid membrane and a DNA cargo encoding kinase cAlvJP-activated catalytic subunit alpha (PRKACA) and a molecule that reactivates an immune cell or reverses a cell's exhaustion state (e.g., transcription factor Jun (c-Jun)) are administered to the cancer patient intratumorally. In this example, the PD-LI-targeting LNPs can preferentially bind to PD-LI expressed on the surface of tumor reactive TILs via the anti-PD-LI antibody and deliver the non-viral DNA cargo encoding PRKACA and c-Jun into tumor reactive TILs. In vivo payload delivery by PD-LI-targeting LNPs can enable expression of PRKACA and c-Jun. After administration of the LNPs, the cancer patient can undergo new courses of lymphodepletion using fludarabine alone or in combination with cyclophosphamide. TILs receiving the DNA cargo can have their exhaustive state reversed and can be re-expanded following fludarabine-based lymphodepletion, thereby amplifying the cancer patient's own existing but overwhelmed antitumor immunity.

### Example 8: Transient In Vivo TIL-Modification Using Lipid Nanoparticles

A cancer patient bearing a solid tumor undergoing checkpoint blockade immunotherapy is selected for transient in vivo TIL-modification. Lipid nanoparticles (LNPs) comprising an anti-CD69 nanobody on the lipid membrane and an RNA cargo encoding for aminoglycoside-3′-phosphotransferase-Ila (APH(3′)-Ila) and a molecule that reactivates an immune cell or reverses a cell's exhaustion state (e.g., interleukin 2 (IL-2)) are administered intratumorally into the cancer patient. In this example, the CD69-targeting LNPs can preferentially bind to CD69 expressed on the surface of TIL-s and deliver the RNA cargo encoding APH(3′)-Ila and IL-2 into the TIL-s. In vivo payload delivery by CD69-targeting LNPs can enable transient expression of APH(3′)-Ila and IL-2. After administration of the LNPs, the cancer patient can undergo new courses of lymphodepletion using fludarabine alone or in combination with cyclophosphamide. TIL-s receiving the RNA cargo may be re-expanded following subsequent courses of fludarabine-based lymphodepletion, thereby amplifying the cancer patient's own existing but overwhelmed antitumor immunity.

### SEQUENCES

```
SEQ ID NO: 1: DNA encoding for aminoglycoside 3′-phosphotransferase-
APH(3′)-IIa-[synthetic construct]
      1 atgattgaac aagatggatt gcacgcaggt tctccggccg cttgggtgga gaggctattc
    61 ggctatgact gggcacaaca gacaatcggc tgctctgatg ccgccgtgtt ccggctgtca
  121 gcgcaggggc gcccggttct ttttgtcaag accgacctgt ccggtgccct gaatgaactg
  181 caagacgagg cagcgcggct atcgtggctg gccacgacgg gcgttccttg cgcagctgtg
  241 ctcgacgttg tcactgaagc gggaagggac tggctgctat tgggcgaagt gccggggcag
  301 gatctcctgt catctcacct tgctcctgcc gagaaagtat ccatcatggc tgatgcaatg
  361 cggcggctgc atacgcttga tccggctacc tgcccattcg accaccaagc gaaacatcgc
  421 atcgagcgag cacgtactcg gatggaagcc ggtcttgtcg atcaggatga tctggacgaa
  481 gagcatcagg ggctcgcgcc agccgaactg ttcgccaggc tcaaggcgag catgcccgac
  541 ggcgaggatc tcgtcgtgac ccatggcgat gcctgcttgc cgaatatcat ggtggaaaat
  601 ggccgctttt ctggattcat cgactgtggc cggctgggtg tggcggaccg ctatcaggac
  661 atagcgttgg ctacccgtga tattgctgaa gagcttggcg gcgaatgggc tgaccgcttc
  721 ctcgtgcttt acggtatcgc cgctcccgat tcgcagcgca tcgccttcta tcgccttctt
  781 gacgagttct tc
SEQ ID NO: 2: RNA encoding for APH(3′)-IIa-[synthetic construct]
AUGAUUGAACAAGAUGGAUUGCACGCAGGUUCUCCGGCCGCUUGGGUGGAGAGGCUAUUCGGCUAUGACUGGGCACA
ACAGACAAUCGGCUGCUCUGAUGCCGCCGUGUUCCGGCUGUCAGCGCAGGGGCGCCCGGUUCUUUUUGUCAAGACCG
ACCUGUCCGGUGCCCUGAAUGAACUGCAAGACGAGGCAGCGCGGCUAUCGUGGCUGGCCACGACGGGCGUUCCUUGC
GCAGCUGUGCUCGACGUUGUCACUGAAGCGGGAAGGGACUGGCUGCUAUUGGGCGAAGUGCCGGGGCAGGAUCUCCU
GUCAUCUCACCUUGCUCCUGCCGAGAAAGUAUCCAUCAUGGCUGAUGCAAUGCGGCGGCUGCAUACGCUUGAUCCGG
CUACCUGCCCAUUCGACCACCAAGCGAAACAUCGCAUCGAGCGAGCACGUACUCGGAUGGAAGCCGGUCUUGUCGAU
CAGGAUGAUCUGGACGAAGAGCAUCAGGGGCUCGCGCCAGCCGAACUGUUCGCCAGGCUCAAGGCGAGCAUGCCCGA
CGGCGAGGAUCUCGUCGUGACCCAUGGCGAUGCCUGCUUGCCGAAUAUCAUGGUGGAAAAUGGCCGCUUUUCUGGAU
UCAUCGACUGUGGCCGGCUGGGUGUGGCGGACCGCUAUCAGGACAUAGCGUUGGCUACCCGUGAUAUUGCUGAAGAG
CUUGGCGGCGAAUGGGCUGACCGCUUCCUCGUGCUUUACGGUAUCGCCGCUCCCGAUUCGCAGCGCAUCGCCUUCUA
UCGCCUUCUUGACGAGUUCUUC
SEQ ID NO: 3: APH(3′)-IIa-Translated Protein
MIEQDGLHAGSPAAWVERLFGYDWAQQTIGCSDAAVFRLSAQGRPVLFVKTDLSGALNELQDEAARLSWLATTGVPC
AAVLDVVTEAGRDWLLLGEVPGQDLLSSHLAPAEKVSIMADAMRRLHTLDPATCPFDHQAKHRIERARTRMEAGLVD
QDDLDEEHQGLAPAELFARLKASMPDGEDLVVTHGDACLPNIMVENGRFSGFIDCGRLGVADRYQDIALATRDIAEE
LGGEWADRFLVLYGIAAPDSQRIAFYRLLDEFF
SEQ ID NO: 4: Escherichia coli APH(3′)-IIa
RLSAQGRPVLFVKTDLSGALNELQDEAARLSWLATTGVPCAAVLDVVTEAGRDWLLLGEVPGQDLLSSHLAPAEKVS
IMADAMRRLHTLDPATCPFDHQAKHRIERARTRMEAGLVDQDDLDEEHQGLAPAELFARLKARMPDGEDLVVTHGDA
CLPNIMVENGRFSGFIDCGRLGVADRYQDIALATRDIAEELGGEWADRFLVL
SEQ ID NO: 5: Klebsiella pneumoniae APH(3′)-IIa
MIEQDGLHAGSPAAWVERLFGYDWAQQTIGCSDAAVFRLSAQGRPVLFVKTDLSGALNELQDEAARLSWLATTGVPC
AAVLDVVTEAGRDWLLLGEVPGQDLLSSHLAPAEKVSIMADAMRRLHTLDPATCPFDHQAKHRIERARTRMEAGLVD
QDDLDEEHQGLAPAELFARLKARMPDGEDLVVTHGDACLPNIMVENGRFSGFIDCGRLGVADRYQDIALATRDIAEE
LGGEWADRFLVLYGIAAPDSQRIAFYRLLDEFF
SEQ ID NO: 6: Salmonella typhimurium APH(3′)-IIa
MIEQDGLHAGSPAAWVERLFGYDWAQQTIGCSDAAVFRLSAQGRPVLFVKTDLSGALNELQDEAARLSWLATTGVPC
AAVLDVVTEAGRDWLLLGEVPGQDLLSSHLAPAEKVSIMADAMRRLHTLDPATCPFDHQAKHRIERARTRMEAGLVD
QDDLDEEHQGLAPAELFARLKARMPDGEDLVVTHGDACLPNIMVENGRFSGFIDCGRLGVADRYQDIALATRDIAEE
LGGEWADRFLVLYGIAAPDSQRIAFYRLLDEFF
SEQ ID NO: 7: Vibrio cholerae Mex1 APH(3′)-IIa
MIEQDGLHAGSPAAWVERLFGYDWAQQTIGCSDAAVFRLSAQGRPVLFVKTDLSGALNELQDEAARLSWLATTGVPC
AAVLDVVTEAGRDWLLLGEVPGQDLLSSHLAPAEKVSIMADAMRRLHTLDPATCPFDHQAKHRIERARTRMEAGLVD
QDDLDEEHQGLAPAELFARLKARMPDGEDLVVTHGDACLPNIMVENGRFSGFIDCGRLGVADRYQDIALATRDIAEE
LGGEWADRFLVLYGIAAPDSQRIAFYRLLDEFF
SEQ ID NO: 8-DNA encoding for PRKACA [synthetic construct]
      1 atgggcaacg ccgccgccgc caagaagggc agcgagcagg agagcgtgaa agaattatta
    61 gccaaagcca aagaagattt tcttaaaaaa tgggaaagtc ccgctcagaa cacagcccac
  121 ttggatcagt ttgaacgaat caagaccctc ggcacgggct ccttcgggcg ggtgatgctg
  181 gtgaaacaca aggagaccgg gaaccactat gccatgaaga tcctcgacaa acagaaggtg
  241 gtgaaactga aacagatcga acacaccctg aatgaaaagc gcatcctgca agctgtcaac
  301 tttccgttcc tcgtcaaact cgagttctcc ttcaaggaca actcaaactt atacatggtc
  361 atggagtacg tgcccggcgg ggagatgttc tcacacctac ggcggatcgg aaggttcagt
  421 gagccccatg cccgtttcta cgcggcccag atcgtcctga cctttgagta tctgcactcg
  481 ctggatctca tctacaggga cctgaagccg gagaatctgc tcattgacca gcagggctac
  541 attcaggtga cagacttcgg tttcgccaag cgcgtgaagg gccgcacttg gaccttgtgc
  601 ggcacccctg agtacctggc ccctgagatt atcctgagca aaggctacaa caaggccgtg
  661 gactggtggg ccctgggggt tcttatctat gaaatggccg ctggctaccc gcccttcttc
  721 gcagaccagc ccatccagat ctatgagaag atcgtctctg ggaaggtgcg cttcccttcc
  781 cacttcagct ctgacttgaa ggacctgctg cggaacctcc tgcaggtaga tctcaccaag
  841 cgctttggga acctcaagaa tggggtcaac gatatcaaga accacaagtg gtttgccaca
  901 actgactgga ttgccatcta ccagaggaag gtggaagctc ccttcatacc aaagtttaaa
  961 ggccctgggg atacgagtaa ctttgacgac tatgaggaag aagaaatccg ggtctccatc
1021 aatgagaagt gtggcaagga gttttctgag ttt
SEQ ID NO: 9-RNA encoding for PRKACA [synthetic construct]
AUGGGCAACGCCGCCGCCGCCAAGAAGGGCAGCGAGCAGGAGAGCGUGAAAGAAUUCUUAGCCAAAGCCAAAGAAGA
UUUUCUUAAAAAAUGGGAAAGUCCCGCUCAGAACACAGCCCACUUGGAUCAGUUUGAACGAAUCAAGACCCUCGGCA
CGGGCUCCUUCGGGCGGGUGAUGCUGGUGAAACACAAGGAGACCGGGAACCACUAUGCCAUGAAGAUCCUCGACAAA
CAGAAGGUGGUGAAACUGAAACAGAUCGAACACACCCUGAAUGAAAAGCGCAUCCUGCAAGCUGUCAACUUUCCGUU
CCUCGUCAAACUCGAGUUCUCCUUCAAGGACAACUCAAACUUAUACAUGGUCAUGGAGUACGUGCCCGGCGGGGAGA
UGUUCUCACACCUACGGCGGAUCGGAAGGUUCAGUGAGCCCCAUGCCCGUUUCUACGCGGCCCAGAUCGUCCUGACC
UUUGAGUAUCUGCACUCGCUGGAUCUCAUCUACAGGGACCUGAAGCCGGAGAAUCUGCUCAUUGACCAGCAGGGCUA
CAUUCAGGUGACAGACUUCGGUUUCGCCAAGCGCGUGAAGGGCCGCACUUGGACCUUGUGCGGCACCCCUGAGUACC
UGGCCCCUGAGAUUAUCCUGAGCAAAGGCUACAACAAGGCCGUGGACUGGUGGGCCCUGGGGGUUCUUAUCUAUGAA
AUGGCCGCUGGCUACCCGCCCUUCUUCGCAGACCAGCCCAUCCAGAUCUAUGAGAAGAUCGUCUCUGGGAAGGUGCG
CUUCCCUUCCCACUUCAGCUCUGACUUGAAGGACCUGCUGCGGAACCUCCUGCAGGUAGAUCUCACCAAGCGCUUUG
GGAACCUCAAGAAUGGGGUCAACGAUAUCAAGAACCACAAGUGGUUUGCCACAACUGACUGGAUUGCCAUCUACCAG
AGGAAGGUGGAAGCUCCCUUCAUACCAAAGUUUAAAGGCCCUGGGGAUACGAGUAACUUUGACGACUAUGAGGAAGA
AGAAAUCCGGGUCUCCAUCAAUGAGAAGUGUGGCAAGGAGUUUUCUGAGUUU
SEQ ID NO: 10: PRKACA-Translated Protein
MGNAAAAKKGSEQESVKEFLAKAKEDFLKKWESPAQNTAHLDQFERIKTLGTGSFGRVMLVKHKETGNHYAMKILDK
QKVVKLKQIEHTLNEKRILQAVNFPFLVKLEFSFKDNSNLYMVMEYVPGGEMFSHLRRIGRFSEPHARFYAAQIVLT
FEYLHSLDLIYRDLKPENLLIDQQGYIQVTDFGFAKRVKGRTWTLCGTPEYLAPEIILSKGYNKAVDWWALGVLIYE
MAAGYPPFFADQPIQIYEKIVSGKVRFPSHFSSDLKDLLRNLLQVDLTKRFGNLKNGVNDIKNHKWFATTDWIAIYQ
RKVEAPFIPKFKGPGDTSNFDDYEEEEIRVSINEKCGKEFSEF
SEQ ID NO: 11: PRKACA-CAMP-dependent protein kinase catalytic subunit
alpha isoform 3 [Homo sapiens]
MFTLGYHGAEEWASGETSLEPALEQCPCDCHGEEQGQLGAKVTAEPPKGTCYPSPRGLHPRPGLPREGAR
GTAKSQGYKYLGWRRPWPWNVVKEFLAKAKEDFLKKWESPAQNTAHLDQFERIKTLGTGSFGRVMLVKHK
ETGNHYAMKILDKQKVVKLKQIEHTLNEKRILQAVNFPFLVKLEFSFKDNSNLYMVMEYVPGGEMFSHLR
RIGRFSEPHARFYAAQIVLTFEYLHSLDLIYRDLKPENLLIDQQGYIQVTDFGFAKRVKGRTWTLCGTPE
YLAPEIILSKGYNKAVDWWALGVLIYEMAAGYPPFFADQPIQIYEKIVSGKVRFPSHFSSDLKDLLRNLL
QVDLTKRFGNLKNGVNDIKNHKWFATTDWIAIYQRKVEAPFIPKFKGPGDTSNFDDYEEEEIRVSINEKC
GKEFSEF
SEQ ID NO: 12: PRKACA-CAMP-dependent protein kinase catalytic subunit
alpha isoform Calpha1 [Homo sapiens]
MGNAAAAKKGSEQESVKEFLAKAKEDFLKKWESPAQNTAHLDQFERIKTLGTGSFGRVMLVKHKETGNHY
AMKILDKQKVVKLKQIEHTLNEKRILQAVNFPFLVKLEFSFKDNSNLYMVMEYVPGGEMFSHLRRIGRFS
EPHARFYAAQIVLTFEYLHSLDLIYRDLKPENLLIDQQGYIQVTDFGFAKRVKGRTWTLCGTPEYLAPEI
ILSKGYNKAVDWWALGVLIYEMAAGYPPFFADQPIQIYEKIVSGKVRFPSHFSSDLKDLLRNLLQVDLTK
RFGNLKNGVNDIKNHKWFATTDWIAIYQRKVEAPFIPKFKGPGDTSNFDDYEEEEIRVSINEKCGKEFSE
F
SEQ ID NO: 13: PRKACA-CAMP-dependent protein kinase catalytic
subunit alpha isoform Calpha2 [Homo sapiens]
MASNSSDVKEFLAKAKEDFLKKWESPAQNTAHLDQFERIKTLGTGSFGRVMLVKHKETGNHYAMKILDKQ
KVVKLKQIEHTLNEKRILQAVNFPFLVKLEFSFKDNSNLYMVMEYVPGGEMFSHLRRIGRFSEPHARFYA
AQIVLTFEYLHSLDLIYRDLKPENLLIDQQGYIQVTDFGFAKRVKGRTWTLCGTPEYLAPEIILSKGYNK
AVDWWALGVLIYEMAAGYPPFFADQPIQIYEKIVSGKVRFPSHFSSDLKDLLRNLLQVDLTKRFGNLKNG
VNDIKNHKWFATTDWIAIYQRKVEAPFIPKFKGPGDTSNFDDYEEEEIRVSINEKCGKEFSEF
SEQ ID NO: 14: PRKACA-CAMP-dependent protein kinase catalytic subunit
alpha isoform 1 [Mus musculus]
MGNAAAAKKGSEQESVKEFLAKAKEDFLKKWETPSQNTAQLDQFDRIKTLGTGSFGRVMLVKHKESGNHY
AMKILDKQKVVKLKQIEHTLNEKRILQAVNFPFLVKLEFSFKDNSNLYMVMEYVAGGEMFSHLRRIGRFS
EPHARFYAAQIVLTFEYLHSLDLIYRDLKPENLLIDQQGYIQVTDFGFAKRVKGRTWTLCGTPEYLAPEI
ILSKGYNKAVDWWALGVLIYEMAAGYPPFFADQPIQIYEKIVSGKVRFPSHFSSDLKDLLRNLLQVDLTK
RFGNLKNGVNDIKNHKWFATTDWIAIYQRKVEAPFIPKFKGPGDTSNFDDYEEEEIRVSINEKCGKEFTE
F
SEQ ID NO: 15: DNA encoding for aldehyde dehydrogenase 3 family, member
A1, ALDH3A1-[synthetic construct]
      1 atgagcaaga tcagcgaggc cgtgaagcgc gcccgcgccg ccttcagctc gggcaggacc
    61 cgtccgctgc agttccggat ccagcagctg gaggcgctgc agcgcctgat ccaggagcag
  121 gagcaggagc tggtgggcgc gctggccgca gacctgcaca agaatgaatg gaacgcctac
  181 tatgaggagg tggtgtacgt cctagaggag atcgagtaca tgatccagaa gctccctgag
  241 tgggccgcgg atgagcccgt ggagaagacg ccccagactc agcaggacga gctctacatc
  301 cactcggagc cactgggcgt ggtcctcgtc attggcacct ggaactaccc cttcaacctc
  361 accatccagc ccatggtggg cgccatcgct gcagggaacg cagtggtcct caagccctcg
  421 gagctgagtg agaacatggc gagcctgctg gctaccatca tcccccagta cctggacaag
  481 gatctgtacc cagtaatcaa tgggggtgtc cctgagacca cggagctgct caaggagagg
  541 ttcgaccata tcctgtacac gggcagcacg ggggtgggga agatcatcat gacggctgct
  601 gccaagcacc tgacccctgt cacgctggag ctgggaggga agagtccctg ctacgtggac
  661 aagaactgtg acctggacgt ggcctgccga cgcatcgcct gggggaaatt catgaacagt
  721 ggccagacct gcgtggcccc agactacatc ctctgtgacc cctcgatcca gaaccaaatt
  781 gtggagaagc tcaagaagtc actgaaagag ttctacgggg aagatgctaa gaaatcccgg
  841 gactatggaa gaatcattag tgcccggcac ttccagaggg tgatgggcct gattgagggc
  901 cagaaggtgg cttatggggg caccggggat gccgccactc gctacatagc ccccaccatc
  961 ctcacggacg tggaccccca gtccccggtg atgcaagagg agatcttcgg gcctgtgctg
1021 cccatcgtgt gcgtgcgcag cctggaggag gccatccagt tcatcaacca gcgtgagaag
1081 cccctggccc tctacatgtt ctccagcaac gacaaggtga ttaagaagat gattgcagag
1141 acatccagtg gctggggggc ggccaacgat gtcatcgtcc acatcacctt gcactctctg
1201 cccttcgggg gcgtggggaa cagcggcatg ggatcctacc atggcaagaa gagcttcgag
1261 actttctctc accgccgctc ttgcctggtg aggcctctga tgaatgatga aggcctgaag
1321 gtcagatacc ccccgagccc ggccaagatg acccagcac
SEQ ID NO: 16: RNA encoding for aldehyde dehydrogenase 3 family, member
A1, ALDH3A1-[synthetic construct]
AUGAGCAAGAUCAGCGAGGCCGUGAAGCGCGCCCGCGCCGCCUUCAGCUCGGGCAGGACCCGUCCGCUGCAGUUCCG
GAUCCAGCAGCUGGAGGCGCUGCAGCGCCUGAUCCAGGAGCAGGAGCAGGAGCUGGUGGGCGCGCUGGCCGCAGACC
UGCACAAGAAUGAAUGGAACGCCUACUAUGAGGAGGUGGUGUACGUCCUAGAGGAGAUCGAGUACAUGAUCCAGAAG
CUCCCUGAGUGGGCCGCGGAUGAGCCCGUGGAGAAGACGCCCCAGACUCAGCAGGACGAGCUCUACAUCCACUCGGA
GCCACUGGGCGUGGUCCUCGUCAUUGGCACCUGGAACUACCCCUUCAACCUCACCAUCCAGCCCAUGGUGGGCGCCA
UCGCUGCAGGGAACGCAGUGGUCCUCAAGCCCUCGGAGCUGAGUGAGAACAUGGCGAGCCUGCUGGCUACCAUCAUC
CCCCAGUACCUGGACAAGGAUCUGUACCCAGUAAUCAAUGGGGGUGUCCCUGAGACCACGGAGCUGCUCAAGGAGAG
GUUCGACCAUAUCCUGUACACGGGCAGCACGGGGGUGGGGAAGAUCAUCAUGACGGCUGCUGCCAAGCACCUGACCC
CUGUCACGCUGGAGCUGGGAGGGAAGAGUCCCUGCUACGUGGACAAGAACUGUGACCUGGACGUGGCCUGCCGACGC
AUCGCCUGGGGGAAAUUCAUGAACAGUGGCCAGACCUGCGUGGCCCCAGACUACAUCCUCUGUGACCCCUCGAUCCA
GAACCAAAUUGUGGAGAAGCUCAAGAAGUCACUGAAAGAGUUCUACGGGGAAGAUGCUAAGAAAUCCCGGGACUAUG
GAAGAAUCAUUAGUGCCCGGCACUUCCAGAGGGUGAUGGGCCUGAUUGAGGGCCAGAAGGUGGCUUAUGGGGGCACC
GGGGAUGCCGCCACUCGCUACAUAGCCCCCACCAUCCUCACGGACGUGGACCCCCAGUCCCCGGUGAUGCAAGAGGA
GAUCUUCGGGCCUGUGCUGCCCAUCGUGUGCGUGCGCAGCCUGGAGGAGGCCAUCCAGUUCAUCAACCAGCGUGAGA
AGCCCCUGGCCCUCUACAUGUUCUCCAGCAACGACAAGGUGAUUAAGAAGAUGAUUGCAGAGACAUCCAGUGGUGGG
GUGGCGGCCAACGAUGUCAUCGUCCACAUCACCUUGCACUCUCUGCCCUUCGGGGGCGUGGGGAACAGCGGCAUGGG
AUCCUACCAUGGCAAGAAGAGCUUCGAGACUUUCUCUCACCGCCGCUCUUGCCUGGUGAGGCCUCUGAUGAAUGAUG
AAGGCCUGAAGGUCAGAUACCCCCCGAGCCCGGCCAAGAUGACCCAGCAC
SEQ ID NO: 17: ALDH3A1-Translated Protein
MSKISEAVKRARAAFSSGRTRPLQFRIQQLEALQRLIQEQEQELVGALAADLHKNEWNAYYEEVVYVLEEIEYMIQK
LPEWAADEPVEKTPQTQQDELYIHSEPLGVVLVIGTWNYPFNLTIQPMVGAIAAGNAVVLKPSELSENMASLLATII
PQYLDKDLYPVINGGVPETTELLKERFDHILYTGSTGVGKIIMTAAAKHLTPVTLELGGKSPCYVDKNCDLDVACRR
IAWGKFMNSGQTCVAPDYILCDPSIQNQIVEKLKKSLKEFYGEDAKKSRDYGRIISARHFQRVMGLIEGQKVAYGGT
GDAATRYIAPTILTDVDPQSPVMQEEIFGPVLPIVCVRSLEEAIQFINQREKPLALYMFSSNDKVIKKMIAETSSGG
VAANDVIVHITLHSLPFGGVGNSGMGSYHGKKSFETFSHRRSCLVRPLMNDEGLKVRYPPSPAKMTQH
SEQ ID NO: 18: aldehyde dehydrogenase, mitochondrial isoform 1 precursor
[Homo sapiens]
MLRAAARFGPRLGRRLLSAAATQAVPAPNQQPEVFCNQIFINNEWHDAVSRKTFPTVNPSTGEVICQVAE
GDKEDVDKAVKAARAAFQLGSPWRRMDASHRGRLLNRLADLIERDRTYLAALETLDNGKPYVISYLVDLD
MVLKCLRYYAGWADKYHGKTIPIDGDFFSYTRHEPVGVCGQIIPWNFPLLMQAWKLGPALATGNVVVMKV
AEQTPLTALYVANLIKEAGFPPGVVNIVPGFGPTAGAAIASHEDVDKVAFTGSTEIGRVIQVAAGSSNLK
RVTLELGGKSPNIIMSDADMDWAVEQAHFALFFNQGQCCCAGSRTFVQEDIYDEFVERSVARAKSRVVGN
PFDSKTEQGPQVDETQFKKILGYINTGKQEGAKLLCGGGIAADRGYFIQPTVFGDVQDGMTIAKEEIFGP
VMQILKFKTIEEVVGRANNSTYGLAAAVFTKDLDKANYLSQALQAGTVWVNCYDVFGAQSPFGGYKMSGS
GRELGEYGLQAYTEVKTVTVKVPQKNS
SEQ ID NO: 19: DNA encoding for Homo sapiens glutathione S-transferase
alpha 4 (GSTA4)-[synthetic construct]
      1 atgtacgtgg aggggacact ggatctgctg gaactgctta tcatgcatcc tttcttaaaa
    61 ccagatgatc agcaaaagga agtggttaac atggcccaga aggctataat tagatacttt
  121 cctgtgtttg aaaagatttt aaggggtcac ggacaaagct ttcttgttgg taatcagctg
  181 agccttgcag atgtgatttt actccaaacc attttagctc tagaagagaa aattcctaat
  241 atcctgtctg catttccttt cctccaggaa tacacagtga aactaagtaa tatccctaca
  301 attaagagat tccttgaacc tggcagcaag aagaagcctc cccctgatga aatttatgtg
  361 agaaccgtct acaacatctt taggcca
SEQ ID NO: 20: RNA encoding for Homo sapiens glutathione S-transferase
alpha 4 (GSTA4)-[synthetic construct]
AUGUACGUGGAGGGGACACUGGAUCUGCUGGAACUGCUUAUCAUGCAUCCUUUCUUAAAACCAGAUGAUCAGCAAAA
GGAAGUGGUUAACAUGGCCCAGAAGGCUAUAAUUAGAUACUUUCCUGUGUUUGAAAAGAUUUUAAGGGGUCACGGAC
AAAGCUUUCUUGUUGGUAAUCAGCUGAGCCUUGCAGAUGUGAUUUUACUCCAAACCAUUUUAGCUCUAGAAGAGAAA
AUUCCUAAUAUCCUGUCUGCAUUUCCUUUCCUCCAGGAAUACACAGUGAAACUAAGUAAUAUCCCUACAAUUAAGAG
AUUCCUUGAACCUGGCAGCAAGAAGAAGCCUCCCCCUGAUGAAAUUUAUGUGAGAACCGUCUACAACAUCUUUAGGC
CA
SEQ ID NO: 21: GSTA4 translated protein
MYVEGTLDLLELLIMHPFLKPDDQQKEVVNMAQKAIIRYFPVFEKILRGHGQSFLVGNQLSLADVILLQTILALEEK
IPNILSAFPFLQEYTVKLSNIPTIKRFLEPGSKKKPPPDEIYVRTVYNIFRP
SEQ ID NO: 22: NP_001503.1 glutathione S-transferase A4 [Homo sapiens]
MAARPKLHYPNGRGRMESVRWVLAAAGVEFDEEFLETKEQLYKLQDGNHLLFQQVPMVEIDGMKLVQTRS
ILHYIADKHNLFGKNLKERTLIDMYVEGTLDLLELLIMHPFLKPDDQQKEVVNMAQKAIIRYFPVFEKIL
RGHGQSFLVGNQLSLADVILLQTILALEEKIPNILSAFPFLQEYTVKLSNIPTIKRFLEPGSKKKPPPDE
IYVRTVYNIFRP
SEQ ID NO: 23: pLX304-APH(3′)-IIa
      1 ttgagatcct ttttttctgc gcgtaatctg ctgcttgcaa acaaaaaaac caccgctacc
    61 agcggtggtt tgtttgccgg atcaagagct accaactctt tttccgaagg taactggctt
  121 cagcagagcg cagataccaa atactgttct tctagtgtag ccgtagttag gccaccactt
  181 caagaactct gtagcaccgc ctacatacct cgctctgcta atcctgttac cagtggctgc
  241 tgccagtggc gataagtcgt gtcttaccgg gttggactca agacgatagt taccggataa
  301 ggcgcagcgg tcgggctgaa cggggggttc gtgcacacag cccagcttgg agcgaacgac
  361 ctacaccgaa ctgagatacc tacagcgtga gctatgagaa agcgccacgc ttcccgaagg
  421 gagaaaggcg gacaggtatc cggtaagcgg cagggtcgga acaggagagc gcacgaggga
  481 gcttccaggg ggaaacgcct ggtatcttta tagtcctgtc gggtttcgcc acctctgact
  541 tgagcgtcga tttttgtgat gctcgtcagg ggggcggagc ctatggaaaa acgccagcaa
  601 cgcggccttt ttacggttcc tggccttttg ctggcctttt gctcacatgt tctttcctgc
  661 gttatcccct gattctgtgg ataaccgtat taccgccttt gagtgagctg ataccgctcg
  721 ccgcagccga acgaccgagc gcagcgagtc agtgagcgag gaagcggaag agcgcccaat
  781 acgcaaaccg cctctccccg cgcgttggcc gattcattaa tgcagctggc acgacaggtt
  841 tcccgactgg aaagcgggca gtgagcgcaa cgcaattaat gtgagttagc tcactcatta
  901 ggcaccccag gctttacact ttatgcttcc ggctcgtatg ttgtgtggaa ttgtgagcgg
  961 ataacaattt cacacaggaa acagctatga ccatgattac gccaagcgcg caattaaccc
1021 tcactaaagg gaacaaaagc tggagctgca agcttaatgt agtcttatgc aatactcttg
1081 tagtcttgca acatggtaac gatgagttag caacatgcct tacaaggaga gaaaaagcac
1141 cgtgcatgcc gattggtgga agtaaggtgg tacgatcgtg ccttattagg aaggcaacag
1201 acgggtctga catggattgg acgaaccact gaattgccgc attgcagaga tattgtattt
1261 aagtgcctag ctcgatacat aaacgggtct ctctggttag accagatctg agcctgggag
1321 ctctctggct aactagggaa cccactgctt aagcctcaat aaagcttgcc ttgagtgctt
1381 caagtagtgt gtgcccgtct gttgtgtgac tctggtaact agagatccct cagacccttt
1441 tagtcagtgt ggaaaatctc tagcagtggc gcccgaacag ggacttgaaa gcgaaaggga
1501 aaccagagga gctctctcga cgcaggactc ggcttgctga agcgcgcacg gcaagaggcg
1561 aggggcggcg actggtgagt acgccaaaaa ttttgactag cggaggctag aaggagagag
1621 atgggtgcga gagcgtcagt attaagcggg ggagaattag atcgcgatgg gaaaaaattc
1681 ggttaaggcc agggggaaag aaaaaatata aattaaaaca tatagtatgg gcaagcaggg
1741 agctagaacg attcgcagtt aatcctggcc tgttagaaac atcagaaggc tgtagacaaa
1801 tactgggaca gctacaacca tcccttcaga caggatcaga agaacttaga tcattatata
1861 atacagtagc aaccctctat tgtgtgcatc aaaggataga gataaaagac accaaggaag
1921 ctttagacaa gatagaggaa gagcaaaaca aaagtaagac caccgcacag caagcggccg
1981 ctgatcttca gacctggagg aggagatatg agggacaatt ggagaagtga attatataaa
2041 tataaagtag taaaaattga accattagga gtagcaccca ccaaggcaaa gagaagagtg
2101 gtgcagagag aaaaaagagc agtgggaata ggagctttgt tccttgggtt cttgggagca
2161 gcaggaagca ctatgggcgc agcgtcaatg acgctgacgg tacaggccag acaattattg
2221 tctggtatag tgcagcagca gaacaatttg ctgagggcta ttgaggcgca acagcatctg
2281 ttgcaactca cagtctgggg catcaagcag ctccaggcaa gaatcctggc tgtggaaaga
2341 tacctaaagg atcaacagct cctggggatt tggggttgct ctggaaaact catttgcacc
2401 actgctgtgc cttggaatgc tagttggagt aataaatctc tggaacagat ttggaatcac
2461 acgacctgga tggagtggga cagagaaatt aacaattaca caagcttaat acactcctta
2521 attgaagaat cgcaaaacca gcaagaaaag aatgaacaag aattattgga attagataaa
2581 tgggcaagtt tgtggaattg gtttaacata acaaattggc tgtggtatat aaaattattc
2641 ataatgatag taggaggctt ggtaggttta agaatagttt ttgctgtact ttctatagtg
2701 aatagagtta ggcagggata ttcaccatta tcgtttcaga cccacctccc aaccccgagg
2761 ggacccttgc gccttttcca aggcagccct gggtttgcgc agggacgcgg ctgctctggg
2821 cgtggttccg ggaaacgcag cggcgccgac cctgggtctc gcacattctt cacgtccgtt
2881 cgcagcgtca cccggatctt cgccgctacc cttgtgggcc ccccggcgac gcttcctgct
2941 ccgcccctaa gtcgggaagg ttccttgcgg ttcgcggcgt gccggacgtg acaaacggaa
3001 gccgcacgtc tcactagtac cctcgcagac ggacagcgcc agggagcaat ggcagcgcgc
3061 cgaccgcgat gggctgtggc caatagcggc tgctcagcag ggcgcgccga gagcagcggc
3121 cgggaagggg cggtgcggga ggcggggtgt ggggcggtag tgtgggccct gttcctgccc
3181 gcgcggtgtt ccgcattctg caagcctccg gagcgcacgt cggcagtcgg ctccctcgtt
3241 gaccgaatca ccgacctctc tccccagggg gtaccaccat ggccaagcct ttgtctcaag
3301 aagaatccac cctcattgaa agagcaacgg ctacaatcaa cagcatcccc atctctgaag
3361 actacagcgt cgccagcgca gctctctcta gcgacggccg catcttcact ggtgtcaatg
3421 tatatcattt tactggggga ccttgtgcag aactcgtggt gctgggcact gctgctgctg
3481 cggcagctgg caacctgact tgtatcgtcg cgatcggaaa tgagaacagg ggcatcttga
3541 gcccctgcgg acggtgccga caggtgcttc tcgatctgca tcctgggatc aaagccatag
3601 tgaaggacag tgatggacag ccgacggcag ttgggattcg tgaattgctg ccctctggtt
3661 atgtgtggga gggcctgcag ctgcagtagt aagaattcta gatcttgaga caaatggcag
3721 tattcatcca caattttaaa agaaaagggg ggattggggg gtacagtgca ggggaaagaa
3781 tagtagacat aatagcaaca gacatacaaa ctaaagaatt acaaaaacaa attacaaaaa
3841 ttcaaaattt tcgggtttat tacagggaca gcagagatcc actttggcgc cggctcgagg
3901 gggcccgggg ttattaatag taatcaatta cggggtcatt agttcatagc ccatatatgg
3961 agttccgcgt tacataactt acggtaaatg gcccgcctgg ctgaccgccc aacgaccccc
4021 gcccattgac gtcaataatg acgtatgttc ccatagtaac gccaataggg actttccatt
4081 gacgtcaatg ggtggagtat ttacggtaaa ctgcccactt ggcagtacat caagtgtatc
4141 atatgccaag tacgccccct attgacgtca atgacggtaa atggcccgcc tggcattatg
4201 cccagtacat gaccttatgg gactttccta cttggcagta catctacgta ttagtcatcg
4261 ctattaccat ggtgatgcgg ttttggcagt acatcaatgg gcgtggatag cggtttgact
4321 cacggggatt tccaagtctc caccccattg acgtcaatgg gagtttgttt tggcaccaaa
4381 atcaacggga ctttccaaaa tgtcgtaaca actccgcccc attgacgcaa atgggcggta
4441 ggcgtgtacg gtgggaggtc tatataagca gagctctctg gctaactgtc gggatcaaca
4501 agtttgtaca aaaaagcagg ctccaccatg attgaacaag atggattgca cgcaggttct
4561 ccggccgctt gggtggagag gctattcggc tatgactggg cacaacagac aatcggctgc
4621 tctgatgccg ccgtgttccg gctgtcagcg caggggcgcc cggttctttt tgtcaagacc
4681 gacctgtccg gtgccctgaa tgaactgcaa gacgaggcag cgcggctatc gtggctggcc
4741 acgacgggcg ttccttgcgc agctgtgctc gacgttgtca ctgaagcggg aagggactgg
4801 ctgctattgg gcgaagtgcc ggggcaggat ctcctgtcat ctcaccttgc tcctgccgag
4861 aaagtatcca tcatggctga tgcaatgcgg cggctgcata cgcttgatcc ggctacctgc
4921 ccattcgacc accaagcgaa acatcgcatc gagcgagcac gtactcggat ggaagccggt
4981 cttgtcgatc aggatgatct ggacgaagag catcaggggc tcgcgccagc cgaactgttc
5041 gccaggctca aggcgagcat gcccgacggc gaggatctcg tcgtgaccca tggcgatgcc
5101 tgcttgccga atatcatggt ggaaaatggc cgcttttctg gattcatcga ctgtggccgg
5161 ctgggtgtgg cggaccgcta tcaggacata gcgttggcta cccgtgatat tgctgaagag
5221 cttggcggcg aatgggctga ccgcttcctc gtgctttacg gtatcgccgc tcccgattcg
5281 cagcgcatcg ccttctatcg ccttcttgac gagttcttct aattggaccc agctttcttg
5341 tacaaagtgg ttggtaagcc tatccctaac cctctcctcg gtctcgattc tacgtagtaa
5401 tgagctagcg ctaaccggtg gcgcgttaag tcgacaatca acctctggat tacaaaattt
5461 gtgaaagatt gactggtatt cttaactatg ttgctccttt tacgctatgt ggatacgctg
5521 ctttaatgcc tttgtatcat gctattgctt cccgtatggc tttcattttc tcctccttgt
5581 ataaatcctg gttgctgtct ctttatgagg agtigtggcc cgttgtcagg caacgtggcg
5641 tggtgtgcac tgtgtttgct gacgcaaccc ccactggttg gggcattgcc accacctgtc
5701 agctcctttc cgggactttc gctttccccc tccctattgc cacggcggaa ctcatcgccg
5761 cctgccttgc ccgctgctgg acaggggctc ggctgttggg cactgacaat tccgtggtgt
5821 tgtcggggaa atcatcgtcc tttccttggc tgctcgcctg tgttgccacc tggattctgc
5881 gcgggacgtc cttctgctac gtcccttcgg ccctcaatcc agcggacctt ccttcccgcg
5941 gcctgctgcc ggctctgcgg cctcttccgc gtcttcgcct tcgccctcag acgagtcgga
6001 tctccctttg ggccgcctcc ccgcgtcgac tttaagacca atgacttaca aggcagctgt
6061 agatcttagc cactttttaa aagaaaaggg gggactggaa gggctaattc actcccaacg
6121 aagacaagat ctgctttttg cttgtactgg gtctctctgg ttagaccaga tctgagcctg
6181 ggagctctct ggctaactag ggaacccact gcttaagcct caataaagct tgccttgagt
6241 gcttcaagta gtgtgtgccc gtctgttgtg tgactctggt aactagagat ccctcagacc
6301 cttttagtca gtgtggaaaa tctctagcag tacgtatagt agttcatgtc atcttattat
6361 tcagtattta taacttgcaa agaaatgaat atcagagagt gagaggaact tgtttattgc
6421 agcttataat ggttacaaat aaagcaatag catcacaaat ttcacaaata aagcattttt
6481 ttcactgcat tctagttgtg gtttgtccaa actcatcaat gtatcttatc atgtctggct
6541 ctagctatcc cgcccctaac tccgcccatc ccgcccctaa ctccgcccag ttccgcccat
6601 tctccgcccc atggctgact aatttttttt atttatgcag aggccgaggc cgcctcggcc
6661 tctgagctat tccagaagta gtgaggaggc ttttttggag gcctagggac gtacccaatt
6721 cgccctatag tgagtcgtat tacgcgcgct cactggccgt cgttttacaa cgtcgtgact
6781 gggaaaaccc tggcgttacc caacttaatc gccttgcagc acatccccct ttcgccagct
6841 ggcgtaatag cgaagaggcc cgcaccgatc gcccttccca acagttgcgc agcctgaatg
6901 gcgaatggga cgcgccctgt agcggcgcat taagcgcggc gggtgtggtg gttacgcgca
6961 gcgtgaccgc tacacttgcc agcgccctag cgcccgctcc tttcgctttc ttcccttcct
7021 ttctcgccac gttcgccggc tttccccgtc aagctctaaa tcgggggctc cctttagggt
7081 tccgatttag tgctttacgg cacctcgacc ccaaaaaact tgattagggt gatggttcac
7141 gtagtgggcc atcgccctga tagacggttt ttcgcccttt gacgttggag tccacgttct
7201 ttaatagtgg actcttgttc caaactggaa caacactcaa ccctatctcg gtctattctt
7261 ttgatttata agggattttg ccgatttcgg cctattggtt aaaaaatgag ctgatttaac
7321 aaaaatttaa cgcgaatttt aacaaaatat taacgcttac aatttaggtg gcacttttcg
7381 gggaaatgtg cgcggaaccc ctatttgttt atttttctaa atacattcaa atatgtatcc
7441 gctcatgaga caataaccct gataaatgct tcaataatat tgaaaaagga agagtatgag
7501 tattcaacat ttccgtgtcg cccttattcc cttttttgcg gcattttgcc ttcctgtttt
7561 tgctcaccca gaaacgctgg tgaaagtaaa agatgctgaa gatcagttgg gtgcacgagt
7621 gggttacatc gaactggatc tcaacagcgg taagatcctt gagagttttc gccccgaaga
7681 acgttttcca atgatgagca cttttaaagt tctgctatgt ggcgcggtat tatcccgtat
7741 tgacgccggg caagagcaac tcggtcgccg catacactat tctcagaatg acttggttga
7801 gtactcacca gtcacagaaa agcatcttac ggatggcatg acagtaagag aattatgcag
7861 tgctgccata accatgagtg ataacactgc ggccaactta cttctgacaa cgatcggagg
7921 accgaaggag ctaaccgctt ttttgcacaa catgggggat catgtaactc gccttgatcg
7981 ttgggaaccg gagctgaatg aagccatacc aaacgacgag cgtgacacca cgatgcctgt
8041 agcaatggca acaacgttgc gcaaactatt aactggcgaa ctacttactc tagcttcccg
8101 gcaacaatta atagactgga tggaggcgga taaagttgca ggaccacttc tgcgctcggc
8161 ccttccggct ggctggttta ttgctgataa atctggagcc ggtgagcgtg ggtctcgcgg
8221 tatcattgca gcactggggc cagatggtaa gccctcccgt atcgtagtta tctacacgac
8281 ggggagtcag gcaactatgg atgaacgaaa tagacagatc gctgagatag gtgcctcact
8341 gattaagcat tggtaactgt cagaccaagt ttactcatat atactttaga ttgatttaaa
8401 acttcatttt taatttaaaa ggatctaggt gaagatcctt tttgataatc tcatgaccaa
8461 aatcccttaa cgtgagtttt cgttccactg agcgtcagac cccgtagaaa agatcaaagg
8521 atcttc
SEQ ID NO: 24: pLX304-PRKACA (synthetic construct)
      1 ttgagatcct ttttttctgc gcgtaatctg ctgcttgcaa acaaaaaaac caccgctacc
    61 agcggtggtt tgtttgccgg atcaagagct accaactctt tttccgaagg taactggctt
  121 cagcagagcg cagataccaa atactgttct tctagtgtag ccgtagttag gccaccactt
  181 caagaactct gtagcaccgc ctacatacct cgctctgcta atcctgttac cagtggctgc
  241 tgccagtggc gataagtcgt gtcttaccgg gttggactca agacgatagt taccggataa
  301 ggcgcagcgg tcgggctgaa cggggggttc gtgcacacag cccagcttgg agcgaacgac
  361 ctacaccgaa ctgagatacc tacagcgtga gctatgagaa agcgccacgc ttcccgaagg
  421 gagaaaggcg gacaggtatc cggtaagcgg cagggtcgga acaggagagc gcacgaggga
  481 gcttccaggg ggaaacgcct ggtatcttta tagtcctgtc gggtttcgcc acctctgact
  541 tgagcgtcga tttttgtgat gctcgtcagg ggggcggagc ctatggaaaa acgccagcaa
  601 cgcggccttt ttacggttcc tggccttttg ctggcctttt gctcacatgt tctttcctgc
  661 gttatcccct gattctgtgg ataaccgtat taccgccttt gagtgagctg ataccgctcg
  721 ccgcagccga acgaccgagc gcagcgagtc agtgagcgag gaagcggaag agcgcccaat
  781 acgcaaaccg cctctccccg cgcgttggcc gattcattaa tgcagctggc acgacaggtt
  841 tcccgactgg aaagcgggca gtgagcgcaa cgcaattaat gtgagttagc tcactcatta
  901 ggcaccccag gctttacact ttatgcttcc ggctcgtatg ttgtgtggaa ttgtgagcgg
  961 ataacaattt cacacaggaa acagctatga ccatgattac gccaagcgcg caattaaccc
1021 tcactaaagg gaacaaaagc tggagctgca agcttaatgt agtcttatgc aatactcttg
1081 tagtcttgca acatggtaac gatgagttag caacatgcct tacaaggaga gaaaaagcac
1141 cgtgcatgcc gattggtgga agtaaggtgg tacgatcgtg ccttattagg aaggcaacag
1201 acgggtctga catggattgg acgaaccact gaattgccgc attgcagaga tattgtattt
1261 aagtgcctag ctcgatacat aaacgggtct ctctggttag accagatctg agcctgggag
1321 ctctctggct aactagggaa cccactgctt aagcctcaat aaagcttgcc ttgagtgctt
1381 caagtagtgt gtgcccgtct gttgtgtgac tctggtaact agagatccct cagacccttt
1441 tagtcagtgt ggaaaatctc tagcagtggc gcccgaacag ggacttgaaa gcgaaaggga
1501 aaccagagga gctctctcga cgcaggactc ggcttgctga agcgcgcacg gcaagaggcg
1561 aggggcggcg actggtgagt acgccaaaaa ttttgactag cggaggctag aaggagagag
1621 atgggtgcga gagcgtcagt attaagcggg ggagaattag atcgcgatgg gaaaaaattc
1681 ggttaaggcc agggggaaag aaaaaatata aattaaaaca tatagtatgg gcaagcaggg
1741 agctagaacg attcgcagtt aatcctggcc tgttagaaac atcagaaggc tgtagacaaa
1801 tactgggaca gctacaacca tcccttcaga caggatcaga agaacttaga tcattatata
1861 atacagtagc aaccctctat tgtgtgcatc aaaggataga gataaaagac accaaggaag
1921 ctttagacaa gatagaggaa gagcaaaaca aaagtaagac caccgcacag caagcggccg
1981 ctgatcttca gacctggagg aggagatatg agggacaatt ggagaagtga attatataaa
2041 tataaagtag taaaaattga accattagga gtagcaccca ccaaggcaaa gagaagagtg
2101 gtgcagagag aaaaaagagc agtgggaata ggagctttgt tccttgggtt cttgggagca
2161 gcaggaagca ctatgggcgc agcgtcaatg acgctgacgg tacaggccag acaattattg
2221 tctggtatag tgcagcagca gaacaatttg ctgagggcta ttgaggcgca acagcatctg
2281 ttgcaactca cagtctgggg catcaagcag ctccaggcaa gaatcctggc tgtggaaaga
2341 tacctaaagg atcaacagct cctggggatt tggggttgct ctggaaaact catttgcacc
2401 actgctgtgc cttggaatgc tagttggagt aataaatctc tggaacagat ttggaatcac
2461 acgacctgga tggagtggga cagagaaatt aacaattaca caagcttaat acactcctta
2521 attgaagaat cgcaaaacca gcaagaaaag aatgaacaag aattattgga attagataaa
2581 tgggcaagtt tgtggaattg gtttaacata acaaattggc tgtggtatat aaaattattc
2641 ataatgatag taggaggctt ggtaggttta agaatagttt ttgctgtact ttctatagtg
2701 aatagagtta ggcagggata ttcaccatta tcgtttcaga cccacctccc aaccccgagg
2761 ggacccttgc gccttttcca aggcagccct gggtttgcgc agggacgcgg ctgctctggg
2821 cgtggttccg ggaaacgcag cggcgccgac cctgggtctc gcacattctt cacgtccgtt
2881 cgcagcgtca cccggatctt cgccgctacc cttgtgggcc ccccggcgac gcttcctgct
2941 ccgcccctaa gtcgggaagg ttccttgcgg ttcgcggcgt gccggacgtg acaaacggaa
3001 gccgcacgtc tcactagtac cctcgcagac ggacagcgcc agggagcaat ggcagcgcgc
3061 cgaccgcgat gggctgtggc caatagcggc tgctcagcag ggcgcgccga gagcagcggc
3121 cgggaagggg cggtgcggga ggcggggtgt ggggcggtag tgtgggccct gttcctgccc
3181 gcgcggtgtt ccgcattctg caagcctccg gagcgcacgt cggcagtcgg ctccctcgtt
3241 gaccgaatca ccgacctctc tccccagggg gtaccaccat ggccaagcct ttgtctcaag
3301 aagaatccac cctcattgaa agagcaacgg ctacaatcaa cagcatcccc atctctgaag
3361 actacagcgt cgccagcgca gctctctcta gcgacggccg catcttcact ggtgtcaatg
3421 tatatcattt tactggggga ccttgtgcag aactcgtggt gctgggcact gctgctgctg
3481 cggcagctgg caacctgact tgtatcgtcg cgatcggaaa tgagaacagg ggcatcttga
3541 gcccctgcgg acggtgccga caggtgcttc tcgatctgca tcctgggatc aaagccatag
3601 tgaaggacag tgatggacag ccgacggcag ttgggattcg tgaattgctg ccctctggtt
3661 atgtgtggga gggcctgcag ctgcagtagt aagaattcta gatcttgaga caaatggcag
3721 tattcatcca caattttaaa agaaaagggg ggattggggg gtacagtgca ggggaaagaa
3781 tagtagacat aatagcaaca gacatacaaa ctaaagaatt acaaaaacaa attacaaaaa
3841 ttcaaaattt tcgggtttat tacagggaca gcagagatcc actttggcgc cggctcgagg
3901 gggcccgggg ttattaatag taatcaatta cggggtcatt agttcatagc ccatatatgg
3961 agttccgcgt tacataactt acggtaaatg gcccgcctgg ctgaccgccc aacgaccccc
4021 gcccattgac gtcaataatg acgtatgttc ccatagtaac gccaataggg actttccatt
4081 gacgtcaatg ggtggagtat ttacggtaaa ctgcccactt ggcagtacat caagtgtatc
4141 atatgccaag tacgccccct attgacgtca atgacggtaa atggcccgcc tggcattatg
4201 cccagtacat gaccttatgg gactttccta cttggcagta catctacgta ttagtcatcg
4261 ctattaccat ggtgatgcgg ttttggcagt acatcaatgg gcgtggatag cggtttgact
4321 cacggggatt tccaagtctc tcaccccatt acgtcaatgg gagtttgttt tggcaccaaa
4381 atcaacggga ctttccaaaa tgtcgtaaca actccgcccc attgacgcaa atgggcggta
4441 ggcgtgtacg gtgggaggtc tatataagca gagctctctg gctaactgtc gggatcaaca
4501 agtttgtaca aaaaagcagg ctccaccatg ggcaacgccg ccgccgccaa gaagggcagc
4561 gagcaggaga gcgtgaaaga attcttagcc aaagccaaag aagattttct taaaaaatgg
4621 gaaagtcccg ctcagaacac agcccacttg gatcagtttg aacgaatcaa gaccctcggc
4681 acgggctcct tcgggcgggt gatgctggtg aaacacaagg agaccgggaa ccactatgcc
4741 atgaagatcc tcgacaaaca gaaggtggtg aaactgaaac agatcgaaca caccctgaat
4801 gaaaagcgca tcctgcaagc tgtcaacttt ccgttcctcg tcaaactcga gttctccttc
4861 aaggacaact caaacttata catggtcatg gagtacgtgc cgcgggggga gatgttctca
4921 cacctacggc ggatcggaag gttcagtgag ccccatgccc gtttctacgc ggcccagatc
4981 gtcctgacct ttgagtatct gcactcgctg gatctcatct acagggacct gaagccggag
5041 aatctgctca ttgaccagca gggctacatt caggtgacag acttcggttt cgccaagcgc
5101 gtgaagggcc gcacttggac cttgtgcggc acccctgagt acctggcccc tgagattatc
5161 ctgagcaaag gctacaacaa ggccgtggac tggtgggccc tgggggttct tatctatgaa
5221 atggccgctg gctacccgcc cttcttcgca gaccagccca tccagatcta tgagaagatc
5281 gtctctggga aggtgcgctt cccttcccac ttcagctctg acttgaagga cctgctgcgg
5341 aacctcctgc aggtagatct caccaagcgc tttgggaacc tcaagaatgg ggtcaacgat
5401 atcaagaacc acaagtggtt tgccacaact gactggattg ccatctacca gaggaaggtg
5461 gaagctccct tcataccaaa gtttaaaggc cctggggata cgagtaactt tgacgactat
5521 gaggaagaag aaatccgggt ctccatcaat gagaagtgtg gcaaggagtt ttctgagttt
5581 taattggacc cagctttctt gtacaaagtg gttggtaagc ctatccctaa ccctctcctc
5641 ggtctcgatt ctacgtagta atgagctagc gctaaccggt ggcgcgttaa gtcgacaatc
5701 aacctctgga ttacaaaatt tgtgaaagat tgactggtat tcttaactat gttgctcctt
5761 ttacgctatg tggatacgct gctttaatgc ctttgtatca tgctattgct tcccgtatgg
5821 ctttcatttt ctcctccttg tataaatcct ggttgctgtc tctttatgag gagttgtggc
5881 ccgttgtcag gcaacgtggc gtggtgtgca ctgtgtttgc tgacgcaacc cccactggtt
5941 ggggcattgc caccacctgt cagctccttt ccgggacttt cgctttcccc ctccctattg
6001 ccacggcgga actcatcgcc gcctgccttg cccgctgctg gacaggggct cggctgttgg
6061 gcactgacaa ttccgtggtg ttgtcgggga aatcatcgtc ctttccttgg ctgctcgcct
6121 gtgttgccac ctggattctg cgcgggacgt ccttctgcta cgtcccttcg gccctcaatc
6181 cagcggacct tccttcccgc ggcctgctgc cggctctgcg gcctcttccg cgtcttcgcc
6241 ttcgccctca gacgagtcgg atctcccttt gggccgcctc cccgcgtcga ctttaagacc
6301 aatgacttac aaggcagctg tagatcttag ccacttttta aaagaaaagg ggggactgga
6361 agggctaatt cactcccaac gaagacaaga tctgcttttt gcttgtactg ggtctctctg
6421 gttagaccag atctgagcct gggagctctc tggctaacta gggaacccac tgcttaagcc
6481 tcaataaagc ttgccttgag tgcttcaagt agtgtgtgcc cgtctgttgt gtgactctgg
6541 taactagaga tccctcagac ccttttagtc agtgtggaaa atctctagca gtacgtatag
6601 tagttcatgt catcttatta ttcagtattt ataacttgca aagaaatgaa tatcagagag
6661 tgagaggaac ttgtttattg cagcttataa tggttacaaa taaagcaata gcatcacaaa
6721 tttcacaaat aaagcatttt tttcactgca ttctagttgt ggtttgtcca aactcatcaa
6781 tgtatcttat catgtctggc tctagctatc ccgcccctaa ctccgcccat cccgccccta
6841 actccgccca gttccgccca ttctccgccc catggctgac taattttttt tatttatgca
6901 gaggccgagg ccgcctcggc ctctgagcta ttccagaagt agtgaggagg cttttttgga
6961 ggcctaggga cgtacccaat tcgccctata gtgagtcgta ttacgcgcgc tcactggccg
7021 tcgttttaca acgtcgtgac tgggaaaacc ctggcgttac ccaacttaat cgccttgcag
7081 cacatccccc tttcgccagc tggcgtaata gcgaagaggc ccgcaccgat cgcccttccc
7141 aacagttgcg cagcctgaat ggcgaatggg acgcgccctg tagcggcgca ttaagcgcgg
7201 cgggtgtggt ggttacgcgc agcgtgaccg ctacacttgc cagcgcccta gcgcccgctc
7261 ctttcgcttt cttcccttcc tttctcgcca cgttcgccgg ctttccccgt caagctctaa
7321 atcgggggct ccctttaggg ttccgattta gtgctttacg gcacctcgac cccaaaaaac
7381 ttgattaggg tgatggttca cgtagtgggc catcgccctg atagacggtt tttcgccctt
7441 tgacgttgga gtccacgttc tttaatagtg gactcttgtt ccaaactgga acaacactca
7501 accctatctc ggtctattct tttgatttat aagggatttt gccgatttcg gcctattggt
7561 taaaaaatga gctgatttaa caaaaattta acgcgaattt taacaaaata ttaacgctta
7621 caatttaggt ggcacttttc ggggaaatgt gcgcggaacc cctatttgtt tatttttcta
7681 aatacattca aatatgtatc cgctcatgag acaataaccc tgataaatgc ttcaataata
7741 ttgaaaaagg aagagtatga gtattcaaca tttccgtgtc gcccttattc ccttttttgc
7801 ggcattttgc cttcctgttt ttgctcaccc agaaacgctg gtgaaagtaa aagatgctga
7861 agatcagttg ggtgcacgag tgggttacat cgaactggat ctcaacagcg gtaagatcct
7921 tgagagtttt cgccccgaag aacgttttcc aatgatgagc acttttaaag ttctgctatg
7981 tggcgcggta ttatcccgta ttgacgccgg gcaagagcaa ctcggtcgcc gcatacacta
8041 ttctcagaat gacttggttg agtactcacc agtcacagaa aagcatctta cggatggcat
8101 gacagtaaga gaattatgca gtgctgccat aaccatgagt gataacactg cggccaactt
8161 acttctgaca acgatcggag gaccgaagga gctaaccgct tttttgcaca acatggggga
8221 tcatgtaact cgccttgatc gttgggaacc ggagctgaat gaagccatac caaacgacga
8281 gcgtgacacc acgatgcctg tagcaatggc aacaacgttg cgcaaactat taactggcga
8341 actacttact ctagcttccc ggcaacaatt aatagactgg atggaggcgg ataaagttgc
8401 aggaccactt ctgcgctcgg cccttccggc tggctggttt attgctgata aatctggagc
8461 cggtgagcgt gggtctcgcg gtatcattgc agcactgggg ccagatggta agccctcccg
8521 tatcgtagtt atctacacga cggggagtca ggcaactatg gatgaacgaa atagacagat
8581 cgctgagata ggtgcctcac tgattaagca ttggtaactg tcagaccaag tttactcata
8641 tatactttag attgatttaa aacttcattt ttaatttaaa aggatctagg tgaagatcct
8701 ttttgataat ctcatgacca aaatccctta acgtgagttt tcgttccact gagcgtcaga
8761 ccccgtagaa aagatcaaag gatcttc
SEQ ID NO: 25: pLX304-ALDH3A1 (synthetic construct)
      1 ttgagatcct ttttttctgc gcgtaatctg ctgcttgcaa acaaaaaaac caccgctacc
    61 agcggtggtt tgtttgccgg atcaagagct accaactctt tttccgaagg taactggctt
  121 cagcagagcg cagataccaa atactgttct tctagtgtag ccgtagttag gccaccactt
  181 caagaactct gtagcaccgc ctacatacct cgctctgcta atcctgttac cagtggctgc
  241 tgccagtggc gataagtcgt gtcttaccgg gttggactca agacgatagt taccggataa
  301 ggcgcagcgg tcgggctgaa cggggggttc gtgcacacag cccagcttgg agcgaacgac
  361 ctacaccgaa ctgagatacc tacagcgtga gctatgagaa agcgccacgc ttcccgaagg
  421 gagaaaggcg gacaggtatc cggtaagcgg cagggtcgga acaggagagc gcacgaggga
  481 gcttccaggg ggaaacgcct ggtatcttta tagtcctgtc gggtttcgcc acctctgact
  541 tgagcgtcga tttttgtgat gctcgtcagg ggggcggagc ctatggaaaa acgccagcaa
  601 cgcggccttt ttacggttcc tggccttttg ctggcctttt gctcacatgt tctttcctgc
  661 gttatcccct gattctgtgg ataaccgtat taccgccttt gagtgagctg ataccgctcg
  721 ccgcagccga acgaccgagc gcagcgagtc agtgagcgag gaagcggaag agcgcccaat
  781 acgcaaaccg cctctccccg cgcgttggcc gattcattaa tgcagctggc acgacaggtt
  841 tcccgactgg aaagcgggca gtgagcgcaa cgcaattaat gtgagttagc tcactcatta
  901 ggcaccccag gctttacact ttatgcttcc ggctcgtatg ttgtgtggaa ttgtgagcgg
  961 ataacaattt cacacaggaa acagctatga ccatgattac gccaagcgcg caattaaccc
1021 tcactaaagg gaacaaaagc tggagctgca agcttaatgt agtcttatgc aatactcttg
1081 tagtcttgca acatggtaac gatgagttag caacatgcct tacaaggaga gaaaaagcac
1141 cgtgcatgcc gattggtgga agtaaggtgg tacgatcgtg ccttattagg aaggcaacag
1201 acgggtctga catggattgg acgaaccact gaattgccgc attgcagaga tattgtattt
1261 aagtgcctag ctcgatacat aaacgggtct ctctggttag accagatctg agcctgggag
1321 ctctctggct aactagggaa cccactgctt aagcctcaat aaagcttgcc ttgagtgctt
1381 caagtagtgt gtgcccgtct gttgtgtgac tctggtaact agagatccct cagacccttt
1441 tagtcagtgt ggaaaatctc tagcagtggc gcccgaacag ggacttgaaa gcgaaaggga
1501 aaccagagga gctctctcga cgcaggactc ggcttgctga agcgcgcacg gcaagaggcg
1561 aggggcggcg actggtgagt acgccaaaaa ttttgactag cggaggctag aaggagagag
1621 atgggtgcga gagcgtcagt attaagcggg ggagaattag atcgcgatgg gaaaaaattc
1681 ggttaaggcc agggggaaag aaaaaatata aattaaaaca tatagtatgg gcaagcaggg
1741 agctagaacg attcgcagtt aatcctggcc tgttagaaac atcagaaggc tgtagacaaa
1801 tactgggaca gctacaacca tcccttcaga caggatcaga agaacttaga tcattatata
1861 atacagtagc aaccctctat tgtgtgcatc aaaggataga gataaaagac accaaggaag
1921 ctttagacaa gatagaggaa gagcaaaaca aaagtaagac caccgcacag caagcggccg
1981 ctgatcttca gacctggagg aggagatatg agggacaatt ggagaagtga attatataaa
2041 tataaagtag taaaaattga accattagga gtagcaccca ccaaggcaaa gagaagagtg
2101 gtgcagagag aaaaaagagc agtgggaata ggagctttgt tccttgggtt cttgggagca
2161 gcaggaagca ctatgggcgc agcgtcaatg acgctgacgg tacaggccag acaattattg
2221 tctggtatag tgcagcagca gaacaatttg ctgagggcta ttgaggcgca acagcatctg
2281 ttgcaactca cagtctgggg catcaagcag ctccaggcaa gaatcctggc tgtggaaaga
2341 tacctaaagg atcaacagct cctggggatt tggggttgct ctggaaaact catttgcacc
2401 actgctgtgc cttggaatgc tagttggagt aataaatctc tggaacagat ttggaatcac
2461 acgacctgga tggagtggga cagagaaatt aacaattaca caagcttaat acactcctta
2521 attgaagaat cgcaaaacca gcaagaaaag aatgaacaag aattattgga attagataaa
2581 tgggcaagtt tgtggaattg gtttaacata acaaattggc tgtggtatat aaaattattc
2641 ataatgatag taggaggctt ggtaggttta agaatagttt ttgctgtact ttctatagtg
2701 aatagagtta ggcagggata ttcaccatta tcgtttcaga cccacctccc aaccccgagg
2761 ggacccttgc gccttttcca aggcagccct gggtttgcgc agggacgcgg ctgctctggg
2821 cgtggttccg ggaaacgcag cggcgccgac cctgggtctc gcacattctt cacgtccgtt
2881 cgcagcgtca cccggatctt cgccgctacc cttgtgggcc ccccggcgac gcttcctgct
2941 ccgcccctaa gtcgggaagg ttccttgcgg ttcgcggcgt gccggacgtg acaaacggaa
3001 gccgcacgtc tcactagtac cctcgcagac ggacagcgcc agggagcaat ggcagcgcgc
3061 cgaccgcgat gggctgtggc caatagcggc tgctcagcag ggcgcgccga gagcagcggc
3121 cgggaagggg cggtgcggga ggcggggtgt ggggcggtag tgtgggccct gttcctgccc
3181 gcgcggtgtt ccgcattctg caagcctccg gagcgcacgt cggcagtcgg ctccctcgtt
3241 gaccgaatca ccgacctctc tccccagggg gtaccaccat ggccaagcct ttgtctcaag
3301 aagaatccac cctcattgaa agagcaacgg ctacaatcaa cagcatcccc atctctgaag
3361 actacagcgt cgccagcgca gctctctcta gcgacggccg catcttcact ggtgtcaatg
3421 tatatcattt tactggggga ccttgtgcag aactcgtggt gctgggcact gctgctgctg
3481 cggcagctgg caacctgact tgtatcgtcg cgatcggaaa tgagaacagg ggcatcttga
3541 gcccctgcgg acggtgccga caggtgcttc tcgatctgca tcctgggatc aaagccatag
3601 tgaaggacag tgatggacag ccgacggcag ttgggattcg tgaattgctg ccctctggtt
3661 atgtgtggga gggcctgcag ctgcagtagt aagaattcta gatcttgaga caaatggcag
3721 tattcatcca caattttaaa agaaaagggg ggattggggg gtacagtgca ggggaaagaa
3781 tagtagacat aatagcaaca gacatacaaa ctaaagaatt acaaaaacaa attacaaaaa
3841 ttcaaaattt tcgggtttat tacagggaca gcagagatcc actttggcgc cggctcgagg
3901 gggcccgggg ttattaatag taatcaatta cggggtcatt agttcatagc ccatatatgg
3961 agttccgcgt tacataactt acggtaaatg gcccgcctgg ctgaccgccc aacgaccccc
4021 gcccattgac gtcaataatg acgtatgttc ccatagtaac gccaataggg actttccatt
4081 gacgtcaatg ggtggagtat ttacggtaaa ctgcccactt ggcagtacat caagtgtatc
4141 atatgccaag tacgccccct attgacgtca atgacggtaa atggcccgcc tggcattatg
4201 cccagtacat gaccttatgg gactttccta cttggcagta catctacgta ttagtcatcg
4261 ctattaccat ggtgatgcgg ttttggcagt acatcaatgg gcgtggatag cggtttgact
4321 cacggggatt tccaagtctc caccccattg acgtcaatgg gagtttgttt tggcaccaaa
4381 atcaacggga ctttccaaaa tgtcgtaaca actccgcccc attgacgcaa atgggcggta
4441 ggcgtgtacg gtgggaggtc tatataagca gagctctctg gctaactgtc gggatcaaca
4501 agtttgtaca aaaaagcagg ctccaccatg agcaagatca gcgaggccgt gaagcgcgcc
4561 cgcgccgcct tcagctcggg caggacccgt ccgctgcagt tccggatcca gcagctggag
4621 gcgctgcagc gcctgatcca ggagcaggag caggagctgg tgggcgcgct ggccgcagac
4681 ctgcacaaga atgaatggaa cgcctactat gaggaggtgg tgtacgtcct agaggagatc
4741 gagtacatga tccagaagct ccctgagtgg gccgcggatg agcccgtgga gaagacgccc
4801 cagactcagc aggacgagct ctacatccac tcggagccac tgggcgtggt cctcgtcatt
4861 ggcacctgga actacccctt caacctcacc atccagccca tggtgggcgc catcgctgca
4921 gggaacgcag tggtcctcaa gccctcggag ctgagtgaga acatggcgag cctgctggct
4981 accatcatcc cccagtacct ggacaaggat ctgtacccag taatcaatgg gggtgtccct
5041 gagaccacgg agctgctcaa ggagaggttc gaccatatcc tgtacacggg cagcacgggg
5101 gtggggaaga tcatcatgac ggctgctgcc aagcacctga cccctgtcac gctggagctg
5161 ggagggaaga gtccctgcta cgtggacaag aactgtgacc tggacgtggc ctgccgacgc
5221 atcgcctggg ggaaattcat gaacagtggc cagacctgcg tggccccaga ctacatcctc
5281 tgtgacccct cgatccagaa ccaaattgtg gagaagctca agaagtcact gaaagagttc
5341 tacggggaag atgctaagaa atcccgggac tatggaagaa tcattagtgc ccggcacttc
5401 cagagggtga tgggcctgat tgagggccag aaggtggctt atgggggcac cggggatgcc
5461 gccactcgct acatagcccc caccatcctc acggacgtgg acccccagtc cccggtgatg
5521 caagaggaga tcttcgggcc tgtgctgccc atcgtgtgcg tgcgcagcct ggaggaggcc
5581 atccagttca tcaaccagcg tgagaagccc ctggccctct acatgttctc cagcaacgac
5641 aaggtgatta agaagatgat tgcagagaca tccagtggtg gggtggcggc caacgatgtc
5701 atcgtccaca tcaccttgca ctctctgccc ttcgggggcg tggggaacag cggcatggga
5761 tcctaccatg gcaagaagag cttcgagact ttctctcacc gccgctcttg cctggtgagg
5821 cctctgatga atgatgaagg cctgaaggtc agataccccc cgagcccggc caagatgacc
5881 cagcactagg acccagcttt cttgtacaaa gtggttggta agcctatccc taaccctctc
5941 ctcggtctcg attctacgta gtaatgagct agcgctaacc ggtggcgcgt taagtcgaca
6001 atcaacctct ggattacaaa atttgtgaaa gattgactgg tattcttaac tatgttgctc
6061 cttttacgct atgtggatac gctgctttaa tgcctttgta tcatgctatt gcttcccgta
6121 tggctttcat tttctcctcc ttgtataaat cctggttgct gtctctttat gaggagttgt
6181 ggcccgttgt caggcaacgt ggcgtggtgt gcactgtgtt tgctgacgca acccccactg
6241 gttggggcat tgccaccacc tgtcagctcc tttccgggac tttcgctttc cccctcccta
6301 ttgccacggc ggaactcatc gccgcctgcc ttgcccgctg ctggacaggg gctcggctgt
6361 tgggcactga caattccgtg gtgttgtcgg ggaaatcatc gtcctttcct tggctgctcg
6421 cctgtgttgc cacctggatt ctgcgcggga cgtccttctg ctacgtccct tcggccctca
6481 atccagcgga ccttccttcc cgcggcctgc tgccggctct gcggcctctt ccgcgtcttc
6541 gccttcgccc tcagacgagt cggatctccc tttgggccgc ctccccgcgt cgactttaag
6601 accaatgact tacaaggcag ctgtagatct tagccacttt ttaaaagaaa aggggggact
6661 ggaagggcta attcactccc aacgaagaca agatctgctt tttgcttgta ctgggtctct
6721 ctggttagac cagatctgag cctgggagct ctctggctaa ctagggaacc cactgcttaa
6781 gcctcaataa agcttgcctt gagtgcttca agtagtgtgt gcccgtctgt tgtgtgactc
6841 tggtaactag agatccctca gaccctttta gtcagtgtgg aaaatctcta gcagtacgta
6901 tagtagttca tgtcatctta ttattcagta tttataactt gcaaagaaat gaatatcaga
6961 gagtgagagg aacttgttta ttgcagctta taatggttac aaataaagca atagcatcac
7021 aaatttcaca aataaagcat ttttttcact gcattctagt tgtggtttgt ccaaactcat
7081 caatgtatct tatcatgtct ggctctagct atcccgcccc taactccgcc catcccgccc
7141 ctaactccgc ccagttccgc ccattctccg ccccatggct gactaatttt ttttatttat
7201 gcagaggccg aggccgcctc ggcctctgag ctattccaga agtagtgagg aggctttttt
7261 ggaggcctag ggacgtaccc aattcgccct atagtgagtc gtattacgcg cgctcactgg
7321 ccgtcgtttt acaacgtcgt gactgggaaa accctggcgt tacccaactt aatcgccttg
7381 cagcacatcc ccctttcgcc agctggcgta atagcgaaga ggcccgcacc gatcgccctt
7441 cccaacagtt gcgcagcctg aatggcgaat gggacgcgcc ctgtagcggc gcattaagcg
7501 cggcgggtgt ggtggttacg cgcagcgtga ccgctacact tgccagcgcc ctagcgcccg
7561 ctcctttcgc tttcttccct tcctttctcg ccacgttcgc cggctttccc cgtcaagctc
7621 taaatcgggg gctcccttta gggttccgat ttagtgcttt acggcacctc gaccccaaaa
7681 aacttgatta gggtgatggt tcacgtagtg ggccatcgcc ctgatagacg gtttttcgcc
7741 ctttgacgtt ggagtccacg ttctttaata gtggactctt gttccaaact ggaacaacac
7801 tcaaccctat ctcggtctat tcttttgatt tataagggat tttgccgatt tcggcctatt
7861 ggttaaaaaa tgagctgatt taacaaaaat ttaacgcgaa ttttaacaaa atattaacgc
7921 ttacaattta ggtggcactt ttcggggaaa tgtgcgcgga acccctattt gtttattttt
7981 ctaaatacat tcaaatatgt atccgctcat gagacaataa ccctgataaa tgcttcaata
8041 atattgaaaa aggaagagta tgagtattca acatttccgt gtcgccctta ttcccttttt
8101 tgcggcattt tgccttcctg tttttgctca cccagaaacg ctggtgaaag taaaagatgc
8161 tgaagatcag ttgggtgcac gagtgggtta catcgaactg gatctcaaca gcggtaagat
8221 ccttgagagt tttcgccccg aagaacgttt tccaatgatg agcactttta aagttctgct
8281 atgtggcgcg gtattatccc gtattgacgc cgggcaagag caactcggtc gccgcataca
8341 ctattctcag aatgacttgg ttgagtactc accagtcaca gaaaagcatc ttacggatgg
8401 catgacagta agagaattat gcagtgctgc cataaccatg agtgataaca ctgcggccaa
8461 cttacttctg acaacgatcg gaggaccgaa ggagctaacc gcttttttgc acaacatggg
8521 ggatcatgta actcgccttg atcgttggga accggagctg aatgaagcca taccaaacga
8581 cgagcgtgac accacgatgc ctgtagcaat ggcaacaacg ttgcgcaaac tattaactgg
8641 cgaactactt actctagctt cccggcaaca attaatagac tggatggagg cggataaagt
8701 tgcaggacca cttctgcgct cggcccttcc ggctggctgg tttattgctg ataaatctgg
8761 agccggtgag cgtgggtctc gcggtatcat tgcagcactg gggccagatg gtaagccctc
8821 ccgtatcgta gttatctaca cgacggggag tcaggcaact atggatgaac gaaatagaca
8881 gatcgctgag ataggtgcct cactgattaa gcattggtaa ctgtcagacc aagtttactc
8941 atatatactt tagattgatt taaaacttca tttttaattt aaaaggatct aggtgaagat
9001 cctttttgat aatctcatga ccaaaatccc ttaacgtgag ttttcgttcc actgagcgtc
9061 agaccccgta gaaaagatca aaggatcttc
SEQ ID NO: 26: pLX304-GSTA4 (synthetic construct)
      1 gcccggggtt attaatagta atcaattacg gggtcattag ttcatagccc atatatggag
    61 ttccgcgtta cataacttac ggtaaatggc ccgcctggct gaccgcccaa cgacccccgc
  121 ccattgacgt caataatgac gtatgttccc atagtaacgc caatagggac tttccattga
  181 cgtcaatggg tggagtattt acggtaaact gcccacttgg cagtacatca agtgtatcat
  241 atgccaagta cgccccctat tgacgtcaat gacggtaaat ggcccgcctg gcattatgcc
  301 cagtacatga ccttatggga ctttcctact tggcagtaca tctacgtatt agtcatcgct
  361 attaccatgg tgatgcggtt ttggcagtac atcaatgggc gtggatagcg gtttgactca
  421 cggggatttc caagtctcca ccccattgac gtcaatggga gtttgttttg gcaccaaaat
  481 caacgggact ttccaaaatg tcgtaacaac tccgccccat tgacgcaaat gggcggtagg
  541 cgtgtacggt gggaggtcta tataagcaga gctctctggc taactgtcgg gatcaacaag
  601 tttgtacaaa aaagttggca tgtacgtgga ggggacactg gatctgctgg aactgcttat
  661 catgcatcct ttcttaaaac cagatgatca gcaaaaggaa gtggttaaca tggcccagaa
  721 ggctataatt agatactttc ctgtgtttga aaagatttta aggggtcacg gacaaagctt
  781 tcttgttggt aatcagctga gccttgcaga tgtgatttta ctccaaacca ttttagctct
  841 agaagagaaa attcctaata tcctgtctgc atttcctttc ctccaggaat acacagtgaa
  901 actaagtaat atccctacaa ttaagagatt ccttgaacct ggcagcaaga agaagcctcc
  961 ccctgatgaa atttatgtga gaaccgtcta caacatcttt aggccatacc caactttctt
1021 gtacaaagtg gttggtaagc ctatccctaa ccctctcctc ggtctcgatt ctacgtagta
1081 atgagctagc gctaaccggt ggcgcgttaa gtcgacaatc aacctctgga ttacaaaatt
1141 tgtgaaagat tgactggtat tcttaactat gttgctcctt ttacgctatg tggatacgct
1201 gctttaatgc ctttgtatca tgctattgct tcccgtatgg ctttcatttt ctcctccttg
1261 tataaatcct ggttgctgtc tctttatgag gagttgtggc ccgttgtcag gcaacgtggc
1321 gtggtgtgca ctgtgtttgc tgacgcaacc cccactggtt ggggcattgc caccacctgt
1381 cagctccttt ccgggacttt cgctttcccc ctccctattg ccacggcgga actcatcgcc
1441 gcctgccttg cccgctgctg gacaggggct cggctgttgg gcactgacaa ttccgtggtg
1501 ttgtcgggga aatcatcgtc ctttccttgg ctgctcgcct gtgttgccac ctggattctg
1561 cgcgggacgt ccttctgcta cgtcccttcg gccctcaatc cagcggacct tccttcccgc
1621 ggcctgctgc cggctctgcg gcctcttccg cgtcttcgcc ttcgccctca gacgagtcgg
1681 atctcccttt gggccgcctc cccgcgtcga ctttaagacc aatgacttac aaggcagctg
1741 tagatcttag ccacttttta aaagaaaagg ggggactgga agggctaatt cactcccaac
1801 gaagacaaga tctgcttttt gcttgtactg ggtctctctg gttagaccag atctgagcct
1861 gggagctctc tggctaacta gggaacccac tgcttaagcc tcaataaagc ttgccttgag
1921 tgcttcaagt agtgtgtgcc cgtctgttgt gtgactctgg taactagaga tccctcagac
1981 ccttttagtc agtgtggaaa atctctagca gtacgtatag tagttcatgt catcttatta
2041 ttcagtattt ataacttgca aagaaatgaa tatcagagag tgagaggaac ttgtttattg
2101 cagcttataa tggttacaaa taaagcaata gcatcacaaa tttcacaaat aaagcatttt
2161 tttcactgca ttctagttgt ggtttgtcca aactcatcaa tgtatcttat catgtctggc
2221 tctagctatc ccgcccctaa ctccgcccat cccgccccta actccgccca gttccgccca
2281 ttctccgccc catggctgac taattttttt tatttatgca gaggccgagg ccgcctcggc
2341 ctctgagcta ttccagaagt agtgaggagg cttttttgga ggcctaggga cgtacccaat
2401 tcgccctata gtgagtcgta ttacgcgcgc tcactggccg tcgttttaca acgtcgtgac
2461 tgggaaaacc ctggcgttac ccaacttaat cgccttgcag cacatccccc tttcgccagc
2521 tggcgtaata gcgaagaggc ccgcaccgat cgcccttccc aacagttgcg cagcctgaat
2581 ggcgaatggg acgcgccctg tagcggcgca ttaagcgcgg cgggtgtggt ggttacgcgc
2641 agcgtgaccg ctacacttgc cagcgcccta gcgcccgctc ctttcgcttt cttcccttcc
2701 tttctcgcca cgttcgccgg ctttccccgt caagctctaa atcgggggct ccctttaggg
2761 ttccgattta gtgctttacg gcacctcgac cccaaaaaac ttgattaggg tgatggttca
2821 cgtagtgggc catcgccctg atagacggtt tttcgccctt tgacgttgga gtccacgttc
2881 tttaatagtg gactcttgtt ccaaactgga acaacactca accctatctc ggtctattct
2941 tttgatttat aagggatttt gccgatttcg gcctattggt taaaaaatga gctgatttaa
3001 caaaaattta acgcgaattt taacaaaata ttaacgctta caatttaggt ggcacttttc
3061 ggggaaatgt gcgcggaacc cctatttgtt tatttttcta aatacattca aatatgtatc
3121 cgctcatgag acaataaccc tgataaatgc ttcaataata ttgaaaaagg aagagtatga
3181 gtattcaaca tttccgtgtc gcccttattc ccttttttgc ggcattttgc cttcctgttt
3241 ttgctcaccc agaaacgctg gtgaaagtaa aagatgctga agatcagttg ggtgcacgag
3301 tgggttacat cgaactggat ctcaacagcg gtaagatcct tgagagtttt cgccccgaag
3361 aacgttttcc aatgatgagc acttttaaag ttctgctatg tggcgcggta ttatcccgta
3421 ttgacgccgg gcaagagcaa ctcggtcgcc gcatacacta ttctcagaat gacttggttg
3481 agtactcacc agtcacagaa aagcatctta cggatggcat gacagtaaga gaattatgca
3541 gtgctgccat aaccatgagt gataacactg cggccaactt acttctgaca acgatcggag
3601 gaccgaagga gctaaccgct tttttgcaca acatggggga tcatgtaact cgccttgatc
3661 gttgggaacc ggagctgaat gaagccatac caaacgacga gcgtgacacc acgatgcctg
3721 tagcaatggc aacaacgttg cgcaaactat taactggcga actacttact ctagcttccc
3781 ggcaacaatt aatagactgg atggaggcgg ataaagttgc aggaccactt ctgcgctcgg
3841 cccttccggc tggctggttt attgctgata aatctggagc cggtgagcgt gggtctcgcg
3901 gtatcattgc agcactgggg ccagatggta agccctcccg tatcgtagtt atctacacga
3961 cggggagtca ggcaactatg gatgaacgaa atagacagat cgctgagata ggtgcctcac
4021 tgattaagca ttggtaactg tcagaccaag tttactcata tatactttag attgatttaa
4081 aacttcattt ttaatttaaa aggatctagg tgaagatcct ttttgataat ctcatgacca
4141 aaatccctta acgtgagttt tcgttccact gagcgtcaga ccccgtagaa aagatcaaag
4201 gatcttcttg agatcctttt tttctgcgcg taatctgctg cttgcaaaca aaaaaaccac
4261 cgctaccagc ggtggtttgt ttgccggatc aagagctacc aactcttttt ccgaaggtaa
4321 ctggcttcag cagagcgcag ataccaaata ctgttcttct agtgtagccg tagttaggcc
4381 accacttcaa gaactctgta gcaccgccta catacctcgc tctgctaatc ctgttaccag
4441 tggctgctgc cagtggcgat aagtcgtgtc ttaccgggtt ggactcaaga cgatagttac
4501 cggataaggc gcagcggtcg ggctgaacgg ggggttcgtg cacacagccc agcttggagc
4561 gaacgaccta caccgaactg agatacctac agcgtgagct atgagaaagc gccacgcttc
4621 ccgaagggag aaaggcggac aggtatccgg taagcggcag ggtcggaaca ggagagcgca
4681 cgagggagct tccaggggga aacgcctggt atctttatag tcctgtcggg tttcgccacc
4741 tctgacttga gcgtcgattt ttgtgatgct cgtcaggggg gcggagccta tggaaaaacg
4801 ccagcaacgc ggccttttta cggttcctgg ccttttgctg gccttttgct cacatgttct
4861 ttcctgcgtt atcccctgat tctgtggata accgtattac cgcctttgag tgagctgata
4921 ccgctcgccg cagccgaacg accgagcgca gcgagtcagt gagcgaggaa gcggaagagc
4981 gcccaatacg caaaccgcct ctccccgcgc gttggccgat tcattaatgc agctggcacg
5041 acaggtttcc cgactggaaa gcgggcagtg agcgcaacgc aattaatgtg agttagctca
5101 ctcattaggc accccaggct ttacacttta tgcttccggc tcgtatgttg tgtggaattg
5161 tgagcggata acaatttcac acaggaaaca gctatgacca tgattacgcc aagcgcgcaa
5221 ttaaccctca ctaaagggaa caaaagctgg agctgcaagc ttaatgtagt cttatgcaat
5281 actcttgtag tcttgcaaca tggtaacgat gagttagcaa catgccttac aaggagagaa
5341 aaagcaccgt gcatgccgat tggtggaagt aaggtggtac gatcgtgcct tattaggaag
5401 gcaacagacg ggtctgacat ggattggacg aaccactgaa ttgccgcatt gcagagatat
5461 tgtatttaag tgcctagctc gatacataaa cgggtctctc tggttagacc agatctgagc
5521 ctgggagctc tctggctaac tagggaaccc actgcttaag cctcaataaa gcttgccttg
5581 agtgcttcaa gtagtgtgtg cccgtctgtt gtgtgactct ggtaactaga gatccctcag
5641 acccttttag tcagtgtgga aaatctctag cagtggcgcc cgaacaggga cttgaaagcg
5701 aaagggaaac cagaggagct ctctcgacgc aggactcggc ttgctgaagc gcgcacggca
5761 agaggcgagg ggcggcgact ggtgagtacg ccaaaaattt tgactagcgg aggctagaag
5821 gagagagatg ggtgcgagag cgtcagtatt aagcggggga gaattagatc gcgatgggaa
5881 aaaattcggt taaggccagg gggaaagaaa aaatataaat taaaacatat agtatgggca
5941 agcagggagc tagaacgatt cgcagttaat cctggcctgt tagaaacatc agaaggctgt
6001 agacaaatac tgggacagct acaaccatcc cttcagacag gatcagaaga acttagatca
6061 ttatataata cagtagcaac cctctattgt gtgcatcaaa ggatagagat aaaagacacc
6121 aaggaagctt tagacaagat agaggaagag caaaacaaaa gtaagaccac cgcacagcaa
6181 gcggccgctg atcttcagac ctggaggagg agatatgagg gacaattgga gaagtgaatt
6241 atataaatat aaagtagtaa aaattgaacc attaggagta gcacccacca aggcaaagag
6301 aagagtggtg cagagagaaa aaagagcagt gggaatagga gctttgttcc ttgggttctt
6361 gggagcagca ggaagcacta tgggcgcagc gtcaatgacg ctgacggtac aggccagaca
6421 attattgtct ggtatagtgc agcagcagaa caatttgctg agggctattg aggcgcaaca
6481 gcatctgttg caactcacag tctggggcat caagcagctc caggcaagaa tcctggctgt
6541 ggaaagatac ctaaaggatc aacagctcct ggggatttgg ggttgctctg gaaaactcat
6601 ttgcaccact gctgtgcctt ggaatgctag ttggagtaat aaatctctgg aacagatttg
6661 gaatcacacg acctggatgg agtgggacag agaaattaac aattacacaa gcttaataca
6721 ctccttaatt gaagaatcgc aaaaccagca agaaaagaat gaacaagaat tattggaatt
6781 agataaatgg gcaagtttgt ggaattggtt taacataaca aattggctgt ggtatataaa
6841 attattcata atgatagtag gaggcttggt aggtttaaga atagtttttg ctgtactttc
6901 tatagtgaat agagttaggc agggatattc accattatcg tttcagaccc acctcccaac
6961 cccgagggga cccttgcgcc ttttccaagg cagccctggg tttgcgcagg gacgcggctg
7021 ctctgggcgt ggttccggga aacgcagcgg cgccgaccct gggtctcgca cattcttcac
7081 gtccgttcgc agcgtcaccc ggatcttcgc cgctaccctt gtgggccccc cggcgacgct
7141 tcctgctccg cccctaagtc gggaaggttc cttgcggttc gcggcgtgcc ggacgtgaca
7201 aacggaagcc gcacgtctca ctagtaccct cgcagacgga cagcgccagg gagcaatggc
7261 agcgcgccga ccgcgatggg ctgtggccaa tagcggctgc tcagcagggc gcgccgagag
7321 cagcggccgg gaaggggcgg tgcgggaggc ggggtgtggg gcggtagtgt gggccctgtt
7381 cctgcccgcg cggtgttccg cattctgcaa gcctccggag cgcacgtcgg cagtcggctc
7441 cctcgttgac cgaatcaccg acctctctcc ccagggggta ccaccatggc caagcctttg
7501 tctcaagaag aatccaccct cattgaaaga gcaacggcta caatcaacag catccccatc
7561 tctgaagact acagcgtcgc cagcgcagct ctctctagcg acggccgcat cttcactggt
7621 gtcaatgtat atcattttac tgggggacct tgtgcagaac tcgtggtgct gggcactgct
7681 gctgctgcgg cagctggcaa cctgacttgt atcgtcgcga tcggaaatga gaacaggggc
7741 atcttgagcc cctgcggacg gtgccgacag gtgcttctcg atctgcatcc tgggatcaaa
7801 gccatagtga aggacagtga tggacagccg acggcagttg ggattcgtga attgctgccc
7861 tctggttatg tgtgggaggg cctgcagctg cagtagtaag aattctagat cttgagacaa
7921 atggcagtat tcatccacaa ttttaaaaga aaagggggga ttggggggta cagtgcaggg
7981 gaaagaatag tagacataat agcaacagac atacaaacta aagaattaca aaaacaaatt
8041 acaaaaattc aaaattttcg ggtttattac agggacagca gagatccact ttggcgccgg
8101 ctcgagggg
SEQ ID NO: 27: Homo sapiens deoxycytidine kinase (DCK), RefSeqGene on
chromosome 4
AGTGTCCTCAGCTGCCTCCGCGCGCCAAAGTCAAACCCCGACACCCGCCGGCGGGCCGGTGAGCTCACTAGCTGACC
CGGCAGGTCAGGATCTGGCTTAGCGGCGCCGCGAGCTCCAGTGCGCGCACCCGTGGCCGCCTCCCAGCCCTCTTTGC
CGGACGAGCTCTGGGCCGCCACAAGACTAAGGAATGGCCACCCCGCCCAAGAGAAGCTGCCCGTCTTTCTCAGCCAG
CTCTGAGGGGACCCGCATCAAGAAAATCTCCATCGAAGGGAACATCGGTAAGGAGCCTCCGGAAATGTGGGACGCAA
GGCTGGGGTGTCGCGGCAGTGGCTGAAGCTTCCCTTCGCCGCATCTCTCTGCAGCAACTCGGTGAGGGCTTTCCCTC
CAGCCTGGCGGTGTGGACGCGGCCTCGGCTTCCTTTCCCACCCAGAGCATCCTTCCCTTACCTCCCGCTCCACGGGG
TGACTGCGGTCCCCGGGGTCAGACTGGGCGCCAGGCCTGCGGTCTCTGTGAGGCGCGCTTTGAACCTCTGCTCCCTC
TTTGTTGGGAACTCGCAAAGGGAAGCGGGGGAAGGGTATTAGATTTCTTGAGGTGTAACTTACATTAAATTCACCCT
TTTTAGGTGTACTCTTCTATGAGGTTTGACAAACATTATGCCTTTTGCTATCACCATGATCGAGATACAGAACATTT
TCATCACCTTAGAAAGTTCCTACCTGCTCCTTTGTATTCAGCCCCCTCCACCCTTCCTATTCCCTGGCACCCACTGA
TTTGATTTCTATCCCTGCAAGTGTTGCCTTTTCCAGAATGCCATATAAATGGAATCATAGGCTATATCGCCTTTTGT
GTCTGGCGTCTTTCATTTAGCATAATGCTTTTGAAATTCATTCATGTTACTTGTGTCTGTAGTTTGTTTCTTTTTAT
TGCTGAGTAGTATTCCATTGCATGGATGTACCATCGTTTATCCATTTTCTAGTTGTGGACCTCAGGGTTGTTTACAG
TTTTTTAGCTATATTATTAGCGAAGCATTGTTTTAGCTATATTATTAACGAAACTCGAAACATTCGAGTACAAGCAA
AGAGGTCTTTAATGATCTTAATAGCATGGAGTTCCTCACCCCTTACTCCACATCCAAGTAAACATTGGATTATGATT
CAAAGACCACTAACGTTTCTGTTAACTCAGATGGGGGGCAGACTAGGCCATGCCTGTCACTTTGCAAGATACACACC
AACGTCCGATTCTTCTGTATCTCCAACCACAGGAAACCTACCCCTGAAACTTCCTGTTTTGATAGATTTAAAAAAAA
ATTTTTTTAGCTCCAGGTTGGGTGCTGGTCATTCATGCATACAGCAAATTTTTTAAAATTCTTACCACATTCAAAGC
ACTGTACCTTCTTGAAAAAGGCCAGGCAAGCAGATGCTTTAAGCATTCAAATGAATAGAAAAACTGCATTCTCAAAG
AGGACTTCTTATTTCATTGCTCCAGAATTAAAGCACCAGTAGAAAGACCATATGATATAATTAGCAATGTAAGTTAT
TTGAGGGTGCATGTTTACTAGTATGGAATCTCTGACAGCTGCTTACTGAATAAAGAAGACCATATATAAAGACAGGA
GAAAGTGGCTGAACAAAGCCATCTGAAAAGTAAATTTGAGGCAGGGCATGGAGGCTCATACTTGTAATCCCAGGATT
TGGGGAGGCTTAGGCAAGAGGATCACTTGAGTCCAGGAGTTCCAGATCAGCCTGGGCAACATAGTGAGAACTCATCT
CCACAAAAAAATTAAAAAATTAGTTGCATGTGTTGGTGTGCCTCCAGCTACTCAGGAGGCTGAGGCAGGAGGATTGC
TTGAGCCCAAAGATCAAGGCCACAGTGAGCCATGATCACACTACTGCACTCTAGCCTGGGTGACAGAGCAAGACTCT
GTTAAAAACACAGAAACAAAACAGTAAAGTTGAATGAATAACCCCTCGGATCAATACCACAATACATTCATCCCCTT
TCCCTCTCTCCAATCCAGTTAGGTAGCATCATATTTAGAAATATTTCCTCAAAGCATCTTTAATCTTTTCAGCAGCT
GAAATAATTTCCTAATATATCATCCCATCTCCAGTTTCTCTTAACTACTTCTGCAACGGTCTTTTTGTATGCAAATG
TCATGTGTTATCCCCTGCTTAAAATTCTTCACTGATTTTGCCTTTACCTACACTGAAAGTAGTTGTGAACTGATAGC
CTGCAATTGCACAATATTGGCCTGCACAGGTTGAAAAAAAATTTTACTGGCCAGTGGCTCAAGCTTGTAATCCCAGT
GCTTTGGGAGGCTGAGGCAGGAGGATTGCTTGAGGCCAGGAGTTTGAGACTAGCATGGCAACATAGTGAGAACTAGT
CTCTAAGAAAAATAAAAATAAAATTAGGCCAGGCACAGTGGATCACGCCTGTAGCTCCAACTACTCAGGCGGCTGAT
GCTGATGTGGGAGGATCACTTGAATGTGGGAGGCAGAAGTTGCAGTGAGCTGAGATGGCACCACTGCACCCCAGCCT
GGGTGACAGAGTCTCAAAAAAAAAAAAAAAGCCAGTTGTGTTAGCATGCACGTTTAGTCCTAGCTACTCAGGAGGCT
GAGGTGGAGATCTTTGAGACCTATAGTTTGAGGCTGCCGTGAGCTGTGGTTGTGCCACTGCCCTACAGCCTGGGCTA
CAGAGTGAGACCCTGTGTCTAAAGAAAAAAAAATTAAATTTGTTGCTAACTCGAAAACGTTTTGAGATTTCCCATAA
ACAATTAGATTCTTGGCTTCTTTTGAAAAATCTGAAGATCTGTTAACACCGGGCCTGCATTTCTGTATGACAACAAT
TGGTGTTTAGATGGAGCATGTTCCTTGCCATTTTTCGTCTCCCTGACACTGAGACTTTGTTAGTTACTGTTCATTAT
TGGTCTTGCACTTTTGTTCATGTCTCCTTCACCTTATTTGCCAGATCCAAATAAGCTGTAAATGCACAAATTGTTAA
GCTGTGCGTAAACTATTTTGCAATTCCTCTATTTTTCCTAGAATTTGAACTTTACTTGGTCGTTTTAGTTTTATATT
TATATAACCCATCTTCTTATGTTCTGTGTTACTCAAATGTTCCCTTATGTTAGATTGATGGAGGACTTACCTGCTTC
TCATCTTAATAGTCTGTTCAGAATGACTTTCAGCATTATAATGCCTGCTTGGCATTAGGAATTCTGTTAGTTTCCAT
GACTTTTTAAAGTATTAACATCAGTGTCTTTGGTTCTTGTTCTCCTTTGCCTTCATAGAAAATTAATTTATTAATTA
GCTTATTTTCTAGCTCCCACTTTGCCCCAATAAGTTAAGGAACTAGATGTGTTAGTGTGATTGGGAACTCTAGCTGT
TATTACTCTGCTTGAACTAAATGTAACTGTTATTACTCTTAGACTTTTTAAGTATGACCATATGAATTTTTCTGCTT
GTAAGCAGTGTCAGAAGAAAGTTGAATGCTGTAGCCTTAAGTGCTTTCAGACTCTGGTGGATGCCTAATTTTTTTAT
TTGTTCCTTGGGTGCTATTTCTGAACATCATGTTCTCCTTTCCCTCTTCCATCTAGCTCAGTGAATGTGGGGTGTTC
TTTCTATATGCTTTCACTTCCACTTAACATTCTTCTAAATGCTGTACAGTCCCTAAGCTTACACTCTCCATACTAAA
TTTTGGTGGCCAGTGTTTTTACCATGATTAAATCTCACTGTTATCCAGTTTATCGTTAAAAGTTTTTCTCCTCTCCT
CTGGATACAGTTTACTTTGTGTTTTTCTTCTAAACTGTCTACCTGTGTCACCTTCTTTACTGCCTCGCTCCTGTTTT
TTCTCTGTGTGTAATTCTTTAAAGTTCTTCCAGGTTTTTCTTCTTTTCTATTGCCCTGTCTAATGTCCTAGATTTAA
ATATAGTTCTGCAAGTCAAAGGTAAGTTCTTTCTACTCCCCATGGATCTAATTTATAAGGAAGAGGAAGCTTTGCTT
GTAAGTATATTGAAGTTATTTATATGTTCATCTGTGTGTGTGTGCACGCACACACAGGGAAAGAGAGAGAACATTAT
GCTAACCAGAGATGTCTAATACAACCACATTTGGTGACTTCAGATATAAAGGGTGTGGTGGCCATTAGGAGTATTAC
TTCTAGGTCAAGACTGTCCCTACTATTAAATATAATATATTGTTAAACTAAACGTAATAAAAAACAAATGTGCTTAA
TGAAATTGGGCAGGGAGCCTTTTCATTTTCTTCTAAAGAAATATGAAGAGAAAGTTTAGAAAGTTGAATGTTTTGGG
CTTTTTATGTTGCTTGCTATTGTAAATCTTGCAAAAGCTAATGACTACTTGACATCTTTTTTTCCTGACAACTTTTC
TTCCTCAATTTTATCTTTCCTCACAACAGCTGCAGGGAAGTCAACATTTGTGAATATCCTTAAACAATTGTGTGAAG
ATTGGGAAGTGGTTCCTGAACCTGTTGCCAGATGGTGCAATGTTCAAAGTACTCAAGATGAATTTGAGGTATGAAAA
TAAAATTTAAGTAGATAAAAGCCTCTTGGTTTAGAGGAGCAGTAGTACTTAATCTCTTTTTCTTTTTCTTTTTCAAT
AAAACTTTCAAATCTCGCTTTAGGTATATATCTTCATCTAGGTGGTGGTTACATGGATATATATACAGTTGGCTCTC
CATATCAATGGGTTTCAGATTGATATGGATTCAACCAATCATGAATAGAAAATATTCAGAAAGAAATAATACAAAAA
AATACAAATAATAAAGGATTACTGTTCATATAGCATTTACATTGTATTAAGTATTATAAGTAATCTAGAGATGATTT
ATACTATTTGGGAGGATATATGCAGATACTACACCATTTTACATAAAGGACTTGAGTATATGTGGATGTTGGTATCT
GTGTGGAACCTGGAATCAATTCTTGAGGTTACTGAGGGGTGACTATATGTTTAAACATGCATTGAGGCCGGGGTTGG
TGGCTCACGCTTGTAATCCCAGCACTTTGGGAGGCTGAGGCGGGCGGATCACTTGAGGTGAGGAGTTCAAGACCAGC
CTCGTTAACATGGTGAAATGCCGTCTCTACTAAAAATACAAAACTTAGCTGAACGTGGTGGTGGGCACCTGAATTCC
ACCTGCTTGTAAAGCCGAGGTAGGAGAATTTCTTGAACCCAGGCAGCGGAGGTTGCAGTGAGCTGAGATGGCGCCAC
TGTGCTCCAGCCTGGGTGACAGAATGAGACACTGTCTCAACAAAAAAAAAAAAAGGAAAGTAAAATTTATCAAACTA
TGCATTCAAGATTTATGCACTTTAAGTAAAGTATAAAAGTTTTCTTTTTCATAAAAATTTCTTTTTTTGTTGTTATC
CTTTAAGTTCTGGGATACATGTGCAGAACGTGCAGGTTTGTTATGTAGATATACACATGCCATGGTGGTTTGCTGCA
CCCGTCAACCCGTCATCTACATTAGGTATTTCTCCTAATGCTAACCCTTCCCTTGCCTCCTACCCCACCGACAGGCC
CTGGTGTGTGATGTTCCCCTCCCTGTGCCCATGTGTTCTCATTGTTCAACTCCCACTTACAAGTTAGAACATGCGGT
GTTTGGTTTCCTGTTCCTGTGTTAGCTTGCTGAGATTGATGGTTTCCAGCTTCATCCATGTCCCTGCAAAGGTTATG
AACTCATTCTTTTATTATGGCTGCATAGTATTCTTTGGTGTATATGTGCCACATTTTGTTTATCCAGTCTATCATTG
ATGGGCATTTGGGTTGGTTCCAAGTCTTTGCTATTGTGAATAGCGCTGGAGTAAACATACATATGCATGTGTCTTTA
TGGTAGAATAATTTATAATCCTTTGGGTATATACCCAGTAATGGGATGGCTGGGTCAAATGGTATTTCTGGTTCTAG
ATCCTTGAGGAATCGCCACACTATCTTCCACAGTGGTTGAACTAATTTACACTCCCACTAACAGTGTAAAAGCATTC
CTATTTCTCCACATCCTCTCCAGCATCTGTTGTTTCCTGACTTTTTAATGATCACCATTCTAACTGGTGTGAGATGG
TATCTCATTGTGGGTTTGGTTTGCATTTCTCTAATGACTGGTGATGATGAGCTTTTTTTCATGTTTGTTGGCTGCAT
AAATGTCTTCTTTTGAAAAGTGTCTGTTCATATCATTTGCCCACTTTCTGATTGGGTTGTTTGTTTTTTTTTCTTGT
AAATTTGTTTAAGTTCCTTGTAGATTTTGGATATTAGCCCTTTGTCTGATGGGTAGATTGCAAAAATTTTCTCCCAT
TTTGTAGGTTGCCTGTTTACTTGGATGATAGTTTCTTTTACTGTGCAGAAGCTCCTTAGTTTAATTAGATCCCATTT
GTCAGTTTTGGCTTTTGTTAACACTGCTTTTGGTGTTTTAGTCATGAAGTCTTTGCTCATGCCTATGTCCTGAATGG
TATTGCCTATGTTTTCTTCAGAGTTTTTATGATTTTAGGTCTTATGTTTAAATCTTTAATCCATCTTGAATTAATTT
TTGTATAAGGTGTAAGGAAGGGGTCCAGTTTCCGTTTTCTGCATATGGCTAGCCAGTTTTCCCAGCACCATTTATTA
AATAGGGAATCCTTTCCCCATTGCTTGTTTTTGTCAGGTTTGTCAAAGATCAGATGGTTGTAGACGTGTGGTATTAT
TTCTGAGGGCTCTGTTCTGTTCCATTGGTCTATAGCTCTGTTTTGGTACAATACCATGCCGTTTTGGTTACTGTAGC
CTTGTAGTATAGTTTGAAGTCAGGTAGCATGAGGCCTCAAGCCTTGTTCTTTTTGCTTAGGATTGTCTTGGCTATAC
GGGCTCTTTTTTGGTTCCATATGAAATTTAAGGTAGTTTTTTCTAATTCTGTGAAGAAAGTCAATGGTAGCTTGATG
GGAATAGCATTGAATCTGTAAATTACTTTGGGCAGTATGGCCATTTTCACAATATTGATTTTTCCTATCTATGAGCA
TGGAATGTTTTACCATTTGTTTGTGTCCTCTCTTCTTTCCTTGAGCAGTGGTTTGTAGTTCTCCTTGAAGAGGTCCT
TCACATCCGTTGTAAGTTGTATTCCTAGGTATTTTATTCTCTTTGTAGCAATTGTGAATGGGAATTCACTCATGATT
TGGTTCTCTGTCTGTTAGTGGTGTATAGGAATGCTTGTGATTTTTGCCCATTGATTTTGTATCCTGAGACTTTACTG
AAGTTGCTTGTCACCATAAGGGATTATGGGCTGAGATGATGGGGTTTTCTAAATATACAGTCATCATCTGCAAACAG
AGATAATTTGACTTCCCCTCTTCCTATCTGAATCCCCTTTATTTCTTTCTCTTGCCTGATTGCCCTGGCCAGACCTT
CCAATACTATGTTGAATAGGAGAGGTGAGAGAGGGCATCCTTATCCTGTGCCGATTTTTGAAGGGAATGGTATCGGC
TGTGGGTTTGTCATAAATAGCTCTTATTTTGAGATACATTTCATCAATACCTAGTTTATTGAGAGTTTTTAGCATGA
AGGGATGTTGAATTTTATTGAAGGCCTTTTTTGCATCTATTGAGATAATCATGCGGTTTTTGTCATTGGTTCTGTTT
ATGTGATAGATTATGTTTTATTGATTTGAGTATGTTGAACCAGCCCTGCATCCCAGGGATGAAGCCGACTTGATCGT
GGTAGATGAGCTTTTTAATGTGCTACTGGATTCGGTTTGCCAGTATTTTATTGAGGATTTTCGCATCGATGTTCATC
AGGGATATTGGCCTGACATTTTCTTTTTTTTGTTGTGTCTCTGCCAGGTTTTTGTATTAGGGTGATGCTGGCCTCAA
AAAATGAGTTAGGGAGGAGTCCCTCTTTTTCTATTGTTTGGAAGTTTCAGAAGGAATAGTACCAGTTCCTCTTTCTA
CCTGTGGTAGAATTCAGCTGTGAATCCATCTGGTCCTGGGCTTTTTTTTGGTTGGTAGGATATTATTGCCTCAATTT
CAGAACTTTTTATTGGTCTGTTCATTAAACCAGAGTTCTTCCTGGTTTAATCTTGGGAGGGCGTATGTGTCCAGGAG
TTTATCCATTTCTTCCAGATTTTCTAGTTTATTTGCGTGGAGGTGTTTATAGTGTTCTCTGATGGTAGTTTGTATTT
CTGTGGGATCAGTGGTGATCCTTTATTGTGTCTATTTGATTCTTCCCGCTTTTCTTCTTTATTATTCTGGCTAGTGG
TCTATCTATTTTGTTAATCTTTTCGAAGAACCAGCTTCTGGATTCATTGATTTTTTGAAGAGTTTTTCGTGTCTCTA
TCTCCTTCAGTTCTGCTCTGATCTTAGTTATTTCTTGTGTTCTGCTAGCTTTTGAATTTGTTTGCTCTTGCTTCTCT
AGTTCTTTTAATTGTGATGTTAGGGTGTCGATTTTAGATCTTTCCTGCTTTCTCATGTGGGCACTTAGTGCTATAAA
TTTCCCTCTAAACACTACTTTAGCTGTGTCCCAGAGATTGTGGTACGTTATGTCTTTGTTCTCATTGGTTGCAAAGA
ACTTATTTATTTCTGCCCTAATTTCGTTATTTACCCGGTAGTCATTCAGGAGTTGTTCAGTTTCCATGTAGTTGTGC
AGTTTTGAGTGAGTTTCTTAATCCTGAGTTCTAATTTGATTGCACTGTGGTCTGACAGACTGTTATGATTTCCATTC
TTTTGCATTTGCTGAGGAATGTTTTACTTCCAATTATGTGGTCAATTTTAGAGTAAGTGCACTGTGGTACTGAGAAT
AATGTATATTCTGTTGATTTGGGGTGGAGAGTTCTCTAGATGTCTTTCAGGTCTGCTTGGTCCAGAGCTGAGTTCAA
GTCCTGAATATCCTTGTTAATTTTCTGTCTTGTTGATCTAATATTGACAGTGGGGTGTTAAAGTCTCCCACTGTTAT
TATGTGGGAGTCTAGGTCTCTTTGTGGGTTTCTAAGAACTTGCATTTTGAATCTGGGTGCTCCTGTATTGGGTGCAT
ATCTATTTAGGATAGTTAGCTCTTGTTGTTGCATTGAACCCTTTACCATTATGTAATGCCCTTTGTCTTTTTTTCAT
GCTTATTGATTTAAAGTCTGTTTTATCTGAGACTAGGATTGCAACCCCTGCCTTTTTTTTGCTTTCCATTTGCTTGG
TAAATCTTCCTCCATCCCTTTATTTTGAGCCTTTGTGTGTCTTTGCATGTGAGATGCGACTCCTGAATACCGCACAC
TGATGGGTCTTAACTCTGTCTAATTTGCCACTCTGTGTCTTTCAACTGGGGCATTTAGCCCATTTACATTTAAGGTT
AATATTGTTATGTGTGAATTTGATCCTGTTATTATGATGCTAGCTGGTTATTTTGCCTGTTAGTTGATGCAGTTTCT
TCATAGTGTCGATGGTCTTTACATTTTGGTATGTTTTTGCAGTGGCTGGTACCGGTTTTTCCTTTCCATATTTAATG
CTTCCTTCAGGAGCTCTTGTAAGGCAGGCCTTGTGGTGATAAAATCCCTCTACATTTGCCTGTCTGTAAAGGATTTT
ATTTCTCCTTCACTTATGAAGCTTAGTTTGACTGGTTATGAAATTCTGGGTTGAAAATTCTTTTCTTTAAGAATGTT
GAATGTTAGTCCCCACTCTCTTCTGGCTTGTAGGGTTTCTGCCGAGAGATCTGCTGTTAGTCTGATGGGCTTCCCTT
TGTGGGTAACCCGACCTTTCTCTCTGGCTGCGCTTAACATTTTTTCCCTCATTTTAACCTTGGTGAATCTGACGATT
ATGTGTCTTGGCGTTGCTCTTCTCAAGGGCATCTTTGTGATGTTTTCTGTATTTCCTGGATTTGAATGTTGGCCTGT
CTTGCTAGGTTGGGGAAGTTCTCCTGGATAATAATCCTGAAATGTGTTTTCCAACTTGGTTCCATTCGTCAGTTTCA
GGTATACCAGTCAAACGTAGGTTTGGTCTTTTCACATAGTCCCATATTTCTTGGGAGGCTTTGTTTGTTCCTTTTCA
TTCTGTTTTCTCTAATCTTATCTTCACGCTTTATTTTTATTAAGTTTTTCTTCAATCTCTGATATCCTTTCTTCCAC
TTAATCGATTCGGCTATTGATACTTGTGTATGCTTCACCAAGTTCTCATGCTGTGTTTTTCAGCTCCATCAGGTCAT
TTATGTTCTTCTGTAAACTGGTTATTCTAGTTAGCAGTTCCTGTAACCGTTTATCAAGGTTCTTAGTTCCTTGCATT
GGGTTAGAACGTGCTGCTTTAGCTCAGTGGAGTTTGTTATTAACCCACCTTCTGAGGCCTACTTCTGTCAATTCGTC
ACACTTATTCTCCATCCAGCTTTGTTCTCTTGCTGGCGAGGAGTTGTGGTCCTTTGGAGGAGAAAAGACATTCTGGT
TTTTGGAATTTTTAGCTTTTTTGCACTGGCTTTTCTTCATCTTTGTGGATTTATCTACCTTTGGTCTTTGATGTTGG
TGAACTTCAGATGGGGTTTTTGCGTGGTTGTCCTTTTTGTTGATGTTGATGCTATTGCTTTTTGTTTGTTAGTTTCC
TTCTAACAGTCAGGCCCCTCTGCTGCAAGTCTGCTGGAGTTTGCTGGAGGTCCACTGCAGATCCTGTTTGCCTGGGT
ATCACCAGCGGAGGCTGCAGAACAGCAAAGATTGCTGCCTGCTCCTTCCTCAGGAAGCTTTGTCCCAGAGGGGCACC
TGCCAGAGGCTAGCCAGAGCTCTCTTATATGAGGTGTCTGCCGACCCCTGCTGGGAGCTGTGTCCTAGTCAGGAGGC
ATGGGGGTCATGGACCCACTTGAGGATGCTGCCTGTCCTTTAGCAGAGCTTGAGCGCTGCGCTGGGAGTTCCACTGC
TCTCTTCAGAGCCAGCAGGCAGGATTGTTTAAGTCTGCTGAGGCTGCACTCACAGCCACGCCTTCCCCCAGATGCTC
TGTCCCAGGGAGATGGGAGTTTGATCTGTAAGTCCCTGACGGGGGCTGCTGCCTTTCTTTCAGAGATGCCCTGCCCA
GAGAGGAGGAATCTAGAGAGGCAGTCTAGCTTCAGGGGTTTGCCAAGCTGTGGTGGGCTCTGCCCAGCTTGAACTTC
CAGGTGGTTTTGTTTACACTGTGAGGGGAAAACCACCTACTGAAGTCTCAGTAATGGCGGGCACCCCTCCCCCCACC
AAGCTTCAGTGTCTCAGGTTGACTTCAGACTGCTGTGCTGGCAGTGAGAATTTCAAGCCAGTGGATCTTAGCTTGCT
GGGCTCCGTGGGGGTGGGATCTGCTGAGCAAGACCACTTGGCTCCCTGGCTTCAGCCCCCTTGGCTCCCTGGCTTCA
GCCCCCTTTCTAGGGGAGTGAACGGTTCTGTCTCGCTGGCATTCCAGGCACCACTGGGGTATGAAAAAAAACTCCTG
CAGCTAACTCCGTGTCTGCCCAAATGGCTGTCCAGTTTTGTGCTTGAAACCCAGGGCCCTTGTGTAGGCACCCGAGG
GAATCTCCTGGTCTGCGGGTTGTGAAGACTGTGGGAAAAAGCCTAGTATCTGGGCTGGATAGCACCATCCCTCAAGG
CTTCCCTTGGCTAGGGGAGGGAGTTCCCCCACCCCTTTCACCTTCCAGGTGAGGCGATGCCCCACCCTGCTTCTGCT
TGGCCTCTGTGGGCTGCACCCACTGTATAACCAGTCCCAATGAGATGAATTGGGTACTTCAGTCGGAAATGCAGAAA
TCACCCGTCTTCTGCATTGGTCTCACTGGGAGCTGCAGACCAGAGCTGTTCCTATTTGGCCATCTTGCCTGGGAATC
TAAAAGTTTTTAAAAAGTTAAATAATAAAAGTTTTAAAAAATCTAACAGCAAATTAAATTCCTTCATTGTCTTTTCT
AATTTTTTTTTTTTTTTGAGATGGAGTTTCGCTTTTGTTGCCCAGGCTGGAGTGCAATGGCTCGATCTTGGCTCACC
ACAACCTCCGCCTCCCGGGTTCAAGCGATTCTCCTGCCTCAGCCTCCCAAGTAGCTGGGATTACAAGCATGGGCCAC
CATGCCTGGCTAATTTTTTTTACTAGAGATGGGGTTTCTCCATGTTGGTCAGGCTGGGCCCAAACTCCTGACCTCAG
GTGATCCGCCTGCCTCAGCCTCCCAAAATGTTTGGATTACAGGCCTGAGCCACTATGCCCAACCCTTCAGTGTCTTT
TCACATTTTGTTTAAAAACAAGCAAATAAGGCCGGGTGCGGTGGCTCACACCTGTAATCCCAGCCCTTTGGGAGGCC
GAGGCAGGTGGATCGCCTGAGGTCAGGAGTTCGAGACCAGCCTGGCCAACATGGTGAAACCCTGTCTCCACTAAACA
TACAAAAATTAGCTGGGCGTGGTGGTGGGCGCCTGTAATCCCAGCTACTCAGGAGGCTGAGGCGGGAGAATTGCTTG
AACCCGGGAGGTGGAGGTTGCAGTGAGCCGAGATCGCGCCATTGCACTCCATCTCAAAAAAAAAAAAAACAGAAAAA
ACAAAAACACCAAAAAAAAAAAAAAAAAAGCAAATAAGAAAAAGAAAAACTACTATTCAGTAACTGAATTTTACCTT
TCAAGCCTTTTGTCATTTACTCTCATTTCTTTTTACTTTCATTCTTGCCAGACTTTCACTCTGGGCAGAAGTTGTAA
TTTTGCAGCATTTTACCGTTCTGCACTGCAAATGAGGAAGAAAGAGAATGACAGAGTTCAGTGTCATTTTTTTAAAT
ACCAAAATGTATGGTTGTATGTGTGAGAGAGACGTTGCTAAGATTTTCGTTTCTGGAGAAATGAGAATTGAAATTGA
AAGCAAGGCCAGGTGTGGTGGTTTATGCATGTAATCCTGGAATTTTTTTTTTTTTAAGTGACAGCAAGTTTATTAGG
AAAGTAAAGCAATAAAGAATGGCTACTCCATAGGCAGAGCAGCCAATCCCAGCACTTCGGGAGGCTGAGGCCAGAGG
ATCGCTTGAGCCCAGGAGTTCAAGACCAGCCTGGGCAACATAGGGTGACCCCGTCTCTACAAAAAATTTAAAAATTA
GCTAGGTGTGGTAGCCTGCACTTGGGGTTACAGCTACTTGGAAGGCTGAGGTTGGAGGACTGCTTGAGCCCAAGAAA
TTGAGACTGTAGTGAGCCATGCTCATGCCACTGTACTCTAGCCTGAGTGACAGCAAGACCCTGTCTAAAAGAAAAAA
AGAAATTGAAAGCAGTAAGGCAGAAACCTTAAAAATGGAAATGTATGCTAACAACTTACTTTGATCTGAGGAGTACA
GAAAATTGAAAAAAAAGTATCTAAAAAAATTTAGCTGTTGGTATAAGGAATTCTGGGAATAATAATTGAATATTTAA
AATGTAAATCACATATGCAAAGAGGAATCACTAGCAAGATATTCCATGGACTTTGAGAATATATGGTTTTGAATAAC
CAAAAATACTTTTTCATACACCTTCTATTTGCCAGGCATAGCTAACTTTAGACCTAGGGCCTAATTCAGTACACCCC
AATCCTTAGGATATCCAGGACTAAATCTTAAAGGGGCTTTCTTCCCATATAGACCTCATTATCTTAAGAAATTGATG
TTTTATATCTGATTTTTGGGGAAAGGCAAGGTTAGTCTACAGTAGAATTTTAAAGTGGGTAAATTTGTTTATTTGAT
TCTTTTTAAAAACATTTTTGACATGATATATCTGTAGAAAAGTAGGTAAAAGCTAATGTACAGTTTGAAGAACAGTT
TTAAAGTTGACATCTTTATAACCATTATCCAGGTCAAGAAATACGACATCATCATTCCAGAAATCCCTGTTCGCCTT
CTCCAATCACTATATCTTCTTTCCTACGTTACTACTCCGCTAGGGGTAATTACTCCCATTCTTTTGTGATAATAATT
TTCCTGCTTCCTAAAAATTGTTTGAGCATACATGAACATACCTCTAAGTATAATTTAGTTTTGTCTATTTTTGAATG
TTATATAAGTGGAATTACATTGTTTGCCTTTTTTCCTCTCGCTTCTTTTACTTAATACATTTATGAAATTTGTCCAT
ATTATATGCAGCTGTAGCTTATTTTCAGCTATATAGTATTGAATTTTGTTTTTTAAAAATATTCTTTTTTTTGAGAC
AGGATTTCACTCTGTTGCCCAGGTAAAGTGCAGTGGCGCAGCCATGGTTCACTGCAGCCTCAACCTCCCTTGGCTCA
GGTGATCCTCTCTCCTCAGCCTCCTGAGTATCTAGGACCATAGGCCACCACGCCTGTGTGCCACCAGGCTTGGCTAA
TTTTTGTATTTTTTATAGAGACGGGGTTTTGCCATGTTACCCAGGCTGGTTTCAAACTCCTGGGCTCAAGCGATCTG
CCCATCTCAGCCTCCCAGAGTGTTGGGATTACAGGCTTGAGCCACTATGTGGGGCCTATAGTATTTAATTTTATTCG
TCTATCACTGTTTGTCCATTTTATACCTTTAATTGATTTTTGAGTGATTTCCAATTTAGTTGCTTATGTAAACAGCA
TTGCTGTGAACATAAATTCACCCGCATACATGCAAAAGTTTCTGTAGGAATAAGAAGTAAAATTACTGGGAATGCTT
ATGATGCCTGTTTTCCAAACTGGCTTTCCAATTTTCTCTCCCCCCAGTGTAATCAAGCTCTCATTAATCTATGTCTT
TGCCAGCATTTAGTAGTGTCTGGCTTTCTCATTTTTGTCATTCTGCTGAGTGTTTACTATTTTTTTGTTTAATTTGT
ATTTCCCCGCTGGCTAATGAGGCTGTGTTCCTTTTCATATGTTTATTAGTCATTTTGATTTTTGCAAAGTGCCCACT
TGGGTCCTTTTGCCCATTTTCCTCTGAGCCATCTGTCTTCTCTCACTGCATTTTTAATGTAGCTTTTCGCTGAAGTA
CCTACTCAAGTTTGCTTAGCTTTTGAAGGAGAGACTGGAGTTCTAAAAGCAACTCTTTTTCCTTTTATTTCTGGGGT
CAGCTGTCAGTGCAGTTATCCTCAGGTAGTAGACAGAAACTAACCAGATTAAACCAATGTTGGAAAATAGAGGGTAT
TCTTAAACAGGGTATTGGACTAGGTGACCTCCATTTAAGGTCCGTTGACATATTTGTCATTTGACTCTCAGCATGCC
TCACATTAACCAAGGGCACAGAAACTGACATATAAAACCAAAATGCCAAAAATTTTAATTTGCAGAAAGAAATGTAA
TCATTAAGGTAATTGAATTTAGATCAGTTTATTCTTTATTTGGTTCGTAATTCTCAACCAGTGGAAGGAAGTCATAA
GAATGTCTGAGAGCTAAACAGTTTGAACACATGGAGATAGAGTGTGGAAAAATAGATAACAATAACTGGGAAGGGTA
GGAGGAGGGGAAGATGAAGAGGAGTGAGTTAAAGTGTACAGATATACAGGAAGCTTGAAGAAATAATTCAGTGTTTT
ATAGCAAGGTAGAATGGAAAAATACATTGTACTCAGGTGATGGACACCCTGAATGATTCGATTACTGCATATTATAT
ACATGTACAAAATTTCTCATGTATTCCATAATTTGTACACACAAGAAAGAATCTCAAGGATTCGGGTGCTTTTTAAA
ATAACAATCCCTTGGAGGTTCAAATATGTGGGATGCCTCATAGTTAAAAGTCATCACCCTAGTGAATCACTGTTAAC
TGTTGGGAGTATACTGAACTCTTTAGTTAGGTTGCAGAGAAGTTGAAAAACATTGCTTTCGTATATAAGACAAGACA
ATTTATAATAAGGTTGTCTGTTGAGCTTGGAATCAGAGCCCAGAGGGATTAGTTAAATCATGTTAATCAGACTGTGT
CATCAGACTGTGATGCATTAATACATTTGTCTTCATTTTAAGACTTAATGGCTTTCTGAATCTGATATCCATGTTTA
TGGGATGGGACATTTTCTTTCCATATACTTGATTCAGAATGCCAAACTGGCCATTCTTTATAACTAGCATGCAGGTG
ATCCTATTATTATTAACATATTCTTTTATCAGGCCGTTTATTGTAATCTGGTCATGTCTTCATGATGTTACAATTTG
ATTACCTTTGTTAGAGTTTGGTGCACATTCTTGGAAGGTAGCTTCAGCTTTTTTGTGTGTATTGGGGACTGCTCCGT
TGTTAATTCTCTGTACTTCTGTACAGAGGGGGTAGTATTTTTGAGATATAGTTGTGGAATATGAGGATGGTTGGGTG
GGTGGCTTCCCTGTATCTTTGGATATTTTCTTCCACTAAACTCAAGAAGTGAGATTTTATTTCTTCATCCAGTAGTC
CTTCTTCCCCAGACTCATCCTTGATCTATTCCTTGAGGTTATAGTATGAATCACCCACTATTCAAATGTCAATTTTT
CTTAGAATGCTGTTAGTGATGAGAAGTGACTGTTTTCAGACCTAACCTTGGCAAGGTCAGTCCTACTTTGATGTTCT
TGTTTCATCACACTTCTTGGCATTTGTAGATTTGGAAGAATTGGGCCTTTGGTACCTCTGATCTCTTCGTTTAGCAA
CTTACTGTGCACCCATATGCTTAGCTTTTGCTGTTTTAGCTTTTTTTTTTTTTTTTTAACCTGCCACCTAGTGGCCG
AAATGTTGCTATACTATTGATAAGGTACTCCTAATTTTGGCAAAATAGTAAGAGGCAAAGCACCAAAGATTATGTTC
TCTCCCTTCTCCAAATCTCTCTTGGTGAGAATGATCTTTAAAACATACCACTCAGATTATTAGCAATCTTGGTATGG
AACGTTTTTAAAAATAATAATAATGTACTTTATGTGGTGATTTATGTTATTATTTAGGCCCAAAGTTTTGATTTAAT
TGTTTCCTTTTAGCTTATTTTTGAGATATGCAGTCTGTTAGGAAGCTGTCTCTGTCTTGTTTTTTCATTGAGCTGAA
TTCTTTTGGAGGATCACTATTATTGTGAAGTACTATTTTACTTTTAATTAAAAAAATTAAGATATTTTTATTTTTAA
TTTACAGATAAAAATTATATTTAATTTTATATTATATTTATATATAAATCATATGTAATGTAAATTATATACATAAG
TTATATATAATATAAAAATTATATACATAATATAAATTATATATAATATAAAAATTATATTTATGGTATGCAACATG
ATGTTTTAAAATATGTCTACATTGTGGAATGGTTAAACCAAGCTAATTAACATACGCATTACCCGTACTATTTTACT
TTTATCCAATACTTCTTACCACTAATAAGCCACTGAAAAACTAGTAATTGTGATAAAGAAGGTGTAAACCATACTTA
TTTATGACAGAACAGCATATTTTTTACTTTTAATAAGTCATTCTTTTTTTTTTTTTTTGAGACGGAGTCTCGTTCTG
TCGCTCAGGCTGAAGTGCAGTGGTGTGATGTTGGCTCACTGCAACCTCTGCCTCCCAGGTTCAAGCGATTTTCCTGC
CTTAGCCTTCCAAGTAGCTGGAGCTGGGATTACAGGCATGTGCCACCACGCCTGGCTAATTTTTGTAATTTTAGTAG
AGATGGGGTTTCACCATGTTGGCCAGGCTGGTCTTGAACTCCTGACCTCAAGTGATCCACCCATCTCAGCCTCCCAA
AGGGCTGGGATTACAGGTGTGAGGTCTCTTTTTTTTTTTTTTTTTTTTTTTTAAGACGGAGTCTTGCTCTGTCCCTT
AGTAAGTCATTCTTGAAAATTAGTTAAGAATGCTTGATAAAGCACTTCCACTTGTACCTTTTGAATAGATGTGATAA
CTTTTGTTGGCTTGTATTTTTTTGAGTCTTGCTTTATTGCCCAGGCTGGAGTGCAATGGTGAACCTCAGCCCACTGC
AGCCTCCGCCTCCTGGGCTCAAGTGATCCTCCCACCTCAGCCTCCTCAGTAGCTGGGACTACAGGCCCACACCAACA
TGCCCGGCTAATTTTTGTATTTTTTATAGCGATAGGATTTTGCCAATGTTGTCTGGATTTGTCTCAAACTCCTGGGA
TCAAGCAATCCGCCTGCCTCGGCCTCCCAAAGTGCTAGAATTAGGCATATGAGTCACCATGCCTGGCCTGTTGGCTA
TTTTATAATTATCTTGTTTGGTAGACTCTGATCTTTTACAGTTGAACTTAATTCAACTCTTTTGCAATGTTTACAAA
GATTAGACCTAGAATTAAAAGATAAATGATTTTATAGCTGACCTTGATATTTCTATTTTTCACTTACCTTGACAATA
ATCTGTACTTCCTATAACAATGAAGTATTCTTTCATAAAGTGAAAATGGACATATATTATAGAGAACAGAATAAATG
TTATTCATAATCCTACCACTCTGTATGTATCTTTTACATAGTTGAATTAATAATGTAATATATTAAATTTTGGTGGA
GGTGATATGTAATTTAAAAAATATTATAAAGTAATACCTGTTTACTGTTTGAAGTTAAGGAAATATTAGAAATTATC
AAAAGAGGGGACTAAAACTTACCTATAACCTTATTATCCAGAGATAACCATAGATCTTACTTGAAAGAACAGTATAC
AGTTTTGTATTTTGTTTCTTCCCCACCCCCCACAATATTTATATGTCAAGAGTCTGAGATCCAACTGCAAGGCGGCA
GTGAGGCTGGGGGAGGGGCGCCCACCATTGCTGAGGCTTGGGTAGGTAAACAAAGTGGGCTGGAAGCTGGAACTGGG
TGGAGCCCACCGCTGCTCAAGGAGGCCTGCCTGCCTCTGTAGACTCCACCTCTGGGGGCAGGGCATAGCTGAACAAA
AGGCAGTAGAAACCTCTACGGACTTAAATGTCCCTGTCTGACAGCTTTGAAGAGAGTAGTGGTTCTCTCAGCACGGA
GTTTGAGATCTGAGAACGGACAGACTGCCTCTGCAAGTGGGTCCCTGACCCCCGAATAGCCTAACTGAGAGGCACCC
CCCAGTAGGGGGAGACTGACACCTCATACGGCTGGGTACCCCTCTGAGACGAAGCTTCCAGAGGAACGATCAGGCAG
CAACAATTGCTGTTCAGCAGTATTCGCTGTTCTGCAGCCTCCGCTGCTGATACCCAGGCAAACAGGGTCTGGAGTGG
ACCTCCAGCAAACTCCAACAGACCAGCAGCTGAGGGTCCTGGCTGTTAGAAGGAAAACTAACAAACAGAAAGGACAT
CCACACCAAAACCCCATCTGTACGTCACCATCATCAAAGACCAAAGGTAGATAAAACCACAAAGATAGGGAAAAACA
GAGCAGAAAAGCTGAAAATTCTAAAAATCAGAGCGCCTCTCACCTCCAAAGGAACACAGCTCCTCGCCAGCAACGGA
ACAAAGCTGGACGGAGAATGACTTTGACGAATTGAGAGAAGAAGGCTTCAGATAATCAAACTTCTCCGAGCTAAAGG
AGGAAGTTCGAACCCATGGCAAAGAAGCTAAAAACCTTGAAAAAAGACGAATGGCTAACTAGAATAACCAGTGTAGA
GAAGTCCTTAAGTGACCTGATGGAGCTGAAAACCATGGCACGAGAACTACATGACGAATGCACAAGCTTCAGTAGCC
GATTCAATCAACTGGAAGAAAGGGTATCAGTGATTGAAGATCAAATGAATGAAATGAAGTGAGAAGAGAAGTTTAGA
GAAAAAAGAGTAAAAAGAAATGAACAAAACCTCCAAGAAATATGGGACTATGTGAAAAGACCAAATCTGCGTCTGAT
TGGTGTACCTGAAAGTGATGGGGAGAATGGAACCAAGTTGGAAAACACACTTCAGGATATTATCCAGGAGAACTTCC
CCAATCTAGCAAGGCAGGCCAACATTCAAATTCAGGAAATACAGAGAACACCACAAAGATACTCCTCGAGAAGAGCA
ACTCCAAGACACATAATTGTCAGATTCACCAAAGTTGAAATGAAGGGAAAAATGTTAATGGCAGCCAGAGAGAAAGG
TCGGGTTACCCACAAAGGGAAGCCCATCAGACTAACAGTGGATCTCGCGGCAGAAACGCTACAAGCCAGGAGAGAGT
GGGGGCCAATATTCAACATTCTTAAAGAAACGAATTTGCAACCCAGAATATCATATCCAGCCAAACTAAACTTCATA
AGTGAAGGAGAAATAAAATCCTTTACAGACAAGCAAATGCTGAGAGATTTTATCACCACCAGGCCTGCCCTACAAGA
GCTCCGGAAGGAAGCACTAAACATAGAAAGGAACAAGCGGTACCAGCCACTGCAAAAACATGCCAAATTGTAAAGAC
CATCGAGGCTAGGAGGAAACTGCATCAACTAATGAGCAAAATAACCAGCTAACATCATAATGACAGGATCAAATTCA
CACATAACAATATTAACCTTAAATGTAAATGGGCTAAATACTCCAATTGAAAGACACAGACTGGAAAATTGGATAAT
TGGATAAAGACTCAAGACCCATCAGTGTGCTGTATTCAGGAGACCCATCTCATGTGCAGAGACATACATAGGCTCAA
AATAAAGGGATGTATGAAGATCTACCAAGCAAATGGAAAACAAAAAAAAGGCAGGTGTTGTAATCCTAGTCTCTGAT
AAAACAGTCTTTAAACCAACAAAGATCAAAAGAGACAAAGAAGGCCATTACATAATGGTAAAGGGATCAATACAACA
ACAAGAGCTAACTATCCTAAATATATATGCACCCAATACAGGAGCACCCAGATTCATAAAGCAAGTCCTTAGAGACC
TACAAAGAGACTTAGACTCCCACACAATAATAATGGGAGACTTTAACACCCCACTGTCGACATTAGACAGATGAACG
AGACAGAAAGTTAACAAGGATATCCAGGAATTGAGCTCAGCTCTGTACCAAGCGGACCTAAAAGACATCTACAGAAC
TCTCCACCCCAAATCAACAGAATATACATTCTTCTCAGCACCACATCGCACTTATTCCAAAATTGACCACATAGTTG
GAAGTAAAGCACTCTTCAGAAAATGTAAAAGAACAGAAATTATAAAAAACTGTCTCTCAGACCACAGTGCAATCGAA
CCTAGAACTCAGGATTAAGAAACTTACTCAAAACCGCTCAACTACATGGAAGCTGAATAACCTGCTCCTGAATGACT
ACTGGGTACATAACGAAATGAAGGCAGAAATAAAGATGTTCTTTGAAACCAATGAGAACAAAGACACAACATACCAG
AATCTCTGGGACACATTTAAAGCAGTGTGTAGAGGGAAATTTATAGCACTAAATGCCCACAAGATAAAGCAGGAGAG
ATCTAAAATTGACACCCTAACATCACAATTAAAAGAACTAGAGAAGCAAGAGTAAACACATTCCAAAGCTAGCAGAA
GGCAAGAAATAACTAAGATCAGAGCAGAACGGAAGGAAATAGAGACACAAAAAACCCTTCAAAAAATCAATGAATCC
AGGAGCTGGTTTTTTGAAAAGATCAACAAAATTGATAGACCGCTAGCAAGACTAATAAAGAAGAAAACAGAGAAGAA
TCAAATAGACTCAATAAAAAATGACAAAGGGAGTATCACCACTGATCCCACAGAAATACAAACTACCATCACAGAAT
ACTATAAACACCTCTACAAACACCTCTACGCAAATAAACTAGAAAATCTAGAAGAAATGGATAAATTCCTGGACACA
TACACTGTCACAAGACTAAACCAGGAAGAAGTTGAATCCCTGATTAAACCCATAACAAGCTCTGGAATTGAGGCAAT
AGTTAAGAGCCTGCCATCCAAAAAAAGTCCAGGACCAGATGGATTCACAGCCGAATTCTACCAGCAGTACAAGGAGG
AGCTGGTACCATTCCTTCTGAAACTATTCCAATCAATAGAAAAAGAGGGAATCCTCCCTAACTCATTTTATGAGGCC
AGTATCATTCTGATACCAAAGCCTGGCAGAGACACAACAAAAAAAGAGAATTTTAGACCAATATCCCTGATGAACAT
CGATGCAAAAATCCTCAGTAGAATACTGGCAAACTGAATCCAGCAGCACATCAAAAAGCTTATCCACCATGATCAAT
TGTGCTTCATCCCTGGGATGCAAGGCTGGTTCAGCATACGCAAATCAATAAACATAATCCAGCATATAAACAGAACC
AAAGACAAAAACCACATGATTATCTCAATAGATGCAGAAAAGGCCTTTGACAAAACTCAACAACCCTTATGCTAAAA
ACTCTCAATAAATTAGGTATTGGTGGGATGTATCTCAAAATAATAGGAGCTATTTATGACAAACCCACAGCCAATAT
CACACTGAATGGGCAAAAACTGGAAACATTCCCTTTGAAAACTGGCACAAGACACGGATGCCCTCTCTCACCACTCC
TATTCAACATAGTGTTGGAAGTTCTGGCCAGGGTAATCAGGTAGTAGAAGGAAATAAAGGGTATTCAATTAGGAAAA
GAGGAAGTCAAATTGTCCCTGTTTGCAGATGACATGATTGTATATTTAGAAAACCCCATCGTCTCAGCCCAAAATCT
CCTTAAGCTGATAAGCAACTTCAGCAAAGTTTCAGGATACAAAGTCAATGTGCAAAAATCACAAGCATTCTTATACA
CCAATAACAGACAAACAGAGAGCCAAATCATGAGTGAACTCCCATTCACAGTTGCTTCAAAGAGAATAAAATACCTA
GGAATCCAACTTACAAGGGATGTGAAGGACCTCTTCAAGGAGAACTACAAACCACTGCTCAATGAAACAAAAGAGGA
CACAAACAAATGGAAGAACATTTCATGCTCGTGGATAGGAAGAATCAATATCGTGAAAATGGCCATACTGCCCAAGG
TAATTTATAGATTCAATGCCATCCCCATCAAGCTACCAATGACTTTCTTCACAGAATTGGAAAAAACTACTTTAAAG
TTCATAAGGAACCAAAAAAGAGCCCGCATTGCCAGGTAAATCCTAAGCCAAAAGAACAAAGCTGGAGGCATCACGCT
ACCTGACTTCAAACTATACTACAAGGCTACAGTAACCAAAACAGCATGGTACTGGTACCAAAACAGAGATATAGACC
AATGGAACAGACCAGAGCCCTCAGAAATAATACCACACATCTACAACCATCTGATCTTTGACAAACCTGAGAAAAAC
AAGAAATGGGGAAAGGATTCCCTATTTAATAAACGGTGCTGGAAAAATGGCTAGCCATATGTAGAAAGCTGAAACTG
GATCCCTTCCTTACACCTTATACAAAAATTAATTCAAGATGGATTAAAGACTTAAATGTTAGACCTAAAACCATAAA
AACCCTAGAAGAAAACCTAGGCAATACTATTTGGGACATAGGCATGGGCAAGGACTTCATGTCTAAAACACCAAAAG
CAATGGCAACAAAAGCCAACATTGACAAATGGGATCTAATTAAACTCAAGAGCTTCTGCACAGCAAAAGAAACTACC
ATCAGAGTGAACAGGCAACCTACAGAATGGGAGAAAATTTTTGCAATCTACTCATCTGACAAAGGGCTAATATCTAG
AATCTACAATGAACTCAAACAAATTTACAAGAAAAAAACAACCCCATCAAAAAGTGGGCAAAGGATATGAACAGACA
CTTTTCAAAAGAAGACATTTATGCAGCCAACAGACACATGAAAAAATGCTCATCATCACTGGCCATCAGAGAAATGC
AAATCAAAACCACAATGAGATACCCTCTCACACCAGTTAGAATGGTGATCATTAAAAAGTCAGGAGACAACAGGTGC
TGCAGAGGATGTGGAGAAATGGGAACACTTTTCCACTGTTGGTGGGACTGTAAACTAGTTCAACCATGTGTAAGACA
GTGTGGTGATTCCTCAAGGATCTAGAACTAGAAATACCATTTGACCCAGCCATCCCATTAGTGGGTATATACCCAAA
GGATTATAAATCATGCTGCTATACAGACACATGCACACATATGTTTATTGCGGCACTGTTCACAATAGCAAAGACTT
GGAACCAACCCAGATGTCCATCAGTAATAGACTGGATTAAGAAAATGTGGCACATATACACCATGGAATACTATGCA
GCCATAAAAAAGGATGAGTTAATGTCCTTTGTAGGGACATAGATGAAGCTGGAAACCATCATTCTCAGCAAACTGTC
GCAGGGAAAAAAAACAAAACACTGCATGTTCTCACTCATAGGTGGGAATTGAACAATGAGAACTCTTGGACACAGGA
ATGTGAACATCACACACTGGGGCCTGTTGTGGGGTGGGGGGAGGGGGGAGGGATAGCATTAGGAGAAATACCTAATG
TAAATGGCGAGTTAATGGGTGCAGTACACCAACATGGCACATGTATACATATGTAACAAACCTGCACGTTGTGCACA
TGTACCCAAGAACTTAAAGTATAAAAAAAAAATTTATATATCAGGGCAATTAAAATCGTTGTTAAAAATTTACTGTA
AATGATTTTTAACAGCAGCCTAATTTATTGTATGCATGTACTATTATTTACTTAATATAAATGCCTAATAATAGTGG
AATGATAACCTATTCGCTTAGCCTTGTGATAAATACCTTTGTGGTTAAATTTGTATTCTAGATTATTTCTTTTTTTT
TTTTTTTTTTGAGACGGAGTCTCGCTCTGTCTCCCAGGCTGGAGTGCAGTGGCGCCATCTCAACTTGCTGCAAGCTC
CGCCTCCCAGGTTCACATCATTCTTGTGCCTCAGCCTCCCGAGTAGCTGAGACTACAGGCGCCTGCCACCATGCCCC
GCTATTTTTTTTGGTATTTTTAGTAGAGACAGGGTTTCACCGTGTTAGCCAGGATGGTCTCGATCTCCTGACCTTGT
GATCCACCCGCCTTGGCCTCCCAAAGTGCTAGGATCACAGGCGTGAGCCACTGCGCCCGGCCTAGATTATTTCTTTA
AAGTAAATTTTCAACAGTGGAATTAATTGGGCCAAAAGGTACAGGTTTTTTTTTTCCCACAAATGTAGCTAAACTGC
TTTCTATCTAACCATGTTCTACCAATTTGTATTTAATGTAGTTTCACTCTGACACATTCATATTTTTTATCTGGATA
GTTTGCAGAGGGGAGAACTTGATAAAATTAACATTCAGTTTGGTAAAGATGATGCACAGTATTTTTTAGATTTTTTT
TTTTTTTTTTTTTTTGAGACAAGAGTCTTGCTCTGTTACCCAGGCTGGAGTGCAGTGGCACAATCTTGGCTCACTGC
AGCCTCCACCTCCCAGGCATAAGCAATTCTCCCACCTCAACCTCCGGAGTAGCTGGGATTACAGGTGCACGCCACCA
TACCTGGCTAATTTTTGTATTTTTTGTAGAGACAGGGTTTCGCCATGTTGCCCAGGCTGGTCTGGAACTCCTGAGCT
TAAGTGATCCGCCTGCCTCGGACTCCCAAAGTGCTGGGATTACAGGCCTGAGCTGCCTCACCTGGCTGGATAAATTT
TTAATATTCTTAACATTTATAATTGCATACAAAAAACCCTAGCAATTTGTCTCAAATTTCATGCATAGGATCAAAAG
AAAATGTTTTATATGACTCATAAGTTATCCTGAGGAACCAGATAATGATACCCTGGCTTACTGTTAATTTTCTAATT
TGTTACACTATTAAACTAAGAGACAATAACATCTTCATAGCTGAAAATTCTATTTCAGATTTTTGATTCAGTTTGAA
TTTACATATTTGTATTTTTAGAACTAGTTTATTATTTTTTTGTTAGGTGATGAATCAAGATTGGTTCTCTAAGTTAT
TTTAATATAGTAAACTTTGAAATTGCTTTGGGCAAGAGAGATTATATTTATTGAGAAATTAAAATAAAAGGAGGTAT
GTGGTCTTTTTTAGTTAATTTTTTTGTTGTGGCAAAAATATAAAATTTATCATCTGAACCATTTTTAAGTATACAGC
TCAGTAGTATTATAAGCATATTTAGGTCATTATGAACAGATCTCCAGAACTTTAGCCTCCTGAAAAGATCCAGAGAC
CCCTGGAGCTTCTCCTACTATACTTTGAAGAACCTCTATCTTAGAGAATATATTACTTCATCTGGAAAAGACAGACT
CACAGAGAACTCTAGAAATATAGTCTTTTAAAACATATCATCTAAAAGATTAATAATTCTTTTTATCATCTTATCCG
GTCTGTGTTCATATTTCCCCATTGTCTAATAATGTTTTCGTAATTTTTTGAAATTAGGAGCCAAACCAGGTTCACAC
ACGTTACATTTGGTGGTTGATATACCTTTTATTTTTTTTTAAATTTATTTTTCTGAGATGAAGTCTTGCTCTGTCAC
CCAGGCTGAAGTACAGTGCATGATCTCAGCTCACGGCAACTTCTGCCTCCCCAGTTCAAGTGATTCTCCTGTCTCAG
CCTCCCGAGTAGCTAGGATTACAGGCGCGTGCCACCACGCCTGGCTAATTTTTTATATTTTTGGTAGAGACAGGGTT
TCACCATGTTGGCCAAGCTGGTCTTGAACTCCTGATCTCAGGTGATCTGCCTGCCTCGGCCTCCCAAAGTGTTGGGA
TTACAGGCGTGAACCACTGGGCCTGCCCTTGATATGCCTTTTAAATTTTAATCTATTGATTGTCTCTTTGTTTATAA
TTTATTTGTTGAAGAAACAGGGTCATTTGTTCTACATAATTTTCCTCATTCTGGTTTTGGCTGAGTGTATCTCTGGT
AAAATTTAACATGTTTTTCAGTTCTCTGTATTTCCAGTAAATTTATAGGTAGAGACAGGCTTGATTAGATTTACTTT
CAATTCTTTTTTGGCAAGATTCCTAGTTGGTGATGTGTATTTCTTTACATCAAGAAGATGTGTTTTGTGATGTTAAA
ATCAAACAGTTTAGCTGTTGTCAGATTGATTCATCCGTTGTAAAATTCCCAATTAACTTTTCTCTAATGATAGCATC
TATTGCTAATTGTTGCCTCATTGCAGTATATTAGTAGGTGTTGTAAAAGGTGATATTCCTTCTCCAGCCTTCTGAAT
TTATTGGCTGGGATTCTTCAGGAAAGAAGACCTCTTCCTCATATACCACTTTATTACCCTGAAAGTATTTATACAGG
AAAGGCCAAAATAAAATGGGACTTTTTTTTTCTTTTTACCTACATATTCCCAGAATAATTAATTTGTGCCTTAGGAG
CCTCAAAACATGACTAGAGTTTTGTTTGTTTGTTTAGAATTATAAACTCATGGATTTTTGCCTCCCATTCATTTTCG
ATGTTCAAATTATTCTTTCTTTGACCAGTGGCAGCTTATTTATGTTGGCTCCTTTGTCCTTGGCACAATCCCAGTGG
TCTGTAGTAGTTTCCTAAAATATACAAGATGAAGCTCATCTTGTATATTTTCTTCCCCAGATTTAGAATCAGAATTC
TAATTCCTTTCAGTGGGAAATGGTATTTAGAGATTATAATCTGGGTGATATTTTTGTATATTCCTACAATGTTGTCA
TTGTTTATAGGCCTTTTGATGGACAGAACTAGGAAAACTTTATAGAAAGAGAAAAATAATTCATGTGTTCCAGATGC
TATTTCTTTTTTTTTTTTTTTTTTTTGAGACGGAGTCTCGCTCTGTCGCCCAGGCTGGACTGCGGACTGCAGTGGCG
CAATCTCGGCTCACTGCAAGCTCCGCTTCCCGGGTTCACGCCATTCTCCTGCCTCAGCCTCCCCAGTAGCTGGGACT
ACAGGCGCCCGCCACCGCGCCCGGCTAATTTTTTGTATTTTTAGTAGAGACGGGGTTTCACCTTGTTAGCCAGGATG
GTCTCGATCTCCTGACCTCATGATCCACCCGCCTCGGCCTCCCAAAGTGCTGGGATTACAGGCGTGAGCCACCGCGC
CCGGCCTGCTATTTCTAACTCAGATAAAAGATGATAAGAGTTTTTACTTCATTTTAAAATTTACATTTGTATTTTTC
CTCAACATGTAAAATGTGGTTCTAACAATAAGATTATTGAATTGCAAAATCTGGGGCAGGAACTATTGTATTATATT
AGAAGATTGGAGGGTTACAAATACAAATGCCTAAAGGGGCCAGACAGCCAAATGAATATATCAGGCCTGTTATGAAA
ATCATCCGGGAATGGTGGGGATTAATAAGCCAGAGAGCATATATCCCATCTAAAGGAGGCATCTGTGACTTGATTCT
AGCTGATTTTTGCCAAGAGGGAATATGGGCATGGTACTGCTTGGTTTTTTCATTTTTAAAAAGAAGACGTAATCCCT
GCACTTTGGGAGGCCAAGGCAGGCAGATCATGAGGTCAGGAGATGGAGACCATCCTGGCTAACACGGTGAAACCCCG
TCTCTACTAAAAATGTAAAAAATTAGTTGGGCATGGTGGCACGCGCCTGTAGTCCCAGCTACTTGGGAGGCTGAGGC
GGGAGAATTGCTTGAACCTGGTAGGTGGAGGTTGCAGTGAGCTGAAATCGTGCCACTGCACTCCAGCTGGGGCAACA
GAGCAAGACTCCATCTCAAAAAAGAAAAAAAAGAAGATGTAAATCCAAATATTTTATGTGAAATTATTTAGCTTTTA
AATATTGACAATACTATTGACAAACTAAGTCAAAGTTTTAAAAAGGACTTTTGGACCAAACAATGATTTCTTCCATA
GGTTATTAGTTTGTAACCTCTTGTGAGAAAGATCTAAGGATTTTCCAGACCTCAGACAATCTGAAAAATATATTGGG
AAAGAGTATGAGAAAGGGAACTTAATCATCTTGGTTTTGCTGATCATGATTACATTTATTTATCTGACTTTTATTTT
TTAGCCTTAAAAATTAAAATAGCCCTATTGACCATTAATTTTGCTTTTTATTTCTTTTTGCACATTCAAAATAGGAA
CTTACAATGTCTCAGAAAAATGGTGGGAATGTTCTTCAGATGATGTATGAGAAACCTGAACGATGGTCTTTTACCTT
CCAAACATATGCCTGTCTCAGTCGAATAAGAGCTCAGCTTGCCTCTCTGAATGGCAAGCTCAAAGATGCAGAGAAAC
CTGTATTATTTTTTGAACGATCTGTGTATAGTGACAGGTATGTATAAATGGCTTGTACGTGGCCATTTGAAGTTTTT
ATCTTAGAACTGGACTTTTTTTTTTTTTTTAATGTTTCCTGGGAATTAGTAATAATGAATGGCTGAGCTGGGGATTT
AAGATGGAAAATGACATTTAAATCCCTCAGCAGTCTGGTACTAATGGCATTAGGGAAGTTATTGCATTTATAATCCT
CAGCTTCCATAGAAACTGTTGGGAATATGGTGGTGCCCATTTCATTTAAATTTGCAGAGAAGTTGTTTTTCCCTCTA
ACCCTTTTAAAATTCTCATCTGCTCTGGTTAAATCTGGATCTATATCCTAGACTCTTACCTGGATCAAGAAGATAAA
AGTCTCTAAGGGGCCTTAGAATTGCCCTACTTATGCTAGGATTCTTTTGACCTCCAAAGTTAGCTCAGAACAGAGGT
TCTCAAACATTTGATCTCAGGGCCCTTTTCAATTCTTAATAACTATCGAGGTGTCCAAAGAGCATGTCCTATTGATA
CTTAACATTACTAGAAATTAAGATGCACAAAAATGATTTAAAATTAGCAATAATAAATTCATTACATATTAATATAA
ATACATTTGACAAAAAGTTATATTTTCTAAAATAAAAAGTAGCTAGAAGAATATCATTGCTTTACATTTTTGCAGAT
ATCTTTAATGCCTCACTTATTAAAAGACAGCTGGATTTTTATTTATGCTTCTGTATTTAATCTGTTATGGTATGTTT
GTTTGTTTGAAGTATATTGGTATATGAAGTAATCTGGCCTCTCACAGAACCCCTTAGAAGATCTTGGGGACTTAATG
GGGTCCTCACTCCGTATTTTGAGAACCATTGGCCAAAAACATGATGGAAAGACTCTTGTCTTTTTCTGCTTTCCACG
GCACTATGTGCCACTGGATTTAGGAGAATGTTTCATGTTCCTGTTCTCTTTTTTATTTCTTTGTTGTTTTTTTTTGA
AATGATACATGTGTTGATGAAGACTCTCTTTTAGGTATATTTTTGCATCTAATTTGTATGAATCTGAATGCATGAAT
GAGACAGAGTGGACAATTTATCAAGACTGGCATGACTGGATGAATAACCAATTTGGCCAAAGCCTTGAATTGGATGG
AATCATTTATCTTCAAGCCACTCCAGAGGTAAAACCCAATAAAAATGTGTTTCACTGAAAATTTAAAGAAATATTTA
GAACTCTTTTCAGTGGTATATAATGAGGGCAATTTAGTTTAACTCCAGCCTCCCAACCTCCCACTCCCCACCTCTAA
ATTAGAAACTTGTAACTGTGTAGATAGAAGCATTCTTTTTTCTCAGCAAACTGGAATCCCAGACTGGGCAAGTTGCT
GTTTTAGGCTCTTTGTGCTTCCTCAATTCTTTTTTATTTTACTCATTATTTCTTTCTAACATTTGCAGACTCTTCTC
ATTCATCACAGTTAAAGTAATGAATGTGGTCAAGCAACTCCTAGAATATAGGCTTCATGAGGTGGGACCTTGGGCAT
TTTGATTACTATTGAATTTCCATTGCCTAGAACACCCAGTGCTGGGCACTGTTGGAGCATATAGTAGGAGCTCAGTA
AATATTTACTGAATGAATGGCAATGTTGATGGAGGTCCATATCAAGAAATATTTTATTATGGTTGTTTGTGCCTTGG
AATGAGTGGATAATGGCAGCTATCGTTGGGAAAAGACTTAGAGGACCCTTGCCATTTTCCATTAAGGGAGCTCACTT
GAAAAGGTGCTTTTTGTGATAGGTTGTAACATAGCTATTCTAGTTACATATGATGTTATATAATGAATACAGGATGA
CCAAATAGATGCCTTTCATAAAACTAACCTAAATATTAAGATCTTACATTTAATCAGAAGCTTTCTGGTTTTGGTGC
GTGCATTTCTATTGTTTCTGTTTTTTAATTCTCATTTTTTCCTTAAGTCACAGGGTAGGCACTGATGAATGTAAATG
TTATGACTTCTGGCCTAAAATTAGAATACTGTGTTGCTGTCGAAGCTGGGTTTTCAAAGAAATTAGGTTTCCTATTC
CAACGGATGATTTTGTTCTTCATTAAGTAAAGATTTTGTTCTTCATTAAGCAAGGATTTAGCAATTTTTTAGCAATT
TCAAAAGTGAATTTTGAAATTCACTTTTATTTTTGAATTTTTTTGTTTTTAATTACAAAAATAGTAAATAAAAATGT
GAAAAAGAACATAAGGAATATGAATAATTTCATTTTGCTTGAAACATTTATAGTCACTATTAAGCTAGAATTTAGCA
AATATAATAATTTTGAAATTCACTTTTATTTTTGAAAAGGTTTCTTAAAGTTATGAAAATAGTACAAACACCAAAAA
CATAAATAATACTAATAATTTCATTTTGATACTGATAATTTCATCAGTATTGTTTCTGATATCTTTGGATGAAAGGA
TTGTTTAATTATTGAAAAGAGAATTTAGAGTAGAGTTTGAAACACAGTTTGTATGCTGCCATGGTCTTATGCATGTC
ATTTAGGTTGGAGGCTTTTATTTCTTGGAATGCATGAAGAGTTTCTCATGGTATATGAGCCCCCACAAATTATACGA
AATTTTTTATGTATGTGTGCTTTTGGGGGACCGTTATCTAGAGCTGCACTGTCCAATGTGGCTATTTAAATTTAGGG
TTATTAAAATTAAATAAAATTTAGATATTTAGTTCCTTGGTCACCACACTAGCCATATTTCAAGTGCTTAAAAACCA
CAGGTGGCTGGTGGCTACCTTATTTGATAATGCAGATATAGGACAGGCATAAAACATTCCAAAGTTGTTTTGGTTGT
GCTGATATAGAGTTTTCATCACATTAAGAACCACTGTCTTTAGTGGTTTTATGTGAACTGAACATAGATATTTTGTA
ATCAAGGTTGGTTAACAGCAAAGTAAGATTATGTTTATAGATTGGCATACCTTTTAGCTGTTGTCTCTGAACATTTT
AAGTAGATGTTTAATTTTTTTCTCCTTTTTATCAAACTTAGTATATTTCATGTGTGGATGGATACCAAAAATTTATA
GGCTTATTTTTGAAAGTACTGCTTGGCTTAGAGCCACCAAGTGGCTGAAAAGCTCATGGAGGGAAAAATGAATTATT
TCAGCTTTTAAGAAGTAATAAGTAGAGATTTTGGCTAGCGTGAGTAGAGAGACACAGAAAAGAAAATTTTGATGGCA
GCAGACAAGAATAATTGTTCCCTGCCTTTTTCTTCCATCTCTTATTACTTGCTTTTAGACATGCTTACATAGAATAT
ATTTACGGGGAAGAAATGAAGAGCAAGGCATTCCTCTTGAATATTTAGAGAAGCTTCATTATAAACATGAAAGCTGG
CTCCTGCATAGGACACTGAAGTAAGACTTATTTTTATTTACTATTCATTTTAAATACCTTTGTTACCTTTGTTAAAT
TTTAATCTAAAATTTGGAAGATATGACTAGCAATATGTAAAATTTCCAGTCGTTTTGCTTATTGGTATCTTCCAGAA
CTAGGTATAGATTTTCATTCTCTTGATTGCAATGAGATCCTATTTACTTAAAGTTTTTATAACTAGTATCCCTTGGT
GGTATTGATAGCGTAATATGTGAAATTTCACTTTAAGTGATAAAGTAGGAATATATCTGAACATTATTTAGGTACTC
TTTCAGAAAGAATTTTGACTTGGGAGATTGTATAGCGTGGTTGTTGAGAGGATAGACTTTGTTATTAGTCAGATCTT
GGCTCAAGTCCAGTCTTACTGGTGTGTGACCTTGGATAGCCTCTGAGCTTCAGAATGACAATTCTGAGCTTTAGTTT
TCTCATCTTTAAAAATGCAGAAATTCCTACTTTATTGACTGCTATTTGTTTTTGAAGAGAATCATATGTTACAGTAC
CCAGCTGTATATATTTTGTACCTAAGTTTTTGGGCTCCATATGGTAAATCTAGATGAGAAAGAAGGGGCTGCCAGAT
GAAGTACTGCAAAGTAACTTTAGTGTAAATGATTGACAACATTTTGATTTTCCAAGGACATACGAATGAATTTTTAA
TGTTTCTCTCTTTTTTTTGTTGAATTTCTGATTATTTTATTAATCTCTCTTTTTAAACAGAACCAACTTCGATTATC
TTCAAGAGGTGCCTATCTTAACACTGGATGTTAATGAAGACTTTAAAGACAAATATGAAAGTCTGGTTGAAAAGGTA
GATTTAGACAGACTACAAACAAATATTTGTTTTTTCTAAAAAAGTGTACTGAGTGGTGAGAAAACAATTTTCTAACA
GAAAAGCTTCCATAAAGTTACATTAAGAAACAGTTAAGAGCTTTAGAAGATTTTGGACTGTTTAAGATTCCAGTCCT
GACTAAATTCTAGCTTTGTGGCCTGAGCTAAAATAAAGATCCTTCGGAATCTCAGAATCTGACAGATCTTAGAAAAA
AATTAAATAAATAAAAATAAAATAAGGATTTTTTTTTATCCTTTAGTGCCTCTATCTGTAGGATAGGTAAAATTATA
ATAACAAAAATTCTTTCTCACCCCCTTTTTCTCTGCTTTGTCCCCATTTCTTCCTCTCTTCCCTACACCACCAATCT
TTAGATACTTTAATAATAAACTGCTTGTGAAATTAAATTTATCTTTTTCAACTAAATTCAATATGGAGGATATTTAA
AGTTACAGGATATGGATCTCATCTATTTTTATGTTGGCCAAAGTCTTGAGATTAAGGGATTTTAGTTTTGAGTTTCA
TTTGGGATCATGTCAAGGATTAATTGACATTACTTAATCATTGAGTCATTGTCTTAATCACAGTTCATTTATTGCCC
AAATCACTGGGAGTTGACTATATTCTGTTAAGTGAGTGGTGCCCATATTGATAAAGGGAATGGGTGAGAATTTATTG
ATGAATATTTGAGAGAGAATAGTAAGGGAATTGGTATAACAAATATCCCTATACCCATGTTGATAACTGAGTGATTA
AGCTATAAAGAAGAAAGCACAATTTGGTTTAGTTTTGAGAAAAATTTGATTTTTCCTTTCTTTTGTGGCATAATGTC
TGCTGTTTAATAGAGACTTGACATTCATAAGGGATATATCAAGGATTTTGAAAATCTTGACATTTTATGTAACTACT
GTAGCGTATACATGTAGTTGTTTTTCCCATGGTATGAACTAAAGCTTATCTGGAAAATTCCAATGACCTCTTCATAC
TACTAATGATGTTAAAAATATAGGATAAATTCACTTACAATACTATTAAATGAAAAGTCAGCATTAAGTATTAACAG
CCAACCATCAATTTAACTGACTTTGTAAATCACTTTTTAATTTTATCTAACACTTCTTTGTTTTGACTAAGAATCAT
TTCACATTATTATATTTACTTTTATTAATAATGCTAGCAGTTGACTTTCTTATTAAGGTATTTTTACAAGGCAATAA
TTTTTGTTTCTTGAGAATTATTGAGTAGCCTAGAATATGTAGAGAACAAGCATATTCTTACATTATGGTCAGTAGGC
TAGGTTCATTGACTCCTTACATGATTTTCTGCACACACTGACATTGCAAAGTATGTGATTCACATATGTGCTTCAGA
AACATGACAAATCTACTACATGTTTGATTTTCAAAGTTGCTTGAACTATTGTGCTTATTAATGTTATCAGTGTTAAA
GCTGCATATGTCAGTGGTCTATGATATTTAAATGAAGAGATTATAAAATTGGTGTTACAGCTATTTTAGAACTATAT
CTATCTTAGATTGATAGATGCAGTGGCTCACACCTGTAATCCCAGCACTTTGGGAGGCTGAGGTGGGAAGACTGCTT
GAGCCCAGGAGTTCCTGACCAGCTTGGGCAACATAGTGAGACCTCATCTCTATAAAAAACAAACAAAAACTAGCCAG
ACATGATGGTGTGCACCTAGCCAGGAGTTCAAGGCTGCAGTGAGCTTTGATTGCACCACTGTACTCAGCCTGGGTGA
CAGAGCAATAAATAACTCCTAAAATGACCTTTTAGGTCATTCATCTTTTACTACTGAAGTATTTAACAATATTTCTT
TTTTTTCTCGAGACAGTCTTGCTCCGTCGCCCAGGCTGGAGTGCAATGGCACAATCTTGGCTCACTGCCATCTCCAC
CTCCCAGGTTCAACCAGTTCTGCCTCAGCCTCCTAAGTAGGTGGGATTACAAGTGCACGCCTCCATGCCTAGCTAAT
TTTTTTTTTTTTTTGAGACGGAGTTTTGCTCTTGTTGCCCAGGCTGGAGTGCGATGGCGCGATCTCAGCTCACCACA
ACCTCTGCCTCCCAGGTTCAAGCGATTCTTCTGCCTTGGCCTCCCTAGTAGCTGGGATTACAGGCATGTGCCACTAT
GCCCGGCTAATTTTGTATTTTTAGTAGAGAAGGGGTTTCTCCATGTTGGTCAGGCAGGTCTCGAACTCCTGACCGCA
GGTGATCTGCCCGCCTCGGCCTCCCAAAGTGCTGGGATTATAGGCATGAGCCACTGCGCCTGGCCTATTTTTGTGTT
TTTAGTAGAGATGGGGGGTTTCACCATGTTGGCCAGGCTGGTCTGAAACTCCTGACCTTAAGTGATCCACCCATCTC
GGCCTCCCAAAATGCTAGAATTACAGGCATGAGCCGCTGCGCCCGGCCTTAACAATATTTCTTTGGAAAGTAAATGC
AATGGCATTGTGGTAGTTACTTATACAGCTGGGGTCTTCAACTATTATATACTTTTAAATGAAGATGAATATTAGAT
ACCTCAAATTAGTCAAGGAATTATACTGATTTTTTTTTCTTCCTTTCCTCAGGTCAAAGAGTTTTTGAGTACTTTGT
GATCTTGCTGAAGACTACAGGCAGCCAAATGGTTCCAGATACTTCAGCTTTGTGTATCTTCGTAACTTCATATTAAT
ATAAGTTTCTTTAGAAAACCCAAGTTTTTAATCGTTTTTGTTTTAAGGAAAAAAGATTTTTAAAATGAATCTTATGC
AAAACTTTTTGACCAGTTTCTTTTCTTTTGTTTTTTTTTTAAAAAAGACATTTAAAGACAAAGACATTATTTCTCAT
AGCAGGAAATGTAGAGGTAGATGGTTCCAGTATCAGCATAGTGACTAAACTACATTATAAAAGATCCAGCTTCCTTC
TGTCATTCCCCTCTTTTGTCTTCCTCAGCAGGTTGGCTTTTTTCCCTGGTGCCTCTCACTTCGTTGGTGACCAGTTT
CTTAAACTGAAAGCTTTAATGTTACATAGTAAATGGTAGTGTGTCCTGTGTAAATTAGTGTACCTATTAAAAGTTGC
AAAGTGGAATTAAAGGAATCCCTAGAATAAGGATTCTGAAGTTTTATTTTAAATTATTATCTTCTTAACAGTTTAGT
CCCACCTCTTACTTCCTGCCTCAGTCTGCTTTCTCTACTGTCTGGATTAATTAGGCAGCCTGCTATAAAGTTAAAGT
CACACATTTCTATTTTGCAAACACTGTGATTACTCTTTGCTTTGTAGTTTGCTTTGCTTTGTAGGGTTCTGCTTTTA
AGTTTTTCTCTTTTTCAGACAAATTACTGATAAAAATGATATTGCTCTATATGTAATATATCCTGAAAGCATTATTT
TTTGTTGAATAGGAAATAAAATTAATGAAGACAGAGGCTAGAAAGCATCCATTAATTAATGAGACACACTTAACTAC
TTATCTCTAAACCATCTATGTGAATATTTGTAAAAATAATGAATGGACTCATCTTAGTTCTGTATATAAATATATTT
TCTTTCTAGTTTGTTTAGTTAAGGTGTGCAGTGTTTTTCCTGTGTATTAAACCTTTCCATTTTACGTTTTAGAAAAT
TTTATGTATTTTAAAATAAGGGGAAGAGTCATTTTCACTTTTAAACTACTATTTTTCTTTCCAAGTCATTTTTGTTT
TTGGTTTCTTATTCAAAGATGATAATTTAGTGGATTAACCAGTCCAGACGCACTGATCTTTGCAAAGGAGACTTAAT
TTCAAATCTGTAATTACCATACATAAACTGTCTCATTATACGTATGCATTTTTTTAGTTTGTTTTTGTTTGGTATAA
ATTAATTTGTTAATTAAATATTTCTTAAGTATAAACCTTATGAACTACAGTGGAGCTACACTCATTGAAATGTAATT
TCAGTTCTAAAAAGATGTAATAATCATTTTAGAATTAAAATTTATTCTACTTTTAAATAAATTATGAATATTAAAGG
TGAAAATTGTATAAATTACTTTGATTCCATTTTAAGTGGAGACATATTTCAGTGATTTTTAGTAACCTTTAAAAATG
TATAATGACTTTTAAAATTTGTAGAATTGAAAAGACGCTAATAAAAATTTATTATTTATTTGTCATGACTCAAAA
SEQ ID NO: 28: Homo sapiens deoxycytidine kinase (DCK), mRNA-NCBI
Reference Sequence: NM_000788.3
AGTGTCCTCAGCTGCCTCCGCGCGCCAAAGTCAAACCCCGACACCCGCCGGCGGGCCGGTGAGCTCACTAGCTGACC
CGGCAGGTCAGGATCTGGCTTAGCGGCGCCGCGAGCTCCAGTGCGCGCACCCGTGGCCGCCTCCCAGCCCTCTTTGC
CGGACGAGCTCTGGGCCGCCACAAGACTAAGGAATGGCCACCCCGCCCAAGAGAAGCTGCCCGTCTTTCTCAGCCAG
CTCTGAGGGGACCCGCATCAAGAAAATCTCCATCGAAGGGAACATCGCTGCAGGGAAGTCAACATTTGTGAATATCC
TTAAACAATTGTGTGAAGATTGGGAAGTGGTTCCTGAACCTGTTGCCAGATGGTGCAATGTTCAAAGTACTCAAGAT
GAATTTGAGGAACTTACAATGTCTCAGAAAAATGGTGGGAATGTTCTTCAGATGATGTATGAGAAACCTGAACGATG
GTCTTTTACCTTCCAAACATATGCCTGTCTCAGTCGAATAAGAGCTCAGCTTGCCTCTCTGAATGGCAAGCTCAAAG
ATGCAGAGAAACCTGTATTATTTTTTGAACGATCTGTGTATAGTGACAGGTATATTTTTGCATCTAATTTGTATGAA
TCTGAATGCATGAATGAGACAGAGTGGACAATTTATCAAGACTGGCATGACTGGATGAATAACCAATTTGGCCAAAG
CCTTGAATTGGATGGAATCATTTATCTTCAAGCCACTCCAGAGACATGCTTACATAGAATATATTTACGGGGAAGAA
ATGAAGAGCAAGGCATTCCTCTTGAATATTTAGAGAAGCTTCATTATAAACATGAAAGCTGGCTCCTGCATAGGACA
CTGAAAACCAACTTCGATTATCTTCAAGAGGTGCCTATCTTAACACTGGATGTTAATGAAGACTTTAAAGACAAATA
TGAAAGTCTGGTTGAAAAGGTCAAAGAGTTTTTGAGTACTTTGTGATCTTGCTGAAGACTACAGGCAGCCAAATGGT
TCCAGATACTTCAGCTTTGTGTATCTTCGTAACTTCATATTAATATAAGTTTCTTTAGAAAACCCAAGTTTTTAATC
GTTTTTGTTTTAAGGAAAAAAGATTTTTAAAATGAATCTTATGCAAAACTTTTTGACCAGTTTCTTTTCTTTTGTTT
TTTTTTTAAAAAAGACATTTAAAGACAAAGACATTATTTCTCATAGCAGGAAATGTAGAGGTAGATGGTTCCAGTAT
CAGCATAGTGACTAAACTACATTATAAAAGATCCAGCTTCCTTCTGTCATTCCCCTCTTTTGTCTTCCTCAGCAGGT
TGGCTTTTTTCCCTGGTGCCTCTCACTTCGTTGGTGACCAGTTTCTTAAACTGAAAGCTTTAATGTTACATAGTAAA
TGGTAGTGTGTCCTGTGTAAATTAGTGTACCTATTAAAAGTTGCAAAGTGGAATTAAAGGAATCCCTAGAATAAGGA
TTCTGAAGTTTTATTTTAAATTATTATCTTCTTAACAGTTTAGTCCCACCTCTTACTTCCTGCCTCAGTCTGCTTTC
TCTACTGTCTGGATTAATTAGGCAGCCTGCTATAAAGTTAAAGTCACACATTTCTATTTTGCAAACACTGTGATTAC
TCTTTGCTTTGTAGTTTGCTTTGCTTTGTAGGGTTCTGCTTTTAAGTTTTTCTCTTTTTCAGACAAATTACTGATAA
AAATGATATTGCTCTATATGTAATATATCCTGAAAGCATTATTTTTTGTTGAATAGGAAATAAAATTAATGAAGACA
GAGGCTAGAAAGCATCCATTAATTAATGAGACACACTTAACTACTTATCTCTAAACCATCTATGTGAATATTTGTAA
AAATAATGAATGGACTCATCTTAGTTCTGTATATAAATATATTTTCTTTCTAGTTTGTTTAGTTAAGGTGTGCAGTG
TTTTTCCTGTGTATTAAACCTTTCCATTTTACGTTTTAGAAAATTTTATGTATTTTAAAATAAGGGGAAGAGTCATT
TTCACTTTTAAACTACTATTTTTCTTTCCAAGTCATTTTTGTTTTTGGTTTCTTATTCAAAGATGATAATTTAGTGG
ATTAACCAGTCCAGACGCACTGATCTTTGCAAAGGAGACTTAATTTCAAATCTGTAATTACCATACATAAACTGTCT
CATTATACGTATGCATTTTTTTAGTTTGTTTTTGTTTGGTATAAATTAATTTGTTAATTAAATATTTCTTAAGTATA
AACCTTATGAACTACAGTGGAGCTACACTCATTGAAATGTAATTTCAGTTCTAAAAAGATGTAATAATCATTTTAGA
ATTAAAATTTATTCTACTTTTAAATAAATTATGAATATTAAAGGTGAAAATTGTATAAATTACTTTGATTCCATTTT
AAGTGGAGACATATTTCAGTGATTTTTAGTAACCTTTAAAAATGTATAATGACTTTTAAAATTTGTAGAATTGAAAA
GACGCTAATAAAAATTTATTATTTATTTGTCATGACTCAAAA
```

What is claimed is:

1. A composition comprising:
an engineered cell comprising at least one exogenous nucleic acid encoding:
a drug resistance protein selected from the group consisting of: an aminoglycoside phosphotransferase, a protein kinase cAMP-activated catalytic subunit alpha (PRKACA), an aldehyde dehydrogenase (ALDH), and a glutathione S-transferase,
wherein the engineered cell is resistant to drug-mediated cell death relative to a comparable cell that does not comprise an exogenous nucleic acid encoding for a drug resistance protein.

2. The composition of claim 1, wherein the aminoglycoside phosphotransferase comprises an aminoglycoside-3-phosphotransferase-4a (APH(3)-Ila) protein.

3. The composition of claim 1, wherein the engineered cell is an ex vivo-engineered cell, an in vivo-engineered cell, or an in vitro-engineered cell.

4. The composition of claim 1, wherein the engineered cell comprises a human leukocyte, a human stem cell, or an in vitro-differentiated human cell.

5. The composition of claim 1, wherein the engineered cell comprises a human leukocyte, wherein the human leukocyte comprises a human T cell, a human natural killer (NK) cell, a human natural killer T cell (NKT), a human macrophage, a human B cell, a human dendritic cell, or a human tumor infiltrating lymphocyte (TIL).

6. The composition of claim 5, wherein the human T cell is a CD3 positive (CD3⁺) T cell.

7. The composition of claim 5, wherein the human TIL is a CD3 positive (CD3⁺) TIL.

8. The composition of claim 1, wherein the engineered cell is a human stem cell, wherein the human stem cell comprises an adult stem cell, an induced pluripotent stem cell (iPSC), or an embryonic stem cell.

9. The composition of claim 1, wherein the engineered cell is an in vitro-differentiated human cell, wherein the in vitro-differentiated human cell comprises an iPSC-derived natural killer T cell (iNKT), an iPSC-derived natural killer cell, an iPSC-derived T cell, an iPSC-derived macrophage, or an iPSC-derived B cell.

10. The composition of claim 1, wherein the at least one exogenous nucleic acid comprises a deoxyribonucleic acid (DNA), a ribonucleic acid (RNA), or a combination thereof.

11. The composition of claim 10, wherein the RNA comprises a poly-A tail, a nucleoside modification, or a combination thereof.

12. The composition of claim 1, wherein the at least one exogenous nucleic acid comprises a nucleotide sequence that is at least 80% identical to any one of SEQ ID NO: 1-SEQ ID NO: 2, SEQ ID NO: 8-SEQ ID NO: 9, SEQ ID NO: 15-SEQ ID NO: 16, SEQ ID NO: 19-SEQ ID NO: 20, or SEQ ID NO: 23-26, wherein the nucleotide sequence encodes a drug resistance protein that confers resistance to drug-mediated cell death.

13. The composition of claim 1, wherein the drug resistance protein comprises a protein kinase cAMP-activated catalytic subunit alpha (PRKACA) protein.

14. The composition of claim 1, wherein the the glutathione S-transferase comprises a glutathione S transferase A4 (GSTA4) protein.

15. The composition of claim 1, wherein the ALDH comprises an ALDH 1 Family Member A2 (ALDH1A2) protein.

16. The composition of claim 1, wherein the engineered cell further comprises an exogenous nucleic acid encoding: a chimeric antigen receptor, an immune checkpoint inhibitor, a gene editing agent, or any combination thereof.

17. The composition of claim 1, wherein the engineered cell further comprises a genomic disruption in a target sequence of a dCK gene, a beta-2 microglobulin gene, a class II major histocompatibility complex transactivator (CIITA) gene, a programmed cell death 1 (PD1) gene, and T cell receptor constant region (TRAC).

18. A composition comprising:
a carrier; and
at least one RNA encoding a drug resistance protein selected from the group consisting of an aminoglycoside phosphotransferase, a protein kinase cAMP-activated catalytic subunit alpha (PRKACA), an aldehyde dehydrogenase (ALDH), and a glutathione S-transferase,
wherein the at least one RNA is encapsulated within the carrier or in complex with the carrier.

19. A method of treating a cancer in a subject in need thereof, the method comprising:
administering to the subject in need thereof a therapeutically effective amount of the composition of claim 1, thereby treating the cancer.

20. A method of treating a cancer in a subject in need thereof, the method comprising:
administering to the subject in need thereof a therapeutically effective amount of the composition of claim 18, thereby treating the cancer.


SEQUENCE LISTING (28 sequences)

SEQ ID NO: 1 - DNA, 792 bp, synthetic construct
  source 1..792: mol_type = other DNA
atgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttc

SEQ ID NO: 2 - RNA, 792 nt, synthetic construct
  source 1..792: mol_type = other RNA
atgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttc

SEQ ID NO: 3 - protein, 264 aa, synthetic construct
MIEQDGLHAGSPAAWVERLFGYDWAQQTIGCSDAAVFRLSAQGRPVLFVKTDLSGALNELQDEAARLSWLATTGVPCAAVLDVVTEAGRDWLLLGEVPGQDLLSSHLAPAEKVSIMADAMRRLHTLDPATCPFDHQAKHRIERARTRMEAGLVDQDDLDEEHQGLAPAELFARLKASMPDGEDLVVTHGDACLPNIMVENGRFSGFIDCGRLGVADRYQDIALATRDIAEELGGEWADRFLVLYGIAAPDSQRIAFYRLLDEFF

SEQ ID NO: 4 - protein, 206 aa, Escherichia coli
RLSAQGRPVLFVKTDLSGALNELQDEAARLSWLATTGVPCAAVLDVVTEAGRDWLLLGEVPGQDLLSSHLAPAEKVSIMADAMRRLHTLDPATCPFDHQAKHRIERARTRMEAGLVDQDDLDEEHQGLAPAELFARLKARMPDGEDLVVTHGDACLPNIMVENGRFSGFIDCGRLGVADRYQDIALATRDIAEELGGEWADRFLVL

SEQ ID NO: 5 - protein, 264 aa, Klebsiella pneumoniae [sequence omitted]

SEQ ID NO: 6 - protein, 264 aa, Salmonella enterica [sequence omitted]

SEQ ID NO: 7 - protein, 264 aa, Vibrio cholerae [sequence omitted]

SEQ ID NO: 8 - DNA, 1053 bp, synthetic construct
  source 1..1053: mol_type = other DNA
atgggcaacgccgccgccgccaagaagggcagcgagcaggagagcgtgaaagaattcttagccaaagccaaagaagattttcttaaaaaatgggaaagtcccgctcagaacacagcccacttggatcagtttgaacgaatcaagaccctcggcacgggctccttcgggcgggtgatgctggtgaaacacaaggagaccgggaaccactatgccatgaagatcctcgacaaacagaaggtggtgaaactgaaacagatcgaacacaccctgaatgaaaagcgcatcctgcaagctgtcaactttccgttcctcgtcaaactcgagttctccttcaaggacaactcaaacttatacatggtcatggagtacgtgcccggcggggagatgttctcacacctacggcggatcggaaggttcagtgagccccatgcccgtttctacgcggcccagatcgtcctgacctttgagtatctgcactcgctggatctcatctacagggacctgaagccggagaatctgctcattgaccagcagggctacattcaggtgacagacttcggtttcgccaagcgcgtgaagggccgcacttggaccttgtgcggcacccctgagtacctggcccctgagattatcctgagcaaaggctacaacaaggccgtggactggtgggccctgggggttcttatctatgaaatggccgctggctacccgcccttcttcgcagaccagcccatccagatctatgagaagatcgtctctgggaaggtgcgcttcccttcccacttcagctctgacttgaaggacctgctgcggaacctcctgcaggtagatctcaccaagcgctttgggaacctcaagaatggggtcaacgatatcaagaaccacaagtggtttgccacaactgactggattgccatctaccagaggaaggtggaagctcccttcataccaaagtttaaaggccctggggatacgagtaactttgacgactatgaggaagaagaaatccgggtctccatcaatgagaagtgtggcaaggagttttctgagttt

SEQ ID NO: 9 - RNA, 1053 nt, synthetic construct
  source 1..1053: mol_type = other RNA
atgggcaacgccgccgccgccaagaagggcagcgagcaggagagcgtgaaagaattcttagccaaagccaaagaagattttcttaaaaaatgggaaagtcccgctcagaacacagcccacttggatcagtttgaacgaatcaagaccctcggcacgggctccttcgggcgggtgatgctggtgaaacacaaggagaccgggaaccactatgccatgaagatcctcgacaaacagaaggtggtgaaactgaaacagatcgaacacaccctgaatgaaaagcgcatcctgcaagctgtcaactttccgttcctcgtcaaactcgagttctccttcaaggacaactcaaacttatacatggtcatggagtacgtgcccggcggggagatgttctcacacctacggcggatcggaaggttcagtgagccccatgcccgtttctacgcggcccagatcgtcctgacctttgagtatctgcactcgctggatctcatctacagggacctgaagccggagaatctgctcattgaccagcagggctacattcaggtgacagacttcggtttcgccaagcgcgtgaagggccgcacttggaccttgtgcggcacccctgagtacctggcccctgagattatcctgagcaaaggctacaacaaggccgtggactggtgggccctgggggttcttatctatgaaatggccgctggctacccgcccttcttcgcagaccagcccatccagatctatgagaagatcgtctctgggaaggtgcgcttcccttcccacttcagctctgacttgaaggacctgctgcggaacctcctgcaggtagatctcaccaagcgctttgggaacctcaagaatggggtcaacgatatcaagaaccacaagtggtttgccacaactgactggattgccatctaccagaggaaggtggaagctcccttcataccaaagtttaaaggccctggggatacgagtaactttgacgactatgaggaagaagaaatccgggtctccatcaatgagaagtgtggcaaggagttttctgagttt

SEQ ID NO: 10 - protein, 351 aa, synthetic construct
MGNAAAAKKGSEQESVKEFLAKAKEDFLKKWESPAQNTAHLDQFERIKTLGTGSFGRVMLVKHKETGNHYAMKILDKQKVVKLKQIEHTLNEKRILQAVNFPFLVKLEFSFKDNSNLYMVMEYVPGGEMFSHLRRIGRFSEPHARFYAAQIVLTFEYLHSLDLIYRDLKPENLLIDQQGYIQVTDFGFAKRVKGRTWTLCGTPEYLAPEIILSKGYNKAVDWWALGVLIYEMAAGYPPFFADQPIQIYEKIVSGKVRFPSHFSSDLKDLLRNLLQVDLTKRFGNLKNGVNDIKNHKWFATTDWIAIYQRKVEAPFIPKFKGPGDTSNFDDYEEEEIRVSINEKCGKEFSEF

SEQ ID NO: 11 - protein, 427 aa, Homo sapiens
MFTLGYHGAEEWASGETSLEPALEQCPCDCHGEEQGQLGAKVTAEPPKGTCYPSPRGLHPRPGLPREGARGTAKSQGYKYLGWRRPWPWNVVKEFLAKAKEDFLKKWESPAQNTAHLDQFERIKTLGTGSFGRVMLVKHKETGNHYAMKILDKQKVVKLKQIEHTLNEKRILQAVNFPFLVKLEFSFKDNSNLYMVMEYVPGGEMFSHLRRIGRFSEPHARFYAAQIVLTFEYLHSLDLIYRDLKPENLLIDQQGYIQVTDFGFAKRVKGRTWTLCGTPEYLAPEIILSKGYNKAVDWWALGVLIYEMAAGYPPFFADQPIQIYEKIVSGKVRFPSHFSSDLKDLLRNLLQVDLTKRFGNLKNGVNDIKNHKWFATTDWIAIYQRKVEAPFIPKFKGPGDTSNFDDYEEEEIRVSINEKCGKEFSEF

SEQ ID NO: 12 - protein, 351 aa, Homo sapiens; identical to SEQ ID NO: 10

SEQ ID NO: 13 - protein, 343 aa, Homo sapiens
MASNSSDVKEFLAKAKEDFLKKWESPAQNTAHLDQFERIKTLGTGSFGRVMLVKHKETGNHYAMKILDKQKVVKLKQIEHTLNEKRILQAVNFPFLVKLEFSFKDNSNLYMVMEYVPGGEMFSHLRRIGRFSEPHARFYAAQIVLTFEYLHSLDLIYRDLKPENLLIDQQGYIQVTDFGFAKRVKGRTWTLCGTPEYLAPEIILSKGYNKAVDWWALGVLIYEMAAGYPPFFADQPIQIYEKIVSGKVRFPSHFSSDLKDLLRNLLQVDLTKRFGNLKNGVNDIKNHKWFATTDWIAIYQRKVEAPFIPKFKGPGDTSNFDDYEEEEIRVSINEKCGKEFSEF

SEQ ID NO: 14 - protein, 351 aa, Mus musculus [sequence omitted]

SEQ ID NO: 15 - DNA, 1359 bp, synthetic construct
  source 1..1359: mol_type = other DNA
atgagcaagatcagcgaggccgtgaagcgcgcccgcgccgccttcagctcgggcaggacccgtccgctgcagttccggatccagcagctggaggcgctgcagcgcctgatccaggagcaggagcaggagctggtgggcgcgctggccgcagacctgcacaagaatgaatggaacgcctactatgaggaggtggtgtacgtcctagaggagatcgagtacatgatccagaagctccctgagtgggccgcggatgagcccgtggagaagacgccccagactcagcaggacgagctctacatccactcggagccactgggcgtggtcctcgtcattggcacctggaactaccccttcaacctcaccatccagcccatggtgggcgccatcgctgcagggaacgcagtggtcctcaagccctcggagctgagtgagaacatggcgagcctgctggctaccatcatcccccagtacctggacaaggatctgtacccagtaatcaatgggggtgtccctgagaccacggagctgctcaaggagaggttcgaccatatcctgtacacgggcagcacgggggtggggaagatcatcatgacggctgctgccaagcacctgacccctgtcacgctggagctgggagggaagagtccctgctacgtggacaagaactgtgacctggacgtggcctgccgacgcatcgcctgggggaaattcatgaacagtggccagacctgcgtggccccagactacatcctctgtgacccctcgatccagaaccaaattgtggagaagctcaagaagtcactgaaagagttctacggggaagatgctaagaaatcccgggactatggaagaatcattagtgcccggcacttccagagggtgatgggcctgattgagggccagaaggtggcttatgggggcaccggggatgccgccactcgctacatagcccccaccatcctcacggacgtggacccccagtccccggtgatgcaagaggagatcttcgggcctgtgctgcccatcgtgtgcgtgcgcagcctggaggaggccatccagttcatcaaccagcgtgagaagcccctggccctctacatgttctccagcaacgacaaggtgattaagaagatgattgcagagacatccagtggtggggtggcggccaacgatgtcatcgtccacatcaccttgcactctctgcccttcgggggcgtggggaacagcggcatgggatcctaccatggcaagaagagcttcgagactttctctcaccgccgctcttgcctggtgaggcctctgatgaatgatgaaggcctgaaggtcagataccccccgagcccggccaagatgacccagcac

SEQ ID NO: 16 - RNA, 1359 nt, synthetic construct
  source 1..1359: mol_type = other RNA
atgagcaagatcagcgaggccgtgaagcgcgcccgcgccgccttcagctcgggcaggacccgtccgctgcagttccggatccagcagctggaggcgctgcagcgcctgatccaggagcaggagcaggagctggtgggcgcgctggccgcagacctgcacaagaatgaatggaacgcctactatgaggaggtggtgtacgtcctagaggagatcgagtacatgatccagaagctccctgagtgggccgcggatgagcccgtggagaagacgccccagactcagcaggacgagctctacatccactcggagccactgggcgtggtcctcgtcattggcacctggaactaccccttcaacctcaccatccagcccatggtgggcgccatcgctgcagggaacgcagtggtcctcaagccctcggagctgagtgagaacatggcgagcctgctggctaccatcatcccccagtacctggacaaggatctgtacccagtaatcaatgggggtgtccctgagaccacggagctgctcaaggagaggttcgaccatatcctgtacacgggcagcacgggggtggggaagatcatcatgacggctgctgccaagcacctgacccctgtcacgctggagctgggagggaagagtccctgctacgtggacaagaactgtgacctggacgtggcctgccgacgcatcgcctgggggaaattcatgaacagtggccagacctgcgtggccccagactacatcctctgtgacccctcgatccagaaccaaattgtggagaagctcaagaagtcactgaaagagttctacggggaagatgctaagaaatcccgggactatggaagaatcattagtgcccggcacttccagagggtgatgggcctgattgagggccagaaggtggcttatgggggcaccggggatgccgccactcgctacatagcccccaccatcctcacggacgtggacccccagtccccggtgatgcaagaggagatcttcgggcctgtgctgcccatcgtgtgcgtgcgcagcctggaggaggccatccagttcatcaaccagcgtgagaagcccctggccctctacatgttctccagcaacgacaaggtgattaagaagatgattgcagagacatccagtggtggggtggcggccaacgatgtcatcgtccacatcaccttgcactctctgcccttcgggggcgtggggaacagcggcatgggatcctaccatggcaagaagagcttcgagactttctctcaccgccgctcttgcctggtgaggcctctgatgaatgatgaaggcctgaaggtcagataccccccgagcccggccaagatgacccagcac

SEQ ID NO: 17 - protein, 453 aa, synthetic construct [sequence omitted]

SEQ ID NO: 18 - protein, 517 aa, Homo sapiens
MLRAAARFGPRLGRRLLSAAATQAVPAPNQQPEVFCNQIFINNEWHDAVSRKTFPTVNPSTGEVICQVAEGDKEDVDKAVKAARAAFQLGSPWRRMDASHRGRLLNRLADLIERDRTYLAALETLDNGKPYVISYLVDLDMVLKCLRYYAGWADKYHGKTIPIDGDFFSYTRHEPVGVCGQIIPWNFPLLMQAWKLGPALATGNVVVMKVAEQTPLTALYVANLIKEAGFPPGVVNIVPGFGPTAGAAIASHEDVDKVAFTGSTEIGRVIQVAAGSSNLKRVTLELGGKSPNIIMSDADMDWAVEQAHFALFFNQGQCCCAGSRTFVQEDIYDEFVERSVARAKSRVVGNPFDSKTEQGPQVDETQFKKILGYINTGKQEGAKLLCGGGIAADRGYFIQPTVFGDVQDGMTIAKEEIFGPVMQILKFKTIEEVVGRANNSTYGLAAAVFTKDLDKANYLSQALQAGTVWVNCYDVFGAQSPFGGYKMSGSGRELGEYGLQAYTEVKTVTVKVPQKNS

SEQ ID NO: 19 - DNA, 387 bp, synthetic construct
  source 1..387: mol_type = other DNA
atgtacgtggaggggacactggatctgctggaactgcttatcatgcatcctttcttaaaaccagatgatcagcaaaaggaagtggttaacatggcccagaaggctataattagatactttcctgtgtttgaaaagattttaaggggtcacggacaaagctttcttgttggtaatcagctgagccttgcagatgtgattttactccaaaccattttagctctagaagagaaaattcctaatatcctgtctgcatttcctttcctccaggaatacacagtgaaactaagtaatatccctacaattaagagattccttgaacctggcagcaagaagaagcctccccctgatgaaatttatgtgagaaccgtctacaacatctttaggcca

SEQ ID NO: 20 - RNA, 387 nt, synthetic construct
  source 1..387: mol_type = other RNA
atgtacgtggaggggacactggatctgctggaactgcttatcatgcatcctttcttaaaaccagatgatcagcaaaaggaagtggttaacatggcccagaaggctataattagatactttcctgtgtttgaaaagattttaaggggtcacggacaaagctttcttgttggtaatcagctgagccttgcagatgtgattttactccaaaccattttagctctagaagagaaaattcctaatatcctgtctgcatttcctttcctccaggaatacacagtgaaactaagtaatatccctacaattaagagattccttgaacctggcagcaagaagaagcctccccctgatgaaatttatgtgagaaccgtctacaacatctttaggcca

SEQ ID NO: 21 - protein, 129 aa, synthetic construct
MYVEGTLDLLELLIMHPFLKPDDQQKEVVNMAQKAIIRYFPVFEKILRGHGQSFLVGNQLSLADVILLQTILALEEKIPNILSAFPFLQEYTVKLSNIPTIKRFLEPGSKKKPPPDEIYVRTVYNIFRP

SEQ ID NO: 22 - protein, 222 aa, Homo sapiens
MAARPKLHYPNGRGRMESVRWVLAAAGVEFDEEFLETKEQLYKLQDGNHLLFQQVPMVEIDGMKLVQTRSILHYIADKHNLFGKNLKERTLIDMYVEGTLDLLELLIMHPFLKPDDQQKEVVNMAQKAIIRYFPVFEKILRGHGQSFLVGNQLSLADVILLQTILALEEKIPNILSAFPFLQEYTVKLSNIPTIKRFLEPGSKKKPPPDEIYVRTVYNIFRP

SEQ ID NO: 23 - DNA, 8526 bp, synthetic construct
  source 1..8526: mol_type = other DNA
ttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccagggggtaccaccatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggcctgcagctgcagtagtaagaattctagatcttgagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccactttggcgccggctcgagggggcccggggttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactgtcgggatcaacaagtttgtacaaaaaagcaggctccaccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctaattggacccagctttcttgtacaaagtggttggtaagcctatccctaaccctctcctcggtctcgattctacgtagtaatgagctagcgctaaccggtggcgcgttaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgtcgactttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtacgtatagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttc

SEQ ID NO: 24 - DNA, 8787 bp, synthetic construct
  source 1..8787: mol_type = other DNA
ttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccagggggtaccaccatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggcctgcagctgcagtagtaagaattctagatcttgagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccactttggcgccggctcgagggggcccggggttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactgtcgggatcaacaagtttgtacaaaaaagcaggctccaccatgggcaacgccgccgccgccaagaagggcagcgagcaggagagcgtgaaagaattcttagccaaagccaaagaagattttcttaaaaaatgggaaagtcccgctcagaacacagcccacttggatcagtttgaacgaatcaagaccctcggcacgggctccttcgggcgggtgatgctggtgaaacacaaggagaccgggaaccactatgccatgaagatcctcgacaaacagaaggtggtgaaactgaaacagatcgaacacaccctgaatgaaaagcgcatcctgcaagctgtcaactttccgttcctcgtcaaactcgagttctccttcaaggacaactcaaacttatacatggtcatggagtacgtgcccggcggggagatgttctcacacctacggcggatcggaaggttcagtgagccccatgcccgtttctacgcggcccagatcgtcctgacctttgagtatctgcactcgctggatctcatctacagggacctgaagccggagaatctgctcattgaccagcagggctacattcaggtgacagacttcggtttcgccaagcgcgtgaagggccgcacttggaccttgtgcggcacccctgagtacctggcccctgagattatcctgagcaaaggctacaacaaggccgtggactggtgggccctgggggttcttatctatgaaatggccgctggctacccgcccttcttcgcagaccagcccatccagatctatgagaagatcgtctctgggaaggtgcgcttcccttcccacttcagctctgacttgaaggacctgctgcggaacctcctgcaggtagatctcaccaagcgctttgggaacctcaagaatggggtcaacgatatcaagaaccacaagtggtttgccacaactgactggattgccatctaccagaggaaggtggaagctcccttcataccaaagtttaaaggccctggggatacgagtaactttgacgactatgaggaagaagaaatccgggtctccatcaatgagaagtgtggcaaggagttttctgagttttaattggacccagctttcttgtacaaagtggttggtaagcctatccctaaccctctcctcggtctcgattctacgtagtaatgagctagcgctaaccggtggcgcgttaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgtcgactttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtacgtatagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttc

SEQ ID NO: 25 - DNA, 9090 bp, synthetic construct
  source 1..9090: mol_type = other DNA
ttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccagggggtaccaccatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggcctgcagctgcagtagtaagaattctagatcttgagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccactttggcgccggctcgagggggcccggggttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactgtcgggatcaacaagtttgtacaaaaaagcaggctccaccatgagcaagatcagcgaggccgtgaagcgcgcccgcgccgccttcagctcgggcaggacccgtccgctgcagttccggatccagcagctggaggcgctgcagcgcctgatccaggagcaggagcaggagctggtgggcgcgctggccgcagacctgcacaagaatgaatggaacgcctactatgaggaggtggtgtacgtcctagaggagatcgagtacatgatccagaagctccctgagtgggccgcggatgagcccgtggagaagacgccccagactcagcaggacgagctctacatccactcggagccactgggcgtggtcctcgtcattggcacctggaactaccccttcaacctcaccatccagcccatggtgggcgccatcgctgcagggaacgcagtggtcctcaagccctcggagctgagtgagaacatggcgagcctgctggctaccatcatcccccagtacctggacaaggatctgtacccagtaatcaatgggggtgtccctgagaccacggagctgctcaaggagaggttcgaccatatcctgtacacgggcagcacgggggtggggaagatcatcatgacggctgctgccaagcacctgacccctgtcacgctggagctgggagggaagagtccctgctacgtggacaagaactgtgacctggacgtggcctgccgacgcatcgcctgggggaaattcatgaacagtggccagacctgcgtggccccagactacatcctctgtgacccctcgatccagaaccaaattgtggagaagctcaagaagtcactgaaagagttctacggggaagatgctaagaaatcccgggactatggaagaatcattagtgcccggcacttccagagggtgatgggcctgattgagggccagaaggtggcttatgggggcaccggggatgccgccactcgctacatagcccccaccatcctcacggacgtggacccccagtccccggtgatgcaagaggagatcttcgggcctgtgctgcccatcgtgtgcgtgcgcagcctggaggaggccatccagttcatcaaccagcgtgagaagcccctggccctctacatgttctccagcaacgacaaggtgattaagaagatgattgcagagacatccagtggtggggtggcggccaacgatgtcatcgtccacatcaccttgcactctctgcccttcgggggcgtggggaacagcggcatgggatcctaccatggcaagaagagcttcgagactttctctcaccgccgctcttgcctggtgaggcctctgatgaatgatgaaggcctgaaggtcagataccccccgagcccggccaagatgacccagcactaggacccagctttcttgtacaaagtggttggtaagcctatccctaaccctctcctcggtctcgattctacgtagtaatgagctagcgctaaccggtggcgcgttaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgtcgactttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtacgtatagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttc

SEQ ID NO: 26 - DNA, 8109 bp, synthetic construct
  source 1..8109: mol_type = other DNA
gcccggggttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactgtcgggatcaacaagtttgtacaaaaaagttggcatgtacgtggaggggacactggatctgctggaactgcttatcatgcatcctttcttaaaaccagatgatcagcaaaaggaagtggttaacatggcccagaaggctataattagatactttcctgtgtttgaaaagattttaaggggtcacggacaaagctttcttgttggtaatcagctgagccttgcagatgtgattttactccaaaccattttagctctagaagagaaaattcctaatatcctgtctgcatttcctttcctccaggaatacacagtgaaactaagtaatatccctacaattaagagattccttgaacctggcagcaagaagaagcctccccctgatgaaatttatgtgagaaccgtctacaacatctttaggccatacccaactttcttgtacaaagtggttggtaagcctatccctaaccctctcctcggtctcgattctacgtagtaatgagctagcgctaaccggtggcgcgttaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgtcgactttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtacgtatagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccagggggtaccaccatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggcctgcagctgcagtagtaagaattctagatcttgagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccactttggcgccggctcgagggg

SEQ ID NO: 27 - DNA, 37266 bp, Homo sapiens
  source 1..37266: mol_type = genomic DNA
agtgtcctcagctgcctccgcgcgccaaagtcaaaccccgacacccgccggcgggccggtgagctcactagctgacccggcaggtcaggatctggcttagcggcgccgcgagctccagtgcgcgcacccgtggccgcctcccagccctctttgccggacgagctctgggccgccacaagactaaggaatggccaccccgcccaagagaagctgcccgtctttctcagccagctctgaggggacccgcatcaagaaaatctccatcgaagggaacatcggtaaggagcctccggaaatgtgggacgcaaggctggggtgtcgcggcagtggctgaagcttcccttcgccgcatctctctgcagcaactcggtgagggctttccctccagcctggcggtgtggacgcggcctcggcttcctttcccacccagagcatccttcccttacctcccgctccacggggtgactgcggtccccggggtcagactgggcgccaggcctgcggtctctgtgaggcgcgctttgaacctctgctccctctttgttgggaactcgcaaagggaagcgggggaagggtattagatttcttgaggtgtaacttacattaaattcaccctttttaggtgtactcttctatgaggtttgacaaacattatgccttttgctatcaccatgatcgagatacagaacattttcatcaccttagaaagttcctacctgctcctttgtattcagccccctccacccttcctattccctggcacccactgatttgatttctatccctgcaagtgttgccttttccagaatgccatataaatggaatcataggctatatcgccttttgtgtctggcgtctttcatttagcataatgcttttgaaattcattcatgttacttgtgtctgtagtttgtttctttttattgctgagtagtattccattgcatggatgtaccatcgtttatccattttctagttgtggacctcagggttgtttacagttttttagctatattattagcgaagcattgttttagctatattattaacgaaactcgaaacattcgagtacaagcaaagaggtctttaatgatcttaatagcatggagttcctcaccccttactccacatccaagtaaacattggattatgattcaaagaccactaacgtttctgttaactcagatggggggcagactaggccatgcctgtcactttgcaagatacacaccaacgtccgattcttctgtatctccaaccacaggaaacctacccctgaaacttcctgttttgatagatttaaaaaaaaatttttttagctccaggttgggtgctggtcattcatgcatacagcaaattttttaaaattcttaccacattcaaagcactgtaccttcttgaaaaaggccaggcaagcagatgctttaagcattcaaatgaatagaaaaactgcattctcaaagaggacttcttatttcattgctccagaattaaagcaccagtagaaagaccatatgatataattagcaatgtaagttatttgagggtgcatgtttactagtatggaatctctgacagctgcttactgaataaagaagaccatatataaagacaggagaaagtggctgaacaaagccatctgaaaagtaaatttgaggcagggcatggaggctcatacttgtaatcccaggatttggggaggcttaggcaagaggatcacttgagtccaggagttccagatcagcctgggcaacatagtgagaactcatctccacaaaaaaattaaaaaattagttgcatgtgttggtgtgcctccagctactcaggaggctgaggcaggaggattgcttgagcccaaagatcaaggccacagtgagccatgatcacactactgcactctagcctgggtgacagagcaagactctgttaaaaacacagaaacaaaacagtaaagttgaatgaataacccctcggatcaataccacaatacattcatcccctttccctctctccaatccagttaggtagcatcatatttagaaatatttcctcaaagcatctttaatcttttcagcagctgaaataatttcctaatatatcatcccatctccagtttctcttaactacttctgcaacggtctttttgtatgcaaatgtcatgtgttatcccctgcttaaaattcttcactgattttgcctttacctacactgaaagtagttgtgaactgatagcctgcaattgcacaatattggcctgcacaggttgaaaaaaaattttactggccagtggctcaagcttgtaatcccagtgctttgggaggctgaggcaggaggattgcttgaggccaggagtttgagactagcatggcaacatagtgagaactagtctctaagaaaaataaaaataaaattaggccaggcacagtggatcacgcctgtagctccaactactcaggcggctgatgctgatgtgggaggatcacttgaatgtgggaggcagaagttgcagtgagctgagatggcaccactgcaccccagcctgggtgacagagtctcaaaaaaaaaaaaaaagccagttgtgttagcatgcacgtttagtcctagctactcaggaggctgaggtggagatctttgagacctatagtttgaggctgccgtgagctgtggttgtgccactgccctacagcctgggctacagagtgagaccctgtgtctaaagaaaaaaaaattaaatttgttgctaactcgaaaacgttttgagatttcccataaacaattagattcttggcttcttttgaaaaatctgaagatctgttaacaccgggcctgcatttctgtatgacaacaattggtgtttagatggagcatgttccttgccatttttcgtctccctgacactgagactttgttagttactgttcattattggtcttgcacttttgttcatgtctccttcaccttatttgccagatccaaataagctgtaaatgcacaaattgttaagctgtgcgtaaactattttgcaattcctctatttttcctagaatttgaactttacttggtcgttttagttttatatttatataacccatcttcttatgttctgtgttactcaaatgttcccttatgttagattgatggaggacttacctgcttctcatcttaatagtctgttcagaatgactttcagcattataatgcctgcttggcattaggaattctgttagtttccatgactttttaaagtattaacatcagtgtctttggttcttgttctcctttgccttcatagaaaattaatttattaattagcttattttctagctcccactttgccccaataagttaaggaactagatgtgttagtgtgattgggaactctagctgttattactctgcttgaactaaatgtaactgttattactcttagactttttaagtatgaccatatgaatttttctgcttgtaagcagtgtcagaagaaagttgaatgctgtagccttaagtgctttcagactctggtggatgcctaatttttttatttgttccttgggtgctatttctgaacatcatgttctcctttccctcttccatctagctcagtgaatgtggggtgttctttctatatgctttcacttccacttaacattcttctaaatgctgtacagtccctaagcttacactctccatactaaattttggtggccagtgtttttaccatgattaaatctcactgttatccagtttatcgttaaaagtttttctcctctcctctggatacagtttactttgtgtttttcttctaaactgtctacctgtgtcaccttctttactgcctcgctcctgttttttctctgtgtgtaattctttaaagttcttccaggtttttcttcttttctattgccctgtctaatgtcctagatttaaatatagttctgcaagtcaaaggtaagttctttctactccccatggatctaatttataaggaagaggaagctttgcttgtaagtatattgaagttatttatatgttcatctgtgtgtgtgtgcacgcacacacagggaaagagagagaacattatgctaaccagagatgtctaatacaaccacatttggtgacttcagatataaagggtgtggtggccattaggagtattacttctaggtcaagactgtccctactattaaatataatatattgttaaactaaacgtaataaaaaacaaatgtgcttaatgaaattgggcagggagccttttcattttcttctaaagaaatatgaagagaaagtttagaaagttgaatgttttgggctttttatgttgcttgctattgtaaatcttgcaaaagctaatgactacttgacatctttttttcctgacaacttttcttcctcaattttatctttcctcacaacagctgcagggaagtcaacatttgtgaatatccttaaacaattgtgtgaagattgggaagtggttcctgaacctgttgccagatggtgcaatgttcaaagtactcaagatgaatttgaggtatgaaaataaaatttaagtagataaaagcctcttggtttagaggagcagtagtacttaatctctttttctttttctttttcaataaaactttcaaatctcgctttaggtatatatcttcatctaggtggtggttacatggatatatatacagttggctctccatatcaatgggtttcagattgatatggattcaaccaatcatgaatagaaaatattcagaaagaaataatacaaaaaaatacaaataataaaggattactgttcatatagcatttacattgtattaagtattataagtaatctagagatgatttatactatttgggaggatatatgcagatactacaccattttacataaaggacttgagtatatgtggatgttggtatctgtgtggaacctggaatcaattcttgaggttactgaggggtgactatatgtttaaacatgcattgaggccggggttggtggctcacgcttgtaatcccagcactttgggaggctgaggcgggcggatcacttgaggtgaggagttcaagaccagcctcgttaacatggtgaaatgccgtctctactaaaaatacaaaacttagctgaacgtggtggtgggcacctgaattccacctgcttgtaaagccgaggtaggagaatttcttgaacccaggcagcggaggttgcagtgagctgagatggcgccactgtgctccagcctgggtgacagaatgagacactgtctcaacaaaaaaaaaaaaaggaaagtaaaatttatcaaactatgcattcaagatttatgcactttaagtaaagtataaaagttttctttttcataaaaatttctttttttgttgttatcctttaagttctgggatacatgtgcagaacgtgcaggtttgttatgtagatatacacatgccatggtggtttgctgcacccgtcaacccgtcatctacattaggtatttctcctaatgctaacccttcccttgcctcctaccccaccgacaggccctggtgtgtgatgttcccctccctgtgcccatgtgttctcattgttcaactcccacttacaagttagaacatgcggtgtttggtttcctgttcctgtgttagcttgctgagattgatggtttccagcttcatccatgtccctgcaaaggttatgaactcattcttttattatggctgcatagtattctttggtgtatatgtgccacattttgtttatccagtctatcattgatgggcatttgggttggttccaagtctttgctattgtgaatagcgctggagtaaacatacatatgcatgtgtctttatggtagaataatttataatcctttgggtatatacccagtaatgggatggctgggtcaaatggtatttctggttctagatccttgaggaatcgccacactatcttccacagtggttgaactaatttacactcccactaacagtgtaaaagcattcctatttctccacatcctctccagcatctgttgtttcctgactttttaatgatcaccattctaactggtgtgagatggtatctcattgtgggtttggtttgcatttctctaatgactggtgatgatgagctttttttcatgtttgttggctgcataaatgtcttcttttgaaaagtgtctgttcatatcatttgcccactttctgattgggttgtttgtttttttttcttgtaaatttgtttaagttccttgtagattttggatattagccctttgtctgatgggtagattgcaaaaattttctcccattttgtaggttgcctgtttacttggatgatagtttcttttactgtgcagaagctccttagtttaattagatcccatttgtcagttttggcttttgttaacactgcttttggtgttttagtcatgaagtctttgctcatgcctatgtcctgaatggtattgcctatgttttcttcagagtttttatgattttaggtcttatgtttaaatctttaatccatcttgaattaatttttgtataaggtgtaaggaaggggtccagtttccgttttctgcatatggctagccagttttcccagcaccatttattaaatagggaatcctttccccattgcttgtttttgtcaggtttgtcaaagatcagatggttgtagacgtgtggtattatttctgagggctctgttctgttccattggtctatagctctgttttggtacaataccatgccgttttggttactgtagccttgtagtatagtttgaagtcaggtagcatgaggcctcaagccttgttctttttgcttaggattgtcttggctatacgggctcttttttggttccatatgaaatttaaggtagttttttctaattctgtgaagaaagtcaatggtagcttgatgggaatagcattgaatctgtaaattactttgggcagtatggccattttcacaatattgatttttcctatctatgagcatggaatgttttaccatttgtttgtgtcctctcttctttccttgagcagtggtttgtagttctccttgaagaggtccttcacatccgttgtaagttgtattcctaggtattttattctctttgtagcaattgtgaatgggaattcactcatgatttggttctctgtctgttagtggtgtataggaatgcttgtgatttttgcccattgattttgtatcctgagactttactgaagttgcttgtcaccataagggattatgggctgagatgatggggttttctaaatatacagtcatcatctgcaaacagagataatttgacttcccctcttcctatctgaatcccctttatttctttctcttgcctgattgccctggccagaccttccaatactatgttgaataggagaggtgagagagggcatccttatcctgtgccgatttttgaagggaatggtatcggctgtgggtttgtcataaatagctcttattttgagatacatttcatcaatacctagtttattgagagtttttagcatgaagggatgttgaattttattgaaggccttttttgcatctattgagataatcatgcggtttttgtcattggttctgtttatgtgatagattatgttttattgatttgagtatgttgaaccagccctgcatcccagggatgaagccgacttgatcgtggtagatgagctttttaatgtgctactggattcggtttgccagtattttattgaggattttcgcatcgatgttcatcagggatattggcctgacattttcttttttttgttgtgtctctgccaggtttttgtattagggtgatgctggcctcaaaaaatgagttagggaggagtccctctttttctattgtttggaagtttcagaaggaatagtaccagttcctctttctacctgtggtagaattcagctgtgaatccatctggtcctgggcttttttttggttggtaggatattattgcctcaatttcagaactttttattggtctgttcattaaaccagagttcttcctggtttaatcttgggagggcgtatgtgtccaggagtttatccatttcttccagattttctagtttatttgcgtggaggtgtttatagtgttctctgatggtagtttgtatttctgtgggatcagtggtgatcctttattgtgtctatttgattcttcccgcttttcttctttattattctggctagtggtctatctattttgttaatcttttcgaagaaccagcttctggattcattgattttttgaagagtttttcgtgtctctatctccttcagttctgctctgatcttagttatttcttgtgttctgctagcttttgaatttgtttgctcttgcttctctagttcttttaattgtgatgttagggtgtcgattttagatctttcctgctttctcatgtgggcacttagtgctataaatttccctctaaacactactttagctgtgtcccagagattgtggtacgttatgtctttgttctcattggttgcaaagaacttatttatttctgccctaatttcgttatttacccggtagtcattcaggagttgttcagtttccatgtagttgtgcagttttgagtgagtttcttaatcctgagttctaatttgattgcactgtggtctgacagactgttatgatttccattcttttgcatttgctgaggaatgttttacttccaattatgtggtcaattttagagtaagtgcactgtggtactgagaataatgtatattctgttgatttggggtggagagttctctagatgtctttcaggtctgcttggtccagagctgagttcaagtcctgaatatccttgttaattttctgtcttgttgatctaatattgacagtggggtgttaaagtctcccactgttattatgtgggagtctaggtctctttgtgggtttctaagaacttgcattttgaatctgggtgctcctgtattgggtgcatatctatttaggatagttagctcttgttgttgcattgaaccctttaccattatgtaatgccctttgtctttttttcatgcttattgatttaaagtctgttttatctgagactaggattgcaacccctgccttttttttgctttccatttgcttggtaaatcttcctccatccctttattttgagcctttgtgtgtctttgcatgtgagatgcgactcctgaataccgcacactgatgggtcttaactctgtctaatttgccactctgtgtctttcaactggggcatttagcccatttacatttaaggttaatattgttatgtgtgaatttgatcctgttattatgatgctagctggttattttgcctgttagttgatgcagtttcttcatagtgtcgatggtctttacattttggtatgtttttgcagtggctggtaccggtttttcctttccatatttaatgcttccttcaggagctcttgtaaggcaggccttgtggtgataaaatccctctacatttgcctgtctgtaaaggattttatttctccttcacttatgaagcttagtttgactggttatgaaattctgggttgaaaattcttttctttaagaatgttgaatgttagtccccactctcttctggcttgtagggtttctgccgagagatctgctgttagtctgatgggcttccctttgtgggtaacccgacctttctctctggctgcgcttaacattttttccctcattttaaccttggtgaatctgacgattatgtgtcttggcgttgctcttctcaagggcatctttgtgatgttttctgtatttcctggatttgaatgttggcctgtcttgctaggttggggaagttctcctggataataatcctgaaatgtgttttccaacttggttccattcgtcagtttcaggtataccagtcaaacgtaggtttggtcttttcacatagtcccatatttcttgggaggctttgtttgttccttttcattctgttttctctaatcttatcttcacgctttatttttattaagtttttcttcaatctctgatatcctttcttccacttaatcgattcggctattgatacttgtgtatgcttcaccaagttctcatgctgtgtttttcagctccatcaggtcatttatgttcttctgtaaactggttattctagttagcagttcctgtaaccgtttatcaaggttcttagttccttgcattgggttagaacgtgctgctttagctcagtggagtttgttattaacccaccttctgaggcctacttctgtcaattcgtcacacttattctccatccagctttgttctcttgctggcgaggagttgtggtcctttggaggagaaaagacattctggtttttggaatttttagcttttttgcactggcttttcttcatctttgtggatttatctacctttggtctttgatgttggtgaacttcagatggggtttttgcgtggttgtcctttttgttgatgttgatgctattgctttttgtttgttagtttccttctaacagtcaggcccctctgctgcaagtctgctggagtttgctggaggtccactgcagatcctgtttgcctgggtatcaccagcggaggctgcagaacagcaaagattgctgcctgctccttcctcaggaagctttgtcccagaggggcacctgccagaggctagccagagctctcttatatgaggtgtctgccgacccctgctgggagctgtgtcctagtcaggaggcatgggggtcatggacccacttgaggatgctgcctgtcctttagcagagcttgagcgctgcgctgggagttccactgctctcttcagagccagcaggcaggattgtttaagtctgctgaggctgcactcacagccacgccttcccccagatgctctgtcccagggagatgggagtttgatctgtaagtccctgacgggggctgctgcctttctttcagagatgccctgcccagagaggaggaatctagagaggcagtctagcttcaggggtttgccaagctgtggtgggctctgcccagcttgaacttccaggtggttttgtttacactgtgaggggaaaaccacctactgaagtctcagtaatggcgggcacccctccccccaccaagcttcagtgtctcaggttgacttcagactgctgtgctggcagtgagaatttcaagccagtggatcttagcttgctgggctccgtgggggtgggatctgctgagcaagaccacttggctccctggcttcagcccccttggctccctggcttcagccccctttctaggggagtgaacggttctgtctcgctggcattccaggcaccactggggtatgaaaaaaaactcctgcagctaactccgtgtctgcccaaatggctgtccagttttgtgcttgaaacccagggcccttgtgtaggcacccgagggaatctcctggtctgcgggttgtgaagactgtgggaaaaagcctagtatctgggctggatagcaccatccctcaaggcttcccttggctaggggagggagttcccccacccctttcaccttccaggtgaggcgatgccccaccctgcttctgcttggcctctgtgggctgcacccactgtataaccagtcccaatgagatgaattgggtacttcagtcggaaatgcagaaatcacccgtcttctgcattggtctcactgggagctgcagaccagagctgttcctatttggccatcttgcctgggaatctaaaagtttttaaaaagttaaataataaaagttttaaaaaatctaacagcaaattaaattccttcattgtcttttctaatttttttttttttttgagatggagtttcgcttttgttgcccaggctggagtgcaatggctcgatcttggctcaccacaacctccgcctcccgggttcaagcgattctcctgcctcagcctcccaagtagctgggattacaagcatgggccaccatgcctggctaattttttttactagagatggggtttctccatgttggtcaggctgggcccaaactcctgacctcaggtgatccgcctgcctcagcctcccaaaatgtttggattacaggcctgagccactatgcccaacccttcagtgtcttttcacattttgtttaaaaacaagcaaataaggccgggtgcggtggctcacacctgtaatcccagccctttgggaggccgaggcaggtggatcgcctgaggtcaggagttcgagaccagcctggccaacatggtgaaaccctgtctccactaaacatacaaaaattagctgggcgtggtggtgggcgcctgtaatcccagctactcaggaggctgaggcgggagaattgcttgaacccgggaggtggaggttgcagtgagccgagatcgcgccattgcactccatctcaaaaaaaaaaaaaacagaaaaaacaaaaacaccaaaaaaaaaaaaaaaaaagcaaataagaaaaagaaaaactactattcagtaactgaattttacctttcaagccttttgtcatttactctcatttctttttactttcattcttgccagactttcactctgggcagaagttgtaattttgcagcattttaccgttctgcactgcaaatgaggaagaaagagaatgacagagttcagtgtcatttttttaaataccaaaatgtatggttgtatgtgtgagagagacgttgctaagattttcgtttctggagaaatgagaattgaaattgaaagcaaggccaggtgtggtggtttatgcatgtaatcctggaatttttttttttttaagtgacagcaagtttattaggaaagtaaagcaataaagaatggctactccataggcagagcagccaatcccagcacttcgggaggctgaggccagaggatcgcttgagcccaggagttcaagaccagcctgggcaacatagggtgaccccgtctctacaaaaaatttaaaaattagctaggtgtggtagcctgcacttggggttacagctacttggaaggctgaggttggaggactgcttgagcccaagaaattgagactgtagtgagccatgctcatgccactgtactctagcctgagtgacagcaagaccctgtctaaaagaaaaaaagaaattgaaagcagtaaggcagaaaccttaaaaatggaaatgtatgctaacaacttactttgatctgaggagtacagaaaattgaaaaaaaagtatctaaaaaaatttagctgttggtataaggaattctgggaataataattgaatatttaaaatgtaaatcacatatgcaaagaggaatcactagcaagatattccatggactttgagaatatatggttttgaataaccaaaaatactttttcatacaccttctatttgccaggcatagctaactttagacctagggcctaattcagtacaccccaatccttaggatatccaggactaaatcttaaaggggctttcttcccatatagacctcattatcttaagaaattgatgttttatatctgatttttggggaaaggcaaggttagtctacagtagaattttaaagtgggtaaatttgtttatttgattctttttaaaaacatttttgacatgatatatctgtagaaaagtaggtaaaagctaatgtacagtttgaagaacagttttaaagttgacatctttataaccattatccaggtcaagaaatacgacatcatcattccagaaatccctgttcgccttctccaatcactatatcttctttcctacgttactactccgctaggggtaattactcccattcttttgtgataataattttcctgcttcctaaaaattgtttgagcatacatgaacatacctctaagtataatttagttttgtctatttttgaatgttatataagtggaattacattgtttgccttttttcctctcgcttcttttacttaatacatttatgaaatttgtccatattatatgcagctgtagcttattttcagctatatagtattgaattttgttttttaaaaatattcttttttttgagacaggatttcactctgttgcccaggtaaagtgcagtggcgcagccatggttcactgcagcctcaacctcccttggctcaggtgatcctctctcctcagcctcctgagtatctaggaccataggccaccacgcctgtgtgccaccaggcttggctaatttttgtattttttatagagacggggttttgccatgttacccaggctggtttcaaactcctgggctcaagcgatctgcccatctcagcctcccagagtgttgggattacaggcttgagccactatgtggggcctatagtatttaattttattcgtctatcactgtttgtccattttatacctttaattgatttttgagtgatttccaatttagttgcttatgtaaacagcattgctgtgaacataaattcacccgcatacatgcaaaagtttctgtaggaataagaagtaaaattactgggaatgcttatgatgcctgttttccaaactggctttccaattttctctccccccagtgtaatcaagctctcattaatctatgtctttgccagcatttagtagtgtctggctttctcatttttgtcattctgctgagtgtttactatttttttgtttaatttgtatttccccgctggctaatgaggctgtgttccttttcatatgtttattagtcattttgatttttgcaaagtgcccacttgggtccttttgcccattttcctctgagccatctgtcttctctcactgcatttttaatgtagcttttcgctgaagtacctactcaagtttgcttagcttttgaaggagagactggagttctaaaagcaactctttttccttttatttctggggtcagctgtcagtgcagttatcctcaggtagtagacagaaactaaccagattaaaccaatgttggaaaatagagggtattcttaaacagggtattggactaggtgacctccatttaaggtccgttgacatatttgtcatttgactctcagcatgcctcacattaaccaagggcacagaaactgacatataaaaccaaaatgccaaaaattttaatttgcagaaagaaatgtaatcattaaggtaattgaatttagatcagtttattctttatttggttcgtaattctcaaccagtggaaggaagtcataagaatgtctgagagctaaacagtttgaacacatggagatagagtgtggaaaaatagataacaataactgggaagggtaggaggaggggaagatgaagaggagtgagttaaagtgtacagatatacaggaagcttgaagaaataattcagtgttttatagcaaggtagaatggaaaaatacattgtactcaggtgatggacaccctgaatgattcgattactgcatattatatacatgtacaaaatttctcatgtattccataatttgtacacacaagaaagaatctcaaggattcgggtgctttttaaaataacaatcccttggaggttcaaatatgtgggatgcctcatagttaaaagtcatcaccctagtgaatcactgttaactgttgggagtatactgaactctttagttaggttgcagagaagttgaaaaacattgctttcgtatataagacaagacaatttataataaggttgtctgttgagcttggaatcagagcccagagggattagttaaatcatgttaatcagactgtgtcatcagactgtgatgcattaatacatttgtcttcattttaagacttaatggctttctgaatctgatatccatgtttatgggatgggacattttctttccatatacttgattcagaatgccaaactggccattctttataactagcatgcaggtgatcctattattattaacatattcttttatcaggccgtttattgtaatctggtcatgtcttcatgatgttacaatttgattacctttgttagagtttggtgcacattcttggaaggtagcttcagcttttttgtgtgtattggggactgctccgttgttaattctctgtacttctgtacagagggggtagtatttttgagatatagttgtggaatatgaggatggttgggtgggtggcttccctgtatctttggatattttcttccactaaactcaagaagtgagattttatttcttcatccagtagtccttcttccccagactcatccttgatctattccttgaggttatagtatgaatcacccactattcaaatgtcaatttttcttagaatgctgttagtgatgagaagtgactgttttcagacctaaccttggcaaggtcagtcctactttgatgttcttgtttcatcacacttcttggcatttgtagatttggaagaattgggcctttggtacctctgatctcttcgtttagcaacttactgtgcacccatatgcttagcttttgctgttttagctttttttttttttttttaacctgccacctagtggccgaaatgttgctatactattgataaggtactcctaattttggcaaaatagtaagaggcaaagcaccaaagattatgttctctcccttctccaaatctctcttggtgagaatgatctttaaaacataccactcagattattagcaatcttggtatggaacgtttttaaaaataataataatgtactttatgtggtgatttatgttattatttaggcccaaagttttgatttaattgtttccttttagcttatttttgagatatgcagtctgttaggaagctgtctctgtcttgttttttcattgagctgaattcttttggaggatcactattattgtgaagtactattttacttttaattaaaaaaattaagatatttttatttttaatttacagataaaaattatatttaattttatattatatttatatataaatcatatgtaatgtaaattatatacataagttatatataatataaaaattatatacataatataaattatatataatataaaaattatatttatggtatgcaacatgatgttttaaaatatgtctacattgtggaatggttaaaccaagctaattaacatacgcattacccgtactattttacttttatccaatacttcttaccactaataagccactgaaaaactagtaattgtgataaagaaggtgtaaaccatacttatttatgacagaacagcatattttttacttttaataagtcattctttttttttttttttgagacggagtctcgttctgtcgctcaggctgaagtgcagtggtgtgatgttggctcactgcaacctctgcctcccaggttcaagcgattttcctgccttagccttccaagtagctggagctgggattacaggcatgtgccaccacgcctggctaatttttgtaattttagtagagatggggtttcaccatgttggccaggctggtcttgaactcctgacctcaagtgatccacccatctcagcctcccaaagggctgggattacaggtgtgaggtctcttttttttttttttttttttttttaagacggagtcttgctctgtcccttagtaagtcattcttgaaaattagttaagaatgcttgataaagcacttccacttgtaccttttgaatagatgtgataacttttgttggcttgtatttttttgagtcttgctttattgcccaggctggagtgcaatggtgaacctcagcccactgcagcctccgcctcctgggctcaagtgatcctcccacctcagcctcctcagtagctgggactacaggcccacaccaacatgcccggctaatttttgtattttttatagcgataggattttgccaatgttgtctggatttgtctcaaactcctgggatcaagcaatccgcctgcctcggcctcccaaagtgctagaattaggcatatgagtcaccatgcctggcctgttggctattttataattatcttgtttggtagactctgatcttttacagttgaacttaattcaactcttttgcaatgtttacaaagattagacctagaattaaaagataaatgattttatagctgaccttgatatttctatttttcacttaccttgacaataatctgtacttcctataacaatgaagtattctttcataaagtgaaaatggacatatattatagagaacagaataaatgttattcataatcctaccactctgtatgtatcttttacatagttgaattaataatgtaatatattaaattttggtggaggtgatatgtaatttaaaaaatattataaagtaatacctgtttactgtttgaagttaaggaaatattagaaattatcaaaagaggggactaaaacttacctataaccttattatccagagataaccatagatcttacttgaaagaacagtatacagttttgtattttgtttcttccccaccccccacaatatttatatgtcaagagtctgagatccaactgcaaggcggcagtgaggctgggggaggggcgcccaccattgctgaggcttgggtaggtaaacaaagtgggctggaagctggaactgggtggagcccaccgctgctcaaggaggcctgcctgcctctgtagactccacctctgggggcagggcatagctgaacaaaaggcagtagaaacctctacggacttaaatgtccctgtctgacagctttgaagagagtagtggttctctcagcacggagtttgagatctgagaacggacagactgcctctgcaagtgggtccctgacccccgaatagcctaactgagaggcaccccccagtagggggagactgacacctcatacggctgggtacccctctgagacgaagcttccagaggaacgatcaggcagcaacaattgctgttcagcagtattcgctgttctgcagcctccgctgctgatacccaggcaaacagggtctggagtggacctccagcaaactccaacagaccagcagctgagggtcctggctgttagaaggaaaactaacaaacagaaaggacatccacaccaaaaccccatctgtacgtcaccatcatcaaagaccaaaggtagataaaaccacaaagatagggaaaaacagagcagaaaagctgaaaattctaaaaatcagagcgcctctcacctccaaaggaacacagctcctcgccagcaacggaacaaagctggacggagaatgactttgacgaattgagagaagaaggcttcagataatcaaacttctccgagctaaaggaggaagttcgaacccatggcaaagaagctaaaaaccttgaaaaaagacgaatggctaactagaataaccagtgtagagaagtccttaagtgacctgatggagctgaaaaccatggcacgagaactacatgacgaatgcacaagcttcagtagccgattcaatcaactggaagaaagggtatcagtgattgaagatcaaatgaatgaaatgaagtgagaagagaagtttagagaaaaaagagtaaaaagaaatgaacaaaacctccaagaaatatgggactatgtgaaaagaccaaatctgcgtctgattggtgtacctgaaagtgatggggagaatggaaccaagttggaaaacacacttcaggatattatccaggagaacttccccaatctagcaaggcaggccaacattcaaattcaggaaatacagagaacaccacaaagatactcctcgagaagagcaactccaagacacataattgtcagattcaccaaagttgaaatgaagggaaaaatgttaatggcagccagagagaaaggtcgggttacccacaaagggaagcccatcagactaacagtggatctcgcggcagaaacgctacaagccaggagagagtgggggccaatattcaacattcttaaagaaacgaatttgcaacccagaatatcatatccagccaaactaaacttcataagtgaaggagaaataaaatcctttacagacaagcaaatgctgagagattttatcaccaccaggcctgccctacaagagctccggaaggaagcactaaacatagaaaggaacaagcggtaccagccactgcaaaaacatgccaaattgtaaagaccatcgaggctaggaggaaactgcatcaactaatgagcaaaataaccagctaacatcataatgacaggatcaaattcacacataacaatattaaccttaaatgtaaatgggctaaatactccaattgaaagacacagactggaaaattggataattggataaagactcaagacccatcagtgtgctgtattcaggagacccatctcatgtgcagagacatacataggctcaaaataaagggatgtatgaagatctaccaagcaaatggaaaacaaaaaaaaggcaggtgttgtaatcctagtctctgataaaacagtctttaaaccaacaaagatcaaaagagacaaagaaggccattacataatggtaaagggatcaatacaacaacaagagctaactatcctaaatatatatgcacccaatacaggagcacccagattcataaagcaagtccttagagacctacaaagagacttagactcccacacaataataatgggagactttaacaccccactgtcgacattagacagatgaacgagacagaaagttaacaaggatatccaggaattgagctcagctctgtaccaagcggacctaaaagacatctacagaactctccaccccaaatcaacagaatatacattcttctcagcaccacatcgcacttattccaaaattgaccacatagttggaagtaaagcactcttcagaaaatgtaaaagaacagaaattataaaaaactgtctctcagaccacagtgcaatcgaacctagaactcaggattaagaaacttactcaaaaccgctcaactacatggaagctgaataacctgctcctgaatgactactgggtacataacgaaatgaaggcagaaataaagatgttctttgaaaccaatgagaacaaagacacaacataccagaatctctgggacacatttaaagcagtgtgtagagggaaatttatagcactaaatgcccacaagataaagcaggagagatctaaaattgacaccctaacatcacaattaaaagaactagagaagcaagagtaaacacattccaaagctagcagaaggcaagaaataactaagatcagagcagaacggaaggaaatagagacacaaaaaacccttcaaaaaatcaatgaatccaggagctggttttttgaaaagatcaacaaaattgatagaccgctagcaagactaataaagaagaaaacagagaagaatcaaatagactcaataaaaaatgacaaagggagtatcaccactgatcccacagaaatacaaactaccatcacagaatactataaacacctctacaaacacctctacgcaaataaactagaaaatctagaagaaatggataaattcctggacacatacactgtcacaagactaaaccaggaagaagttgaatccctgattaaacccataacaagctctggaattgaggcaatagttaagagcctgccatccaaaaaaagtccaggaccagatggattcacagccgaattctaccagcagtacaaggaggagctggtaccattccttctgaaactattccaatcaatagaaaaagagggaatcctccctaactcattttatgaggccagtatcattctgataccaaagcctggcagagacacaacaaaaaaagagaattttagaccaatatccctgatgaacatcgatgcaaaaatcctcagtagaatactggcaaactgaatccagcagcacatcaaaaagcttatccaccatgatcaattgtgcttcatccctgggatgcaaggctggttcagcatacgcaaatcaataaacataatccagcatataaacagaaccaaagacaaaaaccacatgattatctcaatagatgcagaaaaggcctttgacaaaactcaacaacccttatgctaaaaactctcaataaattaggtattggtgggatgtatctcaaaataataggagctatttatgacaaacccacagccaatatcacactgaatgggcaaaaactggaaacattccctttgaaaactggcacaagacacggatgccctctctcaccactcctattcaacatagtgttggaagttctggccagggtaatcaggtagtagaaggaaataaagggtattcaattaggaaaagaggaagtcaaattgtccctgtttgcagatgacatgattgtatatttagaaaaccccatcgtctcagcccaaaatctccttaagctgataagcaacttcagcaaagtttcaggatacaaagtcaatgtgcaaaaatcacaagcattcttatacaccaataacagacaaacagagagccaaatcatgagtgaactcccattcacagttgcttcaaagagaataaaatacctaggaatccaacttacaagggatgtgaaggacctcttcaaggagaactacaaaccactgctcaatgaaacaaaagaggacacaaacaaatggaagaacatttcatgctcgtggataggaagaatcaatatcgtgaaaatggccatactgcccaaggtaatttatagattcaatgccatccccatcaagctaccaatgactttcttcacagaattggaaaaaactactttaaagttcataaggaaccaaaaaagagcccgcattgccaggtaaatcctaagccaaaagaacaaagctggaggcatcacgctacctgacttcaaactatactacaaggctacagtaaccaaaacagcatggtactggtaccaaaacagagatatagaccaatggaacagaccagagccctcagaaataataccacacatctacaaccatctgatctttgacaaacctgagaaaaacaagaaatggggaaaggattccctatttaataaacggtgctggaaaaatggctagccatatgtagaaagctgaaactggatcccttccttacaccttatacaaaaattaattcaagatggattaaagacttaaatgttagacctaaaaccataaaaaccctagaagaaaacctaggcaatactatttgggacataggcatgggcaaggacttcatgtctaaaacaccaaaagcaatggcaacaaaagccaacattgacaaatgggatctaattaaactcaagagcttctgcacagcaaaagaaactaccatcagagtgaacaggcaacctacagaatgggagaaaatttttgcaatctactcatctgacaaagggctaatatctagaatctacaatgaactcaaacaaatttacaagaaaaaaacaaccccatcaaaaagtgggcaaaggatatgaacagacacttttcaaaagaagacatttatgcagccaacagacacatgaaaaaatgctcatcatcactggccatcagagaaatgcaaatcaaaaccacaatgagataccctctcacaccagttagaatggtgatcattaaaaagtcaggagacaacaggtgctgcagaggatgtggagaaatgggaacacttttccactgttggtgggactgtaaactagttcaaccatgtgtaagacagtgtggtgattcctcaaggatctagaactagaaataccatttgacccagccatcccattagtgggtatatacccaaaggattataaatcatgctgctatacagacacatgcacacatatgtttattgcggcactgttcacaatagcaaagacttggaaccaacccagatgtccatcagtaatagactggattaagaaaatgtggcacatatacaccatggaatactatgcagccataaaaaaggatgagttaatgtcctttgtagggacatagatgaagctggaaaccatcattctcagcaaactgtcgcagggaaaaaaaacaaaacactgcatgttctcactcataggtgggaattgaacaatgagaactcttggacacaggaatgtgaacatcacacactggggcctgttgtggggtggggggaggggggagggatagcattaggagaaatacctaatgtaaatggcgagttaatgggtgcagtacaccaacatggcacatgtatacatatgtaacaaacctgcacgttgtgcacatgtacccaagaacttaaagtataaaaaaaaaatttatatatcagggcaattaaaatcgttgttaaaaatttactgtaaatgatttttaacagcagcctaatttattgtatgcatgtactattatttacttaatataaatgcctaataatagtggaatgataacctattcgcttagccttgtgataaatacctttgtggttaaatttgtattctagattatttcttttttttttttttttttgagacggagtctcgctctgtctcccaggctggagtgcagtggcgccatctcaacttgctgcaagctccgcctcccaggttcacatcattcttgtgcctcagcctcccgagtagctgagactacaggcgcctgccaccatgccccgctattttttttggtatttttagtagagacagggtttcaccgtgttagccaggatggtctcgatctcctgaccttgtgatccacccgccttggcctcccaaagtgctaggatcacaggcgtgagccactgcgcccggcctagattatttctttaaagtaaattttcaacagtggaattaattgggccaaaaggtacaggttttttttttcccacaaatgtagctaaactgctttctatctaaccatgttctaccaatttgtatttaatgtagtttcactctgacacattcatattttttatctggatagtttgcagaggggagaacttgataaaattaacattcagtttggtaaagatgatgcacagtattttttagattttttttttttttttttttttgagacaagagtcttgctctgttacccaggctggagtgcagtggcacaatcttggctcactgcagcctccacctcccaggcataagcaattctcccacctcaacctccggagtagctgggattacaggtgcacgccaccatacctggctaatttttgtattttttgtagagacagggtttcgccatgttgcccaggctggtctggaactcctgagcttaagtgatccgcctgcctcggactcccaaagtgctgggattacaggcctgagctgcctcacctggctggataaatttttaatattcttaacatttataattgcatacaaaaaaccctagcaatttgtctcaaatttcatgcataggatcaaaagaaaatgttttatatgactcataagttatcctgaggaaccagataatgataccctggcttactgttaattttctaatttgttacactattaaactaagagacaataacatcttcatagctgaaaattctatttcagatttttgattcagtttgaatttacatatttgtatttttagaactagtttattatttttttgttaggtgatgaatcaagattggttctctaagttattttaatatagtaaactttgaaattgctttgggcaagagagattatatttattgagaaattaaaataaaaggaggtatgtggtcttttttagttaatttttttgttgtggcaaaaatataaaatttatcatctgaaccatttttaagtatacagctcagtagtattataagcatatttaggtcattatgaacagatctccagaactttagcctcctgaaaagatccagagacccctggagcttctcctactatactttgaagaacctctatcttagagaatatattacttcatctggaaaagacagactcacagagaactctagaaatatagtcttttaaaacatatcatctaaaagattaataattctttttatcatcttatccggtctgtgttcatatttccccattgtctaataatgttttcgtaattttttgaaattaggagccaaaccaggttcacacacgttacatttggtggttgatataccttttatttttttttaaatttatttttctgagatgaagtcttgctctgtcacccaggctgaagtacagtgcatgatctcagctcacggcaacttctgcctccccagttcaagtgattctcctgtctcagcctcccgagtagctaggattacaggcgcgtgccaccacgcctggctaattttttatatttttggtagagacagggtttcaccatgttggccaagctggtcttgaactcctgatctcaggtgatctgcctgcctcggcctcccaaagtgttgggattacaggcgtgaaccactgggcctgcccttgatatgccttttaaattttaatctattgattgtctctttgtttataatttatttgttgaagaaacagggtcatttgttctacataattttcctcattctggttttggctgagtgtatctctggtaaaatttaacatgtttttcagttctctgtatttccagtaaatttataggtagagacaggcttgattagatttactttcaattcttttttggcaagattcctagttggtgatgtgtatttctttacatcaagaagatgtgttttgtgatgttaaaatcaaacagtttagctgttgtcagattgattcatccgttgtaaaattcccaattaacttttctctaatgatagcatctattgctaattgttgcctcattgcagtatattagtaggtgttgtaaaaggtgatattccttctccagccttctgaatttattggctgggattcttcaggaaagaagacctcttcctcatataccactttattaccctgaaagtatttatacaggaaaggccaaaataaaatgggactttttttttctttttacctacatattcccagaataattaatttgtgccttaggagcctcaaaacatgactagagttttgtttgtttgtttagaattataaactcatggatttttgcctcccattcattttcgatgttcaaattattctttctttgaccagtggcagcttatttatgttggctcctttgtccttggcacaatcccagtggtctgtagtagtttcctaaaatatacaagatgaagctcatcttgtatattttcttccccagatttagaatcagaattctaattcctttcagtgggaaatggtatttagagattataatctgggtgatatttttgtatattcctacaatgttgtcattgtttataggccttttgatggacagaactaggaaaactttatagaaagagaaaaataattcatgtgttccagatgctatttcttttttttttttttttttttgagacggagtctcgctctgtcgcccaggctggactgcggactgcagtggcgcaatctcggctcactgcaagctccgcttcccgggttcacgccattctcctgcctcagcctccccagtagctgggactacaggcgcccgccaccgcgcccggctaattttttgtatttttagtagagacggggtttcaccttgttagccaggatggtctcgatctcctgacctcatgatccacccgcctcggcctcccaaagtgctgggattacaggcgtgagccaccgcgcccggcctgctatttctaactcagataaaagatgataagagtttttacttcattttaaaatttacatttgtatttttcctcaacatgtaaaatgtggttctaacaataagattattgaattgcaaaatctggggcaggaactattgtattatattagaagattggagggttacaaatacaaatgcctaaaggggccagacagccaaatgaatatatcaggcctgttatgaaaatcatccgggaatggtggggattaataagccagagagcatatatcccatctaaaggaggcatctgtgacttgattctagctgatttttgccaagagggaatatgggcatggtactgcttggttttttcatttttaaaaagaagacgtaatccctgcactttgggaggccaaggcaggcagatcatgaggtcaggagatggagaccatcctggctaacacggtgaaaccccgtctctactaaaaatgtaaaaaattagttgggcatggtggcacgcgcctgtagtcccagctacttgggaggctgaggcgggagaattgcttgaacctggtaggtggaggttgcagtgagctgaaatcgtgccactgcactccagctggggcaacagagcaagactccatctcaaaaaagaaaaaaaagaagatgtaaatccaaatattttatgtgaaattatttagcttttaaatattgacaatactattgacaaactaagtcaaagttttaaaaaggacttttggaccaaacaatgatttcttccataggttattagtttgtaacctcttgtgagaaagatctaaggattttccagacctcagacaatctgaaaaatatattgggaaagagtatgagaaagggaacttaatcatcttggttttgctgatcatgattacatttatttatctgacttttattttttagccttaaaaattaaaatagccctattgaccattaattttgctttttatttctttttgcacattcaaaataggaacttacaatgtctcagaaaaatggtgggaatgttcttcagatgatgtatgagaaacctgaacgatggtcttttaccttccaaacatatgcctgtctcagtcgaataagagctcagcttgcctctctgaatggcaagctcaaagatgcagagaaacctgtattattttttgaacgatctgtgtatagtgacaggtatgtataaatggcttgtacgtggccatttgaagtttttatcttagaactggactttttttttttttttaatgtttcctgggaattagtaataatgaatggctgagctggggatttaagatggaaaatgacatttaaatccctcagcagtctggtactaatggcattagggaagttattgcatttataatcctcagcttccatagaaactgttgggaatatggtggtgcccatttcatttaaatttgcagagaagttgtttttccctctaacccttttaaaattctcatctgctctggttaaatctggatctatatcctagactcttacctggatcaagaagataaaagtctctaaggggccttagaattgccctacttatgctaggattcttttgacctccaaagttagctcagaacagaggttctcaaacatttgatctcagggcccttttcaattcttaataactatcgaggtgtccaaagagcatgtcctattgatacttaacattactagaaattaagatgcacaaaaatgatttaaaattagcaataataaattcattacatattaatataaatacatttgacaaaaagttatattttctaaaataaaaagtagctagaagaatatcattgctttacatttttgcagatatctttaatgcctcacttattaaaagacagctggatttttatttatgcttctgtatttaatctgttatggtatgtttgtttgtttgaagtatattggtatatgaagtaatctggcctctcacagaaccccttagaagatcttggggacttaatggggtcctcactccgtattttgagaaccattggccaaaaacatgatggaaagactcttgtctttttctgctttccacggcactatgtgccactggatttaggagaatgtttcatgttcctgttctcttttttatttctttgttgtttttttttgaaatgatacatgtgttgatgaagactctcttttaggtatatttttgcatctaatttgtatgaatctgaatgcatgaatgagacagagtggacaatttatcaagactggcatgactggatgaataaccaatttggccaaagccttgaattggatggaatcatttatcttcaagccactccagaggtaaaacccaataaaaatgtgtttcactgaaaatttaaagaaatatttagaactcttttcagtggtatataatgagggcaatttagtttaactccagcctcccaacctcccactccccacctctaaattagaaacttgtaactgtgtagatagaagcattcttttttctcagcaaactggaatcccagactgggcaagttgctgttttaggctctttgtgcttcctcaattcttttttattttactcattatttctttctaacatttgcagactcttctcattcatcacagttaaagtaatgaatgtggtcaagcaactcctagaatataggcttcatgaggtgggaccttgggcattttgattactattgaatttccattgcctagaacacccagtgctgggcactgttggagcatatagtaggagctcagtaaatatttactgaatgaatggcaatgttgatggaggtccatatcaagaaatattttattatggttgtttgtgccttggaatgagtggataatggcagctatcgttgggaaaagacttagaggacccttgccattttccattaagggagctcacttgaaaaggtgctttttgtgataggttgtaacatagctattctagttacatatgatgttatataatgaatacaggatgaccaaatagatgcctttcataaaactaacctaaatattaagatcttacatttaatcagaagctttctggttttggtgcgtgcatttctattgtttctgttttttaattctcattttttccttaagtcacagggtaggcactgatgaatgtaaatgttatgacttctggcctaaaattagaatactgtgttgctgtcgaagctgggttttcaaagaaattaggtttcctattccaacggatgattttgttcttcattaagtaaagattttgttcttcattaagcaaggatttagcaattttttagcaatttcaaaagtgaattttgaaattcacttttatttttgaatttttttgtttttaattacaaaaatagtaaataaaaatgtgaaaaagaacataaggaatatgaataatttcattttgcttgaaacatttatagtcactattaagctagaatttagcaaatataataattttgaaattcacttttatttttgaaaaggtttcttaaagttatgaaaatagtacaaacaccaaaaacataaataatactaataatttcattttgatactgataatttcatcagtattgtttctgatatctttggatgaaaggattgtttaattattgaaaagagaatttagagtagagtttgaaacacagtttgtatgctgccatggtcttatgcatgtcatttaggttggaggcttttatttcttggaatgcatgaagagtttctcatggtatatgagcccccacaaattatacgaaattttttatgtatgtgtgcttttgggggaccgttatctagagctgcactgtccaatgtggctatttaaatttagggttattaaaattaaataaaatttagatatttagttccttggtcaccacactagccatatttcaagtgcttaaaaaccacaggtggctggtggctaccttatttgataatgcagatataggacaggcataaaacattccaaagttgttttggttgtgctgatatagagttttcatcacattaagaaccactgtctttagtggttttatgtgaactgaacatagatattttgtaatcaaggttggttaacagcaaagtaagattatgtttatagattggcataccttttagctgttgtctctgaacattttaagtagatgtttaatttttttctcctttttatcaaacttagtatatttcatgtgtggatggataccaaaaatttataggcttatttttgaaagtactgcttggcttagagccaccaagtggctgaaaagctcatggagggaaaaatgaattatttcagcttttaagaagtaataagtagagattttggctagcgtgagtagagagacacagaaaagaaaattttgatggcagcagacaagaataattgttccctgcctttttcttccatctcttattacttgcttttagacatgcttacatagaatatatttacggggaagaaatgaagagcaaggcattcctcttgaatatttagagaagcttcattataaacatgaaagctggctcctgcataggacactgaagtaagacttatttttatttactattcattttaaatacctttgttacctttgttaaattttaatctaaaatttggaagatatgactagcaatatgtaaaatttccagtcgttttgcttattggtatcttccagaactaggtatagattttcattctcttgattgcaatgagatcctatttacttaaagtttttataactagtatcccttggtggtattgatagcgtaatatgtgaaatttcactttaagtgataaagtaggaatatatctgaacattatttaggtactctttcagaaagaattttgacttgggagattgtatagcgtggttgttgagaggatagactttgttattagtcagatcttggctcaagtccagtcttactggtgtgtgaccttggatagcctctgagcttcagaatgacaattctgagctttagttttctcatctttaaaaatgcagaaattcctactttattgactgctatttgtttttgaagagaatcatatgttacagtacccagctgtatatattttgtacctaagtttttgggctccatatggtaaatctagatgagaaagaaggggctgccagatgaagtactgcaaagtaactttagtgtaaatgattgacaacattttgattttccaaggacatacgaatgaatttttaatgtttctctcttttttttgttgaatttctgattattttattaatctctctttttaaacagaaccaacttcgattatcttcaagaggtgcctatcttaacactggatgttaatgaagactttaaagacaaatatgaaagtctggttgaaaaggtagatttagacagactacaaacaaatatttgttttttctaaaaaagtgtactgagtggtgagaaaacaattttctaacagaaaagcttccataaagttacattaagaaacagttaagagctttagaagattttggactgtttaagattccagtcctgactaaattctagctttgtggcctgagctaaaataaagatccttcggaatctcagaatctgacagatcttagaaaaaaattaaataaataaaaataaaataaggatttttttttatcctttagtgcctctatctgtaggataggtaaaattataataacaaaaattctttctcaccccctttttctctgctttgtccccatttcttcctctcttccctacaccaccaatctttagatactttaataataaactgcttgtgaaattaaatttatctttttcaactaaattcaatatggaggatatttaaagttacaggatatggatctcatctatttttatgttggccaaagtcttgagattaagggattttagttttgagtttcatttgggatcatgtcaaggattaattgacattacttaatcattgagtcattgtcttaatcacagttcatttattgcccaaatcactgggagttgactatattctgttaagtgagtggtgcccatattgataaagggaatgggtgagaatttattgatgaatatttgagagagaatagtaagggaattggtataacaaatatccctatacccatgttgataactgagtgattaagctataaagaagaaagcacaatttggtttagttttgagaaaaatttgatttttcctttcttttgtggcataatgtctgctgtttaatagagacttgacattcataagggatatatcaaggattttgaaaatcttgacattttatgtaactactgtagcgtatacatgtagttgtttttcccatggtatgaactaaagcttatctggaaaattccaatgacctcttcatactactaatgatgttaaaaatataggataaattcacttacaatactattaaatgaaaagtcagcattaagtattaacagccaaccatcaatttaactgactttgtaaatcactttttaattttatctaacacttctttgttttgactaagaatcatttcacattattatatttacttttattaataatgctagcagttgactttcttattaaggtatttttacaaggcaataatttttgtttcttgagaattattgagtagcctagaatatgtagagaacaagcatattcttacattatggtcagtaggctaggttcattgactccttacatgattttctgcacacactgacattgcaaagtatgtgattcacatatgtgcttcagaaacatgacaaatctactacatgtttgattttcaaagttgcttgaactattgtgcttattaatgttatcagtgttaaagctgcatatgtcagtggtctatgatatttaaatgaagagattataaaattggtgttacagctattttagaactatatctatcttagattgatagatgcagtggctcacacctgtaatcccagcactttgggaggctgaggtgggaagactgcttgagcccaggagttcctgaccagcttgggcaacatagtgagacctcatctctataaaaaacaaacaaaaactagccagacatgatggtgtgcacctagccaggagttcaaggctgcagtgagctttgattgcaccactgtactcagcctgggtgacagagcaataaataactcctaaaatgaccttttaggtcattcatcttttactactgaagtatttaacaatatttcttttttttctcgagacagtcttgctccgtcgcccaggctggagtgcaatggcacaatcttggctcactgccatctccacctcccaggttcaaccagttctgcctcagcctcctaagtaggtgggattacaagtgcacgcctccatgcctagctaatttttttttttttttgagacggagttttgctcttgttgcccaggctggagtgcgatggcgcgatctcagctcaccacaacctctgcctcccaggttcaagcgattcttctgccttggcctccctagtagctgggattacaggcatgtgccactatgcccggctaattttgtatttttagtagagaaggggtttctccatgttggtcaggcaggtctcgaactcctgaccgcaggtgatctgcccgcctcggcctcccaaagtgctgggattataggcatgagccactgcgcctggcctatttttgtgtttttagtagagatggggggtttcaccatgttggccaggctggtctgaaactcctgaccttaagtgatccacccatctcggcctcccaaaatgctagaattacaggcatgagccgctgcgcccggccttaacaatatttctttggaaagtaaatgcaatggcattgtggtagttacttatacagctggggtcttcaactattatatacttttaaatgaagatgaatattagatacctcaaattagtcaaggaattatactgatttttttttcttcctttcctcaggtcaaagagtttttgagtactttgtgatcttgctgaagactacaggcagccaaatggttccagatacttcagctttgtgtatcttcgtaacttcatattaatataagtttctttagaaaacccaagtttttaatcgtttttgttttaaggaaaaaagatttttaaaatgaatcttatgcaaaactttttgaccagtttcttttcttttgttttttttttaaaaaagacatttaaagacaaagacattatttctcatagcaggaaatgtagaggtagatggttccagtatcagcatagtgactaaactacattataaaagatccagcttccttctgtcattcccctcttttgtcttcctcagcaggttggcttttttccctggtgcctctcacttcgttggtgaccagtttcttaaactgaaagctttaatgttacatagtaaatggtagtgtgtcctgtgtaaattagtgtacctattaaaagttgcaaagtggaattaaaggaatccctagaataaggattctgaagttttattttaaattattatcttcttaacagtttagtcccacctcttacttcctgcctcagtctgctttctctactgtctggattaattaggcagcctgctataaagttaaagtcacacatttctattttgcaaacactgtgattactctttgctttgtagtttgctttgctttgtagggttctgcttttaagtttttctctttttcagacaaattactgataaaaatgatattgctctatatgtaatatatcctgaaagcattattttttgttgaataggaaataaaattaatgaagacagaggctagaaagcatccattaattaatgagacacacttaactacttatctctaaaccatctatgtgaatatttgtaaaaataatgaatggactcatcttagttctgtatataaatatattttctttctagtttgtttagttaaggtgtgcagtgtttttcctgtgtattaaacctttccattttacgttttagaaaattttatgtattttaaaataaggggaagagtcattttcacttttaaactactatttttctttccaagtcatttttgtttttggtttcttattcaaagatgataatttagtggattaaccagtccagacgcactgatctttgcaaaggagacttaatttcaaatctgtaattaccatacataaactgtctcattatacgtatgcatttttttagtttgtttttgtttggtataaattaatttgttaattaaatatttcttaagtataaaccttatgaactacagtggagctacactcattgaaatgtaatttcagttctaaaaagatgtaataatcattttagaattaaaatttattctacttttaaataaattatgaatattaaaggtgaaaattgtataaattactttgattccattttaagtggagacatatttcagtgatttttagtaacctttaaaaatgtataatgacttttaaaatttgtagaattgaaaagacgctaataaaaatttattatttatttgtcatgactcaaaa

SEQ ID NO: 28 - DNA, 2506 bp, Homo sapiens
  source 1..2506: mol_type = genomic DNA
agtgtcctcagctgcctccgcgcgccaaagtcaaaccccgacacccgccggcgggccggtgagctcactagctgacccggcaggtcaggatctggcttagcggcgccgcgagctccagtgcgcgcacccgtggccgcctcccagccctctttgccggacgagctctgggccgccacaagactaaggaatggccaccccgcccaagagaagctgcccgtctttctcagccagctctgaggggacccgcatcaagaaaatctccatcgaagggaacatcgctgcagggaagtcaacatttgtgaatatccttaaacaattgtgtgaagattgggaagtggttcctgaacctgttgccagatggtgcaatgttcaaagtactcaagatgaatttgaggaacttacaatgtctcagaaaaatggtgggaatgttcttcagatgatgtatgagaaacctgaacgatggtcttttaccttccaaacatatgcctgtctcagtcgaataagagctcagcttgcctctctgaatggcaagctcaaagatgcagagaaacctgtattattttttgaacgatctgtgtatagtgacaggtatatttttgcatctaatttgtatgaatctgaatgcatgaatgagacagagtggacaatttatcaagactggcatgactggatgaataaccaatttggccaaagccttgaattggatggaatcatttatcttcaagccactccagagacatgcttacatagaatatatttacggggaagaaatgaagagcaaggcattcctcttgaatatttagagaagcttcattataaacatgaaagctggctcctgcataggacactgaaaaccaacttcgattatcttcaagaggtgcctatcttaacactggatgttaatgaagactttaaagacaaatatgaaagtctggttgaaaaggtcaaagagtttttgagtactttgtgatcttgctgaagactacaggcagccaaatggttccagatacttcagctttgtgtatcttcgtaacttcatattaatataagtttctttagaaaacccaagtttttaatcgtttttgttttaaggaaaaaagatttttaaaatgaatcttatgcaaaactttttgaccagtttcttttcttttgttttttttttaaaaaagacatttaaagacaaagacattatttctcatagcaggaaatgtagaggtagatggttccagtatcagcatagtgactaaactacattataaaagatccagcttccttctgtcattcccctcttttgtcttcctcagcaggttggcttttttccctggtgcctctcacttcgttggtgaccagtttcttaaactgaaagctttaatgttacatagtaaatggtagtgtgtcctgtgtaaattagtgtacctattaaaagttgcaaagtggaattaaaggaatccctagaataaggattctgaagttttattttaaattattatcttcttaacagtttagtcccacctcttacttcctgcctcagtctgctttctctactgtctggattaattaggcagcctgctataaagttaaagtcacacatttctattttgcaaacactgtgattactctttgctttgtagtttgctttgctttgtagggttctgcttttaagtttttctctttttcagacaaattactgataaaaatgatattgctctatatgtaatatatcctgaaagcattattttttgttgaataggaaataaaattaatgaagacagaggctagaaagcatccattaattaatgagacacacttaactacttatctctaaaccatctatgtgaatatttgtaaaaataatgaatggactcatcttagttctgtatataaatatattttctttctagtttgtttagttaaggtgtgcagtgtttttcctgtgtattaaacctttccattttacgttttagaaaattttatgtattttaaaataaggggaagagtcattttcacttttaaactactatttttctttccaagtcatttttgtttttggtttcttattcaaagatgataatttagtggattaaccagtccagacgcactgatctttgcaaaggagacttaatttcaaatctgtaattaccatacataaactgtctcattatacgtatgcatttttttagtttgtttttgtttggtataaattaatttgttaattaaatatttcttaagtataaaccttatgaactacagtggagctacactcattgaaatgtaatttcagttctaaaaagatgtaataatcattttagaattaaaatttattctacttttaaataaattatgaatattaaaggtgaaaattgtataaattactttgattccattttaagtggagacatatttcagtgatttttagtaacctttaaaaatgtataatgacttttaaaatttgtagaattgaaaagacgctaataaaaatttattatttatttgtcatgactcaaaa